SEQUENCE LISTING (548 sequences)

SEQ ID NO: 1 - DNA, 121 bp, Homo sapiens
  source 1..121: /mol_type=“unassigned DNA” /organism=“Homo sapiens”
  variation 61: /allele=“[A/G]”
atgagtctccaggactctatggcttccttcatgtcatcgtccactctgccaagggatttaagcaatcagccagtaagtgccctggccaggacgaggttgggtgggccattgtggattctgc

SEQ ID NO: 2 - DNA, 121 bp, HOMO SAPIENS
  source 1..121: /mol_type=“unassigned DNA” /organism=“HOMO SAPIENS”
  variation 61: /allele=“[A/C]”
atgaagatctacgacagagatgaattgaggggacaaatgtcagagctcacagacgactgtatctctgttcaggaccgcttccacctcactgaaattcactccctcaatgtgctggagggca

SEQ ID NO: 3 - DNA, 121 bp, HOMO SAPIENS
  source 1..121: /mol_type=“unassigned DNA” /organism=“HOMO SAPIENS”
  variation 61: /allele=“[A/G]”
aacagcaggatcagaagcctatttttaatgtcattccaccaattcccgttggttccgaaaattggaataggtgccaaggatctggagatgacaacttgacttctctggggactctgaactt

SEQ ID NO: 4 - DNA, 121 bp, HOMO SAPIENS
  source 1..121: /mol_type=“unassigned DNA” /organism=“HOMO SAPIENS”
  variation 61: /allele=“[A/G]”
ccaaagaatcattaactcctggtagagaagaaaaccccagcagtgtgcttgctcaggagaagggagatgtgatggacttctataaaaccctaagaggaggagctactgtgaaggtttctgc

SEQ ID NO: 5 - DNA, 121 bp, HOMO SAPIENS
  source 1..121: /mol_type=“unassigned DNA” /organism=“HOMO SAPIENS”
  variation 61: /allele=“[A/G]”
atatgtttgacaatttttatttttagctagtcatcaaagctcttacaagtcagaatttcaaacttgaccaggactatagtttatttactggagtgctaggagagaatgcaaaagtgatggt

SEQ ID NO: 6 - DNA, 121 bp, HOMO SAPIENS
  source 1..121: /mol_type=“unassigned DNA” /organism=“HOMO SAPIENS”
  variation 61: /allele=“[A/G]”
ttgatcatgcattcccaatattcgtatatgtatttataaattacataatgggcagggtgcaatggctcacacctgtaatcccagcactgggggaagctgaggtgggtggatcacctgaagt

SEQ ID NO: 7 - DNA, 121 bp, HOMO SAPIENS
  source 1..121: /mol_type=“unassigned DNA” /organism=“HOMO SAPIENS”
  variation 61: /allele=“[A/G]”
cactggatattctaaggagtgttttgaactaatcttgttcccttgaagttcctggagtttattagcagatgtaagtagtatggagtaagttcatacctctcaaaaagcactataatttagg

SEQ ID NO: 8 - DNA, 121 bp, HOMO SAPIENS
  source 1..121: /mol_type=“unassigned DNA” /organism=“HOMO SAPIENS”
  variation 61: /allele=“[A/G]”
cgacgatgtataccagaatatttgttcagattaatattttccttattctggcttattaaaatagtaacgcctgttcttattaaggttgattttgcctgtaattagaagtcatgggcaactt

SEQ ID NO: 9 - DNA, 121 bp, HOMO SAPIENS
  source 1..121: /mol_type=“unassigned DNA” /organism=“HOMO SAPIENS”
  variation 61: /allele=“[A/G]”
aaacactccttagaatatccagtggaaagcactgggactgattttcattcgttgagcattacccaaggtagtgtgccctaaaaagaagtgtaccttatgaacagaatagtagaaactatcc

SEQ ID NO: 10 - DNA, 121 bp, HOMO SAPIENS
  source 1..121: /mol_type=“unassigned DNA” /organism=“HOMO SAPIENS”
  variation 61: /allele=“[A/G]”
gtgcatgtgggatttattcttctgactcaggaaagcaatttgatgaagtgacatgtttctactaaacagcacacatcaagacacgttatgctgcttctgtttatcccacctactggaagga

SEQ ID NO: 11 - DNA, 121 bp, HOMO SAPIENS
  source 1..121: /mol_type=“unassigned DNA” /organism=“HOMO SAPIENS”
  variation 61: /allele=“[A/T]”
catataatatcttaatttaacaagtaaatgcagatgccttaagattccctatcttggagtagttggctgacaccttctccagaaagcatagttaacctgctgcatgacaaagggcaagtta

SEQ ID NO: 12 - DNA, 121 bp, HOMO SAPIENS
  source 1..121: /mol_type=“unassigned DNA” /organism=“HOMO SAPIENS”
  variation 61: /allele=“[A/G]”
catcaggcagccctccagctgaatgatttttgtctgtgcctggcccagtccctgagtccaaagtggtttttaggattcacatcggttacaggaccgggccatggtctgcccacctgaagct

SEQ ID NO: 13 - DNA, 121 bp, HOMO SAPIENS
  source 1..121: /mol_type=“unassigned DNA” /organism=“HOMO SAPIENS”
  variation 61: /allele=“[A/G]”
taaaaaatatgtgaaaatgcattttccccctattccttctggaaagcaacattagggtccagcagttctgtctggaaggagggagatgcaggagcagcatcctggcttatgaccgcgtggc

SEQ ID NO: 14 - DNA, 121 bp, HOMO SAPIENS
  source 1..121: /mol_type=“unassigned DNA” /organism=“HOMO SAPIENS”
  variation 61: /allele=“[A/G]”
cagtatttaagatgaaaggagatcagatttggtttcggagaacagagcagatgtcgtggcatcagtaatcactagggtgtccctttaaggatatgaggactgtggtgagcagggatggcta

SEQ ID NO: 15 - DNA, 121 bp, HOMO SAPIENS
  source 1..121: /mol_type=“unassigned DNA” /organism=“HOMO SAPIENS”
  variation 61: /allele=“[A/C]”
ccggagccagctcggcactggaaccggcgtcctctggtggcagagagagagcgctactggagattttcggaccgaatcggcacgctcgtcagatccaagcaggcgggactggcctggagca

SEQ ID NO: 16 - DNA, 121 bp, HOMO SAPIENS
  source 1..121: /mol_type=“unassigned DNA” /organism=“HOMO SAPIENS”
  variation 61: /allele=“[A/G]”
agaggcaactgtgtctcgggaaggtaaagtgaacatctcagggtcatgtaagtcggaagcaacacagcggtgacttacactcagatccttactctccagagttagtgctcttaaccagtag

SEQ ID NO: 17 - DNA, 121 bp, HOMO SAPIENS
  source 1..121: /mol_type=“unassigned DNA” /organism=“HOMO SAPIENS”
  variation 61: /allele=“[C/G]”
ttcacagcatcaagcagtccacagcagtctgagctggcaggtcatggagcagcccccaaacagctgtggctggggggatgacggccaggctccctgaccaccctgcctgtggaggtgacct

SEQ ID NO: 18 - DNA, 121 bp, HOMO SAPIENS
  source 1..121: /mol_type=“unassigned DNA” /organism=“HOMO SAPIENS”
  variation 61: /allele=“[A/G]”
cttctagttgtatttattgttaaaatgacatcataatattacaggaaatccccccagcctaccctcaccctgctgtgattttactgatcattatctccccctgttctttactcaggtgtat

SEQ ID NO: 19 - DNA, 121 bp, HOMO SAPIENS
  source 1..121: /mol_type=“unassigned DNA” /organism=“HOMO SAPIENS”
  variation 61: /allele=“[A/G]”
tgcatgaacttgggtcccagagggtcctattatgaaagctggatcaattgcaatgggaaaagggctaacgttattgtacctagaatgctgaagtggtcaactacctaaataaagattacac

SEQ ID NO: 20 - DNA, 121 bp, HOMO SAPIENS
  source 1..121: /mol_type=“unassigned DNA” /organism=“HOMO SAPIENS”
  variation 61: /allele=“[A/C]”
aatattagaagtttcctttgtctccccttattttgtcaccaggagtaaaaattaactttaagaaaaggaaatttgctggggtcaccttgtaccttgtcctggctttgttctcgggtgctgg

SEQ ID NO: 21 - DNA, 121 bp, HOMO SAPIENS
  source 1..121: /mol_type=“unassigned DNA” /organism=“HOMO SAPIENS”
  variation 61: /allele=“[A/C]”
aaggaaagaaataacagtaaaaattcaataaaattgaaacaaatatataagaaaatcaacaaaactaaaagtgtttttaaaaagattaatgcagttgataagcttctagcaagactattca

SEQ ID NO: 22 - DNA, 121 bp, HOMO SAPIENS
  source 1..121: /mol_type=“unassigned DNA” /organism=“HOMO SAPIENS”
  variation 61: /allele=“[A/G]”
tctgagggcgggaaccaggtctcctttaccctgggatgcatgggagctcagaaatgtggaatgaagtcgttaatttacacagcacctaccgtgcacctggagaaggtgagaacatggctgg

SEQ ID NO: 23 - DNA, 121 bp, HOMO SAPIENS
  source 1..121: /mol_type=“unassigned DNA” /organism=“HOMO SAPIENS”
  variation 61: /allele=“[A/G]”
caagcaacctgcctcctgctagacaattagctttatccatgagttaccaaagagggagccaaaacccagggaagctgaaagagctgttgattgtcaccctgtgagttggtgatagaaagat

SEQ ID NO: 24 - DNA, 121 bp, HOMO SAPIENS
  source 1..121: /mol_type=“unassigned DNA” /organism=“HOMO SAPIENS”
  variation 61: /allele=“[A/G]”
gattgtacccaccaaaatctataaacaataaggaactgtggttgtttgctgcaaataactatgataaaccacactgtttgtatcacatgtattagcccattgtgacattgtcaattgacca

SEQ ID NO: 25 - DNA, 121 bp, HOMO SAPIENS
  source 1..121: /mol_type=“unassigned DNA” /organism=“HOMO SAPIENS”
  variation 61: /allele=“[A/G]”
gtagggtattgagtgaggggttgttatcttcagtcaatccatcaattaatttgtattagaacattctgtgtgccagtcacagtacatgccctcatcattcccaactcttgaggagcttagt

SEQ ID NO: 26 - DNA, 121 bp, HOMO SAPIENS
  source 1..121: /mol_type=“unassigned DNA” /organism=“HOMO SAPIENS”
  variation 61: /allele=“[A/T]”
attttgcagttatctcagaatattaactagaatatatggctcatgagagcaggccctgtgactgccttcctcactctcatgtcactagggactagcacatagtaggcactcaagaaacgtt

SEQ ID NO: 27 - DNA, 121 bp, HOMO SAPIENS
  source 1..121: /mol_type=“unassigned DNA” /organism=“HOMO SAPIENS”
  variation 61: /allele=“[A/G]”
gcgcttagtttcctgccacagggagacagtaagaaaggtgacgtcaatctgagatgagagagagagcaaaacagttcttttgaccaccttgaccccgaccttgaaataaggtggaactaat

SEQ ID NO: 28 - DNA, 121 bp, HOMO SAPIENS
  source 1..121: /mol_type=“unassigned DNA” /organism=“HOMO SAPIENS”
  variation 61: /allele=“[A/C]”
acgggctttgtgcgaagttcgtcggcgctggtgtccacagcaagtgaagtgggttcagttagtcccaggttccaatggttgaggcggaaaggcaaaggtataaatacccttgatagccttt

SEQ ID NO: 29 - DNA, 121 bp, HOMO SAPIENS
  source 1..121: /mol_type=“unassigned DNA” /organism=“HOMO SAPIENS”
  variation 61: /allele=“[A/C]”
ggcctctgcttcccaccaaggtgctgggggaagtgggctgctgtggacccccaccccggaacactctgctttctgcaggatcctcatgctccccaaggacccagagaggctggggtggggg

SEQ ID NO: 30 - DNA, 121 bp, HOMO SAPIENS
  source 1..121: /mol_type=“unassigned DNA” /organism=“HOMO SAPIENS”
  variation 61: /allele=“[A/G]”
cacaacattgatgctctctgaacactatgacctctgattatttatcaacctccaagagctatcactgtcactggggacagagagcagacaaaataaaacacctgggagtggggtgcagaag

SEQ ID NO: 31 - DNA, 121 bp, HOMO SAPIENS
  source 1..121: /mol_type=“unassigned DNA” /organism=“HOMO SAPIENS”
  variation 61: /allele=“[C/G]”
tgggctgggctcccctcttctgtgagagccaaacagagcccttcctgagtcccatccattcgcagggtcctactgttgtccgccccctcgttcccactgccagctctgggggagctgggct

SEQ ID NO: 32 - DNA, 121 bp, HOMO SAPIENS
  source 1..121: /mol_type=“unassigned DNA” /organism=“HOMO SAPIENS”
  variation 61: /allele=“[A/G]”
cttgcctctatctgggtccttttcattgctctacaaagaatcctttcttcctcccaggccacactaagtaataacaactggggacttttctcacgccaacttctgagccgcttcaagtgtc

SEQ ID NO: 33 - DNA, 121 bp, HOMO SAPIENS
  source 1..121: /mol_type=“unassigned DNA” /organism=“HOMO SAPIENS”
  variation 61: /allele=“[A/G]”
aggcccggcccatcaatgcccacgctacacgaggcatactagacagtcgctgcctaagccaaagtcagatcaccgatattcttccaggaaaaggctcctcttgccccctttcccacaagaa

SEQ ID NO: 34 - DNA, 121 bp, HOMO SAPIENS
  source 1..121: /mol_type=“unassigned DNA” /organism=“HOMO SAPIENS”
  variation 61: /allele=“[A/G]”
tacttttgaagtttctgtcaaagaatgtcagagaatatatagttttgtgtggctatctctatttttcttatatattatccctgttaatgcagggcatactgttactcttgaatgttttaac

SEQ ID NO: 35 - DNA, 121 bp, HOMO SAPIENS
  source 1..121: /mol_type=“unassigned DNA” /organism=“HOMO SAPIENS”
  variation 61: /allele=“[A/G]”
tctgtgaacgtgttggcactaactgaaaatgaatgtttgctacattatagtccattaggtatggtcatagttgccagtggtgagcagaatcctcccaggagtaataaattcatcagtatac

SEQ ID NO: 36 - DNA, 121 bp, HOMO SAPIENS
  source 1..121: /mol_type=“unassigned DNA” /organism=“HOMO SAPIENS”
  variation 61: /allele=“[A/G]”
gcaatcaatacatcatgatgtaatgtagtcatatagactaggacacttagattagcccccatgacgcaaggcgtgttctgagtaacagtctcaaattaagtggagactttgtgatcactgc

SEQ ID NO: 37 - DNA, 121 bp, HOMO SAPIENS
  source 1..121: /mol_type=“unassigned DNA” /organism=“HOMO SAPIENS”
  variation 61: /allele=“[A/G]”
gttttttaaaggttttagtattgcaatgtggaatccaaaactgttatcaatgaacttttgattgttacattgaaatatgtcagtctatcttgcactttgaatgtatcttttacccatgcat

SEQ ID NO: 38 - DNA, 121 bp, HOMO SAPIENS
  source 1..121: /mol_type=“unassigned DNA” /organism=“HOMO SAPIENS”
  variation 61: /allele=“[A/C]”
aagagcaaagctccgtcttaaaaaaaaaaaaaaaaaaaaaaagaacacagcctcccacctaatatttcctgacacggggcctcaggatggcactaacggttccctcacccagggaggtaga

SEQ ID NO: 39 - DNA, 121 bp, HOMO SAPIENS
  source 1..121: /mol_type=“unassigned DNA” /organism=“HOMO SAPIENS”
  variation 61: /allele=“[A/G]”
tctgttccctaaaattttgatgtacccagaaaagcatattgtaaaaaatgttcagatggtaagagttttactttctaataaagcatacagatttgtgatggggagttcagttcatgggcag

SEQ ID NO: 40 - DNA, 121 bp, HOMO SAPIENS
  source 1..121: /mol_type=“unassigned DNA” /organism=“HOMO SAPIENS”
  variation 61: /allele=“[A/G]”
gaaagtggtgagggacaggcctggacagtgtccactgggcagagagagccgattccgtgcagctcctggtgctgatgtgcagcgtctggggatcccgtcgtctgttttactctggggatat

SEQ ID NO: 41 - DNA, 121 bp, HOMO SAPIENS
  source 1..121: /mol_type=“unassigned DNA” /organism=“HOMO SAPIENS”
  variation 61: /allele=“[A/G]”
ttaatgtcagcacactaatattcaaacatccttgacctcatctcatataaataaatccaaatgcaaatatcagtcagtcaatatatgttgtatgtctagctcccacacaatttttatagca

SEQ ID NO: 42 - DNA, 121 bp, HOMO SAPIENS
  source 1..121: /mol_type=“unassigned DNA” /organism=“HOMO SAPIENS”
  variation 61: /allele=“[A/G]”
cctgcactatccaagattctggatgtctttaaggtaacaagtgtccatgttgttccttgaaagcctggagaaatctggtgtgggaaatgtaggactcttggtgtggggggactgttcaaga

SEQ ID NO: 43 - DNA, 121 bp, HOMO SAPIENS
  source 1..121: /mol_type=“unassigned DNA” /organism=“HOMO SAPIENS”
  variation 61: /allele=“[A/G]”
tagcaatgttcctctgtctgcacttaagccataagaactctttttccttgtaagcccatcagtactcaatgaaatgcctgcagagatttggtgcatagctattttcgcttctgctgagaac

SEQ ID NO: 44 - DNA, 121 bp, HOMO SAPIENS
  source 1..121: /mol_type=“unassigned DNA” /organism=“HOMO SAPIENS”
  variation 61: /allele=“[A/G]”
ctccttcatcctcccacagagccttggcatgctcatagaattcctgaaagtgaacacaagaaagtttagagaaaggcaagagcttgaactaatcaacaacactgtcattcaaaccctgaga

SEQ ID NO: 45 - DNA, 121 bp, HOMO SAPIENS
  source 1..121: /mol_type=“unassigned DNA” /organism=“HOMO SAPIENS”
  variation 61: /allele=“[A/C]”
tggatctgaagatcttagccaaggcaggaaagcacacgatcaggtaacctccagattcacagccctggtgccccggttctcctgggaactggtcctgagatcttggacaaatccctggttc

SEQ ID NO: 46 - DNA, 121 bp, HOMO SAPIENS
  source 1..121: /mol_type=“unassigned DNA” /organism=“HOMO SAPIENS”
  variation 61: /allele=“[A/C]”
aaaacattttttactcgcattaactctttcaatttcacaacaaatctaagaaaaatgcaaaaacaggaaaattaaaacaaatggaaacattaaaagtatccatcaatatatacaaatattg

SEQ ID NO: 47 - DNA, 121 bp, HOMO SAPIENS
  source 1..121: /mol_type=“unassigned DNA” /organism=“HOMO SAPIENS”
  variation 61: /allele=“[A/G]”
taaccaaattagtctaaaacactatcatctcctcctggattactgcaacagactccttctatgcttgcccccttcggcctattcacacagtttctatagtgatcctttcaaaatttcagat

SEQ ID NO: 48 - DNA, 121 bp, HOMO SAPIENS
  source 1..121: /mol_type=“unassigned DNA” /organism=“HOMO SAPIENS”
  variation 61: /allele=“[A/G]”
gctttggggctgtttttcctggaaaaacgactgccttctaaggccaaaggtcagtttaaaaagggctgctggaccgccaaaccccaccgaacagggaatatttagggcagaaataaggact

SEQ ID NO: 49 - DNA, 121 bp, HOMO SAPIENS
  source 1..121: /mol_type=“unassigned DNA” /organism=“HOMO SAPIENS”
  variation 61: /allele=“[A/G]”
taatagagcacagtatccaagagagtaggatcttaataaccccctgaaaaagcaagcagaaatggctttctaaaagcaggagaacaagagaaatacttccaacacgcatggtggtcagatt

SEQ ID NO: 50 - DNA, 121 bp, HOMO SAPIENS
  source 1..121: /mol_type=“unassigned DNA” /organism=“HOMO SAPIENS”
  variation 61: /allele=“[A/C]”
tgcgactcaagcccaccctgcctggcctgcaccaggtggaaccccatgcgcttgcctagcaaggaccagacaccgaggggccgttggttctaggacggcgagggtcagaaggagaggcctg

SEQ ID NO: 51 - DNA, 121 bp, HOMO SAPIENS
  source 1..121: /mol_type=“unassigned DNA” /organism=“HOMO SAPIENS”
  variation 61: /allele=“[A/G]”
aatcccaataaaagctctaataccacctaaaaccatttctgttctctacctctgtcattaatgcttaaatgaaacaaggctgaaaatcaaataatgcagaaatgtgccttcgtcaataagt

SEQ ID NO: 52 - DNA, 121 bp, HOMO SAPIENS
  source 1..121: /mol_type=“unassigned DNA” /organism=“HOMO SAPIENS”
  variation 61: /allele=“[A/C]”
atgacacagcacaggttctatatctttagatggtaaattaaaaattcctggctgaatttgattgattgtcatttttaaaaattgttaaagacttgtaagagggaagaataggccagacatt

SEQ ID NO: 53 - DNA, 121 bp, HOMO SAPIENS
  source 1..121: /mol_type=“unassigned DNA” /organism=“HOMO SAPIENS”
  variation 61: /allele=“[A/G]”
aatgatcctgggtaacaaatgtgatatgaatgacaaaagacaagtgtcaaaagaaagaggagagaaggtaaatgtgaatggaatggataaaggttggaatctactcacattaagcatttct

SEQ ID NO: 54 - DNA, 121 bp, HOMO SAPIENS
  source 1..121: /mol_type=“unassigned DNA” /organism=“HOMO SAPIENS”
  variation 61: /allele=“[A/G]”
aaaataataatttctcacgctcacttcggcagcacatatactaaaataataatttctcccagttctttattattagcctccaaagagtatacctgcagcagctttaaacaacatgccactc

SEQ ID NO: 55 - DNA, 121 bp, HOMO SAPIENS
  source 1..121: /mol_type=“unassigned DNA” /organism=“HOMO SAPIENS”
  variation 61: /allele=“[A/G]”
tgctattttacattgggtgatcaggaaatgctcctaggaggaggtggtatttgagcagggatatgggtgaagtaagagaggtcatgcaagggctagaagaagactactccaggcagaggag

SEQ ID NO: 56 - DNA, 121 bp, HOMO SAPIENS
  source 1..121: /mol_type=“unassigned DNA” /organism=“HOMO SAPIENS”
  variation 61: /allele=“[A/C]”
aaaatctggcaagatagcctatacaacatggtgagaccctgtctctacaaaaattaaaaaatatatatatagccgaacatagtggctatattggagtgcagcaggtggaaggagtgcttga

SEQ ID NO: 57 - DNA, 121 bp, HOMO SAPIENS
  source 1..121: /mol_type=“unassigned DNA” /organism=“HOMO SAPIENS”
  variation 61: /allele=“[A/C]”
agttctccagaggacctgcctacctccagacggctcactcactcccactcattctgtataaactgctagtgaagcctttctgacacagcacacccaccacattactctctaattcatggac

SEQ ID NO: 58 - DNA, 121 bp, HOMO SAPIENS
  source 1..121: /mol_type=“unassigned DNA” /organism=“HOMO SAPIENS”
  variation 61: /allele=“[A/G]”
ttaagacctcagaaaactcttgttaaaatggaaatctattccctaaaagagattatacacatatccattatggacattcacatgctagcagtgattcattgatcaaattagttgtcacttt

SEQ ID NO: 59 - DNA, 121 bp, HOMO SAPIENS
  source 1..121: /mol_type=“unassigned DNA” /organism=“HOMO SAPIENS”
  variation 61: /allele=“[A/C]”
gtttatttgttgattttatctgtggaagatcagtccaacgtttaaagtggggtgttgaagactccagctactattttattaggggcttatctctatgtttacctctaataatattttcttt

SEQ ID NO: 60 - DNA, 121 bp, HOMO SAPIENS
  source 1..121: /mol_type=“unassigned DNA” /organism=“HOMO SAPIENS”
  variation 61: /allele=“[A/C]”
tttgctaagaagtgttaattctctaagaggaaaatgtcatttctccaaaacaaaactttaagcaggtgattttttttaaaagccctgtcaggttgacaagtgctataagataataaacctt

SEQ ID NO: 61 - DNA, 121 bp, HOMO SAPIENS
  source 1..121: /mol_type=“unassigned DNA” /organism=“HOMO SAPIENS”
  variation 61: /allele=“[A/G]”
tccaaatcacgtaacaaggttacctccagaaaaaaaggctattgctgaacagaggctttcatttttacttttattccccagaattttttgaatgctttagaacattgattctcaaaccgta

SEQ ID NO: 62 - DNA, 121 bp, HOMO SAPIENS
  source 1..121: /mol_type=“unassigned DNA” /organism=“HOMO SAPIENS”
  variation 61: /allele=“[A/G]”
gagggaagctggctctctttgaatggaaatttaaccagaagttaaaataaattccattcaatcgtatagaatagttttgttccttttcacttaaaaatatttttctctcttttatgtgcct

SEQ ID NO: 63 - DNA, 121 bp, HOMO SAPIENS
  source 1..121: /mol_type=“unassigned DNA” /organism=“HOMO SAPIENS”
  variation 61: /allele=“[A/G]”
ctcgaggaggggctcacaccgagatcaatccatgatgacagcacttcatggcccgtctcaaacacacaggcccactccctggtctggcccaggctggggtgctcagggcctctgtgttgtt

SEQ ID NO: 64 - DNA, 121 bp, HOMO SAPIENS
  source 1..121: /mol_type=“unassigned DNA” /organism=“HOMO SAPIENS”
  variation 61: /allele=“[A/G]”
aaaatgatgggataatctaattcatctaacttgctttacaaatgaggaaactgataatccaaaagatttaatctcataggaaccaggtgacagagcaggaaataggccactggtctcctgc

SEQ ID NO: 65 - DNA, 121 bp, HOMO SAPIENS
  source 1..121: /mol_type=“unassigned DNA” /organism=“HOMO SAPIENS”
  variation 61: /allele=“[A/G]”
catctgcagatttaacgatttcattgaaaaaaaaatcctccagatcaggtattttagaatattaaataacaccaatcctgaggcccgtctgtaaccactcaaagggtccaccttgcccact

SEQ ID NO: 66 - DNA, 121 bp, HOMO SAPIENS
  source 1..121: /mol_type=“unassigned DNA” /organism=“HOMO SAPIENS”
  variation 61: /allele=“[A/G]”
cttggggattccactgggctatgtgtccatttatttattcattcaataaatatttactgaatgtccaccaggccctatagataccatgggaaacagacagtggcccctgttctcaagtggc

SEQ ID NO: 67 - DNA, 121 bp, HOMO SAPIENS
  source 1..121: /mol_type=“unassigned DNA” /organism=“HOMO SAPIENS”
  variation 61: /allele=“[A/G]”
gaaggactaaaggggtcaagatacaaggagtcaccaaagaatgcagaagagacaagttcaagaagactaccacatacgtattggttacccagagagaacctgaaaacagcagcaccattgg

SEQ ID NO: 68 - DNA, 121 bp, HOMO SAPIENS
  source 1..121: /mol_type=“unassigned DNA” /organism=“HOMO SAPIENS”
  variation 61: /allele=“[A/G]”
tggttcaggtggctctggataaggtcagtgaggcttagttcaaaccaacctgatttataaacataagaacattctactactaattcttgttaatattggtcttagaaaaggaaatttctga

SEQ ID NO: 69 - DNA, 121 bp, HOMO SAPIENS
  source 1..121: /mol_type=“unassigned DNA” /organism=“HOMO SAPIENS”
  variation 61: /allele=“[A/G]”
tcaggaggtctgttgctaatcccaaccagcatgatttacgggaagtaaatcatctatgacatgcccaaagagaataaaagtacatacaggatgcttctacttagggcttttttggtagaga

SEQ ID NO: 70 - DNA, 121 bp, HOMO SAPIENS
  source 1..121: /mol_type=“unassigned DNA” /organism=“HOMO SAPIENS”
  variation 61: /allele=“[A/G]”
atcctgatcagcctgtctcacaaacattgggttctatagacgctcctagattgcattttcatttaagctgagccttgatggtctgctggaatatggtaggctacactttacacacacaagg

SEQ ID NO: 71 - DNA, 121 bp, HOMO SAPIENS
  source 1..121: /mol_type=“unassigned DNA” /organism=“HOMO SAPIENS”
  variation 61: /allele=“[A/G]”
tccttttgttcccagtgccttgacagggtatggggggacctgcatgactagcattaaatgaaggactgggctttgccagaatgaagaaatcctctgagaatgtgcagtagagcaaaacaag

SEQ ID NO: 72 - DNA, 121 bp, HOMO SAPIENS
  source 1..121: /mol_type=“unassigned DNA” /organism=“HOMO SAPIENS”
  variation 61: /allele=“[A/C]”
tggacatttataaccagggatctgtgcgttttgctataattcagaaagtagcagactactagacacgtgtcatttggcaagggattttaagagcacatagtatacttagaataatcatgct

SEQ ID NO: 73 - DNA, 121 bp, HOMO SAPIENS
  source 1..121: /mol_type=“unassigned DNA” /organism=“HOMO SAPIENS”
  variation 61: /allele=“[A/G]”
gagagggaggaaaagtcggttcgagaacccaggtggaaaatagattgagggaagcaaaacaagatgttacaggaggaatatgggtgattgtcttttccttttatatttctgcatgttttgt

SEQ ID NO: 74 - DNA, 121 bp, HOMO SAPIENS
  source 1..121: /mol_type=“unassigned DNA” /organism=“HOMO SAPIENS”
  variation 61: /allele=“[A/G]”
taaaaaataattttaaagcagtgtggtctcaatcttagtagaagagtagaaagcaagataatttctacttggaataaacaagtgcacagtggaagtgattaactcttactctcaatgttat

SEQ ID NO: 75 - DNA, 121 bp, HOMO SAPIENS
  source 1..121: /mol_type=“unassigned DNA” /organism=“HOMO SAPIENS”
  variation 61: /allele=“[A/C]”
aaatagagggctctggacatcttcagagggtcccactttagacttcactgatctctttttaacattttttatcaatacataatatttgtacattttatggggtatttgtgatattttgcta

SEQ ID NO: 76 - DNA, 121 bp, HOMO SAPIENS
  source 1..121: /mol_type=“unassigned DNA” /organism=“HOMO SAPIENS”
  variation 61: /allele=“[A/G]”
cctaatctcattcacaaatatatctgaataaaaatggtaaatccaaagacaacaacatcaataactatcttagctatatcccttactggaaataataaatgtaaagtgtgaaagaatcaat

SEQ ID NO: 77 - DNA, 121 bp, HOMO SAPIENS
  source 1..121: /mol_type=“unassigned DNA” /organism=“HOMO SAPIENS”
  variation 61: /allele=“[A/G]”
cctcgcctctctcttctgatttatctggctcttgcctctccccctccatcaaaagaccacactatctctctcctcttttccatttgaacgattttgccattcatcaaactgattgctaatg

SEQ ID NO: 78 - DNA, 121 bp, HOMO SAPIENS
  source 1..121: /mol_type=“unassigned DNA” /organism=“HOMO SAPIENS”
  variation 61: /allele=“[A/G]”
cttcagagacttcgtaattaaaggaacagagtgagagacatcatcaagtggagagaaatcatagtttaaactgcattataaattttataacagaattaaagtagattttaaaagataaaat

SEQ ID NO: 79 - DNA, 121 bp, HOMO SAPIENS
  source 1..121: /mol_type=“unassigned DNA” /organism=“HOMO SAPIENS”
  variation 61: /allele=“[A/C]”
ctggtatgggagccagggttgaagtcactcacgggtcctctccgagaactcgagtggtgaaatggagagccggggcctgcccttgtccctgcagcaggactggggaggagggggtgcctga

SEQ ID NO: 80 - DNA, 121 bp, HOMO SAPIENS
  source 1..121: /mol_type=“unassigned DNA” /organism=“HOMO SAPIENS”
  variation 61: /allele=“[A/G]”
gtctgtgtgacagagcaagaccctgtctcttaaaaaaaaaaaaagtgatgtagccatttcataaagacagttgggcaataactatcaaatttaaaatgcatatcacctttgcacttccagg

SEQ ID NO: 81 - DNA, 121 bp, HOMO SAPIENS
  source 1..121: /mol_type=“unassigned DNA” /organism=“HOMO SAPIENS”
  variation 61: /allele=“[A/G]”
accacacggtccctcaggctgcttgttaccgtggaagcttcctgaactctctccagacccacagacctcccttcttgggggctgccgctgaggagcttctggctagtgagctctgaagcac

SEQ ID NO: 82 - DNA, 121 bp, HOMO SAPIENS
  source 1..121: /mol_type=“unassigned DNA” /organism=“HOMO SAPIENS”
  variation 61: /allele=“[A/G]”
aagggaaatgttttcatttttctcttcccaacccaatcccctctctctaaatcttggtataggtgaggtgctaacagacagtgaaacaagaaagtggttggagtcattccaaaaggggaac

SEQ ID NO: 83 - DNA, 121 bp, HOMO SAPIENS
  source 1..121: /mol_type=“unassigned DNA” /organism=“HOMO SAPIENS”
  variation 61: /allele=“[C/G]”
tatgagttctgtaagaacaggtactggggtcaggcttttcaccactgagtccccctagaacccagcatggtgccttgaacacagaaggtgttcagaaaacatatattgaatgatgaacgaa

SEQ ID NO: 84 - DNA, 121 bp, HOMO SAPIENS
  source 1..121: /mol_type=“unassigned DNA” /organism=“HOMO SAPIENS”
  variation 61: /allele=“[A/G]”
ggaagacccatttgcttcctttccccaattctaccaacacatttattgagcacttactatataactggcaattgagatgaaagtacacataacaaggtgaacatgcaaggggtccaccagt

SEQ ID NO: 85 - DNA, 121 bp, HOMO SAPIENS
  source 1..121: /mol_type=“unassigned DNA” /organism=“HOMO SAPIENS”
  variation 61: /allele=“[C/G]”
ggcatctttagaccaaagaatctactgcacttcaaattcttcatgtgtgtaatgagaatactgatggcactacctttgtagtgtttgggaatgatttggggagatacttttttgtgtaaag

SEQ ID NO: 86 - DNA, 121 bp, HOMO SAPIENS
  source 1..121: /mol_type=“unassigned DNA” /organism=“HOMO SAPIENS”
  variation 61: /allele=“[A/G]”
tcctggactttgccatgtccttctaagtgacccgagcacttccagtctcatttgggcagcatccttcccgaattccattctgtacacttcaagcaaattagttttagagcatagctctgat

SEQ ID NO: 87 - DNA, 121 bp, HOMO SAPIENS
  source 1..121: /mol_type=“unassigned DNA” /organism=“HOMO SAPIENS”
  variation 61: /allele=“[A/G]”
aaaaaaaaaaaaaaaaaaagaatagaatagaatagagagtttggaaaaagatacacataaatatgcttgattcattttgctaatgcaaagacaatttcatggaaaagtgataacctttaag

SEQ ID NO: 88 - DNA, 121 bp, HOMO SAPIENS
  source 1..121: /mol_type=“unassigned DNA” /organism=“HOMO SAPIENS”
  variation 61: /allele=“[A/G]”
ttgttgagtgttggtgatgctgatagttggagatacccagacagataaggtatattgcccactttcaaaacttggctgcgttagttacatccctatcgatgcaattttcttttctttttga

SEQ ID NO: 89 - DNA, 121 bp, HOMO SAPIENS
  source 1..121: /mol_type=“unassigned DNA” /organism=“HOMO SAPIENS”
  variation 61: /allele=“[A/G]”
ttcttctaagactgctcttcctggcttgcagatggccgccttcatgctgggtctttacacaacctttttcactgtgctaccacattcctgaaggacaccagtcatattgattagagtccca

SEQ ID NO: 90 - DNA, 121 bp, HOMO SAPIENS
  source 1..121: /mol_type=“unassigned DNA” /organism=“HOMO SAPIENS”
  variation 61: /allele=“[A/G]”
cgattttggagcagtaggggactggctgccgagggggcatctagattgagataggtgggaaggcaggacaagacccctaagctcactgcctcctcgattccagtcgtcagactccataagt

SEQ ID NO: 91 - DNA, 121 bp, HOMO SAPIENS
  source 1..121: /mol_type=“unassigned DNA” /organism=“HOMO SAPIENS”
  variation 61: /allele=“[A/C]”
tacacttacaaaagaatagagaatcggctataaatttgctgagtcagaacattatactggacatccactttctcacttctttgtttttccagaatgagcacttttgccaatcccggtttgt

SEQ ID NO: 92 - DNA, 121 bp, HOMO SAPIENS
  source 1..121: /mol_type=“unassigned DNA” /organism=“HOMO SAPIENS”
  variation 61: /allele=“[A/G]”
caagaccggcctgggcaacatatcaaggacccatctctacaaaattgaaaaaaaaaaaaaagggggaagcaggaaaaggtgatcatggtggaccacacaaagctttagaatgaattctttt

SEQ ID NO: 93 - DNA, 121 bp, HOMO SAPIENS
  source 1..121: /mol_type=“unassigned DNA” /organism=“HOMO SAPIENS”
  variation 61: /allele=“[A/G]”
gactgaggaagctccattttctttgaggtacatcaacatcaataacagatcaatggacccacttaatggagctcttaattgagtagaaaaaaatatttaagagttttgccgctctacggca

SEQ ID NO: 94 - DNA, 121 bp, HOMO SAPIENS
  source 1..121: /mol_type=“unassigned DNA” /organism=“HOMO SAPIENS”
  variation 61: /allele=“[A/G]”
tgcggcctgcagcctgacctcatggcttagctgtgcctcctggacaccatccctctctgcaatggcgtgtggtcctgagtcactgacagcactgacccgctcctctgagcaccagccctgt

SEQ ID NO: 95 - DNA, 121 bp, HOMO SAPIENS
  source 1..121: /mol_type=“unassigned DNA” /organism=“HOMO SAPIENS”
  variation 61: /allele=“[A/G]”
ttatatggtggtaaggtgttggggagggggaggggaattgttttttaatctttatgattaaatctcagttttttttagtgggtctgaatccctgggctgtgactttcagaaatgagacaag

SEQ ID NO: 96 - DNA, 121 bp, HOMO SAPIENS
  source 1..121: /mol_type=“unassigned DNA” /organism=“HOMO SAPIENS”
  variation 61: /allele=“[A/G]”
gaggcattatttggtgagaatcaccatttaaaaatgcaaaatattgtgtcactggcttaaactgcagattcctaggccagaggcaatcaatacatcatgatgtaatgtagtcatatagact

SEQ ID NO: 97 - DNA, 121 bp, HOMO SAPIENS
  source 1..121: /mol_type=“unassigned DNA” /organism=“HOMO SAPIENS”
  variation 61: /allele=“[A/G]”
cacccctgacccactcatatgtctgttctcactcagaggtgaggccctgtgtcttcagccatggtaaactcaggacctctggacaggcaggcccagggtgtaggcaccatgacttttcctg

SEQ ID NO: 98 - DNA, 121 bp, HOMO SAPIENS
  source 1..121: /mol_type=“unassigned DNA” /organism=“HOMO SAPIENS”
  variation 61: /allele=“[A/G]”
ctgggggttagggggacagagaagtaacgtcacaagattttaagcttgggccagatatggaaaataatttaatcctagatcacattttacacatgaataactgagaacagaaagaagtgat

SEQ ID NO: 99 - DNA, 121 bp, HOMO SAPIENS
  source 1..121: /mol_type=“unassigned DNA” /organism=“HOMO SAPIENS”
  variation 61: /allele=“[A/G]”
atagggttgaaagtagagcagaaagggcaagcagagaactagacagagaagacagatgacagaggagaggaggggaatgactgccagggccaggtcccaggagagtgggaaggtattatta

SEQ ID NO: 100 - DNA, 121 bp, HOMO SAPIENS
  source 1..121: /mol_type=“unassigned DNA” /organism=“HOMO SAPIENS”
  variation 61: /allele=“[A/T]”
tatgtcatttgtaaaattttaatcataaggtacaatttccttgaggcttcttcacaatgaacattgagcccatggtgatatccccagtcttcttgccctagaggcagccacatatgcttat

SEQ ID NO: 101 - DNA, 121 bp, HOMO SAPIENS
  source 1..121: /mol_type=“unassigned DNA” /organism=“HOMO SAPIENS”
  variation 61: /allele=“[A/C]”
aaaaaaaaaaaaaaaaaaaaaaagaacacagcctcccacctcatatttcctgacacggggactcaggatggcactaacggttccctcacccagggaggtagaaggacttggacacaagacg

SEQ ID NO: 102 - DNA, 121 bp, HOMO SAPIENS
  source 1..121: /mol_type=“unassigned DNA” /organism=“HOMO SAPIENS”
  variation 61: /allele=“[A/G]”
caacacacttaatcttggggaatctgagtttattagaggaatgtagggaggaagcaggctacatgccctcccagcttagatttagatttagccagaagaatgtctgcacttctttgctaga

SEQ ID NO: 103 - DNA, 121 bp, HOMO SAPIENS
  source 1..121: /mol_type=“unassigned DNA” /organism=“HOMO SAPIENS”
  variation 61: /allele=“[A/G]”
agcaatcctcccacctcagcttcccaaagtgctgagattacaggcgtgagccactgcaccaggcccatcttcctttagactgtcttgatgaagtcactagagcatatgataaaaggagaga

SEQ ID NO: 104 - DNA, 121 bp, HOMO SAPIENS
  source 1..121: /mol_type=“unassigned DNA” /organism=“HOMO SAPIENS”
  variation 61: /allele=“[A/G]”
aaggaaataaagaacatgacagaaaaaccgtctatcattttaaagaatatatatatatatataatcattaggagaatattcatagaaataaaaacattaaaggtgtttctggtgagatctc

SEQ ID NO: 105 - DNA, 121 bp, HOMO SAPIENS
  source 1..121: /mol_type=“unassigned DNA” /organism=“HOMO SAPIENS”
  variation 61: /allele=“[A/G]”
agaagtaatttgagtatcttttccttgtttttctcttttgtccagcttatatttatccacaattttataaatctggctcagcaaagcatgttggaagggatctcattttaaacaattctgt

SEQ ID NO: 106 - DNA, 121 bp, HOMO SAPIENS
  source 1..121: /mol_type=“unassigned DNA” /organism=“HOMO SAPIENS”
  variation 61: /allele=“[A/G]”
ggtcgtcttctagtacagtaagggcaaagggcactgcaattgctattaaactgtaagaagaaggaaaaaatggacagatttcgtagcctagtccatcaaaatcattactttgtagttgata

SEQ ID NO: 107 - DNA, 121 bp, HOMO SAPIENS
  source 1..121: /mol_type=“unassigned DNA” /organism=“HOMO SAPIENS”
  variation 61: /allele=“[A/G]”
tctggaagggatcccccggaactgggggaatttccaggcacatgaggctctgtcaacccaaccaggaacatccgcccctgccatctgctccagacgtcattgcagagtctgtgtgagagga

SEQ ID NO: 108 - DNA, 121 bp, HOMO SAPIENS
  source 1..121: /mol_type=“unassigned DNA” /organism=“HOMO SAPIENS”
  variation 61: /allele=“[A/G]”
aaaaaaaagtatgaatgaaagtagattttaagtatgccatgttagataaataatacgtacatattttggcactaaatgaataactgctggaaaaatttattttaagtggcttttaaaatgc

SEQ ID NO: 109 - DNA, 121 bp, HOMO SAPIENS
  source 1..121: /mol_type=“unassigned DNA” /organism=“HOMO SAPIENS”
  variation 61: /allele=“[A/G]”
ttaaaattagtagttttacataaaaatctgaatgtctggcttttcttggaaaattggaaaatctggccatgccagacctcatttttaaatggcaattgcatgtgcccctgcaagcagggat

SEQ ID NO: 110 - DNA, 121 bp, HOMO SAPIENS
  source 1..121: /mol_type=“unassigned DNA” /organism=“HOMO SAPIENS”
  variation 61: /allele=“[A/G]”
ataggtgtgtgtaccacagctcccagctgcatgtactttaaaaatgtgtctaagccaggcatggtggctcacgcccgtaatcccagcactttgggaggcggaggcgggtggatcacctgag

SEQ ID NO: 111 - DNA, 121 bp, HOMO SAPIENS
  source 1..121: /mol_type=“unassigned DNA” /organism=“HOMO SAPIENS”
  variation 61: /allele=“[A/G]”
tgcaataatgtgcaaacagaaaaatcagaacctgctcatgctgccatattaataggaaccatcagtcagccagagagggactcacatatcagacttacatattactaaactattttctgtg

SEQ ID NO: 112 - DNA, 121 bp, HOMO SAPIENS
  source 1..121: /mol_type=“unassigned DNA” /organism=“HOMO SAPIENS”
  variation 61: /allele=“[A/G]”
cagagcttggtatctgagcctggggccctttgagccagctgtgttgggggaggtggaggcaggaagttgtaaggtttgagactttgagagggagccttgagtgtgtagttactaagggaaa

SEQ ID NO: 113 - DNA, 121 bp, HOMO SAPIENS
  source 1..121: /mol_type=“unassigned DNA” /organism=“HOMO SAPIENS”
  variation 61: /allele=“[C/G]”
gaaactgggataatacagccatgcgctacctactggcattcccgtcagtgcgtacacgatcatggtcccagactgcaattttttttttttttttttgagacagagtctcactctgtcaccc

SEQ ID NO: 114 - DNA, 121 bp, HOMO SAPIENS
  source 1..121: /mol_type=“unassigned DNA” /organism=“HOMO SAPIENS”
  variation 61: /allele=“[A/G]”
tccttaacttaactgctttcctcattggcttggtctccatagtgattcattttgctgtaaaaagtagacaattatagacaattatgaaaaatatgaatactgtggtctctgagtctgaatt

SEQ ID NO: 115 - DNA, 121 bp, HOMO SAPIENS
  source 1..121: /mol_type=“unassigned DNA” /organism=“HOMO SAPIENS”
  variation 61: /allele=“[A/G]”
aaattagtatactacctaacctgggaaataaattaaaaactgtgatttgattttcataataaagaatagaaactatcctttaggtcatttctaattacaaaaaaatttccattcaaatcat

SEQ ID NO: 116 - DNA, 121 bp, HOMO SAPIENS
  source 1..121: /mol_type=“unassigned DNA” /organism=“HOMO SAPIENS”
  variation 61: /allele=“[A/C]”
atatgaatgaaagaataaaactcatcttaattttcagagacttatctacatagaaaaaataaagtattttagaattaacaagattggaagattgctgacataaaaatcaatttttagtagt

SEQ ID NO: 117 - DNA, 121 bp, HOMO SAPIENS
  source 1..121: /mol_type=“unassigned DNA” /organism=“HOMO SAPIENS”
  variation 61: /allele=“[A/G]”
gggtggtgaggcctaagctgaacctgagaggtgaggaaaacagaccaagctgaccaaaccactccaggcccttcctccactcacagggatgctcctcccgtggtgccttcctctagacact

SEQ ID NO: 118 - DNA, 121 bp, HOMO SAPIENS
  source 1..121: /mol_type=“unassigned DNA” /organism=“HOMO SAPIENS”
  variation 61: /allele=“[A/G]”
tgcacaccatgcgaactgtggagtatctcagtaagagtgttaggaggaatattttataggacttgtgcttgtattaggtgattttggggagtttaagaaagcagagctttctcgattggat

SEQ ID NO: 119 - DNA, 121 bp, HOMO SAPIENS
  source 1..121: /mol_type=“unassigned DNA” /organism=“HOMO SAPIENS”
  variation 61: /allele=“[A/G]”
tctaattgacttttattagggatttatgaatcaggcagtattccatctaggaaatgtctaaaaaggtgctccaccccattggcagaacagttgtttgagatttgttgttgttattttgctt

SEQ ID NO: 120 - DNA, 121 bp, HOMO SAPIENS
  source 1..121: /mol_type=“unassigned DNA” /organism=“HOMO SAPIENS”
  variation 61: /allele=“[A/G]”
aatgggaagcagcagggtgtgatgtggaccctggattgtatgtattccctctcttagggcatggctgctttttatttgcagctttaccattgccatgctggaaaatcatcacatatttcaa

SEQ ID NO: 121 - DNA, 121 bp, HOMO SAPIENS
  source 1..121: /mol_type=“unassigned DNA” /organism=“HOMO SAPIENS”
  variation 61: /allele=“[A/T]”
ggcagtcctaaaggaaggtcaccctgggatcatctcacctttgacgaggctggccagggaagctgctgagaatgaaataaactcttctctcttttgcttggagaaaagaaatcatgggtag

SEQ ID NO: 122 - DNA, 121 bp, HOMO SAPIENS
  source 1..121: /mol_type=“unassigned DNA” /organism=“HOMO SAPIENS”
  variation 61: /allele=“[A/C]”
ctgggctagaggcaaaagcagagatgtgagctgtaaatttgaatgaaggaccagatagaaagtagaaagtggaaaatggaacctagagctttggacagggctcaaaggaaaacaagcattt

SEQ ID NO: 123 - DNA, 121 bp, HOMO SAPIENS
  source 1..121: /mol_type=“unassigned DNA” /organism=“HOMO SAPIENS”
  variation 61: /allele=“[A/G]”
ttgaaatatgattctatatttaataggaaaaggaaacagcagcctattaaaaatgtatcaaaacaataactttttattagtcctactaacatctgaacttttatgttcctacctacaagtc

SEQ ID NO: 124 - DNA, 121 bp, HOMO SAPIENS
  source 1..121: /mol_type=“unassigned DNA” /organism=“HOMO SAPIENS”
  variation 61: /allele=“[A/G]”
ccactgtgggggacggcagactgatgggaacattggttgagtgaccacaagtgctgttgaaagttttttggtaacagttttaagtgttttggttaagctagacctgaaaaaaatggtatag

SEQ ID NO: 125 - DNA, 121 bp, HOMO SAPIENS
  source 1..121: /mol_type=“unassigned DNA” /organism=“HOMO SAPIENS”
  variation 61: /allele=“[A/G]”
ataaatttgtcaaataaataaactttaaagaaatggccaacttgggaaggacattaggccatcagtttgtagtcttacgtcaattcttgatctccaagcaaaattagtttcagttctctga

SEQ ID NO: 126 - DNA, 121 bp, HOMO SAPIENS
  source 1..121: /mol_type=“unassigned DNA” /organism=“HOMO SAPIENS”
  variation 61: /allele=“[A/G]”
agggaggggagaccaagggctctgagcagcccccaaagctccttgtccctcagggtggctatgtggggagcggcctacctctgagatcttctggaactggttgttggactggctgcacttc

SEQ ID NO: 127 - DNA, 121 bp, HOMO SAPIENS
  source 1..121: /mol_type=“unassigned DNA” /organism=“HOMO SAPIENS”
  variation 61: /allele=“[A/C]”
attagaatactttactctacttaattaatcaatcatatttagtttgactcaccttcccagaaccttctagttctttcttatctttcagtgcttgtccagacaacattttcatttcaacaac

SEQ ID NO: 128 - DNA, 121 bp, HOMO SAPIENS
  source 1..121: /mol_type=“unassigned DNA” /organism=“HOMO SAPIENS”
  variation 61: /allele=“[A/C]”
ccagattcatgaagaaccctgtatcattgatatcacctagaccaccacaaaacaaacataacatttatgtctctttagtctccattaaaaataaacatgtaaaaatgaatcaaactcattt

SEQ ID NO: 129 - DNA, 121 bp, HOMO SAPIENS
  source 1..121: /mol_type=“unassigned DNA” /organism=“HOMO SAPIENS”
  variation 61: /allele=“[A/G]”
aatgaacttacattctacctgcctccctgtatattttgctttggttctaattattgttaaatgaatcacaacatgtgatatacctctcagttacttccaattgaatcaagagtttttctga

SEQ ID NO: 130 - DNA, 121 bp, HOMO SAPIENS
  source 1..121: /mol_type=“unassigned DNA” /organism=“HOMO SAPIENS”
  variation 61: /allele=“[A/G]”
tgataaactgtgctccataacacaaataatttcattcttcttcctttcttgccgagtagtaaaaaaaagaggatggctggtttatctcaagtaatcagacatttaataataatatagaaaa

SEQ ID NO: 131 - DNA, 121 bp, HOMO SAPIENS
  source 1..121: /mol_type=“unassigned DNA” /organism=“HOMO SAPIENS”
  variation 61: /allele=“[A/C]”
ccaacagctcatgagcaaggaggccaaaaccctgcgtggacggtctgcttccctgcccttaccccccgacctttattttttttttgagacgaagtctcgctctgtcacctaggctgaagta

SEQ ID NO: 132 - DNA, 121 bp, HOMO SAPIENS
  source 1..121: /mol_type=“unassigned DNA” /organism=“HOMO SAPIENS”
  variation 61: /allele=“[A/G]”
agagactgcccactcttgttaacttcctgggtttgttttgattccatcaagggagtagcaatgtctcatacttttgtcttccccacggggaagggcacatatttggcactcaatacatgta

SEQ ID NO: 133 - DNA, 121 bp, HOMO SAPIENS
  source 1..121: /mol_type=“unassigned DNA” /organism=“HOMO SAPIENS”
  variation 61: /allele=“[A/C]”
ctttctattctatttttagcagcctatggattctaggagtgacccagctccagggataggacttgattaatctaaatttagagaatggatttagattaatccaatcttggtaattccccgt

SEQ ID NO: 134 - DNA, 121 bp, HOMO SAPIENS
  source 1..121: /mol_type=“unassigned DNA” /organism=“HOMO SAPIENS”
  variation 61: /allele=“[A/G]”
cagaggctggatgaagatgtacgcaagctctttcctcctgagacccagtgagggaggcaaaggaggctccctagctaaagagggagctcaaagttgcagcctttcctcatgcaaggcaagg

SEQ ID NO: 135 - DNA, 121 bp, HOMO SAPIENS
  source 1..121: /mol_type=“unassigned DNA” /organism=“HOMO SAPIENS”
  variation 61: /allele=“[A/G]”
gctgtggtaaagcattaatgaagcacagggcctatcacgcagtcaggctcagtataaggtaaggtgttttttttttaatccaggtaacataagaagcacctgttagcatgagttccataca

SEQ ID NO: 136 - DNA, 121 bp, HOMO SAPIENS
  source 1..121: /mol_type=“unassigned DNA” /organism=“HOMO SAPIENS”
  variation 61: /allele=“[A/G]”
tggggtgggtcatactcaaattgatacacagcctttgtcctgagtgtttgtcttccaaaaaaatctctttgcttagagatctcagaaaatatttgctgtgttaggggcagattcctggatt

SEQ ID NO: 137 - DNA, 121 bp, HOMO SAPIENS
  source 1..121: /mol_type=“unassigned DNA” /organism=“HOMO SAPIENS”
  variation 61: /allele=“[A/G]”
ccttattcatagagtagtattgcttaaaaactgctccaaccacttcttaaacctgaaaccatagacagaaacatctcctaagactgataaatcctaagctttatgctgttagagactgggt

SEQ ID NO: 138 - DNA, 121 bp, HOMO SAPIENS
  source 1..121: /mol_type=“unassigned DNA” /organism=“HOMO SAPIENS”
  variation 61: /allele=“[A/G]”
ctgcttgtattacctgaacagttctttgtgttttgattctattgtgttctgtgttgctgaatagcagttggtaagcaatggcatgtcatccttgtccctgattttggccgaatgaggacat

SEQ ID NO: 139 - DNA, 121 bp, HOMO SAPIENS
  source 1..121: /mol_type=“unassigned DNA” /organism=“HOMO SAPIENS”
  variation 61: /allele=“[A/G]”
tcttacagtaaatccccacttatcaaatcttcagatgtgtagagaaggaataaggcagggataatgggggagtgggacagagagatgccctttctggagtttgcacaacggttgcatgctg

SEQ ID NO: 140 - DNA, 121 bp, HOMO SAPIENS
  source 1..121: /mol_type=“unassigned DNA” /organism=“HOMO SAPIENS”
  variation 61: /allele=“[A/G]”
gtcttcgtgaatctgcgtaaattgctgcatctctcttggcctcagttttcttagccacacagacaggactgaactaaatgatctctaaagtacttctcaagtctataattctatgattctc

SEQ ID NO: 141 - DNA, 121 bp, HOMO SAPIENS
  source 1..121: /mol_type=“unassigned DNA” /organism=“HOMO SAPIENS”
  variation 61: /allele=“[A/G]”
agaggattagaatgacttgctcctcacaatttccctgcgtctgtaactgcacccatgtagacctcatcacctagagccttagcctcctaaataatagtagctggcacttactgagaatgct

SEQ ID NO: 142 - DNA, 121 bp, HOMO SAPIENS
  source 1..121: /mol_type=“unassigned DNA” /organism=“HOMO SAPIENS”
  variation 61: /allele=“[A/G]”
agtgattatattttccattatcccatatatatgtaatggtatgtaatttgtatcattctcatttcataggagagttatttcattacacaacaagaatgccatagggtggcatttctgaaag

SEQ ID NO: 143 - DNA, 121 bp, HOMO SAPIENS
  source 1..121: /mol_type=“unassigned DNA” /organism=“HOMO SAPIENS”
  variation 61: /allele=“[A/G]”
gaggccgtgtgaatgcttgggagaagcgcgctttcggccaggggtctggaatgcttgcacagggttcttctctataaacagtgcagaccagggcctcctgggcaagcgcagggggtgggcg

SEQ ID NO: 144 - DNA, 121 bp, HOMO SAPIENS
  source 1..121: /mol_type=“unassigned DNA” /organism=“HOMO SAPIENS”
  variation 61: /allele=“[A/G]”
tattgtggcaataacttctgctgaaaaactaacctgttctactgagaattctatcaatgtaaatagataaacagatgctggattacacatatctgtacagaactttcctaatgctctatct

SEQ ID NO: 145 - DNA, 121 bp, HOMO SAPIENS
  source 1..121: /mol_type=“unassigned DNA” /organism=“HOMO SAPIENS”
  variation 61: /allele=“[A/G]”
tctaatgggaagagagaaacaaggagagagagaaaaacaaacaggcaaattggagaaacaatgcatacaaagtagacatttccaccgtgcgctgcagttttttccatcattatttgtttgg

SEQ ID NO: 146 - DNA, 121 bp, HOMO SAPIENS
  source 1..121: /mol_type=“unassigned DNA” /organism=“HOMO SAPIENS”
  variation 61: /allele=“[A/G]”
acatctaagcccactccagccggcccccagaggtgggagggtccgccacctcccacagcgagcacctgggttaccataggtgcagttacagcagaagcgaataatgaggagaatctccatg

SEQ ID NO: 147 - DNA, 121 bp, HOMO SAPIENS
  source 1..121: /mol_type=“unassigned DNA” /organism=“HOMO SAPIENS”
  variation 61: /allele=“[A/G]”
ccataatttgtattagcacattaaagaccccgagaggttctgcaaaaggaaactagttgtaccaacttggtacaactcagcatttccaaaatatttggttacagagcactttttgcatgtg

SEQ ID NO: 148 - DNA, 121 bp, HOMO SAPIENS
  source 1..121: /mol_type=“unassigned DNA” /organism=“HOMO SAPIENS”
  variation 61: /allele=“[A/G]”
tcagggtggattttgaaatttccattatatgcaaagcccatgaaaggctaaatatcagttaagaggggagaggagggtggctcctaggtcctctaatgggcaggaaagtatttaaaacaac

SEQ ID NO: 149 - DNA, 121 bp, HOMO SAPIENS
  source 1..121: /mol_type=“unassigned DNA” /organism=“HOMO SAPIENS”
  variation 61: /allele=“[A/G]”
tgagattagtgtttgtaaatgcacactgttgggggaaccctcttcctagtccttgtttccatgtttcccaggaatgaacactagtggagcagcacttcccatttccccccactctttactc

SEQ ID NO: 150 - DNA, 121 bp, HOMO SAPIENS
  source 1..121: /mol_type=“unassigned DNA” /organism=“HOMO SAPIENS”
  variation 61: /allele=“[A/G]”
taccacctcttcagcaccctctcgcatccccacccgtccagcagcagcacaaaggggcccaaaggtgcagcattagggaatctaatggcctgaggaataagttctcgcccactgtgactcc

SEQ ID NO: 151 - DNA, 121 bp, HOMO SAPIENS
  source 1..121: /mol_type=“unassigned DNA” /organism=“HOMO SAPIENS”
  variation 61: /allele=“[A/G]”
cccttcccaagccatgggcaaaaacagctcaggtagtaatgaaggtgtggctatagctgaacaattggatttaaatcccacagagccatggtgctgggaagaggggctgccctggccagtc

SEQ ID NO: 152 - DNA, 121 bp, HOMO SAPIENS
  source 1..121: /mol_type=“unassigned DNA” /organism=“HOMO SAPIENS”
  variation 61: /allele=“[A/G]”
aagtggaattacatcaaactaaattacatcatcagagtaaagagacaatttacaaaaaggaaagaaatatttaaaaaccacacatcggataaggggctaatttccaaaatatatgaggaac

SEQ ID NO: 153 - DNA, 121 bp, HOMO SAPIENS
  source 1..121: /mol_type=“unassigned DNA” /organism=“HOMO SAPIENS”
  variation 61: /allele=“[A/G]”
ctccagcgcgcctgaggctcatgcatttggctaatgagctgcggtttctcttcaggtcggaatggatcttgaaggggaccgcaatggaggagcaaagaagaagaacttttttaaactgaac

SEQ ID NO: 154 - DNA, 121 bp, HOMO SAPIENS
  source 1..121: /mol_type=“unassigned DNA” /organism=“HOMO SAPIENS”
  variation 61: /allele=“[A/G]”
taagacaaagctggctccaggcaaagaatactaccagcaacaaagaggaacatttcagataataaaagagacaattcattgggtggatcacaagctcaggagttcgagaccagcctggcca

SEQ ID NO: 155 - DNA, 121 bp, HOMO SAPIENS
  source 1..121: /mol_type=“unassigned DNA” /organism=“HOMO SAPIENS”
  variation 61: /allele=“[A/T]”
tctgcctagaaggactagcctgctgcttcatttcccccctcctctgcagccgatttcagaaggctgcagcagagaaagcgagacccccacaccttgtttgtgtgtacccttccttccgcac

SEQ ID NO: 156 - DNA, 121 bp, HOMO SAPIENS
  source 1..121: /mol_type=“unassigned DNA” /organism=“HOMO SAPIENS”
  variation 61: /allele=“[A/G]”
taaaaagcaaatattagtaacctggaaaacatacatggaggtatgttcattaacggcagtaaaaaaccaaaccaaattttagagatgagcggtaccttagaagatttagtcaggggaaaag

SEQ ID NO: 157 - DNA, 121 bp, HOMO SAPIENS
  source 1..121: /mol_type=“unassigned DNA” /organism=“HOMO SAPIENS”
  variation 61: /allele=“[A/C]”
caggaacatccagctgcctgcatgacttttctaagtgtctaaaaagcatcttaaacttaaattcttgattccctctcctttactccacgacaaaaatccagctcttcccattgtcttctct

SEQ ID NO: 158 - DNA, 121 bp, HOMO SAPIENS
  source 1..121: /mol_type=“unassigned DNA” /organism=“HOMO SAPIENS”
  variation 61: /allele=“[A/G]”
tttatgcttgaaaatcatagaaattgtgtctaaggatatgctttgggatatttggacttcacttttgttttagtttttagttagctgttgagtttaaagtaatttagtgctctgatatttg

SEQ ID NO: 159 - DNA, 121 bp, HOMO SAPIENS
  source 1..121: /mol_type=“unassigned DNA” /organism=“HOMO SAPIENS”
  variation 61: /allele=“[A/G]”
tgcgggtacaccggcaggcaggaaaacccaggcttctctccacatggtgtttacgtcgtgaggggagagagactagggacgcacgagtagagaagatccctttggtttatgttaagtgcag

SEQ ID NO: 160 - DNA, 121 bp, HOMO SAPIENS
  source 1..121: /mol_type=“unassigned DNA” /organism=“HOMO SAPIENS”
  variation 61: /allele=“[A/G]”
tgattcctaggctgcctgtactagtgatagtgaggctcactaccatccaccacctaaattagaaccgcttgatgacacagcacaggttctatatctttagatggtaaattaaaaattcctg

SEQ ID NO: 161 - DNA, 121 bp, HOMO SAPIENS
  source 1..121: /mol_type=“unassigned DNA” /organism=“HOMO SAPIENS”
  variation 61: /allele=“[A/G]”
acaagtcggggtgtagcttacgggagggaagtcaaagtcaggcacgttcatcacactcagaatgtagtccactctgaactggttctcggggttggccagctccacggggggcaccaggttg

SEQ ID NO: 162 - DNA, 121 bp, HOMO SAPIENS
  source 1..121: /mol_type=“unassigned DNA” /organism=“HOMO SAPIENS”
  variation 61: /allele=“[A/C]”
gtagtagatgctcattgtaagattcaaaaacattccagcttacaaaacatatccagcttaaattttaactcatggtctttagcaagtatagattcctcaagtgaaagggcattgaggcaga

SEQ ID NO: 163 - DNA, 121 bp, HOMO SAPIENS
  source 1..121: /mol_type=“unassigned DNA” /organism=“HOMO SAPIENS”
  variation 61: /allele=“[A/G]”
gcaatgctagaaatatggggattaaaataatgggaaaatcagttttagtgtaatacaaggaaaaacattaaacatgaagctgtccagcagtagaacaaattgccttgcaaagagctgcaaa

SEQ ID NO: 164 - DNA, 121 bp, HOMO SAPIENS
  source 1..121: /mol_type=“unassigned DNA” /organism=“HOMO SAPIENS”
  variation 61: /allele=“[A/G]”
gcccaagattctatatttgaacaagcttctgggtaatatttatgacagggaagtcttgagaaaatttggactataggtcgtcttttaaggttcttgccaactctaagactgccatcccata

SEQ ID NO: 165 - DNA, 121 bp, HOMO SAPIENS
  source 1..121: /mol_type=“unassigned DNA” /organism=“HOMO SAPIENS”
  variation 61: /allele=“[A/G]”
agctacctcagagtactttgtcttttaatgggattataatagaatctcatgaccttgttaaacttaaataagtcaataaatggaacatttcaaacagtgcctggttcacagtggtattatc

SEQ ID NO: 166 - DNA, 120 bp, HOMO SAPIENS
  source 1..120: /mol_type=“unassigned DNA” /organism=“HOMO SAPIENS”
  variation 61: /allele=“[-/CACTTACCTTCTTTGTGCCACAGTTTCCCTATCTA AAACACAAGGTTATCAGTTATCAACATCTCTTGGGATTGTGAGGACTAAAGTAATGCACATAAA G]”
ctttgttaaatgttttttctgcatctattgagctgatcatatgctttctcttgctaatgtggctttgtacagtgcctgttacatggtatactttcaacattagtagtagtagatgttgta

SEQ ID NO: 167 - DNA, 120 bp, HOMO SAPIENS
  source 1..120: /mol_type=“unassigned DNA” /organism=“HOMO SAPIENS”
  variation 61: /allele=“[-/AAATTACCCTGTTAGGTTTCAATGAAACACCTTTTC TCTTGTAACAAACATCTCCTCCAAGCTAGAATTTCAAAACAG]”
tttttctaagtttatgtcttaacctaacaataactcaaaagagaaacaagtatctctccatgttaccatccactaggtaataatttttatgctagcaacaaaacccaaaatatgtgttca

SEQ ID NO: 168 - DNA, 121 bp, HOMO SAPIENS
  source 1..121: /mol_type=“unassigned DNA” /organism=“HOMO SAPIENS”
  variation 61: /allele=“[A/C]”
ttggttatgatttttttttcatttgaagtaaatatccacctttgtatctaattttgcattaaaaaaaaaattttttttttttactttaagttgaatccctacaattgtataaccttcaggt

SEQ ID NO: 169 - DNA, 121 bp, HOMO SAPIENS
  source 1..121: /mol_type=“unassigned DNA” /organism=“HOMO SAPIENS”
  variation 61: /allele=“[A/G]”
ccagctagttttattttttaatagtgttcttgcacatgaggagaaagactgaattcaattacactattctataactaattataagttataataaaaatgaaacaaaaacatttcaactgat

SEQ ID NO: 170 - DNA, 121 bp, HOMO SAPIENS
  source 1..121: /mol_type=“unassigned DNA” /organism=“HOMO SAPIENS”
  variation 61: /allele=“[A/G]”
aagaaggtggcgtgtcacttcgtttgacttcagctgggaacatgcatatcagtcgactcaaattttttgctattctgtgcttatccacgaatcgataggaaagcaagtgtggatttggggg

SEQ ID NO: 171 - DNA, 121 bp, HOMO SAPIENS
  source 1..121: /mol_type=“unassigned DNA” /organism=“HOMO SAPIENS”
  variation 61: /allele=“[A/G]”
aatgacacagatcgtcacacagttttaagacaaatgtttttacctatttgacctagtctgacaatccctatttgggcaaaaatcttcatttgcaggtcatgattggaggcaggcacagaaa

SEQ ID NO: 172 - DNA, 121 bp, HOMO SAPIENS
  source 1..121: /mol_type=“unassigned DNA” /organism=“HOMO SAPIENS”
  variation 61: /allele=“[A/C]”
aattctttgatgtgctacaaacctgaaactggtaagacaagcacaaagcaacgtgcaataaaaaaatcgtatctcaagggaaaatactcaaagaaagaaaagtggcagcacttatattagt

SEQ ID NO: 173 - DNA, 121 bp, HOMO SAPIENS
  source 1..121: /mol_type=“unassigned DNA” /organism=“HOMO SAPIENS”
  variation 61: /allele=“[A/G]”
caaaagtgataaaagtaactttcaaggctagatcatgcaagacaaggcaacatagcttctacctggttctatgaagacatttgcctttggctccctgagcctccatccaagaagtcgaagt

SEQ ID NO: 174 - DNA, 121 bp, HOMO SAPIENS
  source 1..121: /mol_type=“unassigned DNA” /organism=“HOMO SAPIENS”
  variation 61: /allele=“[A/G]”
gaggtccaggctgcagtgagctatgatcacatgccttcactccagcctgggtgacagagcaagacactgactcaaaacaaaatacataaattaatttgtttaattcatgattagttactat

SEQ ID NO: 175 - DNA, 121 bp, HOMO SAPIENS
  source 1..121: /mol_type=“unassigned DNA” /organism=“HOMO SAPIENS”
  variation 61: /allele=“[A/G]”
aatgtgggccacatgtgaccaacaagataattatgaaacctgactgctggatatgctgatacagccaaaaaacatcaaggactgtgagtgagtttggaggtgggagcagagaaaatttctg

SEQ ID NO: 176 - DNA, 121 bp, HOMO SAPIENS
  source 1..121: /mol_type=“unassigned DNA” /organism=“HOMO SAPIENS”
  variation 61: /allele=“[A/G]”
gcaatcagaaaggtcctctttaaatgtgagttagatcatgttactgctctgctcaaaataatgcagtggttttccattgcacagagtggcagattgcattttccaaaagacaattgcaatg

SEQ ID NO: 177 - DNA, 121 bp, HOMO SAPIENS
  source 1..121: /mol_type=“unassigned DNA” /organism=“HOMO SAPIENS”
  variation 61: /allele=“[A/G]”
ctagcatgatttattaatattagcctttcttctctccccgtttatgctttggtgggtactagacagaaaccccacaaattttaagacagttttaagagaaatagtaactggttaaatatcc

SEQ ID NO: 178 - DNA, 121 bp, HOMO SAPIENS
  source 1..121: /mol_type=“unassigned DNA” /organism=“HOMO SAPIENS”
  variation 61: /allele=“[A/G]”
aggtcatacagcaaataactggcattcctggaacccaaattccaggtgtcttgttccaaaacccatgttctttattctattctgcctctgccaaacaaaacccaaaccaaaaatgtcttct

SEQ ID NO: 179 - DNA, 121 bp, HOMO SAPIENS
  source 1..121: /mol_type=“unassigned DNA” /organism=“HOMO SAPIENS”
  variation 61: /allele=“[A/C]”
agtcaataaacccaaatgataatttaaaattcaccctgatgatggttccaataaatatataaatagtgtagctctagttcggtttcataagaattgtgcagcaataattctttctgtaatt

SEQ ID NO: 180 - DNA, 121 bp, HOMO SAPIENS
  source 1..121: /mol_type=“unassigned DNA” /organism=“HOMO SAPIENS”
  variation 61: /allele=“[A/G]”
tatgccaccatacccgctaatttttgtatttaatagaaacagggtttcgccatgttgacaagctggacttgaactcctcacttgaactcctcacgtcaagtgatctgcctgctttagcctc

SEQ ID NO: 181 - DNA, 121 bp, HOMO SAPIENS
  source 1..121: /mol_type=“unassigned DNA” /organism=“HOMO SAPIENS”
  variation 61: /allele=“[A/G]”
aaacaaacaaacaaacaaactgaggtttaggtttaggtagctggagtttataggcatggcacataggtcagagcctcaattttctagctaaatgtcaatgtttcccacttattttattgcc

SEQ ID NO: 182 - DNA, 121 bp, HOMO SAPIENS
  source 1..121: /mol_type=“unassigned DNA” /organism=“HOMO SAPIENS”
  variation 61: /allele=“[A/G]”
gcttacagccagggctacacagaagtgagcaaagctggtgaagatggggatgggggagtgaagtgagttgacgctagaaagggatgtagcaaatgtaactattattccagaatccaagtgt

SEQ ID NO: 183 - DNA, 121 bp, HOMO SAPIENS
  source 1..121: /mol_type=“unassigned DNA” /organism=“HOMO SAPIENS”
  variation 61: /allele=“[A/G]”
ctactagctacataatgtgatgccatattaaactgtaatcacctttccaccaaactaataaagacaacatgctaatttttgtattaagacacagtgcaataacacacaattgaatgatgct

SEQ ID NO: 184 - DNA, 121 bp, HOMO SAPIENS
  source 1..121: /mol_type=“unassigned DNA” /organism=“HOMO SAPIENS”
  variation 61: /allele=“[A/G]”
tgtgtactcccaaaattcatatgttgatacctaatctccaaagcaatagtattaagggtgagtgcctttgggaggtgattggataatgagggcagagctttcatgtacagaattagtactc

SEQ ID NO: 185 - DNA, 121 bp, HOMO SAPIENS
  source 1..121: /mol_type=“unassigned DNA” /organism=“HOMO SAPIENS”
  variation 61: /allele=“[A/C]”
gaaacagaggcttagacagtttacttatgtgcccaaggacacaaaatcagaaacaggtacaaggagcacttgaaccaaaaccaatactgtcttgccataccaaacagtatttatttattta

SEQ ID NO: 186 - DNA, 121 bp, HOMO SAPIENS
  source 1..121: /mol_type=“unassigned DNA” /organism=“HOMO SAPIENS”
  variation 61: /allele=“[A/C]”
cctgtggccgttggttttcctgggtggggaagggtgctggcctcattcacaacagcagatactcattcctccagggtcaggctatggggctcaacgtgatcaggacagatctgagccccgt

SEQ ID NO: 187 - DNA, 121 bp, HOMO SAPIENS
  source 1..121: /mol_type=“unassigned DNA” /organism=“HOMO SAPIENS”
  variation 61: /allele=“[A/G]”
actgggcttgtagcttaaattattcacactttactcatgtaatgatgaacagttttaggtacttataatatgtagaggctaactctctctttctctcactctgtctttccctctgtttgtc

SEQ ID NO: 188 - DNA, 121 bp, HOMO SAPIENS
  source 1..121: /mol_type=“unassigned DNA” /organism=“HOMO SAPIENS”
  variation 61: /allele=“[A/C]”
tcaacccaaggcagagagagccctgtctcaaaacagatttctgagtgtggcttctgtccaagcatgtgaattaacatgtaacacaaaagagaagaaagaaatgttaaggaaattataccag

SEQ ID NO: 189 - DNA, 121 bp, HOMO SAPIENS
  source 1..121: /mol_type=“unassigned DNA” /organism=“HOMO SAPIENS”
  variation 61: /allele=“[A/G]”
attttctctctctcgtagctgagagagtcatgactatggcgtgttctctgtactctgaggacctgaacccactcatgggttactctggcctttggtcaggtagttttgccaactcgctatt

SEQ ID NO: 190 - DNA, 121 bp, HOMO SAPIENS
  source 1..121: /mol_type=“unassigned DNA” /organism=“HOMO SAPIENS”
  variation 61: /allele=“[A/C]”
gcagtactcaccatgggccttaaggtgagactcagagatgtgctggcttcaggtataaccaagcacatttgaaactatagcggctatggggagagattccttctgcttgagaaaaggagag

SEQ ID NO: 191 - DNA, 121 bp, HOMO SAPIENS
  source 1..121: /mol_type=“unassigned DNA” /organism=“HOMO SAPIENS”
  variation 61: /allele=“[C/G]”
gtgtatgctttgtgaggataggtagcttttcttactcactgttgttaccagtacctagaaccaagcctgaccttattaggttctttcaaatatttgaaagatattttaaaatattcacata

SEQ ID NO: 192 - DNA, 121 bp, HOMO SAPIENS
  source 1..121: /mol_type=“unassigned DNA” /organism=“HOMO SAPIENS”
  variation 61: /allele=“[A/G]”
tgatctcattaccttaattcctttgcttataaaatgagttcattggtcagaagcaacgctatgtacaataccaagaatatgaatatgtcatttacagaatgacaagctcgtcaatttcagt

SEQ ID NO: 193 - DNA, 121 bp, HOMO SAPIENS
  source 1..121: /mol_type=“unassigned DNA” /organism=“HOMO SAPIENS”
  variation 61: /allele=“[A/G]”
gcaaagaatcactggttaccattatctttgaaatggctcctcataaaacacagaaaataaacattaagacatgaaagctacaaggcccacaatgcgggaattttaaccttgaaaactgtcc

SEQ ID NO: 194 - DNA, 121 bp, HOMO SAPIENS
  source 1..121: /mol_type=“unassigned DNA” /organism=“HOMO SAPIENS”
  variation 61: /allele=“[A/G]”
tcagagtttaggattttggccattctaagagatgtgcagtagtaactcagtgttttatttacaattccctaatgacatatgatgttaagtatcttctaatatgctcatttgtcatctgcat

SEQ ID NO: 195 - DNA, 121 bp, HOMO SAPIENS
  source 1..121: /mol_type=“unassigned DNA” /organism=“HOMO SAPIENS”
  variation 61: /allele=“[A/T]”
ttttaatatttgtttagatatgacatttattcaaagttaaaagcaaacacnnnnagaattatgaagaggtatctgtttaacatttcctcagtcaagttcagagtcttcagagacttcrtaa

SEQ ID NO: 196 - DNA, 121 bp, HOMO SAPIENS
  source 1..121: /mol_type=“unassigned DNA” /organism=“HOMO SAPIENS”
  variation 61: /allele=“[A/G]”
gttttcctttaacattccattatcctattgttcattctttggagctgtgatttgtttaatatatttcaggcttcttaataaatcaagtcatgtaagttattatttggatcatttcgaaact

SEQ ID NO: 197 - DNA, 121 bp, HOMO SAPIENS
  source 1..121: /mol_type=“unassigned DNA” /organism=“HOMO SAPIENS”
  variation 61: /allele=“[A/G]”
gtggttctaaagcttcggtgaatattagaatggcctcaagagctagtaaaaaacacagccagcctggattattcaagtaggctagggtttggccttttatttttataatattccgaggtga

SEQ ID NO: 198 - DNA, 121 bp, HOMO SAPIENS
  source 1..121: /mol_type=“unassigned DNA” /organism=“HOMO SAPIENS”
  variation 61: /allele=“[A/G]”
gttggtgagaaggcatatggggaaaaaataaggcaggaaaggaagacggaaaatgctgtgagtagggtggcattttaaatactgtggtcagggaagcctcaccaaaaatgtgacatctgaa

SEQ ID NO: 199 - DNA, 121 bp, HOMO SAPIENS
  source 1..121: /mol_type=“unassigned DNA” /organism=“HOMO SAPIENS”
  variation 61: /allele=“[A/G]”
ttgtcttttattggttttataaaggatctaagtgtttggaaaggtgtgggaccatgtactattggagatttcagtgtttgactatgagagaaggaaatgttattttttgggaatgttattt

SEQ ID NO: 200 - DNA, 121 bp, HOMO SAPIENS
  source 1..121: /mol_type=“unassigned DNA” /organism=“HOMO SAPIENS”
  variation 61: /allele=“[A/C]”
gaagcatcacctcttttatcatatgaagccttttcacaaaggagggaatgatgattgactaagttttgtgtctattctatacactgtactgtcaaagcatgcagagcatgtattgcatata

SEQ ID NO: 201 - DNA, 121 bp, HOMO SAPIENS
  source 1..121: /mol_type=“unassigned DNA” /organism=“HOMO SAPIENS”
  variation 61: /allele=“[A/G]”
ctctttgtgttaaggttgtatcatctacctgtagtcactgcagtcagctgaattttaccaagagaatctgacagtcgttgcccagtcaaattagtttagatccatctgtaacaggttccta

SEQ ID NO: 202 - DNA, 121 bp, HOMO SAPIENS
  source 1..121: /mol_type=“unassigned DNA” /organism=“HOMO SAPIENS”
  variation 61: /allele=“[A/G]”
ctgattcctgctctgtagccacacagatgccaacagctggcacttgtccaagaaacatgtactcaaggtcaggtgcagtggctcatgcctgtaatcctaggtttttgggaagctgaggagg

SEQ ID NO: 203 - DNA, 121 bp, HOMO SAPIENS
  source 1..121: /mol_type=“unassigned DNA” /organism=“HOMO SAPIENS”
  variation 61: /allele=“[A/G]”
aatagagaccagaaaggattatttgatgttcatttagcaagcaacatagtaaaataatttattccactgtttgtatgtatccttgactgtttctaacaagtgacccattctttcttaatat

SEQ ID NO: 204 - DNA, 121 bp, HOMO SAPIENS
  source 1..121: /mol_type=“unassigned DNA” /organism=“HOMO SAPIENS”
  variation 61: /allele=“[A/G]”
aaaataaagcccagatgcctttccggctccccccacggggttgccctgatggtttaagacaataacagatatgaaaatcctctgtaaacaggaaggcttcaccactcttggaactcaagat

SEQ ID NO: 205 - DNA, 121 bp, HOMO SAPIENS
  source 1..121: /mol_type=“unassigned DNA” /organism=“HOMO SAPIENS”
  variation 61: /allele=“[A/G]”
tgtgacgatgccttatgacaaacaactctacatctcagtgtcttacaccaatgagctcataagcctgcaggttggctgtggtgactgctcctggcttggccccatgggtgtctcatcccag

SEQ ID NO: 206 - DNA, 121 bp, HOMO SAPIENS
  source 1..121: /mol_type=“unassigned DNA” /organism=“HOMO SAPIENS”
  variation 61: /allele=“[A/G]”
ttatgctctttattctaaggaagtgccccctaaaacaaagctcaggagcctcaacccggcagggaagacagtttcctcacgaggcaggcaagcaacaccaggtggctctctttcccaagat

SEQ ID NO: 207 - DNA, 100 bp, HOMO SAPIENS
  source 1..100: /mol_type=“unassigned DNA” /organism=“HOMO SAPIENS”
  variation 40: /allele=“[A/G]”
ttactagttatgcaatgcactcgaatccagtttaagttcagcgctctcatctgtaaaagtggggcaagaatttgccttttgatgttgggagatcaagttc

SEQ ID NO: 208 - DNA, 121 bp, HOMO SAPIENS
  source 1..121: /mol_type=“unassigned DNA” /organism=“HOMO SAPIENS”
  variation 61: /allele=“[A/G]”
gccctatagataccatgggaaacagacagtggcccctgttctcaagtggcttagactctaatgggaaagacatttattttttcttttttttttttttttagagacggagtctcgctctgtc

SEQ ID NO: 209 - DNA, 121 bp, HOMO SAPIENS
  source 1..121: /mol_type=“unassigned DNA” /organism=“HOMO SAPIENS”
  variation 61: /allele=“[A/G]”
agtatctaagcttattggccctaagtaaatcttaggttaggtagagctcagttcccagggacattcaagattcataaagaagtgatatttttcccagctaaaatatttttcttcttaccag

SEQ ID NO: 210 - DNA, 121 bp, HOMO SAPIENS
  source 1..121: /mol_type=“unassigned DNA” /organism=“HOMO SAPIENS”
  variation 61: /allele=“[A/G]”
cttttattttaaactttttcttaagtaattttacaattgctgaaaagtaaaagagcctcaatgaattcctgaataccctttacctgttttcctgaatgttcctaaaaatacctagcaatga

SEQ ID NO: 211 - DNA, 121 bp, HOMO SAPIENS
  source 1..121: /mol_type=“unassigned DNA” /organism=“HOMO SAPIENS”
  variation 61: /allele=“[A/G]”
ctgagcaccttctatttgccaacaactgttttaggcactggggatatagtgataaacagaacaaccacaaatccctgtcctctggaactcaccgtcagagtgaggaaggcctgagtcccct

SEQ ID NO: 212 - DNA, 121 bp, HOMO SAPIENS
  source 1..121: /mol_type=“unassigned DNA” /organism=“HOMO SAPIENS”
  variation 61: /allele=“[A/C]”
gggtgtgatttggttgctaatttctcttcacttctgggaaaccagccccttataaatcaaactataggccagagaggctgccacatgctcccaggctgtttatttgaagagagacttacat

SEQ ID NO: 213 - DNA, 121 bp, HOMO SAPIENS
  source 1..121: /mol_type=“unassigned DNA” /organism=“HOMO SAPIENS”
  variation 61: /allele=“[A/G]”
aaataaaagcaacacagagcagtatgtacaggacagcgttagaatataccagagaacaagaacacaatctacaatcatttccagtgaatgcaggatgttaaagagatgcataaaatcccct

SEQ ID NO: 214 - DNA, 121 bp, HOMO SAPIENS
  source 1..121: /mol_type=“unassigned DNA” /organism=“HOMO SAPIENS”
  variation 61: /allele=“[A/G]”
gactgcccagagcacagcgtggagaaggcgctcggcccccgcccaggcaggcagagcaccatgatgggttcacgatgccctatgccagggtcgtgggtgacaggtgtgtttgccatctcta

SEQ ID NO: 215 - DNA, 121 bp, HOMO SAPIENS
  source 1..121: /mol_type=“unassigned DNA” /organism=“HOMO SAPIENS”
  variation 61: /allele=“[A/T]”
gactaaaaagcatagtattctgttcttcagggagttgtgggttcggatctgtgcaaagataggaggtagctgaataaacatagttgcaaattataacctcccaaatgtgccctgaggacac

SEQ ID NO: 216 - DNA, 121 bp, HOMO SAPIENS
  source 1..121: /mol_type=“unassigned DNA” /organism=“HOMO SAPIENS”
  variation 61: /allele=“[A/G]”
aaataaacacgaagaacaaagccccaccaccgtgctgtgctgtttgtgtggccccactgcatcgaggccacaggctagctgctagacgcatctagagttccctgattcctaaaattattta

SEQ ID NO: 217 - DNA, 121 bp, HOMO SAPIENS
  source 1..121: /mol_type=“unassigned DNA” /organism=“HOMO SAPIENS”
  variation 61: /allele=“[A/G]”
cattttaattttcaaattgcttgattaaaatggcaaacagtttgaaaattgtatacctctatatcattcagttaaaaaacaataaagtgacattcttaaaaacatcaaggactttcccctc

SEQ ID NO: 218 - DNA, 121 bp, HOMO SAPIENS
  source 1..121: /mol_type=“unassigned DNA” /organism=“HOMO SAPIENS”
  variation 61: /allele=“[A/G]”
catttatacaggataatggaaaagggggtttctcccgagtagagaacttaaacagtgtgaagcacagtgtgttccacactatagctgatgggttggcctcaggggggatgttcaggtatac

SEQ ID NO: 219 - DNA, 121 bp, HOMO SAPIENS
  source 1..121: /mol_type=“unassigned DNA” /organism=“HOMO SAPIENS”
  variation 61: /allele=“[A/G]”
taatatatgaagggtgcattattctaatttaatggattaatcatactttttaaaaacagtattactaaattctgtaataacatggtgattttatatacacatgactaggtgaaaggatatt

SEQ ID NO: 220 - DNA, 121 bp, HOMO SAPIENS
  source 1..121: /mol_type=“unassigned DNA” /organism=“HOMO SAPIENS”
  variation 61: /allele=“[A/G]”
gcaaggtggcactcttaggagttgaatccagctctggtgtgtgggacaggcaggaggagaagaagagagggaggaaaagtcggttcgagaacccaggtggaaaatagattgagggaagcaa

SEQ ID NO: 221 - DNA, 121 bp, HOMO SAPIENS
  source 1..121: /mol_type=“unassigned DNA” /organism=“HOMO SAPIENS”
  variation 61: /allele=“[A/C]”
gcaggcgccccctttccccgctccccaggcgcttcagcaccgcggacagcgcccatccgaatcactgaggccaaagcccagcacgtctaaggcagtcccgtaggaagaccccgtgtgcacc

SEQ ID NO: 222 - DNA, 121 bp, HOMO SAPIENS
  source 1..121: /mol_type=“unassigned DNA” /organism=“HOMO SAPIENS”
  variation 61: /allele=“[A/G]”
aggaccattttagaaatctgtgaaccacagtggtgaaagaaggaacacattctctacagaaatgtatattaagtgtctgttaacctggcattgtcctccccaaccaaaactatttctattg

SEQ ID NO: 223 - DNA, 121 bp, HOMO SAPIENS
  source 1..121: /mol_type=“unassigned DNA” /organism=“HOMO SAPIENS”
  variation 61: /allele=“[A/G]”
agagcaggggaaagagagtggaagtaccaggtgggcaaagtttacaattttaagtaggatagtcagggcagacctcattaaggagataactttgagccaagacgggatagagcagaaggaa

SEQ ID NO: 224 - DNA, 121 bp, HOMO SAPIENS
  source 1..121: /mol_type=“unassigned DNA” /organism=“HOMO SAPIENS”
  variation 61: /allele=“[A/G]”
cttcttgcacatgattctttccatgacacctagtgcccttctccatctagagctacctctatatgtccacgttccttctctctaagctcatgatagacctcaggagaaagtcaggtaggcc

SEQ ID NO: 225 - DNA, 121 bp, HOMO SAPIENS
  source 1..121: /mol_type=“unassigned DNA” /organism=“HOMO SAPIENS”
  variation 61: /allele=“[A/C]”
cgtatggaaccttttggcattggctttttctactcagcataatttcctggagagtcatccaaattgctgcatgtatggatagcttgttccatttcattgctgagttcatttgctttttttt

SEQ ID NO: 226 - DNA, 121 bp, HOMO SAPIENS
  source 1..121: /mol_type=“unassigned DNA” /organism=“HOMO SAPIENS”
  variation 61: /allele=“[A/G]”
gcaactttacctgctaatgactatatacacccatttttctcattttttaaaaatatcatcacatattactttaaaatgtcaagaactgcttcaacagccaggcaatgatggctggtatgct

SEQ ID NO: 227 - DNA, 121 bp, HOMO SAPIENS
  source 1..121: /mol_type=“unassigned DNA” /organism=“HOMO SAPIENS”
  variation 61: /allele=“[A/T]”
aagcaaagcaattgctacaaggaggattatgggtgaaagtcatggatggattatgagttaatcacacacctagagaagcatgtaaaatgtgcaggtaaattacacccattcattcaggcag

SEQ ID NO: 228 - DNA, 121 bp, HOMO SAPIENS
  source 1..121: /mol_type=“unassigned DNA” /organism=“HOMO SAPIENS”
  variation 61: /allele=“[A/G]”
cccgcccacctgagcacagtgtccatataggaacatgagtgacagccctgcacatgggcaagagcatccaaaccacacttcaggcaaaactacatttcagtgatgtccatccttaggaaaa

SEQ ID NO: 229 - DNA, 121 bp, HOMO SAPIENS
  source 1..121: /mol_type=“unassigned DNA” /organism=“HOMO SAPIENS”
  variation 61: /allele=“[A/C]”
cttactttaacatccaaaaataactaaaaagtcctagaaaattaaacttttccaaatttcaaaagtacttgtgctgtatgaattctacttcatgtatcatacacaaacaagttatgacaaa

SEQ ID NO: 230 - DNA, 121 bp, HOMO SAPIENS
  source 1..121: /mol_type=“unassigned DNA” /organism=“HOMO SAPIENS”
  variation 61: /allele=“[A/G]”
acatattccagaaattctccataatttctgatccactcttacattcctctcctttccagcactattattgatctcttcttcttcttttgaaaatctttgttccctccatctatcatttcag

SEQ ID NO: 231 - DNA, 121 bp, HOMO SAPIENS
  source 1..121: /mol_type=“unassigned DNA” /organism=“HOMO SAPIENS”
  variation 61: /allele=“[A/G]”
gcagggtccacgggcttctggacacctccctacctgggccggcttcatcctcctacgaccaacagtcgtgttgatgacatgcacctgtcccgggacttcccccagcccccagccagctgcg

SEQ ID NO: 232 - DNA, 121 bp, HOMO SAPIENS
  source 1..121: /mol_type=“unassigned DNA” /organism=“HOMO SAPIENS”
  variation 61: /allele=“[A/G]”
aaaaataatgttcctttctaaatatgctaaattatttccataaaactcataaacttttatacctagaaatttatgaaaacctattgacaacttttatgcctgaaaagatctgaaagattga

SEQ ID NO: 233 - DNA, 121 bp, HOMO SAPIENS
  source 1..121: /mol_type=“unassigned DNA” /organism=“HOMO SAPIENS”
  variation 61: /allele=“[A/G]”
ggccacaaagcccttgcacaggcacagctataatttttgtctctcttctgttggaaaggtacaaagttaactggagtgatgtgtgtaattgatggtataatggtaagcaaaaatcacaaat

SEQ ID NO: 234 - DNA, 121 bp, HOMO SAPIENS
  source 1..121: /mol_type=“unassigned DNA” /organism=“HOMO SAPIENS”
  variation 61: /allele=“[A/G]”
gaagagaggagtgaggtgctatgtacttttaaacaacaagatatcatgagaactcactccatatcacaagaagagcaccctgggggatggtattaaatcattagaaaccacctcatgatcc

SEQ ID NO: 235 - DNA, 121 bp, HOMO SAPIENS
  source 1..121: /mol_type=“unassigned DNA” /organism=“HOMO SAPIENS”
  variation 61: /allele=“[A/G]”
aaactaggaagaatattgaaggtagccagaaaagaaaaaaaggcacattgcatgcagaggaacaaagatgagaatcacagcaaacttctctttagaaacaatataagttgtaagacaatgg

SEQ ID NO: 236 - DNA, 121 bp, HOMO SAPIENS
  source 1..121: /mol_type=“unassigned DNA” /organism=“HOMO SAPIENS”
  variation 61: /allele=“[A/G]”
ggctgagtgaatgaccaccactctgtggttcaccaaaaaaccacatcaggttttccccagacaccttgggacagtttgaaatgtccaaatagtaaagcaatgaactgccataaatgtagtt

SEQ ID NO: 237 - DNA, 121 bp, HOMO SAPIENS
  source 1..121: /mol_type=“unassigned DNA” /organism=“HOMO SAPIENS”
  variation 61: /allele=“[C/G]”
gttgaggggagcctggagaagttggctgggacagatgacaccacttggagaccatatttacgctctcagactttatccaagtgggactgttgtttaaaggtttgaaaaaacatggcttata

SEQ ID NO: 238 - DNA, 121 bp, HOMO SAPIENS
  source 1..121: /mol_type=“unassigned DNA” /organism=“HOMO SAPIENS”
  variation 61: /allele=“[A/G]”
cactcacaggtctcccctggattgtgcagaaccagacattgctgcctttgcctaggcaggataatagatatcatgagggcttgggaagcttcggggggaaagttaggctatctgcccaccc

SEQ ID NO: 239 - DNA, 121 bp, HOMO SAPIENS
  source 1..121: /mol_type=“unassigned DNA” /organism=“HOMO SAPIENS”
  variation 61: /allele=“[A/G]”
agtatagtcctgagaaagttttggccaagccaatggagagccccatagccaaagctgcccattagaggaatcccatatcaagtagaattggatgggtgagaattctcaggtggctgagagt

SEQ ID NO: 240 - DNA, 121 bp, HOMO SAPIENS
  source 1..121: /mol_type=“unassigned DNA” /organism=“HOMO SAPIENS”
  variation 61: /allele=“[A/G]”
caagaagagaggataaccaacacacaaatgagtaaataaaatgattgctgattgctattaatgctaacaaggaaagagatcctgttccatgtgagtgagatcatgcccattgcttcatcat

SEQ ID NO: 241 - DNA, 121 bp, HOMO SAPIENS
  source 1..121: /mol_type=“unassigned DNA” /organism=“HOMO SAPIENS”
  variation 61: /allele=“[A/G]”
catggcacacagtcacagaaacatagcaagcccttgaaatcaggctttctgactttgtctaatctcctgctttagcaaagacatcaattctccctccttttatttaaatggtggctgggtc

SEQ ID NO: 242 - DNA, 121 bp, HOMO SAPIENS
  source 1..121: /mol_type=“unassigned DNA” /organism=“HOMO SAPIENS”
  variation 61: /allele=“[A/G]”
cacctacacacacctacacacatgcatgcacacacacatggcctctctctccaggcttctagagctcaggacaggtcagatccatctctgtcgggcacaacattgatgctctctgaacact

SEQ ID NO: 243 - DNA, 121 bp, HOMO SAPIENS
  source 1..121: /mol_type=“unassigned DNA” /organism=“HOMO SAPIENS”
  variation 61: /allele=“[A/G]”
ctgagagtttcgtacagacctggtccaaaaattccaatttcataggtgtggagttttcatacaagtacttcaattgctacactcaaagagaaagatttaacacctagaaatctagctgtct

SEQ ID NO: 244 - DNA, 121 bp, HOMO SAPIENS
  source 1..121: /mol_type=“unassigned DNA” /organism=“HOMO SAPIENS”
  variation 61: /allele=“[A/C]”
aataatgcataaggtttttgtaagaattagaattaataaagtacttagaccataataactaattagtattagttgttgtctttgctattattttgatgtggtggttgtttggtttcacctg

SEQ ID NO: 245 - DNA, 121 bp, HOMO SAPIENS
  source 1..121: /mol_type=“unassigned DNA” /organism=“HOMO SAPIENS”
  variation 61: /allele=“[A/G]”
tccctgcttcttactaatattgtcactttgtctcttaatacagatattttcttttgatcaatgtttgtaaagtaacatatgtttctgacctcttactttaaaacttactatggccttgtaa

SEQ ID NO: 246 - DNA, 121 bp, HOMO SAPIENS
  source 1..121: /mol_type=“unassigned DNA” /organism=“HOMO SAPIENS”
  variation 61: /allele=“[A/G]”
gccttgcagattatgtagcaggtcctgatgtaacagaattaagattgcaggtgggattggagttgctaatcagctgactttgagatggagaggtgatcctggattattttggtggacccat

SEQ ID NO: 247 - DNA, 121 bp, HOMO SAPIENS
  source 1..121: /mol_type=“unassigned DNA” /organism=“HOMO SAPIENS”
  variation 61: /allele=“[A/G]”
ccccctagggagtagctgcggcggcaccaagagaggggtggggggcgtgctgcgcagaggaggacctcacaaagcggcctcagagtttcgcaggtcctgctgttctagggaagggttaaag

SEQ ID NO: 248 - DNA, 121 bp, HOMO SAPIENS
  source 1..121: /mol_type=“unassigned DNA” /organism=“HOMO SAPIENS”
  variation 61: /allele=“[A/T]”
gaccacttgaacacatccttttaaatagataccttttttaaaatctatggttatgtaacaactgtgtccgagggatttaagcagaaagccctgtgggtttctcttttcaaaagacagacct

SEQ ID NO: 249 - DNA, 121 bp, HOMO SAPIENS
  source 1..121: /mol_type=“unassigned DNA” /organism=“HOMO SAPIENS”
  variation 61: /allele=“[A/G]”
gtgtcctctctaaggatgggacccctactgtccatctcaggctcagcactgccttggggcaggccacttctggcttctttaggcctcgtttccacgggaggggaagctgggtccgatggtg

SEQ ID NO: 250 - DNA, 121 bp, HOMO SAPIENS
  source 1..121: /mol_type=“unassigned DNA” /organism=“HOMO SAPIENS”
  variation 61: /allele=“[A/G]”
tctgtcaccccttctacatcttagctcacctgtcctcacaaataaacatcactcttgaatactacaatctcactttattagattgtaaatttttatgaggaaaaaggtcctgagctatggc

SEQ ID NO: 251 - DNA, 121 bp, HOMO SAPIENS
  source 1..121: /mol_type=“unassigned DNA” /organism=“HOMO SAPIENS”
  variation 61: /allele=“[A/C]”
acacagcattaattaaaaatggaagttttccacttccttgataatttggctatctgaataaatttgtgaatttgctaggttaagacctagttcgtggtcacatttcaacaaaacagcttga

SEQ ID NO: 252 - DNA, 121 bp, HOMO SAPIENS
  source 1..121: /mol_type=“unassigned DNA” /organism=“HOMO SAPIENS”
  variation 61: /allele=“[A/G]”
acttatatctctttctaaacactagcagcccagaattctcaggccacttttgggcattgtagcaacacaataggtgcctcctgtggaccccatgcctccaatcagagcagggattaccggc

SEQ ID NO: 253 - DNA, 121 bp, HOMO SAPIENS
  source 1..121: /mol_type=“unassigned DNA” /organism=“HOMO SAPIENS”
  variation 61: /allele=“[A/G]”
atggaggggacagagtttatctttttcacggtttgtatatatatattttttaatcttttgagagtcccagtttttgaagcattcacttggctgattcaccaattcatagactggagtagaa

SEQ ID NO: 254 - DNA, 121 bp, HOMO SAPIENS
  source 1..121: /mol_type=“unassigned DNA” /organism=“HOMO SAPIENS”
  variation 61: /allele=“[A/G]”
tagtgtaaggtgaccggaaaaatctgattaaaggacaaatgttcagttcaaaggtgtttcaagctgagaatagcatctctatttactcctcacgatgttcatctcaggaggcactgtactt

SEQ ID NO: 255 - DNA, 121 bp, HOMO SAPIENS
  source 1..121: /mol_type=“unassigned DNA” /organism=“HOMO SAPIENS”
  variation 61: /allele=“[A/G]”
agagcttttgcagaacttgttgatgaattgaatttatggcctgggtgaggaaaaggaattagtaatggcacttgggtttttggtgtgaacaactagtgattaatcggagttcccatttaac

SEQ ID NO: 256 - DNA, 121 bp, HOMO SAPIENS
  source 1..121: /mol_type=“unassigned DNA” /organism=“HOMO SAPIENS”
  variation 61: /allele=“[A/C]”
ttctcagtgttcaccaagtctggttgtcccagtctcctatctctgtctgttcctctcctcatctgtctttatgttagttatggccctgaatataaaacagataaaggaagggtctggttga

SEQ ID NO: 257 - DNA, 121 bp, HOMO SAPIENS
  source 1..121: /mol_type=“unassigned DNA” /organism=“HOMO SAPIENS”
  variation 61: /allele=“[A/C]”
taatttctacatttctaccaaaagtcacttcatggcaatctaggctttttctatcacatgactcaaagttctccagcatcagcatctacccattatgcaattccaactcatttccacattt

SEQ ID NO: 258 - DNA, 121 bp, HOMO SAPIENS
  source 1..121: /mol_type=“unassigned DNA” /organism=“HOMO SAPIENS”
  variation 61: /allele=“[A/G]”
agtagaaaataagcacaataattttagatgtttataagttctctgaaaacaatagagtataatgatataacacgtttaggtagtttggaaaattatagtcgagtcaatgaccttagattca

SEQ ID NO: 259 - DNA, 121 bp, HOMO SAPIENS
  source 1..121: /mol_type=“unassigned DNA” /organism=“HOMO SAPIENS”
  variation 61: /allele=“[A/G]”
atatctgagtattaagaaaaattgaaaccctaagcatcaatttcttaggaacttctctgaaccattaagttgttttaaaattactttcctccatcagactcctaatcatcacctagtgata

SEQ ID NO: 260 - DNA, 121 bp, HOMO SAPIENS
  source 1..121: /mol_type=“unassigned DNA” /organism=“HOMO SAPIENS”
  variation 61: /allele=“[A/G]”
tacatacacacatacacatacatgcagatagatagatagatagataaagatctccagtcaataacctaactttacatctaagaaactgggaaaaaagcaaataaaaccccaaagcagcaga

SEQ ID NO: 261 - DNA, 121 bp, HOMO SAPIENS
  source 1..121: /mol_type=“unassigned DNA” /organism=“HOMO SAPIENS”
  variation 61: /allele=“[A/G]”
ctcgcagcgcggaactctgacgcaatccagggccgaggaaaaatgattaaaacccaacaaactcgagtgctggggtccaccaagcgggccgtcttggttagaaggcccgccccacacgtct

SEQ ID NO: 262 - DNA, 121 bp, HOMO SAPIENS
  source 1..121: /mol_type=“unassigned DNA” /organism=“HOMO SAPIENS”
  variation 61: /allele=“[A/G]”
agatcactttttattgcaatatgcaatttactggagagatgaactgctcctgctgagattattagtgtcactgcattttaagcaggtacaacacttgaactcactgcagtagcaacaggaa

SEQ ID NO: 263 - DNA, 121 bp, HOMO SAPIENS
  source 1..121: /mol_type=“unassigned DNA” /organism=“HOMO SAPIENS”
  variation 61: /allele=“[A/C]”
gggggctccaggcagagggaacagcttgtgcaaaggccctggggcaggccaagggcagagaacttaaggtatggaaaaaaaaaaaaaaaggcatggaaaggaggccagcatggctaggagc

SEQ ID NO: 264 - DNA, 121 bp, HOMO SAPIENS
  source 1..121: /mol_type=“unassigned DNA” /organism=“HOMO SAPIENS”
  variation 61: /allele=“[A/G]”
gtcagatgtgttgtggaagaataattactctattttgtgattttataaagtgtattttctatattattattaaatgtctgattacttgagataaaccagccatcctctttttttactactc

SEQ ID NO: 265 - DNA, 121 bp, HOMO SAPIENS
  source 1..121: /mol_type=“unassigned DNA” /organism=“HOMO SAPIENS”
  variation 61: /allele=“[A/C]”
taaatggtgggtgctattttgttgctgttaggtctattttcttcatctgttatttcgcataacagtaaaacagatactcagatgacttatataactttcattagtttcattaggtggtgtc

SEQ ID NO: 266 - DNA, 121 bp, HOMO SAPIENS
  source 1..121: /mol_type=“unassigned DNA” /organism=“HOMO SAPIENS”
  variation 61: /allele=“[A/G]”
gaacgaatgagaagtgaggaaacgcttagcgcaaaaggaaaaagagagaaagacatacagaaacaaggttacgcggaggccggcgaaaagcgattccccgctcccccaggccaagggcccc

SEQ ID NO: 267 - DNA, 121 bp, HOMO SAPIENS
  source 1..121: /mol_type=“unassigned DNA” /organism=“HOMO SAPIENS”
  variation 61: /allele=“[A/G]”
tacaaggtcaggctcaacggaagtgaccgtcccacagttatgcagcactaagtcaatggcacatttgcttgtgtgttggttacatttgtaactcaaagctgatgccttaagaaggttaggg

SEQ ID NO: 268 - DNA, 121 bp, HOMO SAPIENS
  source 1..121: /mol_type=“unassigned DNA” /organism=“HOMO SAPIENS”
  variation 61: /allele=“[A/G]”
cctgaagtgtctgcttagccgcgcacggggtatttatatctcaggctttggagaactatcaggtttggggcccggctagggcgtgcgtgttcacgctgggacctgtcacaacctggtctta

SEQ ID NO: 269 - DNA, 121 bp, HOMO SAPIENS
  source 1..121: /mol_type=“unassigned DNA” /organism=“HOMO SAPIENS”
  variation 61: /allele=“[A/G]”
atggatcttgaaggggaccgcaatggaggagcaaagaagaagaacttttttaaactgaacaataaaaggtaactagcttgtttcattttcatagtttacatagttgcgagatttgagtaat

SEQ ID NO: 270 - DNA, 121 bp, HOMO SAPIENS
  source 1..121: /mol_type=“unassigned DNA” /organism=“HOMO SAPIENS”
  variation 61: /allele=“[A/C]”
gtttaaaacaacactgtgatttcacaatttctgtggatcatgaattaaggagcagcttagatggatggtcctgggttgagtccttcctgtggctgcagtcaagaagtcagctggggctaca

SEQ ID NO: 271 - DNA, 121 bp, HOMO SAPIENS
  source 1..121: /mol_type=“unassigned DNA” /organism=“HOMO SAPIENS”
  variation 61: /allele=“[A/C]”
tggctactctaatttttcaatggtaaacagaccagagttattctaagaaattatgaaaagaaatccatttcgaagtcttaaagcaaatttagagactgacaattgaaaatacatcttcttt

SEQ ID NO: 272 - DNA, 121 bp, HOMO SAPIENS
  source 1..121: /mol_type=“unassigned DNA” /organism=“HOMO SAPIENS”
  variation 61: /allele=“[A/G]”
gcttgaggccaggatttcaagacttgcctgagcaacataatgagatgccctctctcaaaaatttaattaattaatttaaaaagaaaatcccagctactcaggaagctgagatgggaagatc

SEQ ID NO: 273 - DNA, 121 bp, HOMO SAPIENS
  source 1..121: /mol_type=“unassigned DNA” /organism=“HOMO SAPIENS”
  variation 61: /allele=“[A/C]”
aacttaaatcagcaagcagaaaacaaacaacttcattaaaaatgagcagaggacctgaacaaacacttctcagaagaaaacattcttatggccaacaaatacatgaaaaaagcctcatcac

SEQ ID NO: 274 - DNA, 121 bp, HOMO SAPIENS
  source 1..121: /mol_type=“unassigned DNA” /organism=“HOMO SAPIENS”
  variation 61: /allele=“[A/G]”
caagggcagcatgttgcttcatgagcggttctggacagggatggtgggagatgttgctagagggaattgtggccctgggcttagaaacaaaggggcaagaaggtctcagaagctggggcct

SEQ ID NO: 275 - DNA, 50 bp, Artificial Sequence
  source 1..50: /mol_type=“unassigned DNA” /note=“Synthetic” /organism=“Artificial Sequence”
aggactctatggcttccttcatgtcatcgtccactctgccaagggattta

SEQ ID NO: 276 - DNA, 50 bp, Artificial Sequence
  source 1..50: /mol_type=“unassigned DNA” /note=“Synthetic” /organism=“Artificial Sequence”
acgacagagatgaattgaggggacaaatgtcagagctcacagacgactgt

SEQ ID NO: 277 - DNA, 50 bp, Artificial Sequence
  source 1..50: /mol_type=“unassigned DNA” /note=“Synthetic” /organism=“Artificial Sequence”
tcagaagcctatttttaatgtcattccaccaattcccgttggttccgaaa

SEQ ID NO: 278 - DNA, 50 bp, Artificial Sequence
  source 1..50: /mol_type=“unassigned DNA” /note=“Synthetic” /organism=“Artificial Sequence”
tcacagtagctcctcctcttagggttttatagaagtccatcacatctccc

SEQ ID NO: 279 - DNA, 50 bp, Artificial Sequence
  source 1..50: /mol_type=“unassigned DNA” /note=“Synthetic” /organism=“Artificial Sequence”
ttgcattctctcctagcactccagtaaataaactatagtcctggtcaagt

SEQ ID NO: 280 - DNA, 50 bp, Artificial Sequence
  source 1..50: /mol_type=“unassigned DNA” /note=“Synthetic” /organism=“Artificial Sequence”
attcccaatattcgtatatgtatttataaattacataatgggcagggtgc

SEQ ID NO: 281 - DNA, 50 bp, Artificial Sequence
  source 1..50: /mol_type=“unassigned DNA” /note=“Synthetic” /organism=“Artificial Sequence”
agtgctttttgagaggtatgaacttactccatactacttacatctgctaa

SEQ ID NO: 282 - DNA, 50 bp, Artificial Sequence
  source 1..50: /mol_type=“unassigned DNA” /note=“Synthetic” /organism=“Artificial Sequence”
tgacttctaattacaggcaaaatcaaccttaataagaacaggcgttacta

SEQ ID NO: 283 - DNA, 50 bp, Artificial Sequence
  source 1..50: /mol_type=“unassigned DNA” /note=“Synthetic” /organism=“Artificial Sequence”
tactattctgttcataaggtacacttctttttagggcacactaccttggg

SEQ ID NO: 284 - DNA, 50 bp, Artificial Sequence
  source 1..50: /mol_type=“unassigned DNA” /note=“Synthetic” /organism=“Artificial Sequence”
aggtgggataaacagaagcagcataacgtgtcttgatgtgtgctgtttag

SEQ ID NO: 285 - DNA, 50 bp, Artificial Sequence
  source 1..50: /mol_type=“unassigned DNA” /note=“Synthetic” /organism=“Artificial Sequence”
ttgtcatgcagcaggttaactatgctttctggagaaggtgtcagccaact

SEQ ID NO: 286 - DNA, 50 bp, Artificial Sequence
  source 1..50: /mol_type=“unassigned DNA” /note=“Synthetic” /organism=“Artificial Sequence”
ccctccagctgaatgatttttgtctgtgcctggcccagtccctgagtcca

SEQ ID NO: 287 - DNA, 50 bp, Artificial Sequence
  source 1..50: /mol_type=“unassigned DNA” /note=“Synthetic” /organism=“Artificial Sequence”
gtgaaaatgcattttccccctattccttctggaaagcaacattagggtcc

SEQ ID NO: 288 - DNA, 50 bp, Artificial Sequence
  source 1..50: /mol_type=“unassigned DNA” /note=“Synthetic” /organism=“Artificial Sequence”
tgctcaccacagtcctcatatccttaaagggacaccctagtgattactga

SEQ ID NO: 289 - DNA, 50 bp, Artificial Sequence
  source 1..50: /mol_type=“unassigned DNA” /note=“Synthetic” /organism=“Artificial Sequence”
cagtcccgcctgcttggatctgacgagcgtgccgattcggtccgaaaatc

SEQ ID NO: 290 - DNA, 50 bp, Artificial Sequence
  source 1..50: /mol_type=“unassigned DNA” /note=“Synthetic” /organism=“Artificial Sequence”
agagcactaactctggagagtaaggatctgagtgtaagtcaccgctgtgt

SEQ ID NO: 291 - DNA, 50 bp, Artificial Sequence
  source 1..50: /mol_type=“unassigned DNA” /note=“Synthetic” /organism=“Artificial Sequence”
aagcagtccacagcagtctgagctggcaggtcatggagcagcccccaaac

SEQ ID NO: 292 - DNA, 50 bp, Artificial Sequence
  source 1..50: /mol_type=“unassigned DNA” /note=“Synthetic” /organism=“Artificial Sequence”
gtaaagaacagggggagataatgatcagtaaaatcacagcagggtgaggg

SEQ ID NO: 293 - DNA, 50 bp, Artificial Sequence
  source 1..50: /mol_type=“unassigned DNA” /note=“Synthetic” /organism=“Artificial Sequence”
tatttaggtagttgaccacttcagcattctaggtacaataacgttagccc

SEQ ID NO: 294 - DNA, 50 bp, Artificial Sequence
  source 1..50: /mol_type=“unassigned DNA” /note=“Synthetic” /organism=“Artificial Sequence”
agaacaaagccaggacaaggtacaaggtgaccccagcaaatttccttttc

SEQ ID NO: 295 - DNA, 50 bp, Artificial Sequence
  source 1..50: /mol_type=“unassigned DNA” /note=“Synthetic” /organism=“Artificial Sequence”
tgctagaagcttatcaactgcattaatctttttaaaaacacttttagttt

SEQ ID NO: 296 - DNA, 50 bp, Artificial Sequence
  source 1..50: /mol_type=“unassigned DNA” /note=“Synthetic” /organism=“Artificial Sequence”
tctcaccttctccaggtgcacggtaggtgctgtgtaaattaacgacttca

SEQ ID NO: 297 - DNA, 50 bp, Artificial Sequence
  source 1..50: /mol_type=“unassigned DNA” /note=“Synthetic” /organism=“Artificial Sequence”
gcctcctgctagacaattagctttatccatgagttaccaaagagggagcc

SEQ ID NO: 298 - DNA, 50 bp, Artificial Sequence
  source 1..50: /mol_type=“unassigned DNA” /note=“Synthetic” /organism=“Artificial Sequence”
accaaaatctataaacaataaggaactgtggttgtttgctgcaaataact

SEQ ID NO: 299 - DNA, 50 bp, Artificial Sequence
  source 1..50: /mol_type=“unassigned DNA” /note=“Synthetic” /organism=“Artificial Sequence”
tcaagagttgggaatgatgagggcatgtactgtgactggcacacagaatg

SEQ ID NO: 300 - DNA, 50 bp, Artificial Sequence
  source 1..50: /mol_type=“unassigned DNA” /note=“Synthetic” /organism=“Artificial Sequence”
agtgcctactatgtgctagtccctagtgacatgagagtgaggaaggcagt

SEQ ID NO: 301 - DNA, 50 bp, Artificial Sequence
  source 1..50: /mol_type=“unassigned DNA” /note=“Synthetic” /organism=“Artificial Sequence”
ccttatttcaaggtcggggtcaaggtggtcaaaagaactgttttgctctc

SEQ ID NO: 302 - DNA, 50 bp, Artificial Sequence
  source 1..50: /mol_type=“unassigned DNA” /note=“Synthetic” /organism=“Artificial Sequence”
aagggtatttatacctttgcctttccgcctcaaccattggaacctgggac

SEQ ID NO: 303 - DNA, 50 bp, Artificial Sequence
  source 1..50: /mol_type=“unassigned DNA” /note=“Synthetic” /organism=“Artificial Sequence”
agcctctctgggtccttggggagcatgaggatcctgcagaaagcagagtg

SEQ ID NO: 304 - DNA, 50 bp, Artificial Sequence
  source 1..50: /mol_type=“unassigned DNA” /note=“Synthetic” /organism=“Artificial Sequence”
atgctctctgaacactatgacctctgattatttatcaacctccaagagct

SEQ ID NO: 305 - DNA, 50 bp, Artificial Sequence
  source 1..50: /mol_type=“unassigned DNA” /note=“Synthetic” /organism=“Artificial Sequence”
cccctcttctgtgagagccaaacagagcccttcctgagtcccatccattc

SEQ ID NO: 306 - DNA, 50 bp, Artificial Sequence
  source 1..50: /mol_type=“unassigned DNA” /note=“Synthetic” /organism=“Artificial Sequence”
tctgggtccttttcattgctctacaaagaatcctttcttcctcccaggcc

SEQ ID NO: 307 - DNA, 50 bp, Artificial Sequence
  source 1..50: /mol_type=“unassigned DNA” /note=“Synthetic” /organism=“Artificial Sequence”
catcaatgcccacgctacacgaggcatactagacagtcgctgcctaagcc

SEQ ID NO: 308 - DNA, 50 bp, Artificial Sequence
  source 1..50: /mol_type=“unassigned DNA” /note=“Synthetic” /organism=“Artificial Sequence”
tcaagagtaacagtatgccctgcattaacagggataatatataagaaaaa

SEQ ID NO: 309 - DNA, 50 bp, Artificial Sequence
  source 1..50: /mol_type=“unassigned DNA” /note=“Synthetic” /organism=“Artificial Sequence”
gaatttattactcctgggaggattctgctcaccactggcaactatgacca

SEQ ID NO: 310 - DNA, 50 bp, Artificial Sequence
  source 1..50: /mol_type=“unassigned DNA” /note=“Synthetic” /organism=“Artificial Sequence”
catcatgatgtaatgtagtcatatagactaggacacttagattagccccc

SEQ ID NO: 311 - DNA, 50 bp, Artificial Sequence
  source 1..50: /mol_type=“unassigned DNA” /note=“Synthetic” /organism=“Artificial Sequence”
ggttttagtattgcaatgtggaatccaaaactgttatcaatgaacttttg

SEQ ID NO: 312 - DNA, 50 bp, Artificial Sequence
  source 1..50: /mol_type=“unassigned DNA” /note=“Synthetic” /organism=“Artificial Sequence”
tgggtgagggaaccgttagtgccatcctgaggccccgtgtcaggaaatat

SEQ ID NO: 313 - DNA, 50 bp, Artificial Sequence
  source 1..50: /mol_type=“unassigned DNA” /note=“Synthetic” /organism=“Artificial Sequence”
actgaactccccatcacaaatctgtatgctttattagaaagtaaaactct

SEQ ID NO: 314 - DNA, 50 bp, Artificial Sequence
  source 1..50: /mol_type=“unassigned DNA” /note=“Synthetic” /organism=“Artificial Sequence”
agtaaaacagacgacgggatccccagacgctgcacatcagcaccaggagc

SEQ ID NO: 315 - DNA, 50 bp, Artificial Sequence
  source 1..50: /mol_type=“unassigned DNA” /note=“Synthetic” /organism=“Artificial Sequence”
cacactaatattcaaacatccttgacctcatctcatataaataaatccaa

SEQ ID NO: 316 - DNA, 50 bp, Artificial Sequence
  source 1..50: /mol_type=“unassigned DNA” /note=“Synthetic” /organism=“Artificial Sequence”
ccaagattctggatgtctttaaggtaacaagtgtccatgttgttccttga

SEQ ID NO: 317 - DNA, 50 bp, Artificial Sequence
  source 1..50: /mol_type=“unassigned DNA” /note=“Synthetic” /organism=“Artificial Sequence”
gaagcgaaaatagctatgcaccaaatctctgcaggcatttcattgagtac

SEQ ID NO: 318 - DNA, 50 bp, Artificial Sequence
  source 1..50: /mol_type=“unassigned DNA” /note=“Synthetic” /organism=“Artificial Sequence”
tgaatgacagtgttgttgattagttcaagctcttgcctttctctaaactt

SEQ ID NO: 319 - DNA, 50 bp, Artificial Sequence
  source 1..50: /mol_type=“unassigned DNA” /note=“Synthetic” /organism=“Artificial Sequence”
gatcttagccaaggcaggaaagcacacgatcaggtaacctccagattcac

SEQ ID NO: 320 - DNA, 50 bp, Artificial Sequence
  source 1..50: /mol_type=“unassigned DNA” /note=“Synthetic” /organism=“Artificial Sequence”
ttactcgcattaactctttcaatttcacaacaaatctaagaaaaatgcaa

SEQ ID NO: 321 - DNA, 50 bp, Artificial Sequence
  source 1..50: /mol_type=“unassigned DNA” /note=“Synthetic” /organism=“Artificial Sequence”
agtctaaaacactatcatctcctcctggattactgcaacagactccttct

SEQ ID NO: 322 - DNA, 50 bp, Artificial Sequence
  source 1..50: /mol_type=“unassigned DNA” /note=“Synthetic” /organism=“Artificial Sequence”
tctgccctaaatattccctgttcggtggggtttggcggtccagcagccct

SEQ ID NO: 323 - DNA, 50 bp, Artificial Sequence
  source 1..50: /mol_type=“unassigned DNA” /note=“Synthetic” /organism=“Artificial Sequence”
ccatgcgtgttggaagtatttctcttgttctcctgcttttagaaagccat

SEQ ID NO: 324 - DNA, 50 bp, Artificial Sequence
  source 1..50: /mol_type=“unassigned DNA” /note=“Synthetic” /organism=“Artificial Sequence”
cttctgaccctcgccgtcctagaaccaacggcccctcggtgtctggtcct

SEQ ID NO: 325 - DNA, 50 bp, Artificial Sequence
  source 1..50: /mol_type=“unassigned DNA” /note=“Synthetic” /organism=“Artificial Sequence”
aaagctctaataccacctaaaaccatttctgttctctacctctgtcatta

SEQ ID NO: 326 - DNA, 50 bp, Artificial Sequence
  source 1..50: /mol_type=“unassigned DNA” /note=“Synthetic” /organism=“Artificial Sequence”
acaggttctatatctttagatggtaaattaaaaattcctggctgaatttg

SEQ ID NO: 327 - DNA, 50 bp, Artificial Sequence
  source 1..50: /mol_type=“unassigned DNA” /note=“Synthetic” /organism=“Artificial Sequence”
aatgtgagtagattccaacctttatccattccattcacatttaccttctc

SEQ ID NO: 328 - DNA, 50 bp, Artificial Sequence
  source 1..50: /mol_type=“unassigned DNA” /note=“Synthetic” /organism=“Artificial Sequence”
ttgtttaaagctgctgcaggtatactctttggaggctaataataaagaac

SEQ ID NO: 329 - DNA, 50 bp, Artificial Sequence
  source 1..50: /mol_type=“unassigned DNA” /note=“Synthetic” /organism=“Artificial Sequence”
tggagtagtcttcttctagcccttgcatgacctctcttacttcacccata

SEQ ID NO: 330 - DNA, 50 bp, Artificial Sequence
  source 1..50: /mol_type=“unassigned DNA” /note=“Synthetic” /organism=“Artificial Sequence”
cttccacctgctgcactccaatatagccactatgttcggctatatatata

SEQ ID NO: 331 - DNA, 50 bp, Artificial Sequence
  source 1..50: /mol_type=“unassigned DNA” /note=“Synthetic” /organism=“Artificial Sequence”
tagagagtaatgtggtgggtgtgctgtgtcagaaaggcttcactagcagt

SEQ ID NO: 332 - DNA, 50 bp, Artificial Sequence
  source 1..50: /mol_type=“unassigned DNA” /note=“Synthetic” /organism=“Artificial Sequence”
ctaatttgatcaatgaatcactgctagcatgtgaatgtccataatggata

SEQ ID NO: 333 - DNA, 50 bp, Artificial Sequence
  source 1..50: /mol_type=“unassigned DNA” /note=“Synthetic” /organism=“Artificial Sequence”
ttattagaggtaaacatagagataagcccctaataaaatagtagctggag

SEQ ID NO: 334 - DNA, 50 bp, Artificial Sequence
  source 1..50: /mol_type=“unassigned DNA” /note=“Synthetic” /organism=“Artificial Sequence”
agtgttaattctctaagaggaaaatgtcatttctccaaaacaaaacttta

SEQ ID NO: 335 - DNA, 50 bp, Artificial Sequence
  source 1..50: /mol_type=“unassigned DNA” /note=“Synthetic” /organism=“Artificial Sequence”
gtaacaaggttacctccagaaaaaaaggctattgctgaacagaggctttc

SEQ ID NO: 336 - DNA, 50 bp, Artificial Sequence
  source 1..50: /mol_type=“unassigned DNA” /note=“Synthetic” /organism=“Artificial Sequence”
aagagagaaaaatatttttaagtgaaaaggaacaaaactattctatacga

SEQ ID NO: 337 - DNA, 50 bp, Artificial Sequence
  source 1..50: /mol_type=“unassigned DNA” /note=“Synthetic” /organism=“Artificial Sequence”
ggctcacaccgagatcaatccatgatgacagcacttcatggcccgtctca

SEQ ID NO: 338 - DNA, 50 bp, Artificial Sequence
  source 1..50: /mol_type=“unassigned DNA” /note=“Synthetic” /organism=“Artificial Sequence”
gataatctaattcatctaacttgctttacaaatgaggaaactgataatcc

SEQ ID NO: 339 - DNA, 50 bp, Artificial Sequence
  source 1..50: /mol_type=“unassigned DNA” /note=“Synthetic” /organism=“Artificial Sequence”
gtggaccctttgagtggttacagacgggcctcaggattggtgttatttaa

SEQ ID NO: 340 - DNA, 50 bp, Artificial Sequence
  source 1..50: /mol_type=“unassigned DNA” /note=“Synthetic” /organism=“Artificial Sequence”
aacaggggccactgtctgtttcccatggtatctatagggcctggtggaca

SEQ ID NO: 341 - DNA, 50 bp, Artificial Sequence
  source 1..50: /mol_type=“unassigned DNA” /note=“Synthetic” /organism=“Artificial Sequence”
aggggtcaagatacaaggagtcaccaaagaatgcagaagagacaagttca

SEQ ID NO: 342 - DNA, 50 bp, Artificial Sequence
  source 1..50: /mol_type=“unassigned DNA” /note=“Synthetic” /organism=“Artificial Sequence”
ccttttctaagaccaatattaacaagaattagtagtagaatgttcttatg

SEQ ID NO: 343 - DNA, 50 bp, Artificial Sequence
  source 1..50: /mol_type=“unassigned DNA” /note=“Synthetic” /organism=“Artificial Sequence”
tgttgctaatcccaaccagcatgatttacgggaagtaaatcatctatgac

SEQ ID NO: 344 - DNA, 50 bp, Artificial Sequence
  source 1..50: /mol_type=“unassigned DNA” /note=“Synthetic” /organism=“Artificial Sequence”
gcctgtctcacaaacattgggttctatagacgctcctagattgcattttc

SEQ ID NO: 345 - DNA, 50 bp, Artificial Sequence
  source 1..50: /mol_type=“unassigned DNA” /note=“Synthetic” /organism=“Artificial Sequence”
cccagtgccttgacagggtatggggggacctgcatgactagcattaaatg

SEQ ID NO: 346 - DNA, 50 bp, Artificial Sequence
  source 1..50: /mol_type=“unassigned DNA” /note=“Synthetic” /organism=“Artificial Sequence”
taaccagggatctgtgcgttttgctataattcagaaagtagcagactact

SEQ ID NO: 347 - DNA, 50 bp, Artificial Sequence
  source 1..50: /mol_type=“unassigned DNA” /note=“Synthetic” /organism=“Artificial Sequence”
aaaagtcggttcgagaacccaggtggaaaatagattgagggaagcaaaac

SEQ ID NO: 348 - DNA, 50 bp, Artificial Sequence
  source 1..50: /mol_type=“unassigned DNA” /note=“Synthetic” /organism=“Artificial Sequence”
gagtaagagttaatcacttccactgtgcacttgtttattccaagtagaaa

SEQ ID NO: 349 - DNA, 50 bp, Artificial Sequence
  source 1..50: /mol_type=“unassigned DNA” /note=“Synthetic” /organism=“Artificial Sequence”
ctctggacatcttcagagggtcccactttagacttcactgatctcttttt

SEQ ID NO: 350 - DNA, 50 bp, Artificial Sequence
  source 1..50: /mol_type=“unassigned DNA” /note=“Synthetic” /organism=“Artificial Sequence”
tcacactttacatttattatttccagtaagggatatagctaagatagtta

SEQ ID NO: 351 - DNA, 50 bp, Artificial Sequence
  source 1..50: /mol_type=“unassigned DNA” /note=“Synthetic” /organism=“Artificial Sequence”
cagtttgatgaatggcaaaatcgttcaaatggaaaagaggagagagatag

SEQ ID NO: 352 - DNA, 50 bp, Artificial Sequence
  source 1..50: /mol_type=“unassigned DNA” /note=“Synthetic” /organism=“Artificial Sequence”
ttcgtaattaaaggaacagagtgagagacatcatcaagtggagagaaatc

SEQ ID NO: 353 - DNA, 50 bp, Artificial Sequence
  source 1..50: /mol_type=“unassigned DNA” /note=“Synthetic” /organism=“Artificial Sequence”
agccagggttgaagtcactcacgggtcctctccgagaactcgagtggtga

SEQ ID NO: 354 - DNA, 50 bp, Artificial Sequence
  source 1..50: /mol_type=“unassigned DNA” /note=“Synthetic” /organism=“Artificial Sequence”
caaaggtgatatgcattttaaatttgatagttattgcccaactgtcttta

SEQ ID NO: 355 - DNA, 50 bp, Artificial Sequence
  source 1..50: /mol_type=“unassigned DNA” /note=“Synthetic” /organism=“Artificial Sequence”
ccctcaggctgcttgttaccgtggaagcttcctgaactctctccagaccc

SEQ ID NO: 356 - DNA, 50 bp, Artificial Sequence
  source 1..50: /mol_type=“unassigned DNA” /note=“Synthetic” /organism=“Artificial Sequence”
ttttcatttttctcttcccaacccaatcccctctctctaaatcttggtat

SEQ ID NO: 357 - DNA, 50 bp, Artificial Sequence
  source 1..50: /mol_type=“unassigned DNA” /note=“Synthetic” /organism=“Artificial Sequence”
ttcaatatatgttttctgaacaccttctgtgttcaaggcaccatgctggg

SEQ ID NO: 358 - DNA, 50 bp, Artificial Sequence
  source 1..50: /mol_type=“unassigned DNA” /note=“Synthetic” /organism=“Artificial Sequence”
cccttgcatgttcaccttgttatgtgtactttcatctcaattgccagtta

SEQ ID NO: 359 - DNA, 50 bp, Artificial Sequence
  source 1..50: /mol_type=“unassigned DNA” /note=“Synthetic” /organism=“Artificial Sequence”
aaagtatctccccaaatcattcccaaacactacaaaggtagtgccatcag

SEQ ID NO: 360 - DNA, 50 bp, Artificial Sequence
  source 1..50: /mol_type=“unassigned DNA” /note=“Synthetic” /organism=“Artificial Sequence”
tgctctaaaactaatttgcttgaagtgtacagaatggaattcgggaagga

SEQ ID NO: 361 - DNA, 50 bp, Artificial Sequence
  source 1..50: /mol_type=“unassigned DNA” /note=“Synthetic” /organism=“Artificial Sequence”
atcacttttccatgaaattgtctttgcattagcaaaatgaatcaagcata

SEQ ID NO: 362 - DNA, 50 bp, Artificial Sequence
  source 1..50: /mol_type=“unassigned DNA” /note=“Synthetic” /organism=“Artificial Sequence”
ttggtgatgctgatagttggagatacccagacagataaggtatattgccc

SEQ ID NO: 363 - DNA, 50 bp, Artificial Sequence
  source 1..50: /mol_type=“unassigned DNA” /note=“Synthetic” /organism=“Artificial Sequence”
atcaatatgactggtgtccttcaggaatgtggtagcacagtgaaaaaggt

SEQ ID NO: 364 - DNA, 50 bp, Artificial Sequence
  source 1..50: /mol_type=“unassigned DNA” /note=“Synthetic” /organism=“Artificial Sequence”
gcagtaggggactggctgccgagggggcatctagattgagataggtggga

SEQ ID NO: 365 - DNA, 50 bp, Artificial Sequence
  source 1..50: /mol_type=“unassigned DNA” /note=“Synthetic” /organism=“Artificial Sequence”
attggcaaaagtgctcattctggaaaaacaaagaagtgagaaagtggatg

SEQ ID NO: 366 - DNA, 50 bp, Artificial Sequence
  source 1..50: /mol_type=“unassigned DNA” /note=“Synthetic” /organism=“Artificial Sequence”
attctaaagctttgtgtggtccaccatgatcaccttttcctgcttccccc

SEQ ID NO: 367 - DNA, 50 bp, Artificial Sequence
  source 1..50: /mol_type=“unassigned DNA” /note=“Synthetic” /organism=“Artificial Sequence”
gctccattttctttgaggtacatcaacatcaataacagatcaatggaccc

SEQ ID NO: 368 - DNA, 50 bp, Artificial Sequence
  source 1..50: /mol_type=“unassigned DNA” /note=“Synthetic” /organism=“Artificial Sequence”
agcctgacctcatggcttagctgtgcctcctggacaccatccctctctgc

SEQ ID NO: 369 - DNA, 50 bp, Artificial Sequence
  source 1..50: /mol_type=“unassigned DNA” /note=“Synthetic” /organism=“Artificial Sequence”
ttctgaaagtcacagcccagggattcagacccactaaaaaaaactgagat

SEQ ID NO: 370 - DNA, 50 bp, Artificial Sequence
  source 1..50: /mol_type=“unassigned DNA” /note=“Synthetic” /organism=“Artificial Sequence”
actacattacatcatgatgtattgattgcctctggcctaggaatctgcag

SEQ ID NO: 371 - DNA, 50 bp, Artificial Sequence
  source 1..50: /mol_type=“unassigned DNA” /note=“Synthetic” /organism=“Artificial Sequence”
ccactcatatgtctgttctcactcagaggtgaggccctgtgtcttcagcc

SEQ ID NO: 372 - DNA, 50 bp, Artificial Sequence
  source 1..50: /mol_type=“unassigned DNA” /note=“Synthetic” /organism=“Artificial Sequence”
gggggacagagaagtaacgtcacaagattttaagcttgggccagatatgg

SEQ ID NO: 373 - DNA, 50 bp, Artificial Sequence
  source 1..50: /mol_type=“unassigned DNA” /note=“Synthetic” /organism=“Artificial Sequence”
aagtagagcagaaagggcaagcagagaactagacagagaagacagatgac

SEQ ID NO: 374 - DNA, 50 bp, Artificial Sequence
  source 1..50: /mol_type=“unassigned DNA” /note=“Synthetic” /organism=“Artificial Sequence”
tggctgcctctagggcaagaagactggggatatcaccatgggctcaatgt

SEQ ID NO: 375 - DNA, 50 bp, Artificial Sequence
  source 1..50: /mol_type=“unassigned DNA” /note=“Synthetic” /organism=“Artificial Sequence”
ccaagtccttctacctccctgggtgagggaaccgttagtgccatcctgag

SEQ ID NO: 376 - DNA, 50 bp, Artificial Sequence
  source 1..50: /mol_type=“unassigned DNA” /note=“Synthetic” /organism=“Artificial Sequence”
aatcttggggaatctgagtttattagaggaatgtagggaggaagcaggct

SEQ ID NO: 377 - DNA, 50 bp, Artificial Sequence
  source 1..50: /mol_type=“unassigned DNA” /note=“Synthetic” /organism=“Artificial Sequence”
tatcatatgctctagtgacttcatcaagacagtctaaaggaagatgggcc

SEQ ID NO: 378 - DNA, 50 bp, Artificial Sequence
  source 1..50: /mol_type=“unassigned DNA” /note=“Synthetic” /organism=“Artificial Sequence”
cagaaacacctttaatgtttttatttctatgaatattctcctaatgatta

SEQ ID NO: 379 - DNA, 50 bp, Artificial Sequence
  source 1..50: /mol_type=“unassigned DNA” /note=“Synthetic” /organism=“Artificial Sequence”
ttaaaatgagatcccttccaacatgctttgctgagccagatttataaaat

SEQ ID NO: 380 - DNA, 50 bp, Artificial Sequence
  source 1..50: /mol_type=“unassigned DNA” /note=“Synthetic” /organism=“Artificial Sequence”
tagtacagtaagggcaaagggcactgcaattgctattaaactgtaagaag

SEQ ID NO: 381 - DNA, 50 bp, Artificial Sequence
  source 1..50: /mol_type=“unassigned DNA” /note=“Synthetic” /organism=“Artificial Sequence”
atcccccggaactgggggaatttccaggcacatgaggctctgtcaaccca

SEQ ID NO: 382 - DNA, 50 bp, Artificial Sequence
  source 1..50: /mol_type=“unassigned DNA” /note=“Synthetic” /organism=“Artificial Sequence”
agccacttaaaataaatttttccagcagttattcatttagtgccaaaata

SEQ ID NO: 383 - DNA, 50 bp, Artificial Sequence
  source 1..50: /mol_type=“unassigned DNA” /note=“Synthetic” /organism=“Artificial Sequence”
gcaggggcacatgcaattgccatttaaaaatgaggtctggcatggccaga

SEQ ID NO: 384 - DNA, 50 bp, Artificial Sequence
  source 1..50: /mol_type=“unassigned DNA” /note=“Synthetic” /organism=“Artificial Sequence”
gtaccacagctcccagctgcatgtactttaaaaatgtgtctaagccaggc

SEQ ID NO: 385 - DNA, 50 bp, Artificial Sequence
  source 1..50: /mol_type=“unassigned DNA” /note=“Synthetic” /organism=“Artificial Sequence”
tgcaaacagaaaaatcagaacctgctcatgctgccatattaataggaacc

SEQ ID NO: 386 - DNA, 50 bp, Artificial Sequence
  source 1..50: /mol_type=“unassigned DNA” /note=“Synthetic” /organism=“Artificial Sequence”
taactacacactcaaggctccctctcaaagtctcaaaccttacaacttcc

SEQ ID NO: 387 - DNA, 50 bp, Artificial Sequence
  source 1..50: /mol_type=“unassigned DNA” /note=“Synthetic” /organism=“Artificial Sequence”
aatacagccatgcgctacctactggcattcccgtcagtgcgtacacgatc

SEQ ID NO: 388 - DNA, 50 bp, Artificial Sequence
  source 1..50: /mol_type=“unassigned DNA” /note=“Synthetic” /organism=“Artificial Sequence”
aactgctttcctcattggcttggtctccatagtgattcattttgctgtaa

SEQ ID NO: 389 - DNA, 50 bp, Artificial Sequence
  source 1..50: /mol_type=“unassigned DNA” /note=“Synthetic” /organism=“Artificial Sequence”
tggaaatttttttgtaattagaaatgacctaaaggatagtttctattctt

SEQ ID NO: 390 - DNA, 50 bp, Artificial Sequence
  source 1..50: /mol_type=“unassigned DNA” /note=“Synthetic” /organism=“Artificial Sequence”
attgatttttatgtcagcaatcttccaatcttgttaattctaaaatactt

SEQ ID NO: 391 - DNA, 50 bp, Artificial Sequence
  source 1..50: /mol_type=“unassigned DNA” /note=“Synthetic” /organism=“Artificial Sequence”
gcctaagctgaacctgagaggtgaggaaaacagaccaagctgaccaaacc

SEQ ID NO: 392 - DNA, 50 bp, Artificial Sequence
  source 1..50: /mol_type=“unassigned DNA” /note=“Synthetic” /organism=“Artificial Sequence”
gcgaactgtggagtatctcagtaagagtgttaggaggaatattttatagg

SEQ ID NO: 393 - DNA, 50 bp, Artificial Sequence
  source 1..50: /mol_type=“unassigned DNA” /note=“Synthetic” /organism=“Artificial Sequence”
acaacaacaaatctcaaacaactgttctgccaatggggtggagcaccttt

SEQ ID NO: 394 - DNA, 50 bp, Artificial Sequence
  source 1..50: /mol_type=“unassigned DNA” /note=“Synthetic” /organism=“Artificial Sequence”
tgatgattttccagcatggcaatggtaaagctgcaaataaaaagcagcca

SEQ ID NO: 395 - DNA, 50 bp, Artificial Sequence
  source 1..50: /mol_type=“unassigned DNA” /note=“Synthetic” /organism=“Artificial Sequence”
ttcttttctccaagcaaaagagagaagagtttatttcattctcagcagct

SEQ ID NO: 396 - DNA, 50 bp, Artificial Sequence
  source 1..50: /mol_type=“unassigned DNA” /note=“Synthetic” /organism=“Artificial Sequence”
ggcaaaagcagagatgtgagctgtaaatttgaatgaaggaccagatagaa

SEQ ID NO: 397 - DNA, 50 bp, Artificial Sequence
  source 1..50: /mol_type=“unassigned DNA” /note=“Synthetic” /organism=“Artificial Sequence”
taggaacataaaagttcagatgttagtaggactaataaaaagttattgtt

SEQ ID NO: 398 - DNA, 50 bp, Artificial Sequence
  source 1..50: /mol_type=“unassigned DNA” /note=“Synthetic” /organism=“Artificial Sequence”
tttttcaggtctagcttaaccaaaacacttaaaactgttaccaaaaaact

SEQ ID NO: 399 - DNA, 50 bp, Artificial Sequence
  source 1..50: /mol_type=“unassigned DNA” /note=“Synthetic” /organism=“Artificial Sequence”
caaataaataaactttaaagaaatggccaacttgggaaggacattaggcc

SEQ ID NO: 400 - DNA, 50 bp, Artificial Sequence
  source 1..50: /mol_type=“unassigned DNA” /note=“Synthetic” /organism=“Artificial Sequence”
cagtccaacaaccagttccagaagatctcagaggtaggccgctccccaca

SEQ ID NO: 401 - DNA, 50 bp, Artificial Sequence
  source 1..50: /mol_type=“unassigned DNA” /note=“Synthetic” /organism=“Artificial Sequence”
tgaaaatgttgtctggacaagcactgaaagataagaaagaactagaaggt

SEQ ID NO: 402 - DNA, 50 bp, Artificial Sequence
  source 1..50: /mol_type=“unassigned DNA” /note=“Synthetic” /organism=“Artificial Sequence”
gattcatttttacatgtttatttttaatggagactaaagagacataaatg

SEQ ID NO: 403 - DNA, 50 bp, Artificial Sequence
  source 1..50: /mol_type=“unassigned DNA” /note=“Synthetic” /organism=“Artificial Sequence”
tcttgattcaattggaagtaactgagaggtatatcacatgttgtgattca

SEQ ID NO: 404 - DNA, 50 bp, Artificial Sequence
  source 1..50: /mol_type=“unassigned DNA” /note=“Synthetic” /organism=“Artificial Sequence”
tgctccataacacaaataatttcattcttcttcctttcttgccgagtagt

SEQ ID NO: 405 - DNA, 50 bp, Artificial Sequence
  source 1..50: /mol_type=“unassigned DNA” /note=“Synthetic” /organism=“Artificial Sequence”
atgagcaaggaggccaaaaccctgcgtggacggtctgcttccctgccctt

SEQ ID NO: 406 - DNA, 50 bp, Artificial Sequence
  source 1..50: /mol_type=“unassigned DNA” /note=“Synthetic” /organism=“Artificial Sequence”
gagtgccaaatatgtgcccttccccgtggggaagacaaaagtatgagaca

SEQ ID NO: 407 - DNA, 50 bp, Artificial Sequence
  source 1..50: /mol_type=“unassigned DNA” /note=“Synthetic” /organism=“Artificial Sequence”
tatttttagcagcctatggattctaggagtgacccagctccagggatagg

SEQ ID NO: 408 - DNA, 50 bp, Artificial Sequence
  source 1..50: /mol_type=“unassigned DNA” /note=“Synthetic” /organism=“Artificial Sequence”
catgaggaaaggctgcaactttgagctccctctttagctagggagcctcc

SEQ ID NO: 409 - DNA, 50 bp, Artificial Sequence
  source 1..50: /mol_type=“unassigned DNA” /note=“Synthetic” /organism=“Artificial Sequence”
agcattaatgaagcacagggcctatcacgcagtcaggctcagtataaggt

SEQ ID NO: 410 - DNA, 50 bp, Artificial Sequence
  source 1..50: /mol_type=“unassigned DNA” /note=“Synthetic” /organism=“Artificial Sequence”
catactcaaattgatacacagcctttgtcctgagtgtttgtcttccaaaa

SEQ ID NO: 411 - DNA, 50 bp, Artificial Sequence
  source 1..50: /mol_type=“unassigned DNA” /note=“Synthetic” /organism=“Artificial Sequence”
agagtagtattgcttaaaaactgctccaaccacttcttaaacctgaaacc

SEQ ID NO: 412 - DNA, 50 bp, Artificial Sequence
  source 1..50: /mol_type=“unassigned DNA” /note=“Synthetic” /organism=“Artificial Sequence”
tcggccaaaatcagggacaaggatgacatgccattgcttaccaactgcta

SEQ ID NO: 413 - DNA, 50 bp, Artificial Sequence
  source 1..50: /mol_type=“unassigned DNA” /note=“Synthetic” /organism=“Artificial Sequence”
ccgttgtgcaaactccagaaagggcatctctctgtcccactcccccatta

SEQ ID NO: 414 - DNA, 50 bp, Artificial Sequence
  source 1..50: /mol_type=“unassigned DNA” /note=“Synthetic” /organism=“Artificial Sequence”
atctgcgtaaattgctgcatctctcttggcctcagttttcttagccacac

SEQ ID NO: 415 - DNA, 50 bp, Artificial Sequence
  source 1..50: /mol_type=“unassigned DNA” /note=“Synthetic” /organism=“Artificial Sequence”
gtaagtgccagctactattatttaggaggctaaggctctaggtgatgagg

SEQ ID NO: 416 - DNA, 50 bp, Artificial Sequence
  source 1..50: /mol_type=“unassigned DNA” /note=“Synthetic” /organism=“Artificial Sequence”
tgccaccctatggcattcttgttgtgtaatgaaataactctcctatgaaa

SEQ ID NO: 417 - DNA, 50 bp, Artificial Sequence
  source 1..50: /mol_type=“unassigned DNA” /note=“Synthetic” /organism=“Artificial Sequence”
ctgcgcttgcccaggaggccctggtctgcactgtttatagagaagaaccc

SEQ ID NO: 418 - DNA, 50 bp, Artificial Sequence
  source 1..50: /mol_type=“unassigned DNA” /note=“Synthetic” /organism=“Artificial Sequence”
ttaggaaagttctgtacagatatgtgtaatccagcatctgtttatctatt

SEQ ID NO: 419 - DNA, 50 bp, Artificial Sequence
  source 1..50: /mol_type=“unassigned DNA” /note=“Synthetic” /organism=“Artificial Sequence”
aatgatggaaaaaactgcagcgcacggtggaaatgtctactttgtatgca

SEQ ID NO: 420 - DNA, 50 bp, Artificial Sequence
  source 1..50: /mol_type=“unassigned DNA” /note=“Synthetic” /organism=“Artificial Sequence”
ctcctcattattcgcttctgctgtaactgcacctatggtaacccaggtgc

SEQ ID NO: 421 - DNA, 50 bp, Artificial Sequence
  source 1..50: /mol_type=“unassigned DNA” /note=“Synthetic” /organism=“Artificial Sequence”
aagtgctctgtaaccaaatattttggaaatgctgagttgtaccaagttgg

SEQ ID NO: 422 - DNA, 50 bp, Artificial Sequence
  source 1..50: /mol_type=“unassigned DNA” /note=“Synthetic” /organism=“Artificial Sequence”
ttttgaaatttccattatatgcaaagcccatgaaaggctaaatatcagtt

SEQ ID NO: 423 - DNA, 50 bp, Artificial Sequence
  source 1..50: /mol_type=“unassigned DNA” /note=“Synthetic” /organism=“Artificial Sequence”
gtttgtaaatgcacactgttgggggaaccctcttcctagtccttgtttcc

SEQ ID NO: 424 - DNA, 50 bp, Artificial Sequence
  source 1..50: /mol_type=“unassigned DNA” /note=“Synthetic” /organism=“Artificial Sequence”
tgggcgagaacttattcctcaggccattagattccctaatgctgcacctt

SEQ ID NO: 425 - DNA, 50 bp, Artificial Sequence
  source 1..50: /mol_type=“unassigned DNA” /note=“Synthetic” /organism=“Artificial Sequence”
gccatgggcaaaaacagctcaggtagtaatgaaggtgtggctatagctga

SEQ ID NO: 426 - DNA, 50 bp, Artificial Sequence
  source 1..50: /mol_type=“unassigned DNA” /note=“Synthetic” /organism=“Artificial Sequence”
acatcaaactaaattacatcatcagagtaaagagacaatttacaaaaagg

SEQ ID NO: 427 - DNA, 50 bp, Artificial Sequence
  source 1..50: /mol_type=“unassigned DNA” /note=“Synthetic” /organism=“Artificial Sequence”
aaaaagttcttcttctttgctcctccattgcggtccccttcaagatccat

SEQ ID NO: 428 - DNA, 50 bp, Artificial Sequence
  source 1..50: /mol_type=“unassigned DNA” /note=“Synthetic” /organism=“Artificial Sequence”
ctggctccaggcaaagaatactaccagcaacaaagaggaacatttcagat

SEQ ID NO: 429 - DNA, 50 bp, Artificial Sequence
  source 1..50: /mol_type=“unassigned DNA” /note=“Synthetic” /organism=“Artificial Sequence”
ggactagcctgctgcttcatttcccccctcctctgcagccgatttcagaa

SEQ ID NO: 430 - DNA, 50 bp, Artificial Sequence
  source 1..50: /mol_type=“unassigned DNA” /note=“Synthetic” /organism=“Artificial Sequence”
atattagtaacctggaaaacatacatggaggtatgttcattaacggcagt

SEQ ID NO: 431 - DNA, 50 bp, Artificial Sequence
  source 1..50: /mol_type=“unassigned DNA” /note=“Synthetic” /organism=“Artificial Sequence”
atgggaagagctggatttttgtcgtggagtaaaggagagggaatcaagaa

SEQ ID NO: 432 - DNA, 50 bp, Artificial Sequence
  source 1..50: /mol_type=“unassigned DNA” /note=“Synthetic” /organism=“Artificial Sequence”
aaaatcatagaaattgtgtctaaggatatgctttgggatatttggacttc

SEQ ID NO: 433 - DNA, 50 bp, Artificial Sequence
  source 1..50: /mol_type=“unassigned DNA” /note=“Synthetic” /organism=“Artificial Sequence”
cataaaccaaagggatcttctctactcgtgcgtccctagtctctctcccc

SEQ ID NO: 434 - DNA, 50 bp, Artificial Sequence
  source 1..50: /mol_type=“unassigned DNA” /note=“Synthetic” /organism=“Artificial Sequence”
gctgcctgtactagtgatagtgaggctcactaccatccaccacctaaatt

SEQ ID NO: 435 - DNA, 50 bp, Artificial Sequence
  source 1..50: /mol_type=“unassigned DNA” /note=“Synthetic” /organism=“Artificial Sequence”
gtgtagcttacgggagggaagtcaaagtcaggcacgttcatcacactcag

SEQ ID NO: 436 - DNA, 50 bp, Artificial Sequence
  source 1..50: /mol_type=“unassigned DNA” /note=“Synthetic” /organism=“Artificial Sequence”
ctcattgtaagattcaaaaacattccagcttacaaaacatatccagctta

SEQ ID NO: 437 - DNA, 50 bp, Artificial Sequence
  source 1..50: /mol_type=“unassigned DNA” /note=“Synthetic” /organism=“Artificial Sequence”
tttgcaaggcaatttgttctactgctggacagcttcatgtttaatgtttt

SEQ ID NO: 438 - DNA, 50 bp, Artificial Sequence
  source 1..50: /mol_type=“unassigned DNA” /note=“Synthetic” /organism=“Artificial Sequence”
ctatatttgaacaagcttctgggtaatatttatgacagggaagtcttgag

SEQ ID NO: 439 - DNA, 50 bp, Artificial Sequence
  source 1..50: /mol_type=“unassigned DNA” /note=“Synthetic” /organism=“Artificial Sequence”
ctgtgaaccaggcactgtttgaaatgttccatttattgacttatttaagt

SEQ ID NO: 440 - DNA, 50 bp, Artificial Sequence
  source 1..50: /mol_type=“unassigned DNA” /note=“Synthetic” /organism=“Artificial Sequence”
actactactaatgttgaaagtataccatgtaacaggcactgtacaaagcc

SEQ ID NO: 441 - DNA, 50 bp, Artificial Sequence
  source 1..50: /mol_type=“unassigned DNA” /note=“Synthetic” /organism=“Artificial Sequence”
ttttgggttttgttgctagcataaaaattattacctagtggatggtaaca

SEQ ID NO: 442 - DNA, 50 bp, Artificial Sequence
  source 1..50: /mol_type=“unassigned DNA” /note=“Synthetic” /organism=“Artificial Sequence”
tttttttttcatttgaagtaaatatccacctttgtatctaattttgcatt

SEQ ID NO: 443 - DNA, 50 bp, Artificial Sequence
  source 1..50: /mol_type=“unassigned DNA” /note=“Synthetic” /organism=“Artificial Sequence”
ttattttttaatagtgttcttgcacatgaggagaaagactgaattcaatt

SEQ ID NO: 444 - DNA, 50 bp, Artificial Sequence
  source 1..50: /mol_type=“unassigned DNA” /note=“Synthetic” /organism=“Artificial Sequence”
cgtgtcacttcgtttgacttcagctgggaacatgcatatcagtcgactca

SEQ ID NO: 445 - DNA, 50 bp, Artificial Sequence
  source 1..50: /mol_type=“unassigned DNA” /note=“Synthetic” /organism=“Artificial Sequence”
atcgtcacacagttttaagacaaatgtttttacctatttgacctagtctg

SEQ ID NO: 446 - DNA, 50 bp, Artificial Sequence
  source 1..50: /mol_type=“unassigned DNA” /note=“Synthetic” /organism=“Artificial Sequence”
tgtgctacaaacctgaaactggtaagacaagcacaaagcaacgtgcaata

SEQ ID NO: 447 - DNA, 50 bp, Artificial Sequence
  source 1..50: /mol_type=“unassigned DNA” /note=“Synthetic” /organism=“Artificial Sequence”
cttggatggaggctcagggagccaaaggcaaatgtcttcatagaaccagg

SEQ ID NO: 448 - DNA, 50 bp, Artificial Sequence
  source 1..50: /mol_type=“unassigned DNA” /note=“Synthetic” /organism=“Artificial Sequence”
atcatgaattaaacaaattaatttatgtattttgttttgagtcagtgtct

SEQ ID NO: 449 - DNA, 50 bp, Artificial Sequence
  source 1..50: /mol_type=“unassigned DNA” /note=“Synthetic” /organism=“Artificial Sequence”
acatgtgaccaacaagataattatgaaacctgactgctggatatgctgat

SEQ ID NO: 450 - DNA, 50 bp, Artificial Sequence
  source 1..50: /mol_type=“unassigned DNA” /note=“Synthetic” /organism=“Artificial Sequence”
gtcttttggaaaatgcaatctgccactctgtgcaatggaaaaccactgca

SEQ ID NO: 451 - DNA, 50 bp, Artificial Sequence
  source 1..50: /mol_type=“unassigned DNA” /note=“Synthetic” /organism=“Artificial Sequence”
ttattaatattagcctttcttctctccccgtttatgctttggtgggtact

SEQ ID NO: 452 - DNA, 50 bp, Artificial Sequence
  source 1..50: /mol_type=“unassigned DNA” /note=“Synthetic” /organism=“Artificial Sequence”
tttggtttgggttttgtttggcagaggcagaatagaataaagaacatggg

SEQ ID NO: 453 - DNA, 50 bp, Artificial Sequence
  source 1..50: /mol_type=“unassigned DNA” /note=“Synthetic” /organism=“Artificial Sequence”
agaattattgctgcacaattcttatgaaaccgaactagagctacactatt

SEQ ID NO: 454 - DNA, 50 bp, Artificial Sequence
  source 1..50: /mol_type=“unassigned DNA” /note=“Synthetic” /organism=“Artificial Sequence”
caggcagatcacttgacgtgaggagttcaagtgaggagttcaagtccagc

SEQ ID NO: 455 - DNA, 50 bp, Artificial Sequence
  source 1..50: /mol_type=“unassigned DNA” /note=“Synthetic” /organism=“Artificial Sequence”
acaaacaaactgaggtttaggtttaggtagctggagtttataggcatggc

SEQ ID NO: 456 - DNA, 50 bp, Artificial Sequence
  source 1..50: /mol_type=“unassigned DNA” /note=“Synthetic” /organism=“Artificial Sequence”
tctggaataatagttacatttgctacatccctttctagcgtcaactcact

SEQ ID NO: 457 - DNA, 50 bp, Artificial Sequence
  source 1..50: /mol_type=“unassigned DNA” /note=“Synthetic” /organism=“Artificial Sequence”
cataatgtgatgccatattaaactgtaatcacctttccaccaaactaata

SEQ ID NO: 458 - DNA, 50 bp, Artificial Sequence
  source 1..50: /mol_type=“unassigned DNA” /note=“Synthetic” /organism=“Artificial Sequence”
caaaattcatatgttgatacctaatctccaaagcaatagtattaagggtg

SEQ ID NO: 459 - DNA, 50 bp, Artificial Sequence
  source 1..50: /mol_type=“unassigned DNA” /note=“Synthetic” /organism=“Artificial Sequence”
aatactgtttggtatggcaagacagtattggttttggttcaagtgctcct

SEQ ID NO: 460 - DNA, 50 bp, Artificial Sequence
  source 1..50: /mol_type=“unassigned DNA” /note=“Synthetic” /organism=“Artificial Sequence”
ttggttttcctgggtggggaagggtgctggcctcattcacaacagcagat

SEQ ID NO: 461 - DNA, 50 bp, Artificial Sequence
  source 1..50: /mol_type=“unassigned DNA” /note=“Synthetic” /organism=“Artificial Sequence”
gggaaagacagagtgagagaaagagagagttagcctctacatattataag

SEQ ID NO: 462 - DNA, 50 bp, Artificial Sequence
  source 1..50: /mol_type=“unassigned DNA” /note=“Synthetic” /organism=“Artificial Sequence”
gcagagagagccctgtctcaaaacagatttctgagtgtggcttctgtcca

SEQ ID NO: 463 - DNA, 50 bp, Artificial Sequence
  source 1..50: /mol_type=“unassigned DNA” /note=“Synthetic” /organism=“Artificial Sequence”
tctcgtagctgagagagtcatgactatggcgtgttctctgtactctgagg

SEQ ID NO: 464 - DNA, 50 bp, Artificial Sequence
  source 1..50: /mol_type=“unassigned DNA” /note=“Synthetic” /organism=“Artificial Sequence”
ctcaagcagaaggaatctctccccatagccgctatagtttcaaatgtgct

SEQ ID NO: 465 - DNA, 50 bp, Artificial Sequence
  source 1..50: /mol_type=“unassigned DNA” /note=“Synthetic” /organism=“Artificial Sequence”
gtgaggataggtagcttttcttactcactgttgttaccagtacctagaac

SEQ ID NO: 466 - DNA, 50 bp, Artificial Sequence
  source 1..50: /mol_type=“unassigned DNA” /note=“Synthetic” /organism=“Artificial Sequence”
acgagcttgtcattctgtaaatgacatattcatattcttggtattgtaca

SEQ ID NO: 467 - DNA, 50 bp, Artificial Sequence
  source 1..50: /mol_type=“unassigned DNA” /note=“Synthetic” /organism=“Artificial Sequence”
caaggttaaaattcccgcattgtgggccttgtagctttcatgtcttaatg

SEQ ID NO: 468 - DNA, 50 bp, Artificial Sequence
  source 1..50: /mol_type=“unassigned DNA” /note=“Synthetic” /organism=“Artificial Sequence”
ggattttggccattctaagagatgtgcagtagtaactcagtgttttattt

SEQ ID NO: 469 - DNA, 50 bp, Artificial Sequence
  source 1..50: /mol_type=“unassigned DNA” /note=“Synthetic” /organism=“Artificial Sequence”
ctgaagactctgaacttgactgaggaaatgttaaacagatacctcttcat

SEQ ID NO: 470 - DNA, 50 bp, Artificial Sequence
  source 1..50: /mol_type=“unassigned DNA” /note=“Synthetic” /organism=“Artificial Sequence”
aacattccattatcctattgttcattctttggagctgtgatttgtttaat

SEQ ID NO: 471 - DNA, 50 bp, Artificial Sequence
  source 1..50: /mol_type=“unassigned DNA” /note=“Synthetic” /organism=“Artificial Sequence”
agcttcggtgaatattagaatggcctcaagagctagtaaaaaacacagcc

SEQ ID NO: 472 - DNA, 50 bp, Artificial Sequence
  source 1..50: /mol_type=“unassigned DNA” /note=“Synthetic” /organism=“Artificial Sequence”
aggcatatggggaaaaaataaggcaggaaaggaagacggaaaatgctgtg

SEQ ID NO: 473 - DNA, 50 bp, Artificial Sequence
  source 1..50: /mol_type=“unassigned DNA” /note=“Synthetic” /organism=“Artificial Sequence”
ttggttttataaaggatctaagtgtttggaaaggtgtgggaccatgtact

SEQ ID NO: 474 - DNA, 50 bp, Artificial Sequence
  source 1..50: /mol_type=“unassigned DNA” /note=“Synthetic” /organism=“Artificial Sequence”
acatgctctgcatgctttgacagtacagtgtatagaatagacacaaaact

SEQ ID NO: 475 - DNA, 50 bp, Artificial Sequence
  source 1..50: /mol_type=“unassigned DNA” /note=“Synthetic” /organism=“Artificial Sequence”
taaggttgtatcatctacctgtagtcactgcagtcagctgaattttacca

SEQ ID NO: 476 - DNA, 50 bp, Artificial Sequence
  source 1..50: /mol_type=“unassigned DNA” /note=“Synthetic” /organism=“Artificial Sequence”
ctctgtagccacacagatgccaacagctggcacttgtccaagaaacatgt

SEQ ID NO: 477 - DNA, 50 bp, Artificial Sequence
  source 1..50: /mol_type=“unassigned DNA” /note=“Synthetic” /organism=“Artificial Sequence”
agaatgggtcacttgttagaaacagtcaaggatacatacaaacagtggaa

SEQ ID NO: 478 - DNA, 50 bp, Artificial Sequence
  source 1..50: /mol_type=“unassigned DNA” /note=“Synthetic” /organism=“Artificial Sequence”
ccaagagtggtgaagccttcctgtttacagaggattttcatatctgttat

SEQ ID NO: 479 - DNA, 50 bp, Artificial Sequence
  source 1..50: /mol_type=“unassigned DNA” /note=“Synthetic” /organism=“Artificial Sequence”
acacccatggggccaagccaggagcagtcaccacagccaacctgcaggct

SEQ ID NO: 480 - DNA, 50 bp, Artificial Sequence
  source 1..50: /mol_type=“unassigned DNA” /note=“Synthetic” /organism=“Artificial Sequence”
tattctaaggaagtgccccctaaaacaaagctcaggagcctcaacccggc

SEQ ID NO: 481 - DNA, 50 bp, Artificial Sequence
  source 1..50: /mol_type=“unassigned DNA” /note=“Synthetic” /organism=“Artificial Sequence”
tcccaacatcaaaaggcaaattcttgccccacttttacagatgagagcgc

SEQ ID NO: 482 - DNA, 50 bp, Artificial Sequence
  source 1..50: /mol_type=“unassigned DNA” /note=“Synthetic” /organism=“Artificial Sequence”
taccatgggaaacagacagtggcccctgttctcaagtggcttagactcta

SEQ ID NO: 483 - DNA, 50 bp, Artificial Sequence
  source 1..50: /mol_type=“unassigned DNA” /note=“Synthetic” /organism=“Artificial Sequence”
cttattggccctaagtaaatcttaggttaggtagagctcagttcccaggg

SEQ ID NO: 484 - DNA, 50 bp, Artificial Sequence
  source 1..50: /mol_type=“unassigned DNA” /note=“Synthetic” /organism=“Artificial Sequence”
gtatttttaggaacattcaggaaaacaggtaaagggtattcaggaattca

SEQ ID NO: 485 - DNA, 50 bp, Artificial Sequence
  source 1..50: /mol_type=“unassigned DNA” /note=“Synthetic” /organism=“Artificial Sequence”
ggccttcctcactctgacggtgagttccagaggacagggatttgtggttg

SEQ ID NO: 486 - DNA, 50 bp, Artificial Sequence
  source 1..50: /mol_type=“unassigned DNA” /note=“Synthetic” /organism=“Artificial Sequence”
tggttgctaatttctcttcacttctgggaaaccagccccttataaatcaa

SEQ ID NO: 487 - DNA, 50 bp, Artificial Sequence
  source 1..50: /mol_type=“unassigned DNA” /note=“Synthetic” /organism=“Artificial Sequence”
aacacagagcagtatgtacaggacagcgttagaatataccagagaacaag

SEQ ID NO: 488 - DNA, 50 bp, Artificial Sequence
  source 1..50: /mol_type=“unassigned DNA” /note=“Synthetic” /organism=“Artificial Sequence”
aaacacacctgtcacccacgaccctggcatagggcatcgtgaacccatca

SEQ ID NO: 489 - DNA, 50 bp, Artificial Sequence
  source 1..50: /mol_type=“unassigned DNA” /note=“Synthetic” /organism=“Artificial Sequence”
atagtattctgttcttcagggagttgtgggttcggatctgtgcaaagata

SEQ ID NO: 490 - DNA, 50 bp, Artificial Sequence
  source 1..50: /mol_type=“unassigned DNA” /note=“Synthetic” /organism=“Artificial Sequence”
taggaatcagggaactctagatgcgtctagcagctagcctgtggcctcga

SEQ ID NO: 491 - DNA, 50 bp, Artificial Sequence
  source 1..50: /mol_type=“unassigned DNA” /note=“Synthetic” /organism=“Artificial Sequence”
ttcaaattgcttgattaaaatggcaaacagtttgaaaattgtatacctct

SEQ ID NO: 492 - DNA, 50 bp, Artificial Sequence
  source 1..50: /mol_type=“unassigned DNA” /note=“Synthetic” /organism=“Artificial Sequence”
ggataatggaaaagggggtttctcccaagtagagaacttaaacagtgtga

SEQ ID NO: 493 - DNA, 50 bp, Artificial Sequence
  source 1..50: /mol_type=“unassigned DNA” /note=“Synthetic” /organism=“Artificial Sequence”
cacctagtcatgtgtatataaaatcaccatgttattacagaatttagtaa

SEQ ID NO: 494 - DNA, 50 bp, Artificial Sequence
  source 1..50: /mol_type=“unassigned DNA” /note=“Synthetic” /organism=“Artificial Sequence”
caatctattttccacctgggttctcgaaccgacttttcctccctctcttc

SEQ ID NO: 495 - DNA, 50 bp, Artificial Sequence
  source 1..50: /mol_type=“unassigned DNA” /note=“Synthetic” /organism=“Artificial Sequence”
gggtcttcctacgggactgccttagacgtgctgggctttggcctcagtga

SEQ ID NO: 496 - DNA, 50 bp, Artificial Sequence
  source 1..50: /mol_type=“unassigned DNA” /note=“Synthetic” /organism=“Artificial Sequence”
agttttggttggggaggacaatgccaggttaacagacacttaatatacat

SEQ ID NO: 497 - DNA, 50 bp, Artificial Sequence
  source 1..50: /mol_type=“unassigned DNA” /note=“Synthetic” /organism=“Artificial Sequence”
aaagagagtggaagtaccaggtgggcaaagtttacaattttaagtaggat

SEQ ID NO: 498 - DNA, 50 bp, Artificial Sequence
  source 1..50: /mol_type=“unassigned DNA” /note=“Synthetic” /organism=“Artificial Sequence”
atgattctttccatgacacctagtgcccttctccatctagagctacctct

SEQ ID NO: 499 - DNA, 50 bp, Artificial Sequence
  source 1..50: /mol_type=“unassigned DNA” /note=“Synthetic” /organism=“Artificial Sequence”
aaatgaactcagcaatgaaatggaacaagctatccatacatgcagcaatt

SEQ ID NO: 500 - DNA, 50 bp, Artificial Sequence
  source 1..50: /mol_type=“unassigned DNA” /note=“Synthetic” /organism=“Artificial Sequence”
ccatcattgcctggctgttgaagcagttcttgacattttaaagtaatatg

SEQ ID NO: 501 - DNA, 50 bp, Artificial Sequence
  source 1..50: /mol_type=“unassigned DNA” /note=“Synthetic” /organism=“Artificial Sequence”
ttgctacaaggaggattatgggtgaaagtcatggatggattatgagttaa

SEQ ID NO: 502 - DNA, 50 bp, Artificial Sequence
  source 1..50: /mol_type=“unassigned DNA” /note=“Synthetic” /organism=“Artificial Sequence”
gatggacatcactgaaatgtagttttgcctgaagtgtggtttggatgctc

SEQ ID NO: 503 - DNA, 50 bp, Artificial Sequence
  source 1..50: /mol_type=“unassigned DNA” /note=“Synthetic” /organism=“Artificial Sequence”
cttgtttgtgtatgatacatgaagtagaattcatacagcacaagtacttt

SEQ ID NO: 504 - DNA, 50 bp, Artificial Sequence
  source 1..50: /mol_type=“unassigned DNA” /note=“Synthetic” /organism=“Artificial Sequence”
gaaattctccataatttctgatccactcttacattcctctcctttccagc

SEQ ID NO: 505 - DNA, 50 bp, Artificial Sequence
  source 1..50: /mol_type=“unassigned DNA” /note=“Synthetic” /organism=“Artificial Sequence”
gggggctggggggaagtcccgggacaggtgcatgtcatcaacacgactgt

SEQ ID NO: 506 - DNA, 50 bp, Artificial Sequence
  source 1..50: /mol_type=“unassigned DNA” /note=“Synthetic” /organism=“Artificial Sequence”
agatcttttcaggcataaaagttgtcaataggttttcataaatttctagg

SEQ ID NO: 507 - DNA, 50 bp, Artificial Sequence
  source 1..50: /mol_type=“unassigned DNA” /note=“Synthetic” /organism=“Artificial Sequence”
cccttgcacaggcacagctataatttttgtctctcttctgttggaaaggt

SEQ ID NO: 508 - DNA, 50 bp, Artificial Sequence
  source 1..50: /mol_type=“unassigned DNA” /note=“Synthetic” /organism=“Artificial Sequence”
gtggtttctaatgatttaataccatcccccagggtgctcttcttgtgata

SEQ ID NO: 509 - DNA, 50 bp, Artificial Sequence
  source 1..50: /mol_type=“unassigned DNA” /note=“Synthetic” /organism=“Artificial Sequence”
gaatattgaaggtagccagaaaagaaaaaaaggcacattgcatgcagagg

SEQ ID NO: 510 - DNA, 50 bp, Artificial Sequence
  source 1..50: /mol_type=“unassigned DNA” /note=“Synthetic” /organism=“Artificial Sequence”
atggcagttcattgctttactatttggacatttcaaactgtcccaaggtg

SEQ ID NO: 511 - DNA, 50 bp, Artificial Sequence
  source 1..50: /mol_type=“unassigned DNA” /note=“Synthetic” /organism=“Artificial Sequence”
ttttttcaaacctttaaacaacagtcccacttggataaagtctgagagcg

SEQ ID NO: 512 - DNA, 50 bp, Artificial Sequence
  source 1..50: /mol_type=“unassigned DNA” /note=“Synthetic” /organism=“Artificial Sequence”
atagcctaactttccccccgaagcttcccaagccctcatgatatctatta

SEQ ID NO: 513 - DNA, 50 bp, Artificial Sequence
  source 1..50: /mol_type=“unassigned DNA” /note=“Synthetic” /organism=“Artificial Sequence”
acctgagaattctcacccatccaattctacttgatatgggattcctctaa

SEQ ID NO: 514 - DNA, 50 bp, Artificial Sequence
  source 1..50: /mol_type=“unassigned DNA” /note=“Synthetic” /organism=“Artificial Sequence”
aatgggcatgatctcactcacatggaacaggatctctttccttgttagca

SEQ ID NO: 515 - DNA, 50 bp, Artificial Sequence
  source 1..50: /mol_type=“unassigned DNA” /note=“Synthetic” /organism=“Artificial Sequence”
agtcacagaaacatagcaagcccttgaaatcaggctttctgactttgtct

SEQ ID NO: 516 - DNA, 50 bp, Artificial Sequence
  source 1..50: /mol_type=“unassigned DNA” /note=“Synthetic” /organism=“Artificial Sequence”
cacctacacacatgcatgcacacacacatggcctctctctccaggcttct

SEQ ID NO: 517 - DNA, 50 bp, Artificial Sequence
  source 1..50: /mol_type=“unassigned DNA” /note=“Synthetic” /organism=“Artificial Sequence”
cgtacagacctggtccaaaaattccaatttcataggtgtggagttttcat

SEQ ID NO: 518 - DNA, 50 bp, Artificial Sequence
  source 1..50: /mol_type=“unassigned DNA” /note=“Synthetic” /organism=“Artificial Sequence”
caaacaaccaccacatcaaaataatagcaaagacaacaactaatactaat

SEQ ID NO: 519 - DNA, 50 bp, Artificial Sequence
  source 1..50: /mol_type=“unassigned DNA” /note=“Synthetic” /organism=“Artificial Sequence”
atagtaagttttaaagtaagaggtcagaaacatatgttactttacaaaca

SEQ ID NO: 520 - DNA, 50 bp, Artificial Sequence
  source 1..50: /mol_type=“unassigned DNA” /note=“Synthetic” /organism=“Artificial Sequence”
ttatgtagcaggtcctgatgtaacagaattaagattgcaggtgggattgg

SEQ ID NO: 521 - DNA, 50 bp, Artificial Sequence
  source 1..50: /mol_type=“unassigned DNA” /note=“Synthetic” /organism=“Artificial Sequence”
tccctagaacagcaggacctgcgaaactctgaggccgctttgtgaggtcc

SEQ ID NO: 522 - DNA, 50 bp, Artificial Sequence
  source 1..50: /mol_type=“unassigned DNA” /note=“Synthetic” /organism=“Artificial Sequence”
ttgaaaagagaaacccacagggctttctgcttaaatccctcggacacagt

SEQ ID NO: 523 - DNA, 50 bp, Artificial Sequence
  source 1..50: /mol_type=“unassigned DNA” /note=“Synthetic” /organism=“Artificial Sequence”
taaggatgggacccctactgtccatctcaggctcagcactgccttggggc

SEQ ID NO: 524 - DNA, 50 bp, Artificial Sequence
  source 1..50: /mol_type=“unassigned DNA” /note=“Synthetic” /organism=“Artificial Sequence”
cttctacatcttagctcacctgtcctcacaaataaacatcactcttgaat

SEQ ID NO: 525 - DNA, 50 bp, Artificial Sequence
  source 1..50: /mol_type=“unassigned DNA” /note=“Synthetic” /organism=“Artificial Sequence”
ttgttgaaatgtgaccacgaactaggtcttaacctagcaaattcacaaat

SEQ ID NO: 526 - DNA, 50 bp, Artificial Sequence
  source 1..50: /mol_type=“unassigned DNA” /note=“Synthetic” /organism=“Artificial Sequence”
ctttctaaacactagcagcccagaattctcaggccacttttgggcattgt

SEQ ID NO: 527 - DNA, 50 bp, Artificial Sequence
  source 1..50: /mol_type=“unassigned DNA” /note=“Synthetic” /organism=“Artificial Sequence”
gtctatgaattggtgaatcagccaagtgaatgcttcaaaaactgggactc

SEQ ID NO: 528 - DNA, 50 bp, Artificial Sequence
  source 1..50: /mol_type=“unassigned DNA” /note=“Synthetic” /organism=“Artificial Sequence”
cctcctgagatgaacatcgtgaggagtaaatagagatgctattctcagct

SEQ ID NO: 529 - DNA, 50 bp, Artificial Sequence
  source 1..50: /mol_type=“unassigned DNA” /note=“Synthetic” /organism=“Artificial Sequence”
aactccgattaatcactagttgttcacaccaaaaacccaagtgccattac

SEQ ID NO: 530 - DNA, 50 bp, Artificial Sequence
  source 1..50: /mol_type=“unassigned DNA” /note=“Synthetic” /organism=“Artificial Sequence”
tcaccaagtctggttgtcccagtctcctatctctgtctgttcctctcctc

SEQ ID NO: 531 - DNA, 50 bp, Artificial Sequence
  source 1..50: /mol_type=“unassigned DNA” /note=“Synthetic” /organism=“Artificial Sequence”
atgagttggaattgcataatgggtagatgctgatgctggagaactttgag

SEQ ID NO: 532 - DNA, 50 bp, Artificial Sequence
  source 1..50: /mol_type=“unassigned DNA” /note=“Synthetic” /organism=“Artificial Sequence”
gtcattgactcgactataattttccaaactacctaaacgtgttatatcat

SEQ ID NO: 533 - DNA, 50 bp, Artificial Sequence
  source 1..50: /mol_type=“unassigned DNA” /note=“Synthetic” /organism=“Artificial Sequence”
tgatgattaggagtctgatggaggaaagtaattttaaaacaacttaatgg

SEQ ID NO: 534 - DNA, 50 bp, Artificial Sequence
  source 1..50: /mol_type=“unassigned DNA” /note=“Synthetic” /organism=“Artificial Sequence”
tggggttttatttgcttttttcccagtttcttagatgtaaagttaggtta

SEQ ID NO: 535 - DNA, 50 bp, Artificial Sequence
  source 1..50: /mol_type=“unassigned DNA” /note=“Synthetic” /organism=“Artificial Sequence”
ggaactctgacgcaatccagggccgaggaaaaatgattaaaacccaacaa

SEQ ID NO: 536 - DNA, 50 bp, Artificial Sequence
  source 1..50: /mol_type=“unassigned DNA” /note=“Synthetic” /organism=“Artificial Sequence”
tactgcagtgagttcaagtgttgtacctgcttaaaatgcagtgacactaa

SEQ ID NO: 537 - DNA, 50 bp, Artificial Sequence
  source 1..50: /mol_type=“unassigned DNA” /note=“Synthetic” /organism=“Artificial Sequence”
ggcagagggaacagcttgtgcaaaggccctggggcaggccaagggcagag

SEQ ID NO: 538 - DNA, 50 bp, Artificial Sequence
  source 1..50: /mol_type=“unassigned DNA” /note=“Synthetic” /organism=“Artificial Sequence”
aaaagaggatggctggtttatctcaagtaatcagacatttaataataata

SEQ ID NO: 539 - DNA, 50 bp, Artificial Sequence
  source 1..50: /mol_type=“unassigned DNA” /note=“Synthetic” /organism=“Artificial Sequence”
gtgctattttgttgctgttaggtctattttcttcatctgttatttcgcat

SEQ ID NO: 540 - DNA, 50 bp, Artificial Sequence
  source 1..50: /mol_type=“unassigned DNA” /note=“Synthetic” /organism=“Artificial Sequence”
gcctgggggagcggggaatcgcttttcgccggcctccgcgtaaccttgtt

SEQ ID NO: 541 - DNA, 50 bp, Artificial Sequence
  source 1..50: /mol_type=“unassigned DNA” /note=“Synthetic” /organism=“Artificial Sequence”
ggctcaacggaagtgaccgtcccacagttatgcagcactaagtcaatggc

SEQ ID NO: 542 - DNA, 50 bp, Artificial Sequence
  source 1..50: /mol_type=“unassigned DNA” /note=“Synthetic” /organism=“Artificial Sequence”
ttgtgacaggtcccagcgtgaacacgcacgccctagccgggccccaaacc

SEQ ID NO: 543 - DNA, 50 bp, Artificial Sequence
  source 1..50: /mol_type=“unassigned DNA” /note=“Synthetic” /organism=“Artificial Sequence”
aaggggaccgcaatggaggagcaaagaagaagaacttttttaaactgaac

SEQ ID NO: 544 - DNA, 50 bp, Artificial Sequence
  source 1..50: /mol_type=“unassigned DNA” /note=“Synthetic” /organism=“Artificial Sequence”
gctgacttcttgactgcagccacaggaaggactcaacccaggaccatcca

SEQ ID NO: 545 - DNA, 50 bp, Artificial Sequence
  source 1..50: /mol_type=“unassigned DNA” /note=“Synthetic” /organism=“Artificial Sequence”
aatttttcaatggtaaacagaccagagttattctaagaaattatgaaaag

SEQ ID NO: 546 - DNA, 50 bp, Artificial Sequence
  source 1..50: /mol_type=“unassigned DNA” /note=“Synthetic” /organism=“Artificial Sequence”
aggatttcaagacttgcctgagcaacataatgagatgccctctctcaaaa

SEQ ID NO: 547 - DNA, 50 bp, Artificial Sequence
  source 1..50: /mol_type=“unassigned DNA” /note=“Synthetic” /organism=“Artificial Sequence”
agcaagcagaaaacaaacaacttcattaaaaatgagcagaggacctgaac

SEQ ID NO: 548 - DNA, 50 bp, Artificial Sequence
  source 1..50: /mol_type=“unassigned DNA” /note=“Synthetic” /organism=“Artificial Sequence”
ttctgagaccttcttgcccctttgtttctaagcccagggccacaattccc

(19) United States
(12) Patent Application Publication
(10) Pub. No.: US 2018/0355430 A1
(43) Pub. Date: Dec. 13, 2018

(54) Genetic Predictors of a Response to Treatment with CRHR1 Antagonists

(71) Applicant: HMNC Value GmbH, Munich (DE)

(72) Inventor: Florian Holsboer, Munich (DE)

(21) Appl. No.: 15/562,454

(22) PCT Filed: Apr. 1, 2016
(86) PCT No.: PCT/EP2016/057229

§ 371 Date: Sep. 28, 2017

Related U.S. Application Data
(60) Provisional application No. 62/141,879, filed on Apr. 2, 2015.

(51) Int. Cl. C12Q 1/6883

(52) U.S. Cl. CPC C12Q 1/6883; C12Q 2600/156; C12Q 2600/106

(57) ABSTRACT

The present disclosure provides methods for predicting a treatment response of a subject to a treatment with a CRHR1 antagonist and methods of detecting a polymorphism genotype associated with a treatment response of a subject to treatment with a CRHR1 antagonist. Sets of at least one polymorphism genotype useful in such methods are also disclosed. Further, methods of treating a condition which is treatable by a CRHR1 antagonist in a subject in need thereof are provided. Compositions, kits and arrays and uses thereof are also disclosed, which can be used in the methods of the invention.

### BACKGROUND OF THE INVENTION

Corticotropin-releasing hormone (CRH or corticotropin-releasing factor/CRF) is pivotal in modulating the activity of the hypothalamic-pituitary-adrenal (HPA) axis during stress, stress-response and stress-adaptation, as well as in inflammation. CRH is a 41 aa peptide hormone derived from a 196-amino acid pre-prohormone, produced in the hypothalamus and transported in small vessels to the pituitary from which the peripheral stress hormone corticotropin (also known as adrenocorticotropic hormone/ACTH) is released which, in turn, induces secretion of cortisol from the adrenal gland. CRH containing nerve fibers also project to areas in the CNS implicated in behavioral adaptation to stress, including the amygdala, being implied in fear and anxiety, the prefrontal cortex and the hippocampus. Persistent stress is hypothesized to result in anxiety, depressive symptoms and other stress-related disorders in patients with inherited or acquired vulnerability. Among those patients, antagonists of CRH would appear to be the ideally tailored therapy. The effects of CRH in the brain, where CRH acts like a neurotransmitter, are conveyed via the type 1 CRH receptor (CRHR1, or CRF-R1), which mediates a variety of endocrine, behavioural, and autonomic stress-responses (Heinrichs and Koob, J Pharmacol Exp Ther. 2004 November; 311(2):427-40), including, but not being limited to, psychiatric conditions such as anxiety disorders and major depression (Holsboer and Ising, Eur J Pharmacol 2008, 583(2-3):350-7; Koob and Zorilla, Neuropsychopharmacology 2012, 37(1):308-9). In murine models, CRHR1 deletions displayed less depression-related behaviors, while CRH overexpression in the CNS lead to an increase of several behaviors that can, within certain limitations, be extrapolated to human depression.

The World Health Organization (WHO) considers depression as one of the top ten causes of morbidity and mortality, with a lifetime prevalence for depression ranging, e.g., from 12-16% in Germany. Depressive disorders account for a worldwide number of over one million suicides annually, and create a significant burden on costs in health care, work leave, disability pension, early retirement, loss of productivity of workers, by far surmounting direct costs such as inpatient and outpatient treatments. Finally, depression also multiplies the risk for other conditions such as cardiovascular disease, diabetes and neurodegenerative disorders.

Significant effort has been focused on the development of inhibitors of neuropeptide receptor ligands as drugs for psychiatric diseases and related conditions, including CRHR1 antagonists for the treatment of anxiety and depression (Griebel and Holsboer, Nature Reviews Drug Discovery 2012, 11:462-478). However, essentially all randomized controlled trials using CRHR1 antagonists in humans produced negative results, which has lead several originators to stall CRHR1 antagonist development, see Williams, Expert Opin Ther Pat 2013, 23(8):1057-68.

The present invention rests in part on the recognition that several of these earlier trials testing CRHR1 antagonist only failed to show statistically relevant effects due to the lack of appropriate patient stratification and selection according to their individual, underlying pathophysiology. In other words, a CRHR1 antagonist can only be effective in pathologies where the underlying causality is dominated by CRH over-activity or excessive CRH secretion. In the absence of CRH over-activity, a CRHR1-antagonist is not likely to have any significant effect.

Methods and algorithms for predicting an ACTH response to CRHR1 antagonists using the dex/CRH test in patients with depressive symptoms and/or anxiety symptoms, as well as a set of genotypes of single nucleotide polymorphisms (SNPs) for use in such methods and algorithms, have been described in WO 2013/160315 (A2). Correspondingly, CRHR1 antagonists for use in the treatment of depressive symptoms and/or anxiety symptoms in patients having CRH over-activity have been described in WO 2013/160317 (A2), wherein CRH over-activity is detected by determining the status of the same set of genotypes of SNPs as in WO 2013/160315 (A2). However, there remains a need for improved methods of predicting the treatment response to CRHR1 antagonists. In particular, there is a strong need to provide a direct prediction of clinical response in subjects treated with a treatment with a CRHR1 antagonist, e.g., in subjects having depressive symptoms or anxiety symptoms, or another stress-related condition mediated by CRHR1.

The present invention rests on additional evidence unknown in the prior art, according to which many polymorphisms are present in essentially all relevant nodes of the CRH/CRHR1 signaling chain. It is, thus, an object of the present invention to provide a particularly useful set of genomic DNA polymorphisms for predicting a central CRH over-activity and/or a clinical response to treatment with a CRHR1 antagonist, in particular in, but not being limited to, patients with anxiety symptoms or depressive symptoms. Thus, the present invention provides improved methods for predicting treatment response of patients to a treatment with a CRHR1 antagonist, as well as methods of treatment comprising an CRHR1 antagonist, compositions, kits and arrays comprising polynucleotides and uses thereof in methods of predicting the treatment response.

### SUMMARY OF THE INVENTION

The present invention is based, at least in part, on the recognition of polymorphism genotypes, including, but not being limited to, single nucleotide polymorphism (SNP) genotypes that are predictive of a subject's clinical responsiveness or non-responsiveness to treatment with a corticotropin releasing hormone receptor type 1 (CRHR1) antagonist. Specifically, the presence or absence of one or more of the polymorphism genotypes disclosed in Table 2 herein can be used to predict the likelihood that a given subject will or will not respond to treatment with a CRHR1 antagonist. The set and subsets of polymorphism genotypes, compositions, and methods described herein are thus useful in selecting appropriate treatment modalities (e.g., a treatment with a CRHR1 antagonist or a non-CRHR1 antagonist) for a subject having a condition treatable by a CRHR1 antagonist.

Thus, in a first aspect, the invention provides a method for predicting a treatment response of a subject to treatment with a CRHR1 antagonist, the method comprising: providing a biological sample obtained from the subject, and detecting the presence or absence of one or more polymorphism genotypes in the biological sample, wherein the one or more polymorphism genotypes comprise: (a) at least one polymorphism genotype selected from the group consisting of rs34169260 (A/G), rs796287 (A/C), rs56149945 (A/G), rs6190 (T/C), rs7179092 (T/C), rs7614867 (A/G), rs920640 (T/C), rs7167722 (T/C), rs920638 (T/C), rs7165629 (T/C), rs80049044 (T/A), rs16941058 (A/G), rs112015971 (A/G), rs10894873 (T/C), rs117455294 (T/G), rs1170303 (T/C), rs16940681 (C/G), rs968519 (T/C), rs28381866 (T/C), rs79320848 (T/G), rs114653646 (T/G), rs2589496 (T/C), rs10482650 (A/G), rs17614642 (A/G), rs73200317 (T/C), rs1380146 (T/A), rs735164 (T/C), rs730976 (T/G), rs55934524 (T/G), rs4570614 (A/G), rs4458044 (C/G), rs77850169 (A/G), rs35339359 (A/G), rs34800935 (T/C), rs72945439 (T/C), rs113959523 (A/G), rs116798177 (A/G), rs11247577 (T/G), rs75869266 (T/C), rs74372553 (T/C), rs11691508 (A/G), rs6493965 (A/G), rs4869476 (T/C), rs3730170 (T/C), rs2145288 (A/C), rs2935752 (A/C), rs146512400 (A/G), rs62057097 (T/C), rs115061314 (T/C), rs34113594 (T/G), rs61751173 (A/G), rs74338736 (A/C), rs10851726 (T/C), rs4610906 (T/C), rs59485211 (T/C), rs7060015 (T/G), rs75710780 (T/G), rs6520908 (T/C), rs487011 (T/G), rs1383699 (A/C), rs67516871 (A/G), rs114106519 (T/C), rs7220091 (A/G), rs12489026 (A/G), rs876270 (T/C), rs4968161 (T/C), rs62056907 (A/G), rs2235013 (T/C), rs16878812 (A/G), rs6549407 (A/G), rs28381848 (A/G), rs79723704 (A/C), rs72814052 (A/G), rs10152908 (T/C), rs172769 (A/C), rs78596668 (T/C), rs73307922 (T/C), rs3842 (A/G), rs7210584 (A/C), rs62402121 (T/C), rs55709291 (A/G), rs72747088 (A/G), rs929610 (G/C), rs6766242 (T/C), rs1468552 (G/C), rs78838114 (T/C), rs62489862 (T/C), rs894342 (A/G), rs58882373 (T/C), rs3811939 (A/G), rs6984688 (T/G), rs1018160 (T/C), rs76602912 (A/G), rs80067508 (A/G), rs74888440 (T/C), rs12481583 (T/C), rs66794218 (A/G), rs16946701 (A/G), rs75726724 (A/G), rs67959715 (T/A), rs11871392 (T/G), rs2044070 (A/G), rs77612799 (T/C), rs6743702 (T/C), rs616870 (T/C), rs79590198 (A/G), rs75715199 (A/G), rs13087555 (T/C), rs4869618 (T/C), rs117397046 (A/G), rs8042817 (A/G), rs2258097 (T/C), rs2260882 (C/G), rs532996 (A/G), rs11747040 (T/C), rs10034039 (T/G), rs116909369 (A/G), rs79134986 (A/G), rs117615688 (T/C), rs8032253 (T/C), rs12818653 (T/A), rs4587884 (A/C), rs77122853 (T/C), rs117615061 (T/C), rs74682905 (A/G), rs2257468 (T/C), rs2032582 (T/G), rs2235015 (T/G), rs2729794 (T/C), rs77549514 (A/G), rs74790420 (A/C), rs73129579 (T/C), rs12913346 (A/C), rs117560908 (T/C), rs72747091 (A/G), rs2935751 (A/G), rs4331446 (A/G), rs7523266 (T/C), rs7648662 (T/C), rs117034065 (A/G), rs4836256 (T/C), rs80238698 (T/C), rs3730173 (T/C), rs11687884 (T/C), rs72693005 (T/C), rs2589476 (T/C), rs9813396 (T/C), rs10482667 (A/G), rs72784444 (A/G), rs75074511 (T/C), rs7951003 (A/G), rs79584784 (A/G), rs2214102 (T/C), rs28811003 (A/G), rs6100261 (NT), rs77152456 (A/G), rs66624622 (T/G), rs140302965 (A/G), rs11653269 (T/C), rs74405057 (A/G), rs7121 (A/G), rs16977818 (A/C), rs12490095 (T/C), rs118003903 (A/G), rs62377761 (T/C), P1_M_061510_6_34_M (−/CACTTACCTTCTTTGTGCCACAGTTTCCCTATCTAAAACAC AAGGTTATCAGTTATCAACATCTCTTGGGATTGTGAGGACTAAAGTAATGCACATAAA G), rs375115639 (−/AAATTACCCTGTTAGGTTTCAATGAAACACCTTTTCTCTTGTAACA AACATCTCCTCC AAGCTAGAATTTCAAAACAG), rs1002204 (A/C), rs10062367 (A/G), rs10482642 (A/G), rs10482658 (A/G), rs1053989 (A/C), rs10851628 (T/C), rs10947562 (T/C), rs11069612 (A/G), rs11071351 (T/C), rs11091175 (A/G), rs11638450 (T/C), rs11715827 (T/G), rs11745958 (T/C), rs11834041 (A/G), rs1202180 (T/C), rs12054781 (A/G), rs12539395 (A/G), rs12720066 (T/G), rs1279754 (A/C), rs12872047 (T/C), rs12876742 (A/C), rs12917505 (A/G), rs13066950 (T/G), rs13229143 (C/G), rs1383707 (T/C), rs1441824 (T/C), rs1652311 (A/G), rs17064 (T/A), rs17100236 (A/G), rs17149699 (A/G), rs1724386 (A/G), rs17250255 (A/G), rs17327624 (T/G), rs17616338 (A/G), rs17687796 (A/G), rs17740874 (T/C), rs17763104 (T/C), rs1880748 (T/C), rs1882478 (A/G), rs1944887 (T/C), rs2028629 (A/G), rs2143404 (A/G), rs2173530 (T/C), rs220806 (T/C), rs2235047 (A/C), rs2242071 (A/G), rs2257474 (T/C), rs2295583 (NT), rs234629 (T/C), rs234630 (A/G), rs2436401 (A/G), rs258750 (T/C), rs2589487 (T/C), rs28364018 (T/G), rs28381774 (T/C), rs28381784 (A/G), rs2963155 (A/G), rs3133622 (T/G), rs32897 (T/C), rs33388 (NT), rs3730168 (T/C), rs3735833 (T/G), rs3777747 (A/G), rs3786066 (T/C), rs3798346 (T/C), rs3822736 (A/G), rs389035 (T/C), rs3924144 (A/G), rs4148737 (T/C), rs4148749 (G/C), rs417968 (T/C), rs4458144 (T/C), rs4515335 (T/C), rs4728699 (A/G), rs4758040 (A/G), rs4812040 (A/G), rs4912650 (T/G), rs4957891 (T/C), rs5906392 (A/G), rs6026561 (T/C), rs6026565 (T/A), rs6026567 (A/G), rs6026593 (A/G), rs6092704 (T/G), rs6100260 (A/G), rs6128461 (T/C), rs6415328 (T/C), rs6610868 (T/C), rs6686061 (A/C), rs6730350 (T/G), rs6746197 (T/C), rs6963426 (T/C), rs7121326 (T/C), rs7721799 (A/G), rs7787082 (T/C), rs7799592 (A/C), rs796245 (T/C), rs809482 (A/C), rs8125112 (T/C), rs919196 (A/G), rs920750 (T/C), rs9332385 (A/G), rs930473 (T/G), rs9324921 (A/C), rs9348979 (A/G), rs9571939 (A/C), and rs9892359 (T/C); (b) at least one polymorphism genotype being in linkage disequilibrium with any one of the polymorphism genotypes of (a); or (c) a combination of (a) and (b); and predicting the treatment response from the presence or absence of the one or more polymorphism genotypes of (a), (b), or (c).

In another aspect, the invention provides a method for detecting a polymorphism genotype associated with a treatment response of a subject to treatment with a CRHR1 antagonist, the method comprising providing a biological sample obtained from the subject, and detecting the presence or absence of one or more polymorphism genotypes in the biological sample, wherein the one or more polymorphism genotypes comprise: (a) at least one polymorphism genotype selected from the group consisting of rs34169260 (A/G), rs796287 (A/C), rs56149945 (A/G), rs6190 (T/C), rs7179092 (T/C), rs7614867 (A/G), rs920640 (T/C), rs7167722 (T/C), rs920638 (T/C), rs7165629 (T/C), rs80049044 (T/A), rs16941058 (A/G), rs112015971 (A/G), rs10894873 (T/C), rs117455294 (T/G), rs1170303 (T/C), rs16940681 (C/G), rs968519 (T/C), rs28381866 (T/C), rs79320848 (T/G), rs114653646 (T/G), rs2589496 (T/C), rs10482650 (A/G), rs17614642 (A/G), rs73200317 (T/C), rs1380146 (T/A), rs735164 (T/C), rs730976 (T/G), rs55934524 (T/G), rs4570614 (A/G), rs4458044 (C/G), rs77850169 (A/G), rs35339359 (A/G), rs34800935 (T/C), rs72945439 (T/C), rs113959523 (A/G), rs116798177 (A/G), rs11247577 (T/G), rs75869266 (T/C), rs74372553 (T/C), rs11691508 (A/G), rs6493965 (A/G), rs4869476 (T/C), rs3730170 (T/C), rs2145288 (A/C), rs2935752 (A/C), rs146512400 (A/G), rs62057097 (T/C), rs115061314 (T/C), rs34113594 (T/G), rs61751173 (A/G), rs74338736 (A/C), rs10851726 (T/C), rs4610906 (T/C), rs59485211 (T/C), rs7060015 (T/G), rs75710780 (T/G), rs6520908 (T/C), rs487011 (T/G), rs1383699 (A/C), rs67516871 (A/G), rs114106519 (T/C), rs7220091 (A/G), rs12489026 (A/G), rs876270 (T/C), rs4968161 (T/C), rs62056907 (A/G), rs2235013 (T/C), rs16878812 (A/G), rs6549407 (A/G), rs28381848 (A/G), rs79723704 (A/C), rs72814052 (A/G), rs10152908 (T/C), rs172769 (A/C), rs78596668 (T/C), rs73307922 (T/C), rs3842 (A/G), rs7210584 (A/C), rs62402121 (T/C), rs55709291 (A/G), rs72747088 (A/G), rs929610 (G/C), rs6766242 (T/C), rs1468552 (G/C), rs78838114 (T/C), rs62489862 (T/C), rs894342 (A/G), rs58882373 (T/C), rs3811939 (A/G), rs6984688 (T/G), rs1018160 (T/C), rs76602912 (A/G), rs80067508 (A/G), rs74888440 (T/C), rs12481583 (T/C), rs66794218 (A/G), rs16946701 (A/G), rs75726724 (A/G), rs67959715 (T/A), rs11871392 (T/G), rs2044070 (A/G), rs77612799 (T/C), rs6743702 (T/C), rs616870 (T/C), rs79590198 (A/G), rs75715199 (A/G), rs13087555 (T/C), rs4869618 (T/C), rs117397046 (A/G), rs8042817 (A/G), rs2258097 (T/C), rs2260882 (C/G), rs532996 (A/G), rs11747040 (T/C), rs10034039 (T/G), rs116909369 (A/G), rs79134986 (A/G), rs117615688 (T/C), rs8032253 (T/C), rs12818653 (T/A), rs4587884 (A/C), rs77122853 (T/C), rs117615061 (T/C), rs74682905 (A/G), rs2257468 (T/C), rs2032582 (T/G), rs2235015 (T/G), rs2729794 (T/C), rs77549514 (A/G), rs74790420 (A/C), rs73129579 (T/C), rs12913346 (A/C), rs117560908 (T/C), rs72747091 (A/G), rs2935751 (A/G), rs4331446 (A/G), rs7523266 (T/C), rs7648662 (T/C), rs117034065 (A/G), rs4836256 (T/C), rs80238698 (T/C), rs3730173 (T/C), rs11687884 (T/C), rs72693005 (T/C), rs2589476 (T/C), rs9813396 (T/C), rs10482667 (A/G), rs72784444 (A/G), rs75074511 (T/C), rs7951003 (A/G), rs79584784 (A/G), rs2214102 (T/C), rs28811003 (A/G), rs6100261 (A/T), rs77152456 (A/G), rs66624622 (T/G), rs140302965 (A/G), rs11653269 (T/C), rs74405057 (A/G), rs7121 (A/G), rs16977818 (A/C), rs12490095 (T/C), rs118003903 (A/G), rs62377761 (T/C), P1_M_061510_6_34_M (−/CACTTACCTTCTTTGTGCCACAGTTTCCCTATCTAAAACACAAGGTTATCAGTTATC AACATCTCTTGGGATTGTGAGGACTAAAGTAATGCACATAAAG), rs375115639 (−/AAATTACCCTGTTAG GTTTCAATGAAACACCTTTTCTCTTGTAACAAACATCTCCTCCA AGCTAGAATTTCAAAACAG), rs1002204 (A/C), rs10062367 (A/G), rs10482642 (A/G), rs10482658 (A/G), rs1053989 (A/C), rs10851628 (T/C), rs10947562 (T/C), rs11069612 (A/G), rs11071351 (T/C), rs11091175 (A/G), rs11638450 (T/C), rs11715827 (T/G), rs11745958 (T/C), rs11834041 (A/G), rs1202180 (T/C), rs12054781 (A/G), rs12539395 (A/G), rs12720066 (T/G), rs1279754 (A/C), rs12872047 (T/C), rs12876742 (A/C), rs12917505 (A/G), rs13066950 (T/G), rs13229143 (C/G), rs1383707 (T/C), rs1441824 (T/C), rs1652311 (A/G), rs17064 (T/A), rs17100236 (A/G), rs17149699 (A/G), rs1724386 (A/G), rs17250255 (A/G), rs17327624 (T/G), rs17616338 (A/G), rs17687796 (A/G), rs17740874 (T/C), rs17763104 (T/C), rs1880748 (T/C), rs1882478 (A/G), rs1944887 (T/C), rs2028629 (A/G), rs2143404 (A/G), rs2173530 (T/C), rs220806 (T/C), rs2235047 (A/C), rs2242071 (A/G), rs2257474 (T/C), rs2295583 (NT), rs234629 (T/C), rs234630 (A/G), rs2436401 (A/G), rs258750 (T/C), rs2589487 (T/C), rs28364018 (T/G), rs28381774 (T/C), rs28381784 (A/G), rs2963155 (A/G), rs3133622 (T/G), rs32897 (T/C), rs33388 (NT), rs3730168 (T/C), rs3735833 (T/G), rs3777747 (A/G), rs3786066 (T/C), rs3798346 (T/C), rs3822736 (A/G), rs389035 (T/C), rs3924144 (A/G), rs4148737 (T/C), rs4148749 (G/C), rs417968 (T/C), rs4458144 (T/C), rs4515335 (T/C), rs4728699 (A/G), rs4758040 (A/G), rs4812040 (A/G), rs4912650 (T/G), rs4957891 (T/C), rs5906392 (A/G), rs6026561 (T/C), rs6026565 (T/A), rs6026567 (A/G), rs6026593 (A/G), rs6092704 (T/G), rs6100260 (A/G), rs6128461 (T/C), rs6415328 (T/C), rs6610868 (T/C), rs6686061 (A/C), rs6730350 (T/G), rs6746197 (T/C), rs6963426 (T/C), rs7121326 (T/C), rs7721799 (A/G), rs7787082 (T/C), rs7799592 (A/C), rs796245 (T/C), rs809482 (A/C), rs8125112 (T/C), rs919196 (A/G), rs920750 (T/C), rs9332385 (A/G), rs930473 (T/G), rs9324921 (A/C), rs9348979 (A/G), rs9571939 (A/C), and rs9892359 (T/C); (b) at least one polymorphism genotype being in linkage disequilibrium with any one of the polymorphism genotypes of (a); or (c) a combination of (a) and (b). In one embodiment of this aspect, the method further comprises predicting the treatment response from the presence or absence of the polymorphism genotypes of (a), (b), or (c).

In another aspect, the invention provides a method of treating a condition which is treatable by a CRHR1 antagonist in a subject in need thereof, comprising administering an effective amount of a CRHR1 antagonist to the subject, wherein the subject has been predicted to respond, or has an increased likelihood of responding, to treatment with a CRHR1 antagonist, as determined by the method of predicting a treatment response described above, wherein the CRHR1 antagonist is a compound of Formula I, as defined herein, or any one of “formulae I-VI” as disclosed at pages 2-6 and 12-48 of WO 2013/160317 (A2) (PCT/EP2013/058413). In another aspect, the invention provides a method of treating a condition which is treatable by a CRHR1 antagonist in a subject in need thereof, comprising administering an effective amount of a CRHR1 antagonist to the subject, wherein at least one polymorphism genotype associated with a treatment response of the subject to treatment with a CRHR1 antagonist has been detected, as determined by the method for detecting a polymorphism genotype associated with a treatment response, wherein the CRHR1 antagonist is a compound of Formula I, as defined herein, or any one of “formulae I-VI” as disclosed at pages 2-6 and 12-48 of WO 2013/160317 (A2) (PCT/EP2013/058413). Likewise, a CRHR1 antagonist for use in treating a condition which is treatable by a CRHR1 antagonist in a subject in need thereof is provided, wherein the subject has been predicted to respond, or has an increased likelihood of responding, to treatment with a CRHR1 antagonist, as determined by the method of predicting a treatment response described above, wherein the CRHR1 antagonist is a compound of Formula I, as defined herein, or any one of “formulae I-VI” as disclosed at pages 2-6 and 12-48 of WO 2013/160317 (A2) (PCT/EP2013/058413). In an alternative aspect, the invention provides a method of treating a condition which is treatable by a CRHR1 antagonist in a subject in need thereof, comprising administering an effective amount of a CRHR1 antagonist to the subject, wherein the subject has been predicted to respond, or has an increased likelihood of responding, to treatment with a CRHR1 antagonist, as determined by the method of predicting a treatment response described above, wherein the CRHR1 antagonist is selected from the group consisting of a Type I CRHR1 antagonist, a bicyclic Type II CRHR1 antagonist, an atypical CRHR1 antagonist, a cyclohexyl amide CRHR1 antagonist. In another aspect, the invention provides a method of treating a condition which is treatable by a CRHR1 antagonist in a subject in need thereof, comprising administering an effective amount of a CRHR1 antagonist to the subject, wherein at least one polymorphism genotype associated with a treatment response of the subject to treatment with a CRHR1 antagonist has been detected, as determined by the method for detecting a polymorphism genotype associated with a treatment response, wherein the CRHR1 antagonist is selected from the group consisting of a Type I CRHR1 antagonist, a bicyclic Type II CRHR1 antagonist, an atypical CRHR1 antagonist, a cyclohexyl amide CRHR1 antagonist. Likewise, a CRHR1 antagonist for use in treating a condition which is treatable by a CRHR1 antagonist in a subject in need thereof is provided, wherein the subject has been predicted to respond, or has an increased likelihood of responding, to treatment with a CRHR1 antagonist, as determined by the method of predicting a treatment response described above, wherein the CRHR1 antagonist is selected from the group consisting of a Type I CRHR1 antagonist, a bicyclic Type II CRHR1 antagonist, an atypical CRHR1 antagonist, a cyclohexyl amide CRHR1 antagonist.

The above aspects of the invention can be put into practice in any one of the following embodiments.

In one embodiment, providing a biological sample comprises extraction and/or purification of nucleic acids such as DNA or RNA, in particular genomic DNA from the subject's sample. In one embodiment, the detecting step can comprise amplification of nucleic acids extracted and/or purified from the sample obtained from the subject, and optionally clean-up of amplified products. The detecting step can further comprise fragmentation of amplified nucleic acids, or labelling of amplified nucleic acids.

In one embodiment, the detecting step can comprise specific hybridization of at least one polynucleotide to a nucleic acid comprising at least one polymorphism genotype selected from the group disclosed in Table 2 herein. Hybridization can be achieved by mixing and heating the at least one polynucleotide and the sample nucleic acid to a temperature at which denaturation occurs, e.g., at about 90-95° C. and subsequent incubation at a temperature at which hybridization occurs, e.g., at about 45-55° C. in buffer conditions suitable for specific hybridization. In one embodiment the polynucleotide is labelled. The polynucleotide can be a primer or probe. Specifically, in some embodiments, the detecting step comprises a method selected from the group consisting of allele-specific oligonucleotide (ASO)-dot blot analysis, primer extension assays, iPLEX polymorphism/SNP genotyping, dynamic allele-specific hybridization (DASH) genotyping, the use of molecular beacons, tetra primer ARMS PCR, a flap endonuclease invader assay, an oligonucleotide ligase assay, PCR-single strand conformation polymorphism (SSCP) analysis, quantitative real-time PCR assay, polymorphism/SNP microarray based analysis, restriction enzyme fragment length polymorphism (RFLP) analysis, targeted resequencing analysis and/or whole genome sequencing analysis.

In one embodiment, the predicting step comprises: (a) determining whether the subject will respond, or has an increased likelihood of responding to the treatment with a CRHR1 antagonist; and/or (b) determining whether the subject will not respond, or has a decreased likelihood of responding to the treatment with a CRHR1 antagonist. The determining step may further comprise, but is not limited to, one or more statistical analysis methods selected from the group consisting of artificial neural network learning, decision tree learning, decision tree forest learning, linear discriminant analysis, non-linear discriminant analysis, genetic expression programming, relevance vector machines, linear models, generalized linear models, generalized estimating equations, generalized linear mixed models, the elastic net, the lasso support vector machine learning, Bayesian network learning, probabilistic neural network learning, clustering, and regression analysis. The predicting step may also comprise providing a value indicative of the subject being responsive, or having an increased likelihood of responding to the treatment with a CRHR1 antagonist; and/or providing a value indicative of the subject being non-responsive, or having a decreased likelihood of responding to the treatment with a CRHR1 antagonist.

In one embodiment, the one or more polymorphism genotypes comprise at least two, at least three, at least four, at least five, at least six, at least seven, at least eight, at least nine, at least ten, at least 15, at least 20, at least 30, at least 40, at least 50, at least 60, at least 70, at least 80, at least 90, at least 100, at least 200, or all (a) polymorphism genotypes selected from the group consisting of the polymorphism genotypes disclosed in Table 2, (b) polymorphism genotypes being in linkage disequilibrium with the polymorphism genotypes disclosed in Table 2; or (c) a combination of (a) and (b).

In a specific embodiment, the one or more polymorphism genotypes comprise (a) at least two polymorphism genotypes selected from the group consisting of the polymorphism genotypes disclosed in Table 2, (b) at least two polymorphism genotypes being in linkage disequilibrium with the polymorphism genotypes disclosed in Table 2; or (c) a combination of (a) and (b). Exemplary sets of at least two polymorphism genotypes useful in the methods of the invention are disclosed in Table 5. Therefore, the specific combinations of at least two polymorphism genotypes disclosed in Table 5 are used in specific embodiments of the invention, while further combinations of at least two polymorphism genotypes are expressly contemplated.

In another specific embodiment, the one or more polymorphism genotypes comprise (a) at least four polymorphism genotypes selected from the group consisting of the polymorphism genotypes disclosed in Table 2, (b) at least four polymorphism genotypes being in linkage disequilibrium with the polymorphism genotypes disclosed in Table 2, or (c) a combination of (a) and (b). Exemplary sets of at least four polymorphism genotypes useful in the methods of the invention are disclosed in Table 6. Therefore, the specific combinations of at least four polymorphism genotypes disclosed in Table 6 are used in specific embodiments of the invention, while further combinations of at least four polymorphism genotypes are expressly contemplated.

In another specific embodiment, the one or more polymorphism genotypes comprise (a) at least eight polymorphism genotypes selected from the group consisting of the polymorphism genotypes disclosed in Table 2, (b) at least eight polymorphism genotypes being in linkage disequilibrium with the polymorphism genotypes disclosed in Table 2, or (c) a combination of (a) and (b). Exemplary sets of at least eight polymorphism genotypes useful in the methods of the invention are shown in Table 7. Therefore, the specific combinations of at least eight polymorphism genotypes disclosed in Table 7 are used in specific embodiments of the invention, while further combinations are expressly contemplated.

In another embodiment, the one or more polymorphism genotypes comprise (a) at least 16 polymorphism genotypes selected from the group consisting of the polymorphism genotypes disclosed in Table 2, (b) at least 16 polymorphism genotypes being in linkage disequilibrium with the polymorphism genotypes disclosed in Table 2, or (c) a combination of (a) and (b). In another embodiment, the one or more polymorphism genotypes comprise (a) at least 32 polymorphism genotypes selected from the group consisting of the polymorphism genotypes disclosed in Table 2, (b) at least 16 polymorphism genotypes being in linkage disequilibrium with the polymorphism genotypes disclosed in Table 2, or (c) a combination of (a) and (b). In another embodiment, the one or more polymorphism genotypes comprise at least 150 polymorphism genotypes selected from the group consisting of the polymorphism genotypes disclosed in Table 2, (b) at least 16 polymorphism genotypes being in linkage disequilibrium with the polymorphism genotypes disclosed in Table 2, or (c) a combination of (a) and (b). In another embodiment, the one or more polymorphism genotypes comprise all polymorphism genotypes disclosed in Table 2.

In some embodiments, the method can include detecting the presence or absence of (a) one or more of the polymorphism genotypes disclosed in Tables 2, 5, 6, or 7, (b) one or more polymorphism genotypes being in linkage disequilibrium with the polymorphism genotypes disclosed in Tables 2, 5, 6, or 7, or (c) a combination of (a) and (b), predicting that the subject will respond, or is likely to respond to treatment with a CRHR1 antagonist and selecting a treatment with a CRHR1 agent for the subject. The method can further include administering the CRHR1 antagonist to the subject.

In some embodiments, the predicting step can include creating a record indicating that the subject will respond, or is likely to respond to treatment with a CRHR1 antagonist. The record can be created on a computer readable medium.

In some embodiments, the method can include detecting the presence or absence of (a) one or more of any of the polymorphism genotypes disclosed in Tables 2, 5, 6 or 7, (b) one or more polymorphism genotypes being in linkage disequilibrium with the polymorphism genotypes disclosed in Tables 2, 5, 6, or 7, or (c) a combination of (a) and (b), predicting that the subject will not respond, or is not likely to respond to a treatment with a CRHR1 antagonist and selecting a treatment with treatment with a non-CRHR1 antagonist for the subject. The method can further include administering the treatment with the non-CRHR1 antagonist to the subject.

In some embodiments, the method can include creating a record indicating that the subject will not respond, or is not likely to respond to a treatment with a CRHR1 antagonist. The record can be created on a computer readable medium.

In one embodiment, the subject is a mammal. Preferably, in all aspects of the invention, the subject is human.

In one embodiment, the subject has a condition which is treatable by a treatment with a CRHR1 antagonist, as described herein. The condition can be characterized, caused or accompanied by CRH overproduction or over-activity. The condition can be characterized, caused or accompanied by ACTH overproduction or over-activity. The condition can be characterized, caused or accompanied by over-activity of the Hypothalamic-pituitary-adrenal (HPA) axis.

In another embodiment, the subject has and/or the treatment is a treatment of a condition selected from the group consisting of anxiety symptoms, generalized anxiety disorder, panic, phobias, obsessive-compulsive disorder, post-traumatic stress disorder, sleep disorders such as insomnia, hypersomnia, narcolepsy, idiopathic hypersomnia, excessive amounts of sleepiness, lack of alertness, lack of attentiveness, absentmindedness and/or lack of or aversion to movement or exercise, sleep disorders induced by stress, pain perception such as fibromyalgia, mood disorders such as depressive symptoms, including major depression, single episode depression, recurrent depression, child abuse induced depression, mood disorders associated with premenstrual syndrome, and postpartum depression, dysthymia, bipolar disorders, cyclothymia, chronic fatigue syndrome, stress-induced headache, eating disorders such as anorexia and bulimia nervosa, hemorrhagic stress, stress-induced psychotic episodes, endocrine disorders involving ACTH overproduction, ACTH over-activity, e.g., adrenal disorders, including, but not limited to congenital adrenal hyperplasia (CAH), euthyroid sick syndrome, syndrome of inappropriate antidiarrhetic hormone (ADH), obesity, infertility, head traumas, spinal cord trauma, ischemic neuronal damage (e.g., cerebral ischemia such as cerebral hippocampal ischemia), excitotoxic neuronal damage, epilepsy, senile dementia of the Alzheimers type, multi-infarct dementia, amyotrophic lateral sclerosis, chemical dependencies and addictions (e.g., dependencies on alcohol, nicotine, cocaine, heroin, benzodiazepines, or other drugs), drug and alcohol withdrawal symptoms, hypertension, tachycardia, congestive heart failure, osteoporosis, premature birth, and hypoglycaemia, inflammatory disorders such as rheumatoid arthritis and osteoarthritis, pain, asthma, psoriasis and allergies, irritable bowel syndrome, Crohn's disease, spastic colon, post-operative ileus, ulcer, diarrhea, stress-induced fever, human immunodeficiency virus (HIV) infections, neurodegenerative diseases such as Alzheimer's disease, Parkinson's disease and Huntington's disease, gastrointestinal diseases, stroke, stress induced immune dysfunctions, muscular spasms, urinary incontinence.

In a specific embodiment, the subject has and/or the treatment is a treatment of depressive symptoms, anxiety symptoms or both depressive symptoms and anxiety symptoms. In another specific embodiment, the subject has and/or the treatment is a treatment of depressive disorder, anxiety disorder or both depressive disorder and anxiety disorder. In another specific embodiment, the subject has and/or the treatment is a treatment of a sleep disorder.

In contrast to the prior art, the present invention identifies sets of polymorphisms indicative of a clinical response in subjects which are in need of a treatment with a CRHR1 antagonist. Therefore, in all aspects of the invention, the treatment response to treatment with the CRHR1 antagonist is preferably a clinical response. Generally, the clinical response can be a prevention, alteration, alleviation or complete remission of a clinical parameter in any of the above conditions. In particular, the clinical response can be a prevention, alteration, alleviation or complete remission of depressive symptoms and/or anxiety symptoms, or a decrease in adverse effects resulting from the treatment.

In some embodiments, the clinical response is a prevention, alteration, alleviation or complete remission of depressive symptoms, as determined using a scale selected from the group consisting of the Hamilton Depression Rating Scale (HAM-D), the Beck Depression Inventory (BDI), the Montgomery-Asberg Depression Scale (MADRS), the Geriatric Depression Scale (GDS), and/or the Zung Self-Rating Depression Scale (ZSRDS).

In some embodiments, the clinical response is a prevention, alteration, alleviation or complete remission of anxiety symptoms, as determined using a scale selected from the group consisting of Hamilton Anxiety Rating Scale (HAM-A) and/or the State-Trait Anxiety Rating Scale (STAI).

Any of the methods described herein can further include a step of prescribing a treatment with a CRHR1 antagonist or non-CRHR1 antagonist (the choice of which depends upon the outcome of the predictive methods described herein) for the subject.

In all aspects, the sample obtained from the subject can comprise any type of cells containing nucleic acids from the subject, in particular genomic DNA. Specifically, the sample can be, e.g., a buccal sample, a blood sample, a tissue sample, a formalin-fixed, paraffin-embedded tissue sample, or a hair follicle. The sample obtained from the subject can comprise purified nucleic acids, such as purified genomic DNA.

In any of the above aspects, a CRHR1 antagonist can be any compound capable of binding directly or indirectly to a CRHR1 so as to modulate any of its known biological activity receptor mediated activity, as is commonly known in the art. For instance, CRHR1 antagonists will specifically bind to CRHR1 with a K_{D} of 1 μM or less, preferably with a K_{D} of 100 nM or less and/or specifically inhibit CRH binding to CRHR1 in vitro with Kᵢ values of 1 μM or less, preferably with Kᵢ values of 100 nM or less. Alternatively or in addition, a CRHR1 antagonist will also inhibit cAMP accumulation and adrenocorticotropic hormone (ACTH) production in vitro and/or attenuate CRH and ACTH production in vivo.

In some embodiments of any of the above aspects, the CRHR1 antagonist can be a compound of Formula (I)

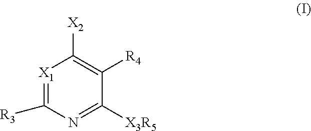

or a pharmaceutically acceptable salt thereof, wherein
X₁ is —CR₆ or N;
X₂ is —NR₁R₂, —CR₁R₂R₉, —C(═CR₂R₁₀)R₁, —NHCHR₁R₂, —OCHR₁R₂, —SCHR₁R₂, —CHR₂OR₁₀, —CHR₂SR₁₀, —C(S)R₂ or —C(O)R₂;
X₃ is NH, O, S, —N(C₁-C₂ alkyl) or —C(R₁₁R₁₂), wherein R₁₁ and R₁₂ are each, independently, hydrogen, trifluoromethyl or methyl, or one of R₁₁ and R₁₂ is cyano and the other is hydrogen or methyl, or
X₃ is N and X₃ and R₄ form a 5-membered ring substituted at X₃ with R₅;
R₁ is C₁-C₆ alkyl which may optionally be substituted with one or two substituents R₇ independently selected from the group consisting of hydroxy, fluoro, chloro, bromo, iodo, CF₃, C₃-C₈ cycloalkyl, C₁-C₄ alkoxy, —O—CO—(C₁-C₄ alkyl), —O—CO—NH(C₁-C₄ alkyl), —O—CO—N(C₁-C₄ alkyl)(C₁-C₂ alkyl), —NH(C₁-C₄ alkyl), —N(C₁-C₂ alkyl)(C₁-C₄ alkyl), —S(C₁-C₄ alkyl), —N(C₁-C₄ alkyl)CO(C₁-C₄ alkyl), —NHCO(C₁-C₄ alkyl), —COO(C₁-C₄ alkyl), —CONH(C₁-C₄ alkyl), —CON(C₁-C₄ alkyl)(C₁-C₂ alkyl), CN, NO₂, —SO(C₁-C₄ alkyl) and —SO₂(C₁-C₄ alkyl), and wherein said C₁-C₆ alkyl and the (C₁-C₄) alkyl moieties in the foregoing R₇ groups may optionally contain one carbon-carbon double or triple bond;
R₂ is C₁-C₁₂ alkyl, C₁-C₁₂ alkoxyalkyl, aryl or —(C₁-C₄ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, benzisoxazolyl, benzimidazolyl, indolyl, oxadiazolyl or benzoxazolyl;
3- to 8-membered cycloalkyl or —(C₁-C₆ alkylene)cycloalkyl, wherein one or two of the ring carbons of said cycloalkyl having at least 4 ring members and the cycloalkyl moiety of said —(C₁-C₆ alkylene)cycloalkyl having at least 4 ring members may optionally be replaced by an oxygen or sulfur atom or by N—R₈ wherein R₈ is hydrogen or C₁-C₄ alkyl;
and wherein each of the foregoing R₂ groups may optionally be substituted with from one to three substituents independently selected from chloro, fluoro and Cᵣ C₄ alkyl, or with one substituent selected from bromo, iodo, C₁-C₆ alkoxy, —O—CO—(C₁-C₆ alkyl), —O—CO—N(C₁-C₄ alkyl)(C₁-C₂ alkyl), —S(C₁-C₆ alkyl), CN, NO₂, —SO(C₁-C₄ alkyl), and —SO₂(C₁-C₄ alkyl), and wherein said C₁-C₁₂ alkyl and/or the C₁-C₄ alkylene moiety of said —(C₁-C₄ alkylene)aryl may optionally contain one carbon-carbon double or triple bond;
or —NR₁R₂ or —CR₁R₂R₈ may form a saturated 5- to 8-membered ring which may optionally contain one or two carbon-carbon double bonds and/or in which one or two of the ring carbons may optionally be replaced by an oxygen, nitrogen or sulfur atom and which may be substituted with at least one substituent;
R₃ is methyl, ethyl, fluoro, chloro, bromo, iodo, cyano, methoxy, OCF₃, methylthio, methylsulfonyl, CH₂OH, or CH₂OCH₃;
R₄ is hydrogen, C₁-C₄ alkyl, fluoro, chloro, bromo, iodo, C₁-C₄ alkoxy, trifluoromethoxy, —CH₂OCH₃, —CH₂OCH₂CH₃, —CH₂CH₂OCH₃, —CH₂OCF₃, CF₃, amino, nitro, —NH(C₁-C₄ alkyl), —N(CH₃)₂, —NHCOCH₃—NHCONHCH₃, —SOₙ(C₁-C₄ alkyl) wherein n is 0, 1 or 2, hydroxy, —CO(C₁-C₄ alkyl), —CHO, cyano or —COO(C₁-C₄ alkyl) wherein said C₁-C₄ alkyl may optionally contain one double or triple bond and/or may optionally be substituted with one substituent selected from hydroxy, amino, —NHCOCH₃, —NH(C₁-C₂ alkyl), —N(C₁-C₂ alkyl)₂, —COO(C₁-C₄ alkyl), —CO(C₁-C₄ alkyl), C₁-C₃ alkoxy, C₁-C₃ thioalkyl, fluoro, chloro, cyano and nitro;
R₅ is phenyl, naphthyl, thienyl, benzothienyl, pyridyl, quinolyl, pyrazinyl, pyrimidyl, furanyl, benzofuranyl, benzothiazolyl, or indolyl, wherein each of the above groups R₅ is substituted with from one to three substituents independently selected from fluoro, chloro, C₁-C₆ alkyl and C₁-C₆ alkoxy, or with one substituent selected from hydroxy, iodo, bromo, formyl, cyano, nitro, trifluoromethyl, amino, (C₁-C₆ alkyl)O(C₁-C₆)alkyl, —NHCH₃, —N(CH₃)₂, —COOH, —COO(C₁-C₄ alkyl), —CO(C₁-C₄ alkyl), —SO₂NH(C₁-C₄ alkyl), —SO₂N(C₁-C₄ alkyl)(C₁-C₂ alkyl), —SO₂NH₂, —NHSO₂(C₁-C₄ alkyl), —S(C₁-C₆ alkyl) and SO₂(C₁-C₆ alkyl), and wherein the C₁-C₄ alkyl and the C₁-C₆ alkyl moieties of the foregoing R₅ groups may optionally be substituted with one or two fluoro groups or with one substituent selected from hydroxy, amino, methylamino, dimethylamino and acetyl;
R₆ is hydrogen, methyl, fluoro, chloro, bromo, iodo, cyano, hydroxy, —O(C₁-C₄ alkyl), —C(O)(C₁-C₄ alkyl), —C(O)O(C₁-C₄ alkyl), —OCF₃, CF₃, —CH₂OH, —CH₂OCH₃ or —CH₂OCH₂CH₃;
R₉ is hydrogen, hydroxy, fluoro, or methoxy;
R₁₀ is hydrogen or C₁-C₄ alkyl.

Further, CRHR1 antagonists can encompass compounds of Formula (I) as defined above, wherein X₁ is —CR₆. Optionally, X₁ is —CR₆, wherein R₆ is hydrogen.

In a further embodiment, the 5- to 8-membered ring formed by —NR₁R₂ or —CR₁R₂R₉ of the compound of Formula (I) as defined above is substituted with at least one substituent selected from C₁-C4 alkyl or with a 4-8 membered ring, which may be saturated or may contain one to three double bonds and in which one carbon atom may be replaced by CO or SO₂ and one to four carbon atoms may optionally be replaced by nitrogen.

In a specific embodiment, X₂ of the compound of Formula (I) as defined above is —NHCHR₁R₂, —OCHR₁R₂ or —NR₁R₂.

Optionally, in the compounds of Formula (I) as defined above —NHCHR₁R₂ is —NHCH(CH₂OCH₃)₂, —NHCH(CH₂OCH₃)(CH₂CH₃), —NHCH(CH₂CH₃)₂, —NHCH(CH₂CH₂OCH₃)₂ or —NHCHR₁R₂, wherein R₁ is ethyl and R₂ is oxadiazolyl substituted with methyl, or —NR₁R₂ is —N(CH₂CH₃)(CH₃), —N(CH₂CH₂CH₃)₂, —N(CH₂CH₂CH₃)(CH₂-cyclopropyl), —N(CH₂CH₃)(CH₂CH₂CH₂CH₃), —N(CH₂CH₂OCH₃)₂, or —N(CH₂CH₂OCH₃)(CH₂CH₂CH₃), or —OCHR₁R₂ is —OCH(CH₂CH₃)₂, —OCH(CH₂CH₃)CH₃, —OCH(CH₂CH₃)(CH₂CH₂CH₃), —OCH(CH₂CH₃)(CH₂OCH₃).

In another embodiment, R₃ and R₄ of the compound of Formula (I) as defined above are methyl.

In a further embodiment, X₃ of the compound of Formula (I) as defined above is O.

In another embodiment, R₅ of the compound of Formula (I) as defined above is phenyl substituted with from one to three substituent(s) independently selected from the group CH₃, CH₂CH₃, OCH₃, CI, F, CF₃.

In a specific embodiment of any of the above aspects, the CRHR1 antagonist is a compound of the Formula (VI)

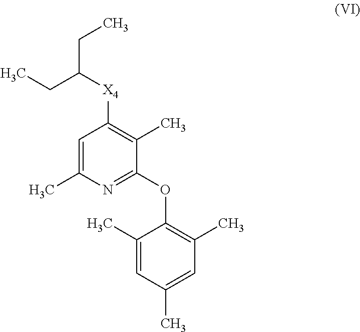

or a pharmaceutically acceptable salt thereof, wherein

X₄ is O or NH.

In some embodiments of the methods of predicting, or the methods of detecting as described herein, the CRHR1 antagonist is a compound of Formulae I or VI, as defined above, or any one of “formulae I-VI” as disclosed at pages 2-6 and 12-48 of WO 2013/160317 (A2) (PCT/EP2013/058413) or a pharmaceutically acceptable salt thereof.

In alternative embodiments of the methods of predicting, or the methods of detecting as described herein, the CRHR1 antagonist is selected from the group consisting of GW876008 (Emicerfont), GSK-561679 (NBI-77860, Verucerfont), GSK586529, BMS-562,086 (Pexacerfont), NBI-30775 (R-121919), NBI-34101, CP-316,311, CP-376,395, PF-00572778, NVP-AAG561, Ono-2333MS, E2508, E2009, R317573 (JNJ19567470, CRA5626), R278995 (CRA0450), CRA-1000, CRA-1001, CP154,526, Antalarmin, DMP-695, DMP-696, DMP-904, SC-241, BMS-561388, NB130545, PD-171729, NB134041, NB135965, SN003, NBI-27914, trans-2-chloro-N-(4-((5-fluoro-4-methyl-pyridin-2-ylamino)-methyl)-cyclohexyl)-5-(trifluoromethyl)-benzamide, SSR-125543, or a pharmaceutically acceptable salt thereof.

In preferred embodiments of the methods of predicting, or the methods of detecting, as described herein, the CRHR1 antagonist is selected from the group consisting of BMS-562,086 (Pexacerfont), CP-316,311, Ono-2333MS, GW876008 (Emicerfont), GSK-561679 (NBI-77860, Verucerfont), NBI-30775 (R-121919), DMP-696, and SSR-125543, or a pharmaceutically acceptable salt thereof. In preferred embodiments of the methods of predicting, or the methods of detecting, as described herein, the CRHR1 antagonist is SSR-125543. In one embodiment of the method of treatment, the CRHR1 antagonist is not SSR-125543. In a preferred embodiment of all aspects, the CRHR1 antagonist is BMS-562,086 (Pexacerfont). In another preferred embodiment of all aspects, the CRHR1 antagonist is CP-316,311. In another preferred embodiment of all aspects, the CRHR1 antagonist is ONO-2333MS. In another preferred embodiment of all aspects, the CRHR1 antagonist is GW876008 (Emicerfont). In another preferred embodiment of all aspects, the CRHR1 antagonist is GSK-561679 (Verucerfont). In another preferred embodiment of all aspects, the CRHR1 antagonist is NBI-30775 (R121919). In another preferred embodiment of all aspects, the CRHR1 antagonist is DMP-696.

In another aspect, the disclosure provides a composition comprising at least one polynucleotide capable of specifically hybridizing to nucleic acids comprising: (a) at least one polymorphism genotype selected from the group consisting of rs34169260 (A/G), rs796287 (A/C), rs56149945 (A/G), rs6190 (T/C), rs7179092 (T/C), rs7614867 (A/G), rs920640 (T/C), rs7167722 (T/C), rs920638 (T/C), rs7165629 (T/C), rs80049044 (T/A), rs16941058 (A/G), rs112015971 (A/G), rs10894873 (T/C), rs117455294 (T/G), rs1170303 (T/C), rs16940681 (C/G), rs968519 (T/C), rs28381866 (T/C), rs79320848 (T/G), rs114653646 (T/G), rs2589496 (T/C), rs10482650 (A/G), rs17614642 (A/G), rs73200317 (T/C), rs1380146 (T/A), rs735164 (T/C), rs730976 (T/G), rs55934524 (T/G), rs4570614 (A/G), rs4458044 (C/G), rs77850169 (A/G), rs35339359 (A/G), rs34800935 (T/C), rs72945439 (T/C), rs113959523 (A/G), rs116798177 (A/G), rs11247577 (T/G), rs75869266 (T/C), rs74372553 (T/C), rs11691508 (A/G), rs6493965 (A/G), rs4869476 (T/C), rs3730170 (T/C), rs2145288 (A/C), rs2935752 (A/C), rs146512400 (A/G), rs62057097 (T/C), rs115061314 (T/C), rs34113594 (T/G), rs61751173 (A/G), rs74338736 (A/C), rs10851726 (T/C), rs4610906 (T/C), rs59485211 (T/C), rs7060015 (T/G), rs75710780 (T/G), rs6520908 (T/C), rs487011 (T/G), rs1383699 (A/C), rs67516871 (A/G), rs114106519 (T/C), rs7220091 (A/G), rs12489026 (A/G), rs876270 (T/C), rs4968161 (T/C), rs62056907 (A/G), rs2235013 (T/C), rs16878812 (A/G), rs6549407 (A/G), rs28381848 (A/G), rs79723704 (A/C), rs72814052 (A/G), rs10152908 (T/C), rs172769 (A/C), rs78596668 (T/C), rs73307922 (T/C), rs3842 (A/G), rs7210584 (A/C), rs62402121 (T/C), rs55709291 (A/G), rs72747088 (A/G), rs929610 (G/C), rs6766242 (T/C), rs1468552 (G/C), rs78838114 (T/C), rs62489862 (T/C), rs894342 (A/G), rs58882373 (T/C), rs3811939 (A/G), rs6984688 (T/G), rs1018160 (T/C), rs76602912 (A/G), rs80067508 (A/G), rs74888440 (T/C), rs12481583 (T/C), rs66794218 (A/G), rs16946701 (A/G), rs75726724 (A/G), rs67959715 (T/A), rs11871392 (T/G), rs2044070 (A/G), rs77612799 (T/C), rs6743702 (T/C), rs616870 (T/C), rs79590198 (A/G), rs75715199 (A/G), rs13087555 (T/C), rs4869618 (T/C), rs117397046 (A/G), rs8042817 (A/G), rs2258097 (T/C), rs2260882 (C/G), rs532996 (A/G), rs11747040 (T/C), rs10034039 (T/G), rs116909369 (A/G), rs79134986 (A/G), rs117615688 (T/C), rs8032253 (T/C), rs12818653 (T/A), rs4587884 (A/C), rs77122853 (T/C), rs117615061 (T/C), rs74682905 (A/G), rs2257468 (T/C), rs2032582 (T/G), rs2235015 (T/G), rs2729794 (T/C), rs77549514 (A/G), rs74790420 (A/C), rs73129579 (T/C), rs12913346 (A/C), rs117560908 (T/C), rs72747091 (A/G), rs2935751 (A/G), rs4331446 (A/G), rs7523266 (T/C), rs7648662 (T/C), rs117034065 (A/G), rs4836256 (T/C), rs80238698 (T/C), rs3730173 (T/C), rs11687884 (T/C), rs72693005 (T/C), rs2589476 (T/C), rs9813396 (T/C), rs10482667 (A/G), rs72784444 (A/G), rs75074511 (T/C), rs7951003 (A/G), rs79584784 (A/G), rs2214102 (T/C), rs28811003 (A/G), rs6100261 (A/T), rs77152456 (A/G), rs66624622 (T/G), rs140302965 (A/G), rs11653269 (T/C), rs74405057 (A/G), rs7121 (A/G), rs16977818 (A/C), rs12490095 (T/C), rs118003903 (A/G), rs62377761 (T/C), P1_M_061510_6_34_M (−/CACTTAC CTTCTTTGTGCCACAGTTTCCCTATCTAAAACACAAGGTTATCAGTTATCAACATCTCT TGGGATTGTGAGGACTAAAGTAATGCACATAAAG), rs375115639 (−/AAATTACCCTGTTAG GTTTCAATGAAACACCTTTTCTCTTGTAACAAACATCTCC TCCAAGCTAGAATTTCAAAACAG), rs1002204 (A/C), rs10062367 (A/G), rs10482642 (A/G), rs10482658 (A/G), rs1053989 (A/C), rs10851628 (T/C), rs10947562 (T/C), rs11069612 (A/G), rs11071351 (T/C), rs11091175 (A/G), rs11638450 (T/C), rs11715827 (T/G), rs11745958 (T/C), rs11834041 (A/G), rs1202180 (T/C), rs12054781 (A/G), rs12539395 (A/G), rs12720066 (T/G), rs1279754 (A/C), rs12872047 (T/C), rs12876742 (A/C), rs12917505 (A/G), rs13066950 (T/G), rs13229143 (C/G), rs1383707 (T/C), rs1441824 (T/C), rs1652311 (A/G), rs17064 (T/A), rs17100236 (A/G), rs17149699 (A/G), rs1724386 (A/G), rs17250255 (A/G), rs17327624 (T/G), rs17616338 (A/G), rs17687796 (A/G), rs17740874 (T/C), rs17763104 (T/C), rs1880748 (T/C), rs1882478 (A/G), rs1944887 (T/C), rs2028629 (A/G), rs2143404 (A/G), rs2173530 (T/C), rs220806 (T/C), rs2235047 (A/C), rs2242071 (A/G), rs2257474 (T/C), rs2295583 (A/T), rs234629 (T/C), rs234630 (A/G), rs2436401 (A/G), rs258750 (T/C), rs2589487 (T/C), rs28364018 (T/G), rs28381774 (T/C), rs28381784 (A/G), rs2963155 (A/G), rs3133622 (T/G), rs32897 (T/C), rs33388 (NT), rs3730168 (T/C), rs3735833 (T/G), rs3777747 (A/G), rs3786066 (T/C), rs3798346 (T/C), rs3822736 (A/G), rs389035 (T/C), rs3924144 (A/G), rs4148737 (T/C), rs4148749 (G/C), rs417968 (T/C), rs4458144 (T/C), rs4515335 (T/C), rs4728699 (A/G), rs4758040 (A/G), rs4812040 (A/G), rs4912650 (T/G), rs4957891 (T/C), rs5906392 (A/G), rs6026561 (T/C), rs6026565 (T/A), rs6026567 (A/G), rs6026593 (A/G), rs6092704 (T/G), rs6100260 (A/G), rs6128461 (T/C), rs6415328 (T/C), rs6610868 (T/C), rs6686061 (A/C), rs6730350 (T/G), rs6746197 (T/C), rs6963426 (T/C), rs7121326 (T/C), rs7721799 (A/G), rs7787082 (T/C), rs7799592 (A/C), rs796245 (T/C), rs809482 (A/C), rs8125112 (T/C), rs919196 (A/G), rs920750 (T/C), rs9332385 (A/G), rs930473 (T/G), rs9324921 (A/C), rs9348979 (A/G), rs9571939 (A/C), and rs9892359 (T/C), as disclosed in Table 2; (b) at least one polymorphism genotype being in linkage disequilibrium with any one of the polymorphism genotypes of (a), or (c) a combination of (a) and (b). In one embodiment, the composition comprises less than 100,000, less than 90,000, less than 80,000, less than 70,000, less than 60,000, less than 50,000, less than 40,000, less than 30,000, less than 20,000, less than 15,000, less than 10,000, less than 5,000, less than 4,000, less than 3,000, less than 2,000, less than 1,500, less than 1,000, less than 750, less than 500, less than 200, less than 100, or less than 50 different polynucleotides in total. In some embodiments, the composition comprises at least two, at least three, at least four, at least five, at least six, at least seven, at least eight, at least nine, at least 10, at least 11, at least 12, at least 15, at least 20, or at least 30, or at least 50, or at least 100, or at least 200, or 274 polynucleotides capable of specifically hybridizing to nucleic acids comprising each of at least two, at least three, at least four, at least five, at least six, at least seven, at least eight, at least nine, at least 10, at least 11, at least 12, at least 15, at least 20, or at least 30, or at least 50, or at least 100, or at least 200, or 274 (a) polymorphism genotypes as disclosed in Table 2, (b) polymorphism genotypes being in linkage disequilibrium with any one of the polymorphism genotypes of (a), or (c) a combination of (a) and (b).

In a specific embodiment, the disclosure provides a composition containing at least two polynucleotides capable of specifically hybridizing to nucleic acids comprising (a) each of at least two polymorphism genotypes selected from the group consisting of the polymorphism genotypes as disclosed in Table 2, (b) each of at least two polymorphism genotypes being in linkage disequilibrium with any one of the polymorphism genotypes of (a), or (c) a combination of (a) and (b). In another specific embodiment, the disclosure provides a composition containing at least four polynucleotides capable of specifically hybridizing to (a) each of at least four polymorphism genotypes selected from the group consisting of the polymorphism genotypes as disclosed in Table 2, (b) each of at least four polymorphism genotypes being in linkage disequilibrium with any one of the polymorphism genotypes of (a), or (c) a combination of (a) and (b). In another specific embodiment, the disclosure provides a composition containing at least eight polynucleotides capable of specifically hybridizing to (a) each of at least eight polymorphism genotypes selected from the group consisting of the polymorphism genotypes as disclosed in Table 2, (b) each of at least eight polymorphism genotypes being in linkage disequilibrium with any one of the polymorphism genotypes of (a), or (c) a combination of (a) and (b). In another specific embodiment, the disclosure provides a composition containing at least 16 polynucleotides capable of specifically hybridizing to each of at least 16 polymorphism genotypes selected from the group consisting of the polymorphism genotypes as disclosed in Table 2, (b) each of at least 16 polymorphism genotypes being in linkage disequilibrium with any one of the polymorphism genotypes of (a), or (c) a combination of (a) and (b). In another specific embodiment, the disclosure provides a composition containing at least 32 polynucleotides capable of specifically hybridizing to (a) each of at least 32 polymorphism genotypes selected from the group consisting of the polymorphism genotypes as disclosed in Table 2, (b) each of at least 32 polymorphism genotypes being in linkage disequilibrium with any one of the polymorphism genotypes of (a), or (c) a combination of (a) and (b). In another specific embodiment, the disclosure provides a composition containing at least 150 polynucleotides capable of specifically hybridizing to (a) each of at least 150 polymorphism genotypes selected from the group consisting of the polymorphism genotypes as disclosed in Table 2, (b) each of at least 150 polymorphism genotypes being in linkage disequilibrium with any one of the polymorphism genotypes of (a), or (c) a combination of (a) and (b). In another specific embodiment, the disclosure provides a composition containing 274 polynucleotides capable of specifically hybridizing to each of the 274 polymorphism genotypes as disclosed in Table 2.

In some embodiments, the at least one polynucleotide capable of specifically hybridizing to the polymorphism genotypes is selected from the group consisting of the polynucleotides disclosed as “AlleleA Probe” in Table 2. In particular, the composition can comprise all of the polynucleotides disclosed as “AlleleA Probe” in Table 2.

In any of the compositions described above, the at least one polynucleotide can be bound to a solid support. The solid support can be in the form of an array on a microarray chip, a particle (e.g., an encoded, magnetic, or magnetic and encoded particle), or any other solid support described herein.

In yet another aspect, the disclosure provides a kit useful in determining the presence or absence of one or more polymorphism genotypes. The kit can include any of the compositions described above and, optionally, instructions for detecting one or more polymorphism genotypes. The kit can also include, e.g., one or more additional reagents for detecting the presence or absence of the one or more polymorphism genotypes described herein. For example, the kit can include primers (e.g., random hexamers or oligo(dT) primers), reverse transcriptase, a DNA polymerase (e.g., Taq polymerase), T4 polynucleotide kinase, one or more detectable labels (such as any described herein), or any other reagents described herein.

In some embodiments, the compositions, kits or arrays described above contain less than 100,000 different polynucleotides. In some embodiments, the compositions, kits or arrays described above contain less than 90,000; less than 80,000; less than 70,000; less than 60,000; less than 50,000; less than 40,000; less than 30,000; less than 20,000; less than 15,000; less than 10,000; less than 5,000; less than 4,000; less than 3,000; less than 2,000; less than 1,500; less than 1,000; less than 750; less than 500, less than 200, less than 100, or less than 50 different polynucleotides.

In yet another aspect, the disclosure provides a use of a composition, kit or array as described herein for predicting the treatment response of a subject to a treatment with a CRHR1 antagonist, wherein the composition, kit or array is used to detect the presence or absence of one or more polymorphism genotypes within a sample obtained from a subject. In some embodiments, the composition, kit or array is used in a method of predicting a treatment response of a subject to a treatment with a CRHR1 antagonist as described herein. In some embodiments, the composition, kit or array is used in a method of detecting a polymorphism genotype associated with a treatment response of a subject to treatment with a CRHR1 antagonist as described herein.

### BRIEF DESCRIPTION OF THE DRAWINGS

FIG. 1 shows a time course curve of the clinical response of depressive patients as measured by the HAM-D scale upon treatment using placebo, or using an exemplary CRHR1 antagonist, wherein the surveyed subjects were predicted to positively respond to CRHR1 antagonist treatment using the method of prediction. The dashed line indicates a significant effect in treatment response at day 42 (p-value <0.01).

### DETAILED DESCRIPTION OF THE INVENTION

### General Definitions

The term “comprise” or “comprising” as used herein is to be construed as “containing” or “including” and does generally not exclude other elements or steps, but encompasses the term “consisting of” as an optional, specific embodiment. Thus, a group defined as comprising a certain number of embodiments, is also to be construed as a disclosure of a group which optionally consists only of these embodiments. Where an indefinite or a definite article is used when referring to a singular noun such as “a” or “an” or “the”, it includes a plural form of that noun unless specifically stated. Vice versa, when the plural form of a noun is used it refers also to the singular form. For example, when polymorphism genotypes are mentioned, this is also to be understood as a single polymorphism genotype.

Furthermore, the terms “first”, “second”, “third” or “(a)”, “(b)”, “(c)” and the like in the description and in the claims are used for distinguishing between elements and not necessarily for describing a sequential or chronological order. It is to be understood that the terms so used can be interchangeable under appropriate circumstances and that the embodiments of the invention described herein are capable of operation in other sequences than described or illustrated herein.

“Corticotropin releasing hormone” or “CRH” is used synonymously to the term “corticotropin releasing factor” or “CRF” herein, and refers to the known human 41 aa peptide or its mammalian homologues. The term “corticotropin releasing hormone receptor 1” or “CRHR1” refers to the receptor which binds to CRH and is used synonymously to the term “corticotropin-releasing factor receptor 1”, or CRF-R1, or CRFR-1 herein.

A “CRHR1 antagonist”, as used herein, refers to a compound capable of binding directly or indirectly to CRHR1 so as to modulate the receptor mediated activity. The CRHR1 mediated activity may be exerted on a downstream target within the signalling pathway of CRHR1. A “downstream target” may refer to a molecule such as an endogenous molecule (e.g. peptide, protein, lipid, nucleic acid or oligonucleotide), that is regulated by CRHR1 directly or indirectly, comprising direct or indirect modulation of the activity and/or expression level and/or localization, degradation or stability of the downstream target. A CRHR1 antagonist can be tested by any in vitro or in vivo test known in the art to be indicative of CRHR1 inhibition. For instance, CRHR1 antagonists will specifically bind to CRHR1 with a K_{D} of 1 pM or less, preferably with a K_{D} of 100 nM or less and/or specifically inhibit CRH binding to CRHR1 in vitro with an IC₅₀ value of 1 μM or less, preferably with IC₅₀ value of 100 nM or less. Alternatively or in addition, a CRHR1 antagonist can inhibit cellular, CRHR1-mediated cAMP accumulation and/or attenuate CRH and ACTH production in vivo.

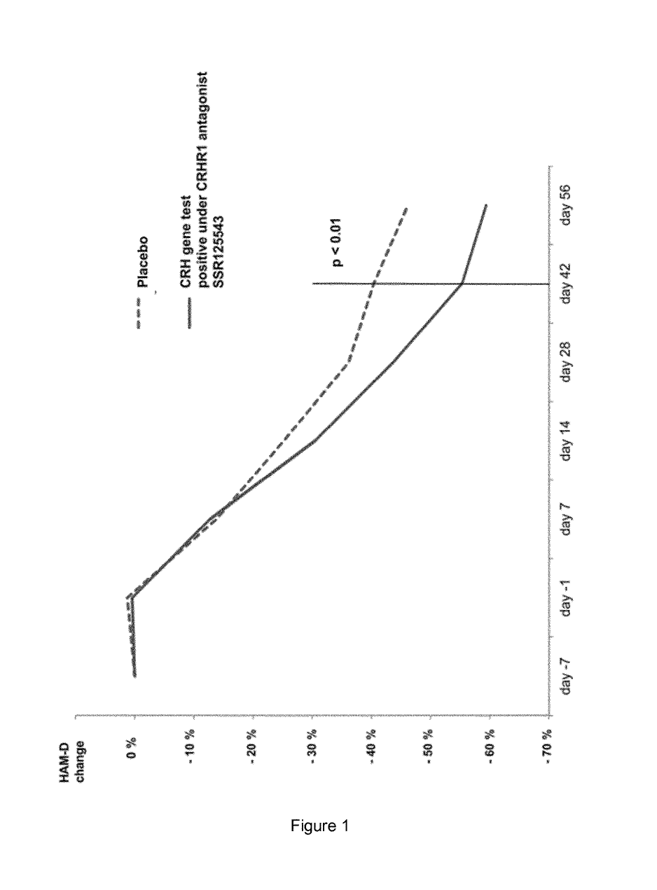

Several groups of CRHR1 antagonists are well known in the literature and are disclosed in the patent literature, e.g., in WO 94/13676, EP 0 773 023, WO 2004/047866, WO 2004/094420, WO 98/03510, WO 97/029109, WO 2006/044958, WO 2001/005776 and WO 95/033750, WO 2009/008552, WO 2010/015655 all of which are herein incorporated by reference. Further exemplary groups of CRHR1 antagonists are reviewed and disclosed in the scientific literature, e.g., in Williams, Expert Opin Ther Pat. 2013; 23(8):1057-68 (in particular compounds 1-48 as disclosed therein); Zorilla and Koob, Drug Discovery Today, 2010, 371-383 (in particular FIG. 1 and Tables 1-3 thereof); all of which are herein incorporated by reference in their entirety. Exemplary CRHR1 antagonist comprise, but are not limited to, GW876008 (Emicerfont), GSK-561679 (NBI-77860, Verucerfont), GSK586529, BMS-562,086 (Pexacerfont), NBI-30775 (R-121919), NBI-34101, CP-316,311, CP-376,395, PF-00572778, NVP-AAG561, Ono-2333MS, E2508, E2009, R317573 (JNJ19567470, CRA5626), R278995 (CRA0450), CRA-1000, CRA-1001, CP154,526, Antalarmin, DMP-695, DMP-696, DMP-904, SC-241, BMS-561388, NB130545, PD-171729, NB134041, NB135965, SN003, NBI-27914, trans-2-chloro-N-(4-((5-fluoro-4-methyl-pyridin-2-ylamino)-methyl)-cyclohexyl)-5-(trifluoromethyl)-benzamide, SSR-125543. Ono-2333Ms, NBI-34101, PF-00572778, GSK-561579 and GSK586529 are described by Zorilla and Koob (Drug Discovery Today, 2010, 371-383) as corticotropin releasing factor receptor antagonists tested in clinical trials. Exemplary CRHR1 antagonists are depicted in Table 1.

TABLE 1
Exemplary CRHR1 antagonists

| Structure | Name (synonym)/reference |
|---|---|
| 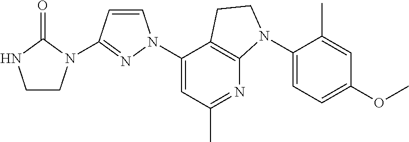 [SMILES: COc1ccc(N2CCc3c(-n4ccc(N5CCNC5=O)n4)cc(C)nc32)c(C)c1] | GW876008 (Emicerfont) |
| 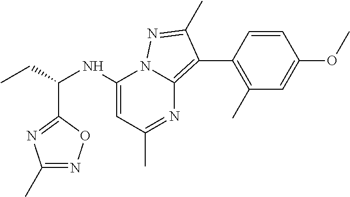 [SMILES: CC[C@H](Nc1cc(C)nc2c(-c3ccc(OC)cc3C)c(C)nn12)c1nc(C)no1] | GSK-561679 (NBI-77860, Verucerfont) |
| 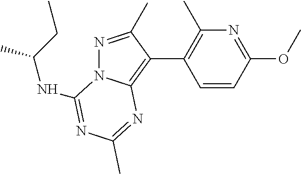 [SMILES: CC[C@@H](C)Nc1nc(C)nc2c(-c3ccc(OC)nc3C)c(C)nn12] | BMS-562,086 (Pexacerfont) |
| 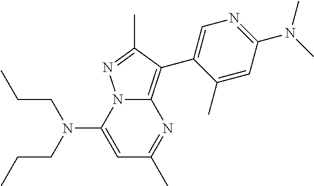 [SMILES: CCCN(CCC)c1cc(C)nc2c(-c3cnc(N(C)C)cc3C)c(C)nn12] | NBI-30775 (R-121919) |
| 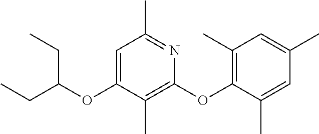 [SMILES: CCC(CC)Oc1cc(C)nc(Oc2c(C)cc(C)cc2C)c1C] | CP-316,311 |
| 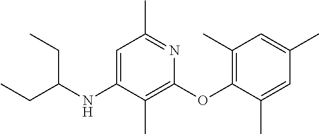 [SMILES: CCC(CC)Nc1cc(C)nc(Oc2c(C)cc(C)cc2C)c1C] | CP-376,395 |
| 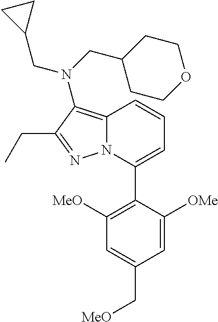 [SMILES: CCc1nn2c(-c3c(OC)cc(COC)cc3OC)cccc2c1N(CC1CCOCC1)CC1CC1] | E2508 |
| 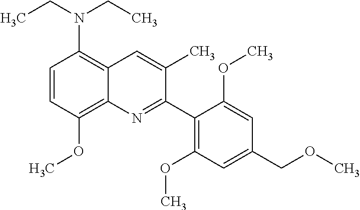 [SMILES: CCN(CC)c1ccc(OC)c2nc(-c3c(OC)cc(COC)cc3OC)c(C)cc12] | E2009 Terauchi et al., 244th ACS National Meeting, Aug. 19-23, 2012, Poster Presentation & Abstract, 2 pp. |
| 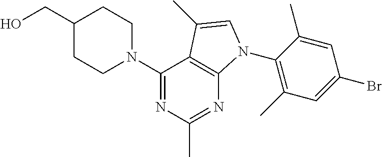 [SMILES: Cc1nc(N2CCC(CO)CC2)c2c(C)cn(-c3c(C)cc(Br)cc3C)c2n1] | R317573 (JNJ19567470, CRA5626, TAI-041) |
| 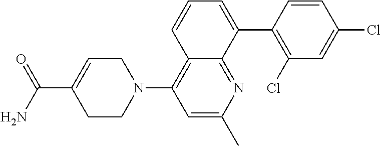 [SMILES: Cc1cc(N2CC=C(C(N)=O)CC2)c2cccc(-c3ccc(Cl)cc3Cl)c2n1] | R278995 (CRA0450) |
| 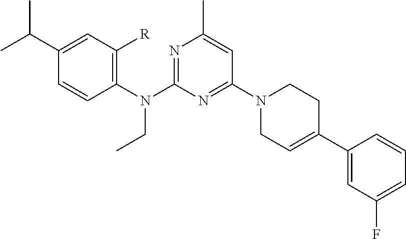 | CRA-1000 (R = S—CH₃) CRA-1001 (R = Br) |
| 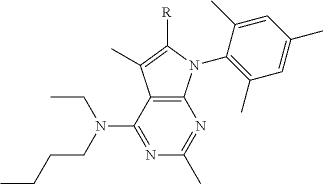 | CP154,526 (R = H) Antalarmin (R = CH3) |
| 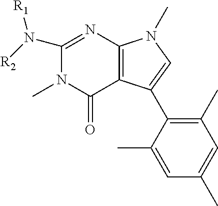 | Compounds 16a-e K. Aso et al./Bioorg. Med. Chem. Lett. 21 (2011) 2365- 2371 |
| 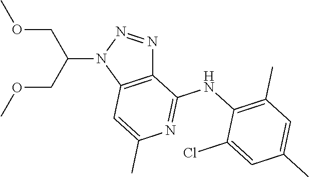 | DMP-695 |
| 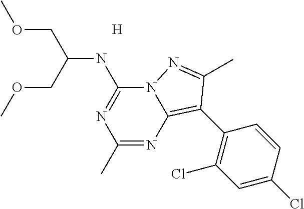 | DMP-696 |
| 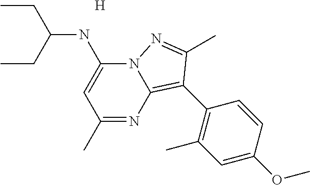 | DMP-904 |
| 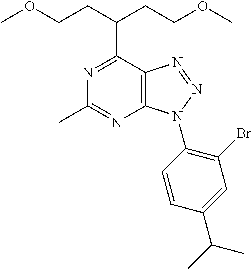 [SMILES: COCCC(CCOC)c1nc(C)nc2c1nnn2-c1ccc(C(C)C)cc1Br] | SC-241 |
| 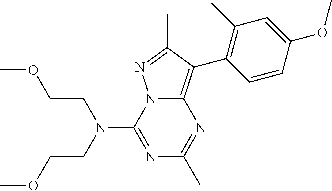 | BMS-561388 |
| 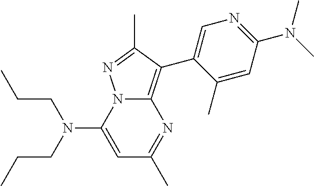 [SMILES: CCCN(CCC)c1cc(C)nc2c(-c3cnc(N(C)C)cc3C)c(C)nn12] | NBI30545 |
| 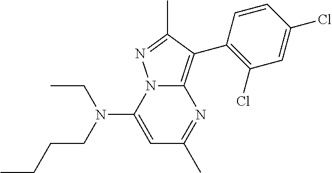 [SMILES: CCCCN(CC)c1cc(C)nc2c(-c3ccc(Cl)cc3Cl)c(C)nn12] | PD-171729 |
| 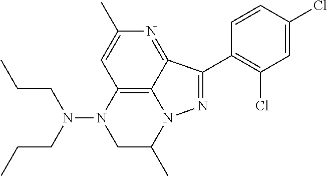 [SMILES: CCCN(CCC)N1CC(C)n2nc(-c3ccc(Cl)cc3Cl)c3nc(C)cc1c32] | NBI34041 |
| 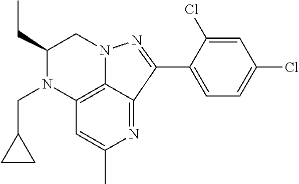 [SMILES: CC[C@H]1Cn2nc(-c3ccc(Cl)cc3Cl)c3nc(C)cc(c32)N1CC1CC1] | NBI35965 |
| 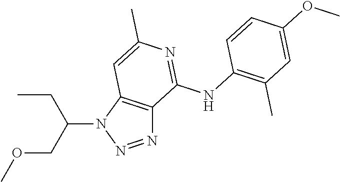 | SN003 |
| 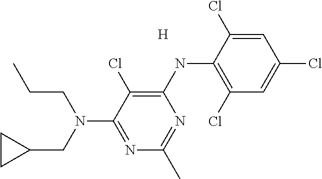 | NBI-27914 |
| 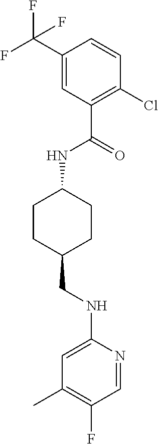 [SMILES: Cc1cc(NC[C@H]2CC[C@H](NC(=O)c3cc(C(F)(F)F)ccc3Cl)CC2)ncc1F] | trans-2-chloro-N-(4-((5-fluoro-4- methyl-pyridin-2-ylamino)- methyl)-cyclohexyl)-5- (trifluoromethyl)-benzamide Compound 46 in Williams Expert Opin Ther Pat, 2013, 23(8): 1057-68); WO 2010/015655 |
| 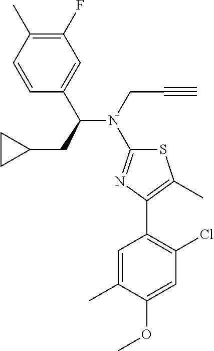 [SMILES: C#CCN(c1nc(-c2cc(C)c(OC)cc2Cl)c(C)s1)[C@@H](CC1CC1)c1ccc(C)c(F)c1] | SSR-125543 |

Most of the non-peptidic CRHR1 antagonists can be described by a pharmacophore model comprising a lipophilic top group, a heterocyclic core containing an invariable hydrogen bond acceptor, which is almost always a heterocyclic nitrogen, and a lipophilic, usually aromatic, bottom group. The terms “Type I CRHR1 antagonist”, “Type II CRHR1 antagonist”, “monocyclic Type II CRHR1 antagonist”, “bicyclic Type II CRHR1 antagonist”, “atypical CRHR1 antagonist” or “cyclohexyl amide CRHR1 antagonist”, as used herein, is synonymous to “Type I CRF₁ antagonist”, “Type II CRF₁ antagonist”, “monocyclic Type II CRF₁ antagonist”, “bicyclic Type II CRF₁ antagonist”, “atypical CRF₁ antagonist” or “cyclohexyl amide CRF₁ antagonist”, respectively, referring to known and readily available compounds as defined in Williams, Expert Opin Ther Pat. 2013; 23(8):1057-68, incorporated herein by reference in its entirety. In specific embodiments, Type I CRHR1 antagonists are compounds 1-33, monocyclic Type II CRHR1 antagonists are compounds 34-36, bicyclic Type II CRHR1 antagonists are compounds 37-41, atypical CRHR1 antagonists are compounds 42-45, or cyclohexyl amide CRHR1 antagonists are compounds 46-48, respectively, as disclosed in FIGS. 1-11 of Williams, Expert Opin Ther Pat. 2013; 23(8):1057-68, incorporated herein by reference in its entirety. Exemplary “Type I CRHR1 antagonists” may be characterized in that the heterocyclic hydrogen bond acceptor and the bottom group are connected by a two-atom linker as exemplified by CRHR1 antagonists R-121919, NBI-30545, CP-154526, DMP-696, pexacerfont (BMS-562086), emicerfont (GW876008), or verucerfont (GSK561679). Type II CRH1R antagonists may be characterized by a one-atom linker between hydrogen bond acceptor and the bottom group. Any pharmaceutically acceptable salt of a CRHR1 antagonist described herein is encompassed by the present disclosure.

### Methods of Predicting Treatment Response to CRHR1 Treatment

In one aspect, the present invention provides methods for predicting a treatment response of a subject (such as a human patient) to a treatment with a CRHR1 antagonist, which can include one or more CRHR1 antagonists. Thus, methods of the invention are useful in selecting appropriate therapeutic modalities (e.g., a treatment with a CRHR1 antagonist or a treatment with a non-CRHR1 antagonist) for subjects suffering from conditions generally treatable by a CRHR1 antagonist, for instance psychiatric disorders such as depressive symptoms or anxiety symptoms.

Specifically, in this aspect, the method of the invention can be used for predicting a treatment response of a subject to treatment with a CRHR1 antagonist, the method comprising providing a biological sample obtained from the subject, detecting the presence or absence of one or more polymorphism genotypes in the biological sample, wherein the one or more polymorphism genotypes comprise: (a) at least one polymorphism genotype selected from the group consisting of the polymorphism genotypes disclosed in Table 2, (b) at least one polymorphism genotype being in linkage disequilibrium with any one of the polymorphism genotypes of (a); or (c) a combination of (a) and (b), and predicting the treatment response from the presence or absence of the one or more polymorphism genotypes of (a), (b), or (c).

A “subject”, as used herein, can generally be any mammal, in which one or more polymorphism genotypes as disclosed in Table 2 or polymorphism genotypes being in linkage disequilibrium with any one of the polymorphism genotypes of Table 2 are conserved or homologous. In particular, the term “subject” includes a human subject, and any model organism such as mice, rats, cats, dogs, simians, cattle. Preferably, the subject is a human subject.

A “treatment with a CRHR1 antagonist”, as used herein, refers to the treatment of a condition in the subject which can be treated by administration of a CRHR1 antagonist, as is made plausible herein or in the prior art. “Conditions treatable with a CRHR1 antagonist”, as used herein, are conditions which can generally be treated by administration of a CRHR1 antagonist and/or are commonly characterized, caused or accompanied by CRH over-activity, by ACTH over-activity and/or by over-activity of the Hypothalamic-pituitary-adrenal (HPA) axis.

The term “CRH over-activity” is used herein synonymously to the terms “CRH system over-activity”, “CRH hyperactivity”, “CRH hyperdrive” or “central CRH hyperdrive”. An indication for CRH over-activity may be an increase in activity or concentration of CRH or of one or several molecules downstream of the CRHR1 receptor, that are activated or whose concentration is increased based on the activation of CRHR1 receptor upon CRH binding, for instance, but not being limited to, ACTH. A further indication for CRH over-activity may be a decrease in activity or concentration of one or several molecules downstream of the CRHR1 receptor, that are inactivated or whose concentration is decreased resulting from the activation of CRHR1 receptor upon CRH binding. For instance, the concentrations or activities of adrenocorticotrophin (ACTH) and/or cortisol can be used for determining a value indicative for CRH over-activity. The CRH over-activity in each patient may be determined by a CRH test as described in Holsboer et al., N Engl J Med. 1984; 311(17):1127, or by a combined dexamethasone suppression/CRH stimulation test (dex/CRH test) as described in Heuser et al., J Psychiatr Res 1994, 28(4):341-56; both incorporated herein by reference in their entirety.

In particular, conditions which can be treated using a CRHR1 antagonist in a subject comprise, but are not limited to, behavioural disorders, neuropsychiatric disorders, mood disorders, neurological disorders, neurodegenerative disorders, endocrine disorders, inflammatory or stress-induced immune disorders, CRH-related cardiovascular diseases or metabolic diseases. Specifically, such conditions comprise anxiety symptoms, generalized anxiety disorder, panic, phobias, obsessive-compulsive disorder, post-traumatic stress disorder, sleep disorders such as insomnia, hypersomnia, narcolepsy, idiopathic hypersomnia, excessive amounts of sleepiness, lack of alertness, lack of attentiveness, absentmindedness and/or lack of or aversion to movement or exercise, sleep disorders induced by stress, pain perception such as fibromyalgia, mood disorders such as depressive symptoms, including major depression, single episode depression, recurrent depression, child abuse induced depression, mood disorders associated with premenstrual syndrome, and postpartum depression, dysthymia, bipolar disorders, cyclothymia, chronic fatigue syndrome, stress-induced headache, eating disorders such as anorexia and bulimia nervosa, hemorrhagic stress, stress-induced psychotic episodes, endocrine disorders involving ACTH overproduction, ACTH over-activity, e.g., adrenal disorders, including, but not limited to congenital adrenal hyperplasia (CAH), euthyroid sick syndrome, syndrome of inappropriate antidiarrhetic hormone (ADH), obesity, infertility, head traumas, spinal cord trauma, ischemic neuronal damage (e.g., cerebral ischemia such as cerebral hippocampal ischemia), excitotoxic neuronal damage, epilepsy, senile dementia of the Alzheimers type, multi-infarct dementia, amyotrophic lateral sclerosis, chemical dependencies and addictions (e.g., dependencies on alcohol, nicotine, cocaine, heroin, benzodiazepines, or other drugs), drug and alcohol withdrawal symptoms, hypertension, tachycardia, congestive heart failure, osteoporosis, premature birth, and hypoglycaemia, inflammatory disorders such as rheumatoid arthritis and osteoarthritis, pain, asthma, psoriasis and allergies, irritable bowel syndrome, Crohn's disease, spastic colon, post-operative ileus, ulcer, diarrhea, stress-induced fever, human immunodeficiency virus (HIV) infections, neurodegenerative diseases such as Alzheimer's disease, Parkinson's disease and Huntington's disease, gastrointestinal diseases, stroke, stress induced immune dysfunctions, muscular spasms, urinary incontinence. In a specific embodiment, the subject has and/or the treatment is a treatment of depressive symptoms, anxiety symptoms or both depressive symptoms and anxiety symptoms. The depressive and/or anxiety symptoms can be symptoms of a depressive disorder, an anxiety disorder or both a depressive disorder and anxiety disorder. In another specific embodiment, the subject has and/or the treatment is a treatment of a sleep disorder.

A “treatment response”, as used herein, generally refers to any measurable response specific for the treatment with one or more CRHR1 antagonist and/or the condition being treated, during and/or shortly after treatment as compared to before said treatment. Generally, the treatment response can be a biological response or a clinical response. A biological response would include, for example, any alteration in CRH over-activity, as defined above.

Preferably, according to the invention, the treatment response is a clinical treatment response. A “clinical treatment response”, as used herein, refers to a prevention, alteration, alleviation or complete remission, as measured by the alteration in severity and/or frequency of relapse of individual symptoms and/or the mean change on a diagnostic marker or scale of any type commonly used in assessing clinical responses in the conditions described herein, see, for instance, Harrison's Principles of Internal Medicine, 18^{th} ed./editors Longo et al., Mcgraw-Hill Publ. Comp, NY, US (2011), as incorporated herein by reference in its entirety. A clinical treatment response can also include an alteration, increase or decrease in adverse effects resulting from the treatment with a CRHR1 antagonist. Predicting a clinical response, or lack thereof, is expressly distinguished from predicting merely biological responses, since a clinical response is to be seen as target variable directly linked to treatment success, or failure, respectively. Therefore, while biological responses can also be predicted by the methods described herein, the methods of the invention are particularly suited for predicting a clinical response, as defined above.

In preferred embodiments, the clinical response can be a prevention, alteration, alleviation or complete remission of depressive symptoms and/or anxiety symptoms. In preferred embodiments, the clinical response can be a prevention, alteration, alleviation or complete remission of a sleep disorder. Depressive symptoms comprise, but are not limited to, low mood, low self-esteem, loss of interest or pleasure, psychosis, poor concentration and memory, social isolation, psychomotor agitation/retardation, thoughts of death or suicide, significant weight change (loss/gain), fatigue, and feeling of worthlessness. The depressive symptoms can last for weeks to lifelong with periodic reoccurring depressive episodes. For the diagnosis of the depression mode (e.g. moderate or severe depression) the Hamilton Depression Rating Scale (HAM-D) (Hamilton, J Neurol Neurosurg Psychiatry, 1960) may be used. In addition or alternatively, the depression mode may be also rated by alternative scales as the Beck Depression Inventory (BDI), the Montgomery-Asberg Depression Scale (MADRS), the Geriatric Depression Scale (GDS), and/or the Zung Self-Rating Depression Scale (ZSRDS). Therefore, in some embodiments, the clinical response is a prevention, alteration, alleviation or complete remission of depressive symptoms as determined using a scale selected from the group consisting of HAM-D, BDI, MADRS, GDS, ZSRDS.

Anxiety symptoms comprise, but are not limited to, panic disorders, generalized anxiety disorder, phobias and posttraumatic stress disorder, avoidance behavior which may lead to social isolation, physical ailments like tachycardia, dizziness and sweating, mental apprehension, stress and/or tensions. The severity of anxiety symptoms ranges from nervousness and discomfort to panic and terror in subjects. Anxiety symptoms may persist for several days, weeks, or even months and years, if not suitably treated. The severity of anxiety symptoms may be measured by the Hamilton Anxiety Rating Scale (HAM-A) and/or the State-Trait Anxiety Rating Scale (STAI). Therefore, in some embodiments, the clinical response is a prevention, alteration, alleviation or complete remission of anxiety symptoms as determined using a scale selected from the group consisting of HAM-A and STAI. Sleep disorders comprise, but are not limited to, insomnia, hypersomnia, narcolepsy, idiopathic hypersomnia, excessive amounts of sleepiness, lack of alertness, lack of attentiveness, absentmindedness and/or lack of or aversion to movement or exercise, sleep disorders induced by stress.

“Alteration”, as used herein, refers to any change in a clinical response as defined above. “Alleviation”, as used herein, refers to any amelioration in a clinical response, including partial amelioration of one or more symptoms, temporary disappearance of one or more symptoms, wherein relapse is not excluded, as well as complete remission of one or more symptoms. “Complete remission” refers to disappearance of all manifestations and symptoms of a disease to be treated, as described herein.

The present disclosure identifies sets of polymorphism genotypes that are predictive for the treatment response of a subject to treatment with a CRHR1 antagonist. Thus, the presence of one or more of these polymorphism genotypes can be used to predict the likelihood of responding or not responding to treatment with a CRHR1 antagonist in a subject.

The term “polymorphism”, as used herein, refers to a sequential variation of a genomic allele at the same locus within a population of subjects and having a certain frequency in the population, including deletions/insertions (designated “[−I]” herein), point mutations and translocations. The term “polymorphism”, as used herein, in particular includes, but is not limited to, single nucleotide polymorphisms (SNPs). For instance, as used herein, the term “polymorphism” can also include polymorphic deletions, or insertions, respectively, of more than one nucleotide. The term “single nucleotide polymorphism” or “SNP” is well understood by the skilled person and refers to a point mutation of a genomic allele at the same locus within a population of subjects and having a certain frequency in a population. The term “genotype”, as used herein, encompasses one or both genomic alleles at the same locus of a subject. The term “polymorphism genotype” or “SNP genotype”, as used herein, refers to the presence of a polymorphism or SNP within the genotype of a subject, either in one or both genomic alleles at the same locus. The allele being present in the majority of the population, is also referred to herein as wild-type allele or major allele. As used herein, this state is defined as the “absence of one or more polymorphism genotypes”. The nucleotide being present in the minority of the population is also referred to herein as the variation, point mutation, mutated nucleotide or minor allele. As used herein this state is defined as “presence of one or more polymorphism genotype”. For instance, P_ID 1 as identified in Table 2 below, (r534169260, TOP, [NG]) exhibits a variation to nucleotide G instead of the wild-type nucleotide A. Typically, a polymorphism or SNP genotype occurs in a certain percentage of a population, for example in at least 5% or at least 10% of a population. In other words, the minor allele frequency (MAF) is equal or higher than about 0.05 or about 0.10 (MAF>0.05 or MAF>0.10).

Theoretically, a wild-type allele could be mutated to three alternative nucleotides. However, a mutation to a first nucleotide within germline cells of an individual which persists within a population occurs very rarely. The chance of the same nucleotide being mutated to yet another nucleotide and again persisting within a population is virtually non-existent and can be therefore neglected. Therefore, as used herein, a certain nucleotide position in the genome of an individual can only have the above two states, namely the wild-type state (absence of a polymorphism genotype from both alleles of a single subject) and the mutated state (presence of a polymorphism genotype in one or both alleles of a single subject). The presence of a polymorphism genotype in both alleles may have a higher predictive value than the presence of a polymorphism genotype in one allele only, the other allele comprising a wild-type genotype. The presence or absence of a polymorphism genotype on one or two alleles may be associated with an algorithm for predicting the treatment response to CRHR1 antagonists as described herein.

Sets of polymorphism genotypes useful in methods for predicting a treatment response are disclosed in Table 2. Table 2 provides a consecutively numbered identifier (P_ID) for internal reference, an rs-identifier (rs_ID), as commonly known in polymorphism databases such as NCBI's dbSNP, the polymorphism (P, indicated in bold and defined as [wild-type/variation]), the strand designation (Str, see, e.g., Illumine Inc. “TOP/BOT” Strand and “A/B” Allele-A guide to Illumina's method for determining Strand and Allele for the Golden Gate® and Infinium™ Assays”, Technical Note, © 2006; http://www.illumina.com/documents/products/technotes/technote_topbot.pdf; incorporated by reference herein in its entirety), specific probe sequences for the respective allele in humans (AlleleA Probe, see also SEQ ID NOs: 275-548), a human chromosomal identifier (Chr), and a reference to the sequence of the genomic flanking sequence in humans (TopGenomicSequence), as disclosed in SEQ ID NOs: 1-274. A person skilled in the art is able to derive the exact position and polymorphism genotype sequence from the rs-nomenclature identified in Table 2 from suitable database entries and associated information systems, e.g. the NCBI's Single Nucleotide Polymorphism database (dbSNP; http://www.ncbi.nlm.nih.gov/SNP/), even where the nomenclature, or the surrounding sequence elements were subject to alterations over time.

TABLE 2
Polymorphism genotypes as used herein

| | | | | | | TopGenomic |
|---|---|---|---|---|---|---|
| P_ID | rs_ID | Str | P | AlleleA Probe | Chr | Sequence |
| 1 | rs34169260 | TOP | [A/G] | AGGACTCTATGGCTTCCTTCATGTCATCGTCCA | 17 | SEQ ID NO: 1 |
| | | | | CTCTGCCAAGGGATTTA | | |
| 2 | rs796287 | TOP | [A/C] | ACGACAGAGATGAATTGAGGGGACAAATGTCAG | 2 | SEQ ID NO: 2 |
| | | | | AGCTCACAGACGACTGT | | |
| 3 | rs56149945 | TOP | [A/G] | TCAGAAGCCTATTTTTAATGTCATTCCACCAAT | 5 | SEQ ID NO: 3 |
| | | | | TCCCGTTGGTTCCGAAA | | |
| 4 | rs6190 | BOT | [T/C] | TCACAGTAGCTCCTCCTCTTAGGGTTTTATAGA | 5 | SEQ ID NO: 4 |
| | | | | AGTCCATCACATCTCCC | | |
| 5 | rs7179092 | BOT | [T/C] | TTGCATTCTCTCCTAGCACTCCAGTAAATAAAC | 15 | SEQ ID NO: 5 |
| | | | | TATAGTCCTGGTCAAGT | | |
| 6 | rs7614867 | TOP | [A/G] | ATTCCCAATATTCGTATATGTATTTATAAATTA | 3 | SEQ ID NO: 6 |
| | | | | CATAATGGGCAGGGTGC | | |
| 7 | rs920640 | BOT | [T/C] | AGTGCTTTTTGAGAGGTATGAACTTACTCCATA | 15 | SEQ ID NO: 7 |
| | | | | CTACTTACATCTGCTAA | | |
| 8 | rs7167722 | BOT | [T/C] | TGACTTCTAATTACAGGCAAAATCAACCTTAAT | 15 | SEQ ID NO: 8 |
| | | | | AAGAACAGGCGTTACTA | | |
| 9 | rs920638 | BOT | [T/C] | TACTATTCTGTTCATAAGGTACACTTCTTTTTA | 15 | SEQ ID NO: 9 |
| | | | | GGGCACACTACCTTGGG | | |
| 10 | rs7165629 | BOT | [T/C] | AGGTGGGATAAACAGAAGCAGCATAACGTGTCT | 15 | SEQ ID NO: 10 |
| | | | | TGATGTGTGCTGTTTAG | | |
| 11 | rs80049044 | BOT | [T/A] | TTGTCATGCAGCAGGTTAACTATGCTTTCTGGA | 4 | SEQ ID NO: 11 |
| | | | | GAAGGTGTCAGCCAACT | | |
| 12 | rs16941058 | TOP | [A/G] | CCCTCCAGCTGAATGATTTTTGTCTGTGCCTGG | 17 | SEQ ID NO: 12 |
| | | | | CCCAGTCCCTGAGTCCA | | |
| 13 | rs112015971 | TOP | [A/G] | GTGAAAATGCATTTTCCCCCTATTCCTTCTGGA | 20 | SEQ ID NO: 13 |
| | | | | AAGCAACATTAGGGTCC | | |
| 14 | rs10894873 | BOT | [T/C] | TGCTCACCACAGTCCTCATATCCTTAAAGGGAC | 11 | SEQ ID NO: 14 |
| | | | | ACCCTAGTGATTACTGA | | |
| 15 | rs117455294 | BOT | [T/G] | CAGTCCCGCCTGCTTGGATCTGACGAGCGTGCC | 20 | SEQ ID NO: 15 |
| | | | | GATTCGGTCCGAAAATC | | |
| 16 | rs1170303 | BOT | [T/C] | AGAGCACTAACTCTGGAGAGTAAGGATCTGAGT | 4 | SEQ ID NO: 16 |
| | | | | GTAAGTCACCGCTGTGT | | |
| 17 | rs16940681 | TOP | [C/G] | AAGCAGTCCACAGCAGTCTGAGCTGGCAGGTCA | 17 | SEQ ID NO: 17 |
| | | | | TGGAGCAGCCCCCAAAC | | |
| 18 | rs968519 | BOT | [T/C] | GTAAAGAACAGGGGGAGATAATGATCAGTAAAA | 20 | SEQ ID NO: 18 |
| | | | | TCACAGCAGGGTGAGGG | | |
| 19 | rs28381866 | BOT | [T/C] | TATTTAGGTAGTTGACCACTTCAGCATTCTAGG | 7 | SEQ ID NO: 19 |
| | | | | TACAATAACGTTAGCCC | | |
| 20 | rs79320848 | BOT | [T/G] | AGAACAAAGCCAGGACAAGGTACAAGGTGACCC | 20 | SEQ ID NO: 20 |
| | | | | CAGCAAATTTCCTTTTC | | |
| 21 | rs114653646 | BOT | [T/G] | TGCTAGAAGCTTATCAACTGCATTAATCTTTTT | 7 | SEQ ID NO: 21 |
| | | | | AAAAACACTTTTAGTTT | | |
| 22 | rs2589496 | BOT | [T/C] | TCTCACCTTCTCCAGGTGCACGGTAGGTGCTGT | 17 | SEQ ID NO: 22 |
| | | | | GTAAATTAACGACTTCA | | |
| 23 | rs10482650 | TOP | [A/G] | GCCTCCTGCTAGACAATTAGCTTTATCCATGAG | 5 | SEQ ID NO: 23 |
| | | | | TTACCAAAGAGGGAGCC | | |
| 24 | rs17614642 | TOP | [A/G] | ACCAAAATCTATAAACAATAAGGAACTGTGGTT | 6 | SEQ ID NO: 24 |
| | | | | GTTTGCTGCAAATAACT | | |
| 25 | rs73200317 | BOT | [T/C] | TCAAGAGTTGGGAATGATGAGGGCATGTACTGT | 7 | SEQ ID NO: 25 |
| | | | | GACTGGCACACAGAATG | | |
| 26 | rs1380146 | BOT | [T/A] | AGTGccTAcTATGTGcTAGTcccTAGTGAcATG | 12 | SEQ ID NO: 26 |
| | | | | AGAGTGAGGAAGGCAGT | | |
| 27 | rs735164 | BOT | [T/C] | CCTTATTTCAAGGTCGGGGTCAAGGTGGTCAAA | 16 | SEQ ID NO: 27 |
| | | | | AGAACTGTTTTGCTCTC | | |
| 28 | rs730976 | BOT | [T/G] | AAGGGTATTTATACCTTTGCCTTTCCGCCTCAA | 5 | SEQ ID NO: 28 |
| | | | | CCATTGGAACCTGGGAC | | |
| 29 | rs55934524 | BOT | [T/G] | AGCCTCTCTGGGTCCTTGGGGAGCATGAGGATC | 17 | SEQ ID NO: 29 |
| | | | | CTGCAGAAAGCAGAGTG | | |
| 30 | rs4570614 | TOP | [A/G] | ATGCTCTCTGAACACTATGACCTCTGATTATTT | 11 | SEQ ID NO: 30 |
| | | | | ATCAACCTCCAAGAGCT | | |
| 31 | rs4458044 | TOP | [C/G] | CCCCTCTTCTGTGAGAGCCAAACAGAGCCCTTC | 17 | SEQ ID NO: 31 |
| | | | | CTGAGTCCCATCCATTC | | |
| 32 | rs77850169 | TOP | [A/G] | TCTGGGTCCTTTTCATTGCTCTACAAAGAATCC | 17 | SEQ ID NO: 32 |
| | | | | TTTCTTCCTCCCAGGCC | | |
| 33 | rs35339359 | TOP | [A/G] | CATCAATGCCCACGCTACACGAGGCATACTAGA | 17 | SEQ ID NO: 33 |
| | | | | CAGTCGCTGCCTAAGCC | | |
| 34 | rs34800935 | BOT | [T/C] | TCAAGAGTAACAGTATGCCCTGCATTAACAGGG | 7 | SEQ ID NO: 34 |
| | | | | ATAATATATAAGAAAAA | | |
| 35 | rs72945439 | BOT | [T/C] | GAATTTATTACTCCTGGGAGGATTCTGCTCACC | 2 | SEQ ID NO: 35 |
| | | | | ACTGGCAACTATGACCA | | |
| 36 | rs113959523 | TOP | [A/G] | CATCATGATGTAATGTAGTCATATAGACTAGGA | 20 | SEQ ID NO: 36 |
| | | | | CACTTAGATTAGCCCCC | | |
| 37 | rs116798177 | TOP | [A/G] | GGTTTTAGTATTGCAATGTGGAATCCAAAACTG | 5 | SEQ ID NO: 37 |
| | | | | TTATCAATGAACTTTTG | | |
| 38 | rs11247577 | BOT | [T/G] | TGGGTGAGGGAACCGTTAGTGCCATCCTGAGGC | 17 | SEQ ID NO: 38 |
| | | | | CCCGTGTCAGGAAATAT | | |
| 39 | rs75869266 | BOT | [T/C] | ACTGAACTCCCCATCACAAATCTGTATGCTTTA | 15 | SEQ ID NO: 39 |
| | | | | TTAGAAAGTAAAACTCT | | |
| 40 | rs74372553 | BOT | [T/C] | AGTAAAACAGACGACGGGATCCCCAGACGCTGC | 17 | SEQ ID NO: 40 |
| | | | | ACATCAGCACCAGGAGC | | |
| 41 | rs11691508 | TOP | [A/G] | CACACTAATATTCAAACATCCTTGACCTCATCT | 2 | SEQ ID NO: 41 |
| | | | | CATATAAATAAATCCAA | | |
| 42 | rs6493965 | TOP | [A/G] | CCAAGATTCTGGATGTCTTTAAGGTAACAAGTG | 15 | SEQ ID NO: 42 |
| | | | | TCCATGTTGTTCCTTGA | | |
| 43 | rs4869476 | BOT | [T/C] | GAAGCGAAAATAGCTATGCACCAAATCTCTGCA | 5 | SEQ ID NO: 43 |
| | | | | GGCATTTCATTGAGTAC | | |
| 44 | rs3730170 | BOT | [T/C] | TGAATGACAGTGTTGTTGATTAGTTCAAGCTCT | 20 | SEQ ID NO: 44 |
| | | | | TGCCTTTCTCTAAACTT | | |
| 45 | rs2145288 | TOP | [A/C] | GATCTTAGCCAAGGCAGGAAAGCACACGATCAG | 20 | SEQ ID NO: 45 |
| | | | | GTAACCTCCAGATTCAC | | |
| 46 | rs2935752 | TOP | [A/C] | TTACTCGCATTAACTCTTTCAATTTCACAACAA | 8 | SEQ ID NO: 46 |
| | | | | ATCTAAGAAAAATGCAA | | |
| 47 | rs146512400 | TOP | [A/G] | AGTCTAAAACACTATCATCTCCTCCTGGATTAC | 7 | SEQ ID NO: 47 |
| | | | | TGCAACAGACTCCTTCT | | |
| 48 | rs62057097 | BOT | [T/C] | TCTGCCCTAAATATTCCCTGTTCGGTGGGGTTT | 17 | SEQ ID NO: 48 |
| | | | | GGCGGTCCAGCAGCCCT | | |
| 49 | rs115061314 | BOT | [T/C] | CCATGCGTGTTGGAAGTATTTCTCTTGTTCTCC | 6 | SEQ ID NO: 49 |
| | | | | TGCTTTTAGAAAGCCAT | | |
| 50 | rs34113594 | BOT | [T/G] | CTTCTGACCCTCGCCGTCCTAGAACCAACGGCC | 17 | SEQ ID NO: 50 |
| | | | | CCTCGGTGTCTGGTCCT | | |
| 51 | rs61751173 | TOP | [A/G] | AAAGCTCTAATACCACCTAAAACCATTTCTGTT | 5 | SEQ ID NO: 51 |
| | | | | CTCTACCTCTGTCATTA | | |
| 52 | rs74338736 | TOP | [A/C] | ACAGGTTCTATATCTTTAGATGGTAAATTAAAA | 20 | SEQ ID NO: 52 |
| | | | | ATTCCTGGCTGAATTTG | | |
| 53 | rs10851726 | BOT | [T/C] | AATGTGAGTAGATTCCAACCTTTATCCATTCCA | 15 | SEQ ID NO: 53 |
| | | | | TTCACATTTACCTTCTC | | |
| 54 | rs4610906 | BOT | [T/C] | TTGTTTAAAGCTGCTGCAGGTATACTCTTTGGA | X | SEQ ID NO: 54 |
| | | | | GGCTAATAATAAAGAAC | | |
| 55 | rs59485211 | BOT | [T/C] | TGGAGTAGTCTTCTTCTAGCCCTTGCATGACCT | X | SEQ ID NO: 55 |
| | | | | CTCTTACTTCACCCATA | | |
| 56 | rs7060015 | BOT | [T/G] | CTTCCACCTGCTGCACTCCAATATAGCCACTAT | X | SEQ ID NO: 56 |
| | | | | GTTCGGCTATATATATA | | |
| 57 | rs75710780 | BOT | [T/G] | TAGAGAGTAATGTGGTGGGTGTGCTGTGTCAGA | 6 | SEQ ID NO: 57 |
| | | | | AAGGCTTCACTAGCAGT | | |
| 58 | rs6520908 | BOT | [T/C] | CTAATTTGATCAATGAATCACTGCTAGCATGTG | X | SEQ ID NO: 58 |
| | | | | AATGTCCATAATGGATA | | |
| 59 | rs487011 | BOT | [T/G] | TTATTAGAGGTAAACATAGAGATAAGCCCCTAA | X | SEQ ID NO: 59 |
| | | | | TAAAATAGTAGCTGGAG | | |
| 60 | rs1383699 | TOP | [A/C] | AGTGTTAATTCTCTAAGAGGAAAATGTCATTTC | 4 | SEQ ID NO: 60 |
| | | | | TCCAAAACAAAACTTTA | | |
| 61 | rs67516871 | TOP | [A/G] | GTAACAAGGTTACCTCCAGAAAAAAAGGCTATT | X | SEQ ID NO: 61 |
| | | | | GCTGAACAGAGGCTTTC | | |
| 62 | rs114106519 | BOT | [T/C] | AAGAGAGAAAAATATTTTTAAGTGAAAAGGAAC | 7 | SEQ ID NO: 62 |
| | | | | AAAACTATTCTATACGA | | |
| 63 | rs7220091 | TOP | [A/G] | GGCTCACACCGAGATCAATCCATGATGACAGCA | 17 | SEQ ID NO: 63 |
| | | | | CTTCATGGCCCGTCTCA | | |
| 64 | rs12489026 | TOP | [A/G] | GATAATCTAATTCATCTAACTTGCTTTACAAAT | 3 | SEQ ID NO: 64 |
| | | | | GAGGAAACTGATAATCC | | |
| 65 | rs876270 | BOT | [T/C] | GTGGACCCTTTGAGTGGTTACAGACGGGCCTCA | 12 | SEQ ID NO: 65 |
| | | | | GGATTGGTGTTATTTAA | | |
| 66 | rs4968161 | BOT | [T/C] | AACAGGGGCCACTGTCTGTTTCCCATGGTATCT | 17 | SEQ ID NO: 66 |
| | | | | ATAGGGCCTGGTGGACA | | |
| 67 | rs62056907 | TOP | [A/G] | AGGGGTCAAGATACAAGGAGTCACCAAAGAATG | 17 | SEQ ID NO: 67 |
| | | | | CAGAAGAGACAAGTTCA | | |
| 68 | rs2235013 | BOT | [T/C] | CCTTTTCTAAGACCAATATTAACAAGAATTAGT | 7 | SEQ ID NO: 68 |
| | | | | AGTAGAATGTTCTTATG | | |
| 69 | rs16878812 | TOP | [A/G] | TGTTGCTAATCCCAACCAGCATGATTTACGGGA | 6 | SEQ ID NO: 69 |
| | | | | AGTAAATCATCTATGAC | | |
| 70 | rs6549407 | TOP | [A/G] | GCCTGTCTCACAAACATTGGGTTCTATAGACGC | 3 | SEQ ID NO: 70 |
| | | | | TCCTAGATTGCATTTTC | | |
| 71 | rs28381848 | TOP | [A/G] | CCCAGTGCCTTGACAGGGTATGGGGGGACCTGC | 7 | SEQ ID NO: 71 |
| | | | | ATGACTAGCATTAAATG | | |
| 72 | rs79723704 | TOP | [A/C] | TAACCAGGGATCTGTGCGTTTTGCTATAATTCA | 20 | SEQ ID NO: 72 |
| | | | | GAAAGTAGCAGACTACT | | |
| 73 | rs72814052 | TOP | [A/G] | AAAAGTCGGTTCGAGAACCCAGGTGGAAAATAG | 17 | SEQ ID NO: 73 |
| | | | | ATTGAGGGAAGCAAAAC | | |
| 74 | rs10152908 | BOT | [T/C] | GAGTAAGAGTTAATCACTTCCACTGTGCACTTG | 15 | SEQ ID NO: 74 |
| | | | | TTTATTCCAAGTAGAAA | | |
| 75 | rs172769 | TOP | [A/C] | CTCTGGACATCTTCAGAGGGTCCCACTTTAGAC | 2 | SEQ ID NO: 75 |
| | | | | TTCACTGATCTCTTTTT | | |
| 76 | rs78596668 | BOT | [T/C] | TCACACTTTACATTTATTATTTCCAGTAAGGGA | 6 | SEQ ID NO: 76 |
| | | | | TATAGCTAAGATAGTTA | | |
| 77 | rs73307922 | BOT | [T/C] | CAGTTTGATGAATGGCAAAATCGTTCAAATGGA | 20 | SEQ ID NO: 77 |
| | | | | AAAGAGGAGAGAGATAG | | |
| 78 | rs3842 | TOP | [A/G] | TTCGTAATTAAAGGAACAGAGTGAGAGACATCA | 7 | SEQ ID NO: 78 |
| | | | | TCAAGTGGAGAGAAATC | | |
| 79 | rs7210584 | TOP | [A/C] | AGCCAGGGTTGAAGTCACTCACGGGTCCTCTCC | 17 | SEQ ID NO: 79 |
| | | | | GAGAACTCGAGTGGTGA | | |
| 80 | rs62402121 | BOT | [T/C] | CAAAGGTGATATGCATTTTAAATTTGATAGTTA | 6 | SEQ ID NO: 80 |
| | | | | TTGCCCAACTGTCTTTA | | |
| 81 | rs55709291 | TOP | [A/G] | CCCTCAGGCTGCTTGTTACCGTGGAAGCTTCCT | 17 | SEQ ID NO: 81 |
| | | | | GAACTCTCTCCAGACCC | | |
| 82 | rs72747088 | TOP | [A/G] | TTTTCATTTTTCTCTTCCCAACCCAATCCCCTC | 15 | SEQ ID NO: 82 |
| | | | | TCTCTAAATCTTGGTAT | | |
| 83 | rs929610 | BOT | [G/C] | TTCAATATATGTTTTCTGAACACCTTCTGTGTT | 14 | SEQ ID NO: 83 |
| | | | | CAAGGCACCATGCTGGG | | |
| 84 | rs6766242 | BOT | [T/C] | CCCTTGCATGTTCACCTTGTTATGTGTACTTTC | 3 | SEQ ID NO: 84 |
| | | | | ATCTCAATTGCCAGTTA | | |
| 85 | rs1468552 | BOT | [G/C] | AAAGTATCTCCCCAAATCATTCCCAAACACTAC | 16 | SEQ ID NO: 85 |
| | | | | AAAGGTAGTGCCATCAG | | |
| 86 | rs78838114 | BOT | [T/C] | TGCTCTAAAACTAATTTGCTTGAAGTGTACAGA | 15 | SEQ ID NO: 86 |
| | | | | ATGGAATTCGGGAAGGA | | |
| 87 | rs62489862 | BOT | [T/C] | ATCACTTTTCCATGAAATTGTCTTTGCATTAGC | 7 | SEQ ID NO: 87 |
| | | | | AAAATGAATCAAGCATA | | |
| 88 | rs894342 | TOP | [A/G] | TTGGTGATGCTGATAGTTGGAGATACCCAGACA | 15 | SEQ ID NO: 88 |
| | | | | GATAAGGTATATTGCCC | | |
| 89 | rs58882373 | BOT | [T/C] | ATCAATATGACTGGTGTCCTTCAGGAATGTGGT | 3 | SEQ ID NO: 89 |
| | | | | AGCACAGTGAAAAAGGT | | |
| 90 | rs3811939 | TOP | [A/G] | GCAGTAGGGGACTGGCTGCCGAGGGGGCATCTA | 5 | SEQ ID NO: 90 |
| | | | | GATTGAGATAGGTGGGA | | |
| 91 | rs6984688 | BOT | [T/G] | ATTGGCAAAAGTGCTCATTCTGGAAAAACAAAG | 8 | SEQ ID NO: 91 |
| | | | | AAGTGAGAAAGTGGATG | | |
| 92 | rs1018160 | BOT | [T/C] | ATTCTAAAGCTTTGTGTGGTCCACCATGATCAC | 5 | SEQ ID NO: 92 |
| | | | | CTTTTCCTGCTTCCCCC | | |
| 93 | rs76602912 | TOP | [A/G] | GCTCCATTTTCTTTGAGGTACATCAACATCAAT | 20 | SEQ ID NO: 93 |
| | | | | AACAGATCAATGGACCC | | |
| 94 | rs80067508 | TOP | [A/G] | AGCCTGACCTCATGGCTTAGCTGTGCCTCCTGG | 17 | SEQ ID NO: 94 |
| | | | | ACACCATCCCTCTCTGC | | |
| 95 | rs74888440 | BOT | [T/C] | TTCTGAAAGTCACAGCCCAGGGATTCAGACCCA | 5 | SEQ ID NO: 95 |
| | | | | CTAAAAAAAACTGAGAT | | |
| 96 | rs12481583 | BOT | [T/C] | ACTACATTACATCATGATGTATTGATTGCCTCT | 20 | SEQ ID NO: 96 |
| | | | | GGCCTAGGAATCTGCAG | | |
| 97 | rs66794218 | TOP | [A/G] | CCACTCATATGTCTGTTCTCACTCAGAGGTGAG | 17 | SEQ ID NO: 97 |
| | | | | GCCCTGTGTCTTCAGCC | | |
| 98 | rs16946701 | TOP | [A/G] | GGGGGACAGAGAAGTAACGTCACAAGATTTTAA | 15 | SEQ ID NO: 98 |
| | | | | GCTTGGGCCAGATATGG | | |
| 99 | rs75726724 | TOP | [A/G] | AAGTAGAGCAGAAAGGGCAAGCAGAGAACTAGA | 15 | SEQ ID NO: 99 |
| | | | | CAGAGAAGACAGATGAC | | |
| 100 | rs67959715 | BOT | [T/A] | TGGCTGCCTCTAGGGCAAGAAGACTGGGGATAT | 13 | SEQ ID NO: 100 |
| | | | | CACCATGGGCTCAATGT | | |
| 101 | rs11871392 | BOT | [T/G] | CCAAGTCCTTCTACCTCCCTGGGTGAGGGAACC | 17 | SEQ ID NO: 101 |
| | | | | GTTAGTGCCATCCTGAG | | |
| 102 | rs2044070 | TOP | [A/G] | AATCTTGGGGAATCTGAGTTTATTAGAGGAATG | 15 | SEQ ID NO: 102 |
| | | | | TAGGGAGGAAGCAGGCT | | |
| 103 | rs77612799 | BOT | [T/C] | TATCATATGCTCTAGTGACTTCATCAAGACAGT | 6 | SEQ ID NO: 103 |
| | | | | CTAAAGGAAGATGGGCC | | |
| 104 | rs6743702 | BOT | [T/C] | CAGAAACACCTTTAATGTTTTTATTTCTATGAA | 2 | SEQ ID NO: 104 |
| | | | | TATTCTCCTAATGATTA | | |
| 105 | rs616870 | BOT | [T/C] | TTAAAATGAGATCCCTTCCAACATGCTTTGCTG | 3 | SEQ ID NO: 105 |
| | | | | AGCCAGATTTATAAAAT | | |
| 106 | rs79590198 | TOP | [A/G] | TAGTACAGTAAGGGCAAAGGGCACTGCAATTGC | 5 | SEQ ID NO: 106 |
| | | | | TATTAAACTGTAAGAAG | | |
| 107 | rs75715199 | TOP | [A/G] | ATCCCCCGGAACTGGGGGAATTTCCAGGCACAT | 17 | SEQ ID NO: 107 |
| | | | | GAGGCTCTGTCAACCCA | | |
| 108 | rs13087555 | BOT | [T/C] | AGCCACTTAAAATAAATTTTTCCAGCAGTTATT | 3 | SEQ ID NO: 108 |
| | | | | CATTTAGTGCCAAAATA | | |
| 109 | rs4869618 | BOT | [T/C] | GCAGGGGCACATGCAATTGCCATTTAAAAATGA | 5 | SEQ ID NO: 109 |
| | | | | GGTCTGGCATGGCCAGA | | |
| 110 | rs117397046 | TOP | [A/G] | GTACCACAGCTCCCAGCTGCATGTACTTTAAAA | 17 | SEQ ID NO: 110 |
| | | | | ATGTGTCTAAGCCAGGC | | |
| 111 | rs8042817 | TOP | [A/G] | TGCAAACAGAAAAATCAGAACCTGCTCATGCTG | 15 | SEQ ID NO: 111 |
| | | | | CCATATTAATAGGAACC | | |
| 112 | rs2258097 | BOT | [T/C] | TAACTACACACTCAAGGCTCCCTCTCAAAGTCT | 17 | SEQ ID NO: 112 |
| | | | | CAAACCTTACAACTTCC | | |
| 113 | rs2260882 | TOP | [C/G] | AATACAGCCATGCGCTACCTACTGGCATTCCCG | 17 | SEQ ID NO: 113 |
| | | | | TCAGTGCGTACACGATC | | |
| 114 | rs532996 | TOP | [A/G] | AACTGCTTTCCTCATTGGCTTGGTCTCCATAGT | 13 | SEQ ID NO: 114 |
| | | | | GATTCATTTTGCTGTAA | | |
| 115 | rs11747040 | BOT | [T/C] | TGGAAATTTTTTTGTAATTAGAAATGACCTAAA | 5 | SEQ ID NO: 115 |
| | | | | GGATAGTTTCTATTCTT | | |
| 116 | rs10034039 | BOT | [T/G] | ATTGATTTTTATGTCAGCAATCTTCCAATCTTG | 4 | SEQ ID NO: 116 |
| | | | | TTAATTCTAAAATACTT | | |
| 117 | rs116909369 | TOP | [A/G] | GCCTAAGCTGAACCTGAGAGGTGAGGAAAACAG | 17 | SEQ ID NO: 117 |
| | | | | ACCAAGCTGACCAAACC | | |
| 118 | rs79134986 | TOP | [A/G] | GCGAACTGTGGAGTATCTCAGTAAGAGTGTTAG | 6 | SEQ ID NO: 118 |
| | | | | GAGGAATATTTTATAGG | | |
| 119 | rs117615688 | BOT | [T/C] | ACAACAACAAATCTCAAACAACTGTTCTGCCAA | 17 | SEQ ID NO: 119 |
| | | | | TGGGGTGGAGCACCTTT | | |
| 120 | rs8032253 | BOT | [T/C] | TGATGATTTTCCAGCATGGCAATGGTAAAGCTG | 15 | SEQ ID NO: 120 |
| | | | | CAAATAAAAAGCAGCCA | | |
| 121 | rs12818653 | BOT | [T/A] | TTCTTTTCTCCAAGCAAAAGAGAGAAGAGTTTA | 12 | SEQ ID NO: 121 |
| | | | | TTTCATTCTCAGCAGCT | | |
| 122 | rs4587884 | TOP | [A/C] | GGCAAAAGCAGAGATGTGAGCTGTAAATTTGAA | 14 | SEQ ID NO: 122 |
| | | | | TGAAGGACCAGATAGAA | | |
| 123 | rs77122853 | BOT | [T/C] | TAGGAACATAAAAGTTCAGATGTTAGTAGGACT | 20 | SEQ ID NO: 123 |
| | | | | AATAAAAAGTTATTGTT | | |
| 124 | rs117615061 | BOT | [T/C] | TTTTTCAGGTCTAGCTTAACCAAAACACTTAAA | 20 | SEQ ID NO: 124 |
| | | | | ACTGTTACCAAAAAACT | | |
| 125 | rs74682905 | TOP | [A/G] | CAAATAAATAAACTTTAAAGAAATGGCCAACTT | 7 | SEQ ID NO: 125 |
| | | | | GGGAAGGACATTAGGCC | | |
| 126 | rs2257468 | BOT | [T/C] | CAGTCCAACAACCAGTTCCAGAAGATCTCAGAG | 17 | SEQ ID NO: 126 |
| | | | | GTAGGCCGCTCCCCACA | | |
| 127 | rs2032582 | BOT | [T/G] | TGAAAATGTTGTCTGGACAAGCACTGAAAGATA | 7 | SEQ ID NO: 127 |
| | | | | AGAAAGAACTAGAAGGT | | |
| 128 | rs2235015 | BOT | [T/G] | GATTCATTTTTACATGTTTATTTTTAATGGAGA | 7 | SEQ ID NO: 128 |
| | | | | CTAAAGAGACATAAATG | | |
| 129 | rs2729794 | BOT | [T/C] | TCTTGATTCAATTGGAAGTAACTGAGAGGTATA | 15 | SEQ ID NO: 129 |
| | | | | TCACATGTTGTGATTCA | | |
| 130 | rs77549514 | TOP | [A/G] | TGCTCCATAACACAAATAATTTCATTCTTCTTC | 2 | SEQ ID NO: 130 |
| | | | | CTTTCTTGCCGAGTAGT | | |
| 131 | rs74790420 | TOP | [A/C] | ATGAGCAAGGAGGCCAAAACCCTGCGTGGACGG | 17 | SEQ ID NO: 131 |
| | | | | TCTGCTTCCCTGCCCTT | | |
| 132 | rs73129579 | BOT | [T/C] | GAGTGCCAAATATGTGCCCTTCCCCGTGGGGAA | 20 | SEQ ID NO: 132 |
| | | | | GACAAAAGTATGAGACA | | |
| 133 | rs12913346 | TOP | [A/C] | TATTTTTAGCAGCCTATGGATTCTAGGAGTGAC | 15 | SEQ ID NO: 133 |
| | | | | CCAGCTCCAGGGATAGG | | |
| 134 | rs117560908 | BOT | [T/C] | CATGAGGAAAGGCTGCAACTTTGAGCTCCCTCT | 17 | SEQ ID NO: 134 |
| | | | | TTAGCTAGGGAGCCTCC | | |
| 135 | rs72747091 | TOP | [A/G] | AGCATTAATGAAGCACAGGGCCTATCACGCAGT | 15 | SEQ ID NO: 135 |
| | | | | CAGGCTCAGTATAAGGT | | |
| 136 | rs2935751 | TOP | [A/G] | CATACTCAAATTGATACACAGCCTTTGTCCTGA | 8 | SEQ ID NO: 136 |
| | | | | GTGTTTGTCTTCCAAAA | | |
| 137 | rs4331446 | TOP | [A/G] | AGAGTAGTATTGCTTAAAAACTGCTCCAACCAC | 2 | SEQ ID NO: 137 |
| | | | | TTCTTAAACCTGAAACC | | |
| 138 | rs7523266 | BOT | [T/C] | TCGGCCAAAATCAGGGACAAGGATGACATGCCA | 1 | SEQ ID NO: 138 |
| | | | | TTGCTTACCAACTGCTA | | |
| 139 | rs7648662 | BOT | [T/C] | CCGTTGTGCAAACTCCAGAAAGGGCATCTCTCT | 3 | SEQ ID NO: 139 |
| | | | | GTCCCACTCCCCCATTA | | |
| 140 | rs117034065 | TOP | [A/G] | ATCTGCGTAAATTGCTGCATCTCTCTTGGCCTC | 15 | SEQ ID NO: 140 |
| | | | | AGTTTTCTTAGCCACAC | | |
| 141 | rs4836256 | BOT | [T/C] | GTAAGTGCCAGCTACTATTATTTAGGAGGCTAA | 5 | SEQ ID NO: 141 |
| | | | | GGCTCTAGGTGATGAGG | | |
| 142 | rs80238698 | BOT | [T/C] | TGCCACCCTATGGCATTCTTGTTGTGTAATGAA | 7 | SEQ ID NO: 142 |
| | | | | ATAACTCTCCTATGAAA | | |
| 143 | rs3730173 | BOT | [T/C] | CTGCGCTTGCCCAGGAGGCCCTGGTCTGCACTG | 20 | SEQ ID NO: 143 |
| | | | | TTTATAGAGAAGAACCC | | |
| 144 | rs11687884 | BOT | [T/C] | TTAGGAAAGTTCTGTACAGATATGTGTAATCCA | 2 | SEQ ID NO: 144 |
| | | | | GCATCTGTTTATCTATT | | |
| 145 | rs72693005 | BOT | [T/C] | AATGATGGAAAAAACTGCAGCGCACGGTGGAAA | 4 | SEQ ID NO: 145 |
| | | | | TGTCTACTTTGTATGCA | | |
| 146 | rs2589476 | BOT | [T/C] | CTCCTCATTATTCGCTTCTGCTGTAACTGCACC | 17 | SEQ ID NO: 146 |
| | | | | TATGGTAACCCAGGTGC | | |
| 147 | rs9813396 | BOT | [T/C] | AAGTGCTCTGTAACCAAATATTTTGGAAATGCT | 3 | SEQ ID NO: 147 |
| | | | | GAGTTGTACCAAGTTGG | | |
| 148 | rs10482667 | TOP | [A/G] | TTTTGAAATTTCCATTATATGCAAAGCCCATGA | 5 | SEQ ID NO: 148 |
| | | | | AAGGCTAAATATCAGTT | | |
| 149 | rs72784444 | TOP | [A/G] | GTTTGTAAATGCACACTGTTGGGGGAACCCTCT | 5 | SEQ ID NO: 149 |
| | | | | TCCTAGTCCTTGTTTCC | | |
| 150 | rs75074511 | BOT | [T/C] | TGGGCGAGAACTTATTCCTCAGGCCATTAGATT | 17 | SEQ ID NO: 150 |
| | | | | CCCTAATGCTGCACCTT | | |
| 151 | rs7951003 | TOP | [A/G] | GCCATGGGCAAAAACAGCTCAGGTAGTAATGAA | 11 | SEQ ID NO: 151 |
| | | | | GGTGTGGCTATAGCTGA | | |
| 152 | rs79584784 | TOP | [A/G] | ACATCAAACTAAATTACATCATCAGAGTAAAGA | 7 | SEQ ID NO: 152 |
| | | | | GACAATTTACAAAAAGG | | |
| 153 | rs2214102 | BOT | [T/C] | AAAAAGTTCTTCTTCTTTGCTCCTCCATTGCGG | 7 | SEQ ID NO: 153 |
| | | | | TCCCCTTCAAGATCCAT | | |
| 154 | rs28811003 | TOP | [A/G] | CTGGCTCCAGGCAAAGAATACTACCAGCAACAA | 15 | SEQ ID NO: 154 |
| | | | | AGAGGAACATTTCAGAT | | |
| 155 | rs6100261 | TOP | [A/T] | GGACTAGCCTGCTGCTTCATTTCCCCCCTCCTC | 20 | SEQ ID NO: 155 |
| | | | | TGCAGCCGATTTCAGAA | | |
| 156 | rs77152456 | TOP | [A/G] | ATATTAGTAACCTGGAAAACATACATGGAGGTA | 15 | SEQ ID NO: 156 |
| | | | | TGTTCATTAACGGCAGT | | |
| 157 | rs66624622 | BOT | [T/G] | ATGGGAAGAGCTGGATTTTTGTCGTGGAGTAAA | 5 | SEQ ID NO: 157 |
| | | | | GGAGAGGGAATCAAGAA | | |
| 158 | rs140302965 | TOP | [A/G] | AAAATCATAGAAATTGTGTCTAAGGATATGCTT | 7 | SEQ ID NO: 158 |
| | | | | TGGGATATTTGGACTTC | | |
| 159 | rs11653269 | BOT | [T/C] | CATAAACCAAAGGGATCTTCTCTACTCGTGCGT | 17 | SEQ ID NO: 159 |
| | | | | CCCTAGTCTCTCTCCCC | | |
| 160 | rs74405057 | TOP | [A/G] | GCTGCCTGTACTAGTGATAGTGAGGCTCACTAC | 20 | SEQ ID NO: 160 |
| | | | | CATCCACCACCTAAATT | | |
| 161 | rs7121 | TOP | [A/G] | GTGTAGCTTACGGGAGGGAAGTCAAAGTCAGGC | 20 | SEQ ID NO: 161 |
| | | | | ACGTTCATCACACTCAG | | |
| 162 | rs16977818 | TOP | [A/C] | CTCATTGTAAGATTCAAAAACATTCCAGCTTAC | 15 | SEQ ID NO: 162 |
| | | | | AAAACATATCCAGCTTA | | |
| 163 | rs12490095 | BOT | [T/C] | TTTGCAAGGCAATTTGTTCTACTGCTGGACAGC | 3 | SEQ ID NO: 163 |
| | | | | TTCATGTTTAATGTTTT | | |
| 164 | rs118003903 | TOP | [A/G] | CTATATTTGAACAAGCTTCTGGGTAATATTTAT | 17 | SEQ ID NO: 164 |
| | | | | GACAGGGAAGTCTTGAG | | |
| 165 | rs62377761 | BOT | [T/C] | CTGTGAACCAGGCACTGTTTGAAATGTTCCATT | 5 | SEQ ID NO: 165 |
| | | | | TATTGACTTATTTAAGT | | |
| 166 | P1_M_061510_ | MINUS | [−/I*] | ACTACTACTAATGTTGAAAGTATACCATGTAAC | 6 | SEQ ID NO: 166 |
| | 6_34_M | | | AGGCACTGTACAAAGCC | | |
| 167 | rs375115639 | MINUS | [−/I*] | TTTTGGGTTTTGTTGCTAGCATAAAAATTATTA | 6 | SEQ ID NO: 167 |
| | | | | CCTAGTGGATGGTAACA | | |
| 168 | rs1002204 | TOP | [A/C] | TTTTTTTTTCATTTGAAGTAAATATCCACCTTT | 7 | SEQ ID NO: 168 |
| | | | | GTATCTAATTTTGCATT | | |
| 169 | rs10062367 | TOP | [A/G] | TTATTTTTTAATAGTGTTCTTGCACATGAGGAG | 5 | SEQ ID NO: 169 |
| | | | | AAAGACTGAATTCAATT | | |
| 170 | rs10482642 | TOP | [A/G] | CGTGTCACTTCGTTTGACTTCAGCTGGGAACAT | 5 | SEQ ID NO: 170 |
| | | | | GCATATCAGTCGACTCA | | |
| 171 | rs10482658 | TOP | [A/G] | ATCGTCACACAGTTTTAAGACAAATGTTTTTAC | 5 | SEQ ID NO: 171 |
| | | | | CTATTTGACCTAGTCTG | | |
| 172 | rs1053989 | TOP | [A/C] | TGTGCTACAAACCTGAAACTGGTAAGACAAGCA | 5 | SEQ ID NO: 172 |
| | | | | CAAAGCAACGTGCAATA | | |
| 173 | rs10851628 | BOT | [T/C] | CTTGGATGGAGGCTCAGGGAGCCAAAGGCAAAT | 15 | SEQ ID NO: 173 |
| | | | | GTCTTCATAGAACCAGG | | |
| 174 | rs10947562 | BOT | [T/C] | ATCATGAATTAAACAAATTAATTTATGTATTTT | 6 | SEQ ID NO: 174 |
| | | | | GTTTTGAGTCAGTGTCT | | |
| 175 | rs11069612 | TOP | [A/G] | ACATGTGACCAACAAGATAATTATGAAACCTGA | 13 | SEQ ID NO: 175 |
| | | | | CTGCTGGATATGCTGAT | | |
| 176 | rs11071351 | BOT | [T/C] | GTCTTTTGGAAAATGCAATCTGCCACTCTGTGC | 15 | SEQ ID NO: 176 |
| | | | | AATGGAAAACCACTGCA | | |
| 177 | rs11091175 | TOP | [A/G] | TTATTAATATTAGCCTTTCTTCTCTCCCCGTTT | X | SEQ ID NO: 177 |
| | | | | ATGCTTTGGTGGGTACT | | |
| 178 | rs11638450 | BOT | [T/C] | TTTGGTTTGGGTTTTGTTTGGCAGAGGCAGAAT | 15 | SEQ ID NO: 178 |
| | | | | AGAATAAAGAACATGGG | | |
| 179 | rs11715827 | BOT | [T/G] | AGAATTATTGCTGCACAATTCTTATGAAACCGA | 3 | SEQ ID NO: 179 |
| | | | | ACTAGAGCTACACTATT | | |
| 180 | rs11745958 | BOT | [T/C] | CAGGCAGATCACTTGACGTGAGGAGTTCAAGTG | 5 | SEQ ID NO: 180 |
| | | | | AGGAGTTCAAGTCCAGC | | |
| 181 | rs11834041 | TOP | [A/G] | ACAAACAAACTGAGGTTTAGGTTTAGGTAGCTG | 12 | SEQ ID NO: 181 |
| | | | | GAGTTTATAGGCATGGC | | |
| 182 | rs1202180 | BOT | [T/C] | TCTGGAATAATAGTTACATTTGCTACATCCCTT | 7 | SEQ ID NO: 182 |
| | | | | TCTAGCGTCAACTCACT | | |
| 183 | rs12054781 | TOP | [A/G] | CATAATGTGATGCCATATTAAACTGTAATCACC | 5 | SEQ ID NO: 183 |
| | | | | TTTCCACCAAACTAATA | | |
| 184 | rs12539395 | TOP | [A/G] | CAAAATTCATATGTTGATACCTAATCTCCAAAG | 7 | SEQ ID NO: 184 |
| | | | | CAATAGTATTAAGGGTG | | |
| 185 | rs12720066 | BOT | [T/G] | AATACTGTTTGGTATGGCAAGACAGTATTGGTT | 7 | SEQ ID NO: 185 |
| | | | | TTGGTTCAAGTGCTCCT | | |
| 186 | rs1279754 | TOP | [A/C] | TTGGTTTTCCTGGGTGGGGAAGGGTGCTGGCCT | 5 | SEQ ID NO: 186 |
| | | | | CATTCACAACAGCAGAT | | |
| 187 | rs12872047 | BOT | [T/C] | GGGAAAGACAGAGTGAGAGAAAGAGAGAGTTAG | 13 | SEQ ID NO: 187 |
| | | | | CCTCTACATATTATAAG | | |
| 188 | rs12876742 | TOP | [A/C] | GCAGAGAGAGCCCTGTCTCAAAACAGATTTCTG | 13 | SEQ ID NO: 188 |
| | | | | AGTGTGGCTTCTGTCCA | | |
| 189 | rs12917505 | TOP | [A/G] | TCTCGTAGCTGAGAGAGTCATGACTATGGCGTG | 15 | SEQ ID NO: 189 |
| | | | | TTCTCTGTACTCTGAGG | | |
| 190 | rs13066950 | BOT | [T/G] | CTCAAGCAGAAGGAATCTCTCCCCATAGCCGCT | 3 | SEQ ID NO: 190 |
| | | | | ATAGTTTCAAATGTGCT | | |
| 191 | rs13229143 | TOP | [C/G] | GTGAGGATAGGTAGCTTTTCTTACTCACTGTTG | 7 | SEQ ID NO: 191 |
| | | | | TTACCAGTACCTAGAAC | | |
| 192 | rs1383707 | BOT | [T/C] | ACGAGCTTGTCATTCTGTAAATGACATATTCAT | 4 | SEQ ID NO: 192 |
| | | | | ATTCTTGGTATTGTACA | | |
| 193 | rs1441824 | BOT | [T/C] | CAAGGTTAAAATTCCCGCATTGTGGGCCTTGTA | 15 | SEQ ID NO: 193 |
| | | | | GCTTTCATGTCTTAATG | | |
| 194 | rs1652311 | TOP | [A/G] | GGATTTTGGCCATTCTAAGAGATGTGCAGTAGT | 2 | SEQ ID NO: 194 |
| | | | | AACTCAGTGTTTTATTT | | |
| 195 | rs17064 | BOT | [T/A] | CTGAAGACTCTGAACTTGACTGAGGAAATGTTA | 7 | SEQ ID NO: 195 |
| | | | | AACAGATACCTCTTCAT | | |
| 196 | rs17100236 | TOP | [A/G] | AACATTCCATTATCCTATTGTTCATTCTTTGGA | 5 | SEQ ID NO: 196 |
| | | | | GCTGTGATTTGTTTAAT | | |
| 197 | rs17149699 | TOP | [A/G] | AGCTTCGGTGAATATTAGAATGGCCTCAAGAGC | 7 | SEQ ID NO: 197 |
| | | | | TAGTAAAAAACACAGCC | | |
| 198 | rs1724386 | TOP | [A/G] | AGGCATATGGGGAAAAAATAAGGCAGGAAAGGA | 17 | SEQ ID NO: 198 |
| | | | | AGACGGAAAATGCTGTG | | |
| 199 | rs17250255 | TOP | [A/G] | TTGGTTTTATAAAGGATCTAAGTGTTTGGAAAG | 7 | SEQ ID NO: 199 |
| | | | | GTGTGGGACCATGTACT | | |
| 200 | rs17327624 | BOT | [T/G] | ACATGCTCTGCATGCTTTGACAGTACAGTGTAT | 7 | SEQ ID NO: 200 |
| | | | | AGAATAGACACAAAACT | | |
| 201 | rs17616338 | TOP | [A/G] | TAAGGTTGTATCATCTACCTGTAGTCACTGCAG | 4 | SEQ ID NO: 201 |
| | | | | TCAGCTGAATTTTACCA | | |
| 202 | rs17687796 | TOP | [A/G] | CTCTGTAGCCACACAGATGCCAACAGCTGGCAC | 17 | SEQ ID NO: 202 |
| | | | | TTGTCCAAGAAACATGT | | |
| 203 | rs17740874 | BOT | [T/C] | AGAATGGGTCACTTGTTAGAAACAGTCAAGGAT | 2 | SEQ ID NO: 203 |
| | | | | ACATACAAACAGTGGAA | | |
| 204 | rs17763104 | BOT | [T/C] | CCAAGAGTGGTGAAGCCTTCCTGTTTACAGAGG | 17 | SEQ ID NO: 204 |
| | | | | ATTTTCATATCTGTTAT | | |
| 205 | rs1880748 | BOT | [T/C] | ACACCCATGGGGCCAAGCCAGGAGCAGTCACCA | 17 | SEQ ID NO: 205 |
| | | | | CAGCCAACCTGCAGGCT | | |
| 206 | rs1882478 | TOP | [A/G] | TATTCTAAGGAAGTGCCCCCTAAAACAAAGCTC | 7 | SEQ ID NO: 206 |
| | | | | AGGAGCCTCAACCCGGC | | |
| 207 | rs1944887 | BOT | [T/C] | TCCCAACATCAAAAGGCAAATTCTTGCCCCACT | 11 | SEQ ID NO: 207 |
| | | | | TTTACAGATGAGAGCGC | | |
| 208 | rs2028629 | TOP | [A/G] | TACCATGGGAAACAGACAGTGGCCCCTGTTCTC | 17 | SEQ ID NO: 208 |
| | | | | AAGTGGCTTAGACTCTA | | |
| 209 | rs2143404 | TOP | [A/G] | CTTATTGGCCCTAAGTAAATCTTAGGTTAGGTA | 6 | SEQ ID NO: 209 |
| | | | | GAGCTCAGTTCCCAGGG | | |
| 210 | rs2173530 | BOT | [T/C] | GTATTTTTAGGAACATTCAGGAAAACAGGTAAA | 13 | SEQ ID NO: 210 |
| | | | | GGGTATTCAGGAATTCA | | |
| 211 | rs220806 | BOT | [T/C] | GGCCTTCCTCACTCTGACGGTGAGTTCCAGAGG | 6 | SEQ ID NO: 211 |
| | | | | ACAGGGATTTGTGGTTG | | |
| 212 | rs2235047 | TOP | [A/C] | TGGTTGCTAATTTCTCTTCACTTCTGGGAAACC | 7 | SEQ ID NO: 212 |
| | | | | AGCCCCTTATAAATCAA | | |
| 213 | rs2242071 | TOP | [A/G] | AACACAGAGCAGTATGTACAGGACAGCGTTAGA | 2 | SEQ ID NO: 213 |
| | | | | ATATACCAGAGAACAAG | | |
| 214 | rs2257474 | BOT | [T/C] | AAACACACCTGTCACCCACGACCCTGGCATAGG | 17 | SEQ ID NO: 214 |
| | | | | GCATCGTGAACCCATCA | | |
| 215 | rs2295583 | TOP | [A/T] | ATAGTATTCTGTTCTTCAGGGAGTTGTGGGTTC | 20 | SEQ ID NO: 215 |
| | | | | GGATCTGTGCAAAGATA | | |
| 216 | rs234629 | BOT | [T/C] | TAGGAATCAGGGAACTCTAGATGCGTCTAGCAG | 20 | SEQ ID NO: 216 |
| | | | | CTAGCCTGTGGCCTCGA | | |
| 217 | rs234630 | TOP | [A/G] | TTCAAATTGCTTGATTAAAATGGCAAACAGTTT | 20 | SEQ ID NO: 217 |
| | | | | GAAAATTGTATACCTCT | | |
| 218 | rs2436401 | TOP | [A/G] | GGATAATGGAAAAGGGGGTTTCTCCCAAGTAGA | 5 | SEQ ID NO: 218 |
| | | | | GAACTTAAACAGTGTGA | | |
| 219 | rs258750 | BOT | [T/C] | CACCTAGTCATGTGTATATAAAATCACCATGTT | 5 | SEQ ID NO: 219 |
| | | | | ATTACAGAATTTAGTAA | | |
| 220 | rs2589487 | BOT | [T/C] | CAATCTATTTTCCACCTGGGTTCTCGAACCGAC | 17 | SEQ ID NO: 220 |
| | | | | TTTTCCTCCCTCTCTTC | | |
| 221 | rs28364018 | BOT | [T/G] | GGGTCTTCCTACGGGACTGCCTTAGACGTGCTG | 8 | SEQ ID NO: 221 |
| | | | | GGCTTTGGCCTCAGTGA | | |
| 222 | rs28381774 | BOT | [T/C] | AGTTTTGGTTGGGGAGGACAATGCCAGGTTAAC | 7 | SEQ ID NO: 222 |
| | | | | AGACACTTAATATACAT | | |
| 223 | rs28381784 | TOP | [A/G] | AAAGAGAGTGGAAGTACCAGGTGGGCAAAGTTT | 7 | SEQ ID NO: 223 |
| | | | | ACAATTTTAAGTAGGAT | | |
| 224 | rs2963155 | TOP | [A/G] | ATGATTCTTTCCATGACACCTAGTGCCCTTCTC | 5 | SEQ ID NO: 224 |
| | | | | CATCTAGAGCTACCTCT | | |
| 225 | rs3133622 | BOT | [T/G] | AAATGAACTCAGCAATGAAATGGAACAAGCTAT | 8 | SEQ ID NO: 225 |
| | | | | CCATACATGCAGCAATT | | |
| 226 | rs32897 | BOT | [T/C] | CCATCATTGCCTGGCTGTTGAAGCAGTTCTTGA | 5 | SEQ ID NO: 226 |
| | | | | CATTTTAAAGTAATATG | | |
| 227 | rs33388 | TOP | [A/T] | TTGCTACAAGGAGGATTATGGGTGAAAGTCATG | 5 | SEQ ID NO: 227 |
| | | | | GATGGATTATGAGTTAA | | |
| 228 | rs3730168 | BOT | [T/C] | GATGGACATCACTGAAATGTAGTTTTGCCTGAA | 20 | SEQ ID NO: 228 |
| | | | | GTGTGGTTTGGATGCTC | | |
| 229 | rs3735833 | BOT | [T/G] | CTTGTTTGTGTATGATACATGAAGTAGAATTCA | 8 | SEQ ID NO: 229 |
| | | | | TACAGCACAAGTACTTT | | |
| 230 | rs3777747 | TOP | [A/G] | GAAATTCTCCATAATTTCTGATCCACTCTTACA | 6 | SEQ ID NO: 230 |
| | | | | TTCCTCTCCTTTCCAGC | | |
| 231 | rs3786066 | BOT | [T/C] | GGGGGCTGGGGGGAAGTCCCGGGACAGGTGCAT | 17 | SEQ ID NO: 231 |
| | | | | GTCATCAACACGACTGT | | |
| 232 | rs3798346 | BOT | [T/C] | AGATCTTTTCAGGCATAAAAGTTGTCAATAGGT | 6 | SEQ ID NO: 232 |
| | | | | TTTCATAAATTTCTAGG | | |
| 233 | rs3822736 | TOP | [A/G] | CCCTTGCACAGGCACAGCTATAATTTTTGTCTC | 5 | SEQ ID NO: 233 |
| | | | | TCTTCTGTTGGAAAGGT | | |
| 234 | rs389035 | BOT | [T/C] | GTGGTTTCTAATGATTTAATACCATCCCCCAGG | 2 | SEQ ID NO: 234 |
| | | | | GTGCTCTTCTTGTGATA | | |
| 235 | rs3924144 | TOP | [A/G] | GAATATTGAAGGTAGCCAGAAAAGAAAAAAAGG | 2 | SEQ ID NO: 235 |
| | | | | CACATTGCATGCAGAGG | | |
| 236 | rs4148737 | BOT | [T/C] | ATGGCAGTTCATTGCTTTACTATTTGGACATTT | 7 | SEQ ID NO: 236 |
| | | | | CAAACTGTCCCAAGGTG | | |
| 237 | rs4148749 | BOT | [G/C] | TTTTTTCAAACCTTTAAACAACAGTCCCACTTG | 7 | SEQ ID NO: 237 |
| | | | | GATAAAGTCTGAGAGCG | | |
| 238 | rs417968 | BOT | [T/C] | ATAGCCTAACTTTCCCCCCGAAGCTTCCCAAGC | 17 | SEQ ID NO: 238 |
| | | | | CCTCATGATATCTATTA | | |
| 239 | rs4458144 | BOT | [T/C] | ACCTGAGAATTCTCACCCATCCAATTCTACTTG | 2 | SEQ ID NO: 239 |
| | | | | ATATGGGATTCCTCTAA | | |
| 240 | rs4515335 | BOT | [T/C] | AATGGGCATGATCTCACTCACATGGAACAGGAT | 5 | SEQ ID NO: 240 |
| | | | | CTCTTTCCTTGTTAGCA | | |
| 241 | rs4728699 | TOP | [A/G] | AGTCACAGAAACATAGCAAGCCCTTGAAATCAG | 7 | SEQ ID NO: 241 |
| | | | | GCTTTCTGACTTTGTCT | | |
| 242 | rs4758040 | TOP | [A/G] | CACCTACACACATGCATGCACACACACATGGCC | 11 | SEQ ID NO: 242 |
| | | | | TCTCTCTCCAGGCTTCT | | |
| 243 | rs4812040 | TOP | [A/G] | CGTACAGACCTGGTCCAAAAATTCCAATTTCAT | 20 | SEQ ID NO: 243 |
| | | | | AGGTGTGGAGTTTTCAT | | |
| 244 | rs4912650 | BOT | [T/G] | CAAACAACCACCACATCAAAATAATAGCAAAGA | 5 | SEQ ID NO: 244 |
| | | | | CAACAACTAATACTAAT | | |
| 245 | rs4957891 | BOT | [T/C] | ATAGTAAGTTTTAAAGTAAGAGGTCAGAAACAT | 5 | SEQ ID NO: 245 |
| | | | | ATGTTACTTTACAAACA | | |
| 246 | rs5906392 | TOP | [A/G] | TTATGTAGCAGGTCCTGATGTAACAGAATTAAG | X | SEQ ID NO: 246 |
| | | | | ATTGCAGGTGGGATTGG | | |
| 247 | rs6026561 | BOT | [T/C] | TCCCTAGAACAGCAGGACCTGCGAAACTCTGAG | 20 | SEQ ID NO: 247 |
| | | | | GCCGCTTTGTGAGGTCC | | |
| 248 | rs6026565 | BOT | [T/A] | TTGAAAAGAGAAACCCACAGGGCTTTCTGCTTA | 20 | SEQ ID NO: 248 |
| | | | | AATCCCTCGGACACAGT | | |
| 249 | rs6026567 | TOP | [A/G] | TAAGGATGGGACCCCTACTGTCCATCTCAGGCT | 20 | SEQ ID NO: 249 |
| | | | | CAGCACTGCCTTGGGGC | | |
| 250 | rs6026593 | TOP | [A/G] | CTTCTACATCTTAGCTCACCTGTCCTCACAAAT | 20 | SEQ ID NO: 250 |
| | | | | AAACATCACTCTTGAAT | | |
| 251 | rs6092704 | BOT | [T/G] | TTGTTGAAATGTGACCACGAACTAGGTCTTAAC | 20 | SEQ ID NO: 251 |
| | | | | CTAGCAAATTCACAAAT | | |
| 252 | rs6100260 | TOP | [A/G] | CTTTCTAAACACTAGCAGCCCAGAATTCTCAGG | 20 | SEQ ID NO: 252 |
| | | | | CCACTTTTGGGCATTGT | | |
| 253 | rs6128461 | BOT | [T/C] | GTCTATGAATTGGTGAATCAGCCAAGTGAATGC | 20 | SEQ ID NO: 253 |
| | | | | TTCAAAAACTGGGACTC | | |
| 254 | rs6415328 | BOT | [T/C] | CCTCCTGAGATGAACATCGTGAGGAGTAAATAG | 7 | SEQ ID NO: 254 |
| | | | | AGATGCTATTCTCAGCT | | |
| 255 | rs6610868 | BOT | [T/C] | AACTCCGATTAATCACTAGTTGTTCACACCAAA | X | SEQ ID NO: 255 |
| | | | | AACCCAAGTGCCATTAC | | |
| 256 | rs6686061 | TOP | [A/C] | TCACCAAGTCTGGTTGTCCCAGTCTCCTATCTC | 1 | SEQ ID NO: 256 |
| | | | | TGTCTGTTCCTCTCCTC | | |
| 257 | rs6730350 | BOT | [T/G] | ATGAGTTGGAATTGCATAATGGGTAGATGCTGA | 2 | SEQ ID NO: 257 |
| | | | | TGCTGGAGAACTTTGAG | | |
| 258 | rs6746197 | BOT | [T/C] | GTCATTGACTCGACTATAATTTTCCAAACTACC | 2 | SEQ ID NO: 258 |
| | | | | TAAACGTGTTATATCAT | | |
| 259 | rs6963426 | BOT | [T/C] | TGATGATTAGGAGTCTGATGGAGGAAAGTAATT | 7 | SEQ ID NO: 259 |
| | | | | TTAAAACAACTTAATGG | | |
| 260 | rs7121326 | BOT | [T/C] | TGGGGTTTTATTTGCTTTTTTCCCAGTTTCTTA | 11 | SEQ ID NO: 260 |
| | | | | GATGTAAAGTTAGGTTA | | |
| 261 | rs7721799 | TOP | [A/G] | GGAACTCTGACGCAATCCAGGGCCGAGGAAAAA | 5 | SEQ ID NO: 261 |
| | | | | TGATTAAAACCCAACAA | | |
| 262 | rs7787082 | BOT | [T/C] | TACTGCAGTGAGTTCAAGTGTTGTACCTGCTTA | 7 | SEQ ID NO: 262 |
| | | | | AAATGCAGTGACACTAA | | |
| 263 | rs7799592 | TOP | [A/C] | GGCAGAGGGAACAGCTTGTGCAAAGGCCCTGGG | 7 | SEQ ID NO: 263 |
| | | | | GCAGGCCAAGGGCAGAG | | |
| 264 | rs796245 | BOT | [T/C] | AAAAGAGGATGGCTGGTTTATCTCAAGTAATCA | 2 | SEQ ID NO: 264 |
| | | | | GACATTTAATAATAATA | | |
| 265 | rs809482 | TOP | [A/C] | GTGCTATTTTGTTGCTGTTAGGTCTATTTTCTT | 2 | SEQ ID NO: 265 |
| | | | | CATCTGTTATTTCGCAT | | |
| 266 | rs8125112 | BOT | [T/C] | GCCTGGGGGAGCGGGGAATCGCTTTTCGCCGGC | 20 | SEQ ID NO: 266 |
| | | | | CTCCGCGTAACCTTGTT | | |
| 267 | rs919196 | TOP | [A/G] | GGCTCAACGGAAGTGACCGTCCCACAGTTATGC | 20 | SEQ ID NO: 267 |
| | | | | AGCACTAAGTCAATGGC | | |
| 268 | rs920750 | BOT | [T/C] | TTGTGACAGGTCCCAGCGTGAACACGCACGCCC | 17 | SEQ ID NO: 268 |
| | | | | TAGCCGGGCCCCAAACC | | |
| 269 | rs9332385 | TOP | [A/G] | AAGGGGACCGCAATGGAGGAGCAAAGAAGAAGA | 7 | SEQ ID NO: 269 |
| | | | | ACTTTTTTAAACTGAAC | | |
| 270 | rs930473 | BOT | [T/G] | GCTGACTTCTTGACTGCAGCCACAGGAAGGACT | 15 | SEQ ID NO: 270 |
| | | | | CAACCCAGGACCATCCA | | |
| 271 | rs9324921 | TOP | [A/C] | AATTTTTCAATGGTAAACAGACCAGAGTTATTC | 5 | SEQ ID NO: 271 |
| | | | | TAAGAAATTATGAAAAG | | |
| 272 | rs9348979 | TOP | [A/G] | AGGATTTCAAGACTTGCCTGAGCAACATAATGA | 6 | SEQ ID NO: 272 |
| | | | | GATGCCCTCTCTCAAAA | | |
| 273 | rs9571939 | TOP | [A/C] | AGCAAGCAGAAAACAAACAACTTCATTAAAAAT | 13 | SEQ ID NO: 273 |
| | | | | GAGCAGAGGACCTGAAC | | |
| 274 | rs9892359 | BOT | [T/C] | TTCTGAGACCTTCTTGCCCCTTTGTTTCTAAGC | 17 | SEQ ID NO: 274 |
| | | | | CCAGGGCCACAATTCCC | | |

*[−/I*] designates an allelic deletion/insertion polymorphism as defined in the respective SEQ ID NOs: 166 and 167

Further useful combinations of more than one polymorphism genotype are disclosed in Tables 5, 6, and 7 below, which all refer to the consecutively numbered, internal polymorphism-identifier (P_ID) of Table 2 to specify the genotype identity.

For the purposes of the present invention, the one or more polymorphism genotypes described above may be represented, for instance, within a nucleic acid of a length of, e.g., 1 nt, 2 nt, 3 nt, 4 nt, 5 nt, 10 nt, 15 nt, 20 nt, 25 nt, 30 nt, 35 nt, 40 nt, 45 nt, 50 nt, 60 nt, 70 nt, 80 nt, 90 nt, 100 nt, 200 nt, 300 nt, 400 nt, 500 nt, 1000 nt, 2000 nt, or more or any length in between these lengths. The nucleic acid may be any nucleic acid molecule, e.g. a DNA molecule, e.g., a genomic DNA molecule or a cDNA molecule, an RNA molecule, or a derivative thereof. The one or more polymorphism genotypes may further be represented by translated forms of the nucleic acid, e.g. a peptide or protein, as long as the polymorphic modification leads to a corresponding modification of the peptide or protein. Corresponding information is readily available in the art, e.g., from databases such as the NCBI dbSNP repository or the NCBI Genbank.

The polymorphism genotypes as described herein may be present on both strands of genomic DNA or its derivatives, i.e. on the chromosomal/genomic 5′→3′ strand and/or the chromosomal/genomic 3′→5′ strand. For example, a polymorphism can be present on the 5′→3′ strand as A, it is present on the 3′→5′ strand as T and vice versa. Also the insertion or deletion of bases may be detected on both DNA strands, with correspondence as defined above. For analytic purposes, the strand identity may be defined, or fixed, or may be chosen at will, e.g. in dependence on factors such the availability of binding elements, GC-content etc. Furthermore, for more universally applicable designation, a polymorphism genotype may be defined on both strands at the same time, or using the commonly known designations, such as the “probe/target”-designation, the “plus(+)/minus(−)”-designation, the “TOP/BOT”-designation or the “forward/reverse”-designation, as described in Nelson et al., Trends Genet. 2012, 28(8):361-3, or Illumina Inc. “TOP/BOT” Strand and “A/B” Allele-A guide to Illumina's method for determining Strand and Allele for the Golden Gate® and Infinium™ Assays”, Technical Note, © 2006; http://www.iliumina.com/documents/products/technotes/technote_topbot.pdf, both incorporated by reference herein in their entirety. For the sake of unambiguity in polymorphism genotype designation, e.g., the “TOP/BOT”-designation can be used to identify the polymorphism genotypes in Table 2 above. In the alternative, the probe sequence or the genomic flanking sequences can be used to identify the polymorphism genotypes in Table 2 above.

A “polymorphic site” or “polymorphic variant” as used herein relates to the position of a polymorphism or SNP as described herein within the genome or portion of a genome of a subject, or within a genetic element derived from the genome or portion of a genome of a subject.

“Linkage disequilibrium” as used herein refers to co-inheritance of two or more alleles at frequencies greater than would be expected from the separate frequencies of occurrence of each allele in the corresponding control population. The expected frequency of occurrence of two or more alleles that are inherited independently is the population frequency of the first allele multiplied by the population frequency of the second allele. Alleles or polymorphisms that co-occur at expected frequencies are said to be in linkage equilibrium. Polymorphisms in linkage disequilibrium with a polymorphism of Table 2 can be identified by methods known to one skilled in the art. For example, Devlin and Risch (Genomics 1995, 29(2):311-22; incorporated herein by reference in its entirety) provide guidance for determining the parameter delta (also referred to as “r”) as a standard measure of the linkage disequilibrium. Gabriel et al. (Science 2002, 296(5576):2225-9; incorporated herein by reference in its entirety) provides instructions for finding the maximal r² value in populations for disease gene mapping. Further, Carlson et al. (Am J Hum Genet 2004; 74(1): 106-120) disclose methods for selecting and analyzing polymorphisms based on linkage disequilibrium for disease gene association mapping. Stoyanovich and Pe'er (Bioinformatics, 2008, 24(3):440-2; incorporated herein by reference in its entirety) show that polymorphisms in linkage disequilibrium with identified polymorphisms have virtually identical response profiles. Currently, several databases provide datasets that can be searched for polymorphisms in strong linkage disequilibrium, which can be accessed by the following addresses: http://1000.genomes.org, http://www.hapmap.org, http://www.broadinsitute.org/mpg/snap. An example workflow for determining polymorphisms in linkage disequilibrium to a specific polymorphism is outlined in Uhr et al. (Neuron 2008, 57(2):203-9; incorporated herein by reference in its entirety). Preferably, the linkage disequilibrium referred to herein is strong linkage disequilibrium. “Strong linkage disequilibrium”, as used herein, means that the polymorphism is in linkage disequilibrium with an r² higher than 0.7 or higher than 0.8 in the tested population or an ethnically close reference population with the identified polymorphism.

A “sample obtained from a subject” as used herein may be any sample any biological sample comprising a bodily fluid, cell, tissue, or fraction thereof, which includes analyte biomolecules of interest such as nucleic acids (e.g., DNA or RNA). For instance, the sample obtained from the subject can be a buccal sample, a blood sample, plasma, serum, semen, sputum, cerebral spinal fluid, tears, a tissue sample, a formalin-fixed, paraffin-embedded tissue sample, or a hair follicle. Such samples are routinely collected, processed, preserved and/or stored by methods well known in the art. A biological sample can be further fractionated, if desired, to a fraction containing particular cell types. If desired, a sample can be a combination of samples from a subject such as a combination of a tissue and fluid sample.

In some embodiments, the subject's nucleic acid or DNA is extracted, isolated, and/or purified from the sample by any method commonly known in the art prior to polymorphism and/or SNP genotyping analysis. The term “isolated nucleic acid molecule”, as used herein, refers to a nucleic acid entity, e.g. DNA, RNA etc, being substantially free of other biological molecules, such as, proteins, lipids, carbohydrates, other nucleic acids or other material, such as cellular debris and growth media. Generally, the term “isolated” is not intended to refer to the complete absence of such material, or to the absence of water, buffers, or salts, unless they are present in amounts which substantially interfere with the methods of the present invention. In alternative embodiments, detection of one or more polymorphism genotypes may also be performed by using a non-extracted, non-isolated or non-purified sample. In some embodiments, DNA amplification by any suitable method known in the art is used prior to the detection of one or more polymorphism genotypes.

The term “detecting the presence or absence of one or more polymorphism/SNP genotypes” is used herein synonymously to a “polymorphism/SNP genotyping analysis” and refers to a step of determining in one or several patients the presence or absence of at least one polymorphism/SNP genotype, typically several polymorphism/SNP genotypes, or all polymorphism/SNP genotypes disclosed in Table 2, or, in some embodiments, all (known) polymorphism/SNP genotypes of the human genome, including endogenous and exogenous regions. In particular, detecting the presence or absence of one or more polymorphism genotypes as used herein may not be limited to the CRHR1 gene or to genes of the CRH pathway, but can encompass a genome-wide screening for polymorphism genotypes.

A detection step or polymorphism/SNP genotyping analysis can be performed by any suitable method known in the art. Such methods include, but are not limited to, PCR-related methods using polymorphism/SNP-specific primers and/or probes, a primer extension reaction, polymorphism/SNP microarrays analysis, sequencing analysis, mass spectrometry, 5′-nuclease assays, allele specific hybridization, high-throughput/multiplex variants of these techniques or combinations thereof, as described in the prior art, for example in Rampal, DNA Arrays: Methods and Protocols (Methods in Molecular Biology) 2010; Graham & Hill, DNA Sequencing Protocols (Methods in Molecular Biology) 2001; Schuster, Nat. Methods, 2008 and Brenner, Nat. Biotech., 2000; Mardis, Annu Rev Genomics Hum Genet., 2008, which are incorporated herein by reference. Genome-wide arrays can be purchased from different suppliers such as Illumina or Affymetrix. For primer selection, multiplexing and assay design, and the mass-extension for producing primer extension products the MassARRAY Assay Designer software may be used using the sequences presented in Table 2 as input. The MassARRAY Typer 3.4 software may be used for genotype calling.

For example, the presence or absence of a polymorphism genotype can be detected by determining the nucleotide sequence at the respective locus and may be carried out by allele-specific oligonucleotide (ASO)-dot blot analysis, primer extension assays, iPLEX polymorphism/SNP genotyping, dynamic allele-specific hybridization (DASH) genotyping, the use of molecular beacons, tetra primer ARMS PCR, a flap endonuclease invader assay, an oligonucleotide ligase assay, PCR-single strand conformation polymorphism (SSCP) analysis, quantitative real-time PCR assay, polymorphism/SNP microarray based analysis, restriction enzyme fragment length polymorphism (RFLP) analysis, targeted resequencing analysis and/or whole genome sequencing analysis. In some embodiments, any of the methods described herein can comprise the determination of the haplotype for two copies of the chromosome comprising the polymorphism genotypes identified herein.

In another example, genomic DNA isolated from a biological sample can be amplified using PCR as described above. The amplicons can be detectably-labeled during the amplification (e.g., using one or more detectably labeled dNTPs) or subsequent to the amplification. Following amplification and labeling, the detectably-labeled-amplicons are then contacted with a plurality of polynucleotides, containing one or more of a polynucleotide (e.g., an oligonucleotide) being capable of specifically hybridizing to a corresponding amplicon containing a specific polymorphism, and where the plurality contains many probe sets each corresponding to a different, specific polymorphism. Generally, the probe sets are bound to a solid support and the position of each probe set is predetermined on the solid support. The binding of a detectably-labeled amplicon to a corresponding probe of a probe set indicates the presence of a nucleic acid containing the polymorphism so amplified in the biological sample. Suitable conditions and methods for detecting a polymorphism or SNP using nucleic acid arrays are further described in, e.g., Lamy et al. (2006) Nucleic Acids Research 34(14): e100; European Patent Publication No. 1234058; U.S. Publication Nos. 2006/0008823 and 2003/0059813; and U.S. Pat. No. 6,410,231; the disclosures of each of which are incorporated herein by reference in their entirety.

In yet another example, MALDI-TOF (matrix-assisted laser desorption ionization time of flight) mass spectrometry on the Sequenom platform (San Diego, USA) may be used to detect one or more polymorphism genotypes.

Polynucleotides for use in detection of one or more of the polymorphism genotypes disclosed in Tables 2, 5, 6 or 7 are capable of specifically hybridizing to nucleic acids comprising said one or more polymorphism genotypes and can comprise the nucleic acid sequences of the polymorphism genotypes themselves, including up and/or downstream, flanking sequences, e.g., as hybridization polynucleotide probes or primers (e.g., for amplification or reverse transcription). “Capable of specifically hybridizing”, as used herein, refers to capability of hybridization under stringent conditions in any one of the methods of detection involving hybridization disclosed herein, as known to one skilled in the art. In that sense, primers and probes useful in such detection methods are particular polynucleotides capable of specifically hybridizing.

Primers or probes should contain a sequence of sufficient length and complementarity to a corresponding polymorphism locus to specifically hybridize with that locus under suitable hybridization conditions. For example, the polymorphism probes can include at least one (e.g., at least two, at least three, at least four, at least five, at least six, at least seven, at least eight, at least nine, at least 10, at least 11, at least 12, at least 15, at least 20, at least 25, at least 30, at least 35, at least 40, at least 45, at least 50, or 55 or more) nucleotides 5′ or 3′ to the polymorphism of interest. The polymorphic site of each probe (i.e., the polymorphism region) is generally flanked on one or both sides by sequence that is common among the different alleles. In specific embodiments, the polynucleotides capable of specifically hybridizing to the polymorphism genotypes are selected from the group consisting of the polynucleotides disclosed as “AlleleA Probe” in Table 2. The term “primer” may denote an oligo- or polynucleotide that acts as an initiation point of nucleotide synthesis under conditions in which synthesis of a primer extension product complementary to a nucleic acid strand is induced. The term “probe” may denote an oligonucleotide that is capable of specifically hybridizing to a target nucleic acid under suitable conditions, e.g., stringent conditions suitable for allele-specific hybridization. Primers and probes can be designed such are suitable for discriminating between wild-type allele or mutated allele of the position of a polymorphism to be analyzed, as described, e.g., by Coleman, and Tsongalis, Molecular Diagnostics: For the Clinical Laboratorian, 2007; Weiner et al. Genetic Variation: A Laboratory Manual, 2010, which are incorporated herein by reference.

Any of the methods of detecting a polymorphism can, optionally, be performed in multiplex formats that allow for rapid preparation, processing, and analysis of multiple samples, see above.

The detected polymorphism genotypes may be represented by values 0, 1 or 2. The value “0” may indicate that the polymorphism is present on none of the two homologous chromosomes, or in no allele, or is absent. The value “1” may indicate that the polymorphism is present on one of the two homologous chromosomes, or in one allele, or that the polymorphism genotype is heterozygous. The value “2” may indicate that the polymorphism is present on both homologous chromosomes, or in both alleles, or that the polymorphism genotype is homozygous.

The term “predicting a treatment response from the presence or absence of the one or more polymorphism genotypes”, as used herein, generally refers to a prediction step that provides a reasonably high prediction performance by associating the presence or absence of a polymorphism genotype with a treatment response. Similarly, the term “polymorphism genotype associated with a treatment response of a subject to treatment with a CRHR1 antagonist”, as used herein, generally refers to a polymorphism genotype being predicted to be associated with a treatment response with a reasonably high prediction performance. Specifically, the predicting step may comprise determining whether the subject will respond, or has an increased likelihood of responding to the treatment with a CRHR1 antagonist; and/or (b) determining whether the subject will not respond, or has a decreased likelihood of responding to the treatment with a CRHR1 antagonist. This is generally achieved herein by associating the presence or absence of the one or more polymorphism genotypes as a variable with a value indicative for treatment response within an algorithm for predicting a treatment response to a treatment with a CRHR1 antagonist, which is commonly a computer-implemented algorithm. The evaluation of the performance of the algorithm may depend on the problem the algorithm is applied for. If the algorithm is used to identify patients that are likely to respond to treatment with CRHR1 antagonists, the performance is preferably expressed by a high accuracy and/or sensitivity and/or precision. If patients should be identified which are likely not to respond to the treatment with CRHR1 antagonists, specificity and/or negative predictive value can be statistical measures to describe the performance of the prediction algorithm. For optimizing the prediction performance of the method of predicting a treatment response, a step of determining and/or optimizing the algorithm by a machine-learning method in a first subset of the test set and testing the prediction performance in an second independent subset of the test set may be carried out and repeated based on different numbers and groups of polymorphism genotypes, until the desired prediction performance is reached. Specifically, the algorithm for predicting may comprise a classification function (also known as binary classification test), which can comprise one or more statistical analysis methods and/or machine learning methods which are available to one of skill in the art. Specifically, statistical analysis methods and/or machine learning methods to be used in the invention may be selected from the group consisting of artificial neural network learning, decision tree learning, decision tree forest learning, linear discriminant analysis, non-linear discriminant analysis, genetic expression programming, relevance vector machines, linear models, generalized linear models, generalized estimating equations, generalized linear mixed models, the elastic net, the lasso support vector machine learning, Bayesian network learning, probabilistic neural network learning, clustering, and regression analysis, e.g., as described and exemplified herein. Statistical methods and/or machine learning methods from the group mentioned above may exist in different variants, especially applying or not applying prior and posterior weights in the analysis leading to solutions which may be applicable in different settings and may lead to models with more or less explanatory variables. The results of single methods may be used in a method called “ENSEMBLE learning” in which the results of several single analysis with one of the methods mentioned above are combined to arrive at a better prediction using either simply majority vote or using one of the machine learning algorithms with the results of the single analyses again as input to that specific algorithm.

In an exemplary embodiment of the method of the invention, the number of minor alleles for both polymorphism rs74888440 (P1) and rs9813396 (P2) is coded as a numeric variable, which can take one of the following values: 0, 1 or 2, denoting the two variables thus created as V1 for rs74888440 and V2 for rs9813396. Each subject is designated a value of the predictive quantitative variable PQV such that PQV=0.3205619+(0.2923413*V1)+(0.2362708*V2)+(−0.0104643*V1*V2). Depending on whether a subject's PQV is above or below a value of 0.5, that person is then predicted to not to respond, or to have a decreased likelihood of responding to a treatment with a CRHR1 antagonist (if PQV<=0.5), or to respond, or to have an increased likelihood of responding to a treatment with a CRHR1 antagonist (if PQV>0.5). For example, a subject who has no minor alleles at either of the two polymorphisms (homozygous for the common allele at both loci, such that V1=V2=0) is designated a PQV of 0.3205619 and is consequently predicted to be a non-responder. In another example, a subject who is heterozygous at P1 (V1=1) and homozygous for P2 (V2=2) is then designated a PQV of (0.3205619)+(0.2923413*1)+(0.2362708*2)+(−0.0104643*1*2)=1.064516 and is, in consequence, predicted to be a responder. In this example, a sensitivity of 0.6285714 and a specificity of 0.6626506 is reached.

In another exemplary embodiment of the method of the invention, the number of minor alleles for both SNPs rs74888440 (P1) and rs220806 (P2) is coded as a numeric variable, which can take one of the following values: 0, 1 or 2, denoting the two variables thus created as V1 for rs74888440 and V2 for rs220806. Each subject is designated a value of the predictive quantitative variable PQV such that PQV=0.539548+(0.460452*V1)+(−0.1765537*V2)+(−0.1567797*V1*V2). Depending on whether a subject's PQV is above or below a value of 0.5, that subject is then predicted to not to respond, or to have a decreased likelihood of responding to a treatment with a CRHR1 antagonist (if PQV<=0.5), or to respond, or to have an increased likelihood of responding to a treatment with a CRHR1 antagonist (if PQV>0.5). For example, a subject who has no minor alleles at either of the two SNPs (homozygous for the common allele at both loci, such that V1=V2=0) is designated a PQV of 0.539548 and is consequently predicted to be a responder. In another example, a subject who is heterozygous at SNP1 (V1=1) and homozygous for SNP2 (V2=2) is then designated a PQV of (0.539548)+(0.460452*1)+(−0.1765537*2)+(−0.1567797*1*2)=0.3333333 and is, in consequence, predicted to be a non-responder. In this example, a sensitivity of 0.6857143 and a specificity of 0.626506 is reached.

In a similar manner, one of skill in the art, having the polymorphisms of Table 2 and the additional information above at hand, will readily derive suitable methods, combinations of methods, parameters and/or coefficients such as those exemplified herein, for use in the methods of the invention, achieving a high performance of prediction.

Preferably, the prediction of the treatment response is made with a high accuracy, sensitivity, precision, specificity and/or negative predictive value.

“Accuracy”, “sensitivity”, “precision”, “specificity” and “negative predictive value” are exemplary statistical measure of the performance of the prediction algorithm. In the following, examples are given for determining the performance of the prediction algorithm.

As used herein, accuracy may be calculated as (number of true positives+number of true negatives)/(number of true positives+number of false positives+number of true negatives+number of false negatives), e.g., (number of patients correctly diagnosed as responding to CRHR1 antagonist+number of patients correctly diagnosed as not responding to CRHR1 antagonist)/(number of patients correctly diagnosed as responding to CRHR1 antagonist+number of patients wrongly diagnosed as responding to CRHR1 antagonist+number of patients correctly diagnosed as not responding to CRHR1 antagonist+number of patients wrongly diagnosed as not responding to CRHR1 antagonist). In some embodiments, the accuracy of prediction is higher than 50%, at least 60%, at least 70%, at least 80% or at least 90%.

As used herein, sensitivity may be calculated as (true positives)/(true positives+false negatives), e.g., (number of patients correctly diagnosed as responding to CRHR1 antagonist)/(number of patients correctly diagnosed as responding to CRHR1 antagonist+number of patients wrongly diagnosed as not responding to CRHR1 antagonist). In some embodiments, the sensitivity of prediction is higher than 50%, at least 60%, at least 70%, at least 80% or at least 90%.

As used herein, precision (also referred to as positive predictive value) may be calculated as (true positives)/(true positives+false positives), e.g.: (number of patients correctly diagnosed as responding to CRHR1 antagonist)/(number of patients correctly diagnosed as responding to CRHR1 antagonist+number of patients wrongly diagnosed as responding to CRHR1 antagonist). In some embodiments, the precision of prediction is higher than 50%, at least 60%, at least 70%, at least 80% or at least 90%.

As used herein, specificity is calculated as (true negatives)/(true negatives+false positives), e.g.: (number of patients correctly diagnosed as not responding to CRHR1 antagonist)/(number of patients correctly diagnosed as not responding to CRHR1 antagonist+number of patients wrongly diagnosed as responding to CRHR1 antagonist). In some embodiments, the specificity of prediction is higher than 50%, at least 60%, at least 70%, at least 80% or at least 90%.

As used herein, negative predictive value is calculated as (true negatives)/(true negatives+false negatives), e.g.: (number of patients correctly diagnosed as not responding to CRHR1 antagonist)/(number of patients correctly diagnosed as not responding to CRHR1 antagonist+number of patients wrongly diagnosed as not responding to CRHR1 antagonist). In some embodiments, the negative predictive value is higher than 50%, at least 60%, at least 70%, at least 80% or at least 90%.

Other statistical measures useful for describing the performance of the prediction algorithm are geometric mean of sensitivity and specificity, geometric mean of positive predictive value and negative predictive value, F-measure and area under ROC curve, and the positive and negative likelihood ratios, the false discovery rate and Matthews correlation coefficient. These measures and method for their determination are well known in the art.

In general, a prediction algorithm with high sensitivity may have low specificity and vice versa. For the purposes of the present invention, it is generally preferable that the prediction algorithm is based on a number of polymorphism genotypes selected from Table 2 sufficient to achieve a sensitivity and specificity of higher than 50% each, optionally at least 60% each, at least 70% each, at least 80% each, or at least 90% each.

For a prediction whether a patient will respond, or has an increased likelihood of responding to a treatment with a CRHR1 antagonist, the prediction algorithm may be based on a number of polymorphisms sufficient to achieve a prediction sensitivity and/or precision of higher than 50%, optionally at least 60%, at least 70%, at least 80%, or at least 90%.

For the prediction whether the subject will not respond, or has a decreased likelihood of responding to a treatment with a CRHR1 antagonist, the prediction algorithm may be based on a number of polymorphisms sufficient to achieve a prediction specificity and/or negative predictive value of higher than 50%, optionally at least 60%, at least 70%, at least 80%, or at least 90%.

For a prediction whether a patient responds to a treatment with CRHR1 antagonists or not, the prediction algorithm may be based on a number of polymorphisms sufficient to achieve sensitivity and/or precision and/or specificity and/or negative predictive value of higher than 50%, optionally at least 60%, at least 70%, at least 80%, or at least 90%.

Based on the disclosure of the present invention, in particular of the highly useful set of polymorphism genotypes disclosed in Table 2, the skilled person in the art is enabled to employ the statistical analysis methods and/or machine-learning methods disclosed herein and to identify suitable parameters for further improving the prediction performance, as defined above. The whole statistical work-flow can be automated by the use of an algorithm as described above, implemented and/or stored on a machine-readable medium, e.g., implemented and/or stored on a computer.

Typically, at least 1, at least 2, at least 3, at least 4, at least 5, at least 6, at least 7, at least 8, at least 9, at least 10, at least 20, at least 30, at least 50, at least 100, at least 100, at least 200 or all polymorphism genotypes disclosed in Table 2 are used for predicting the treatment response to a CRHR1 antagonist.

Using various such polymorphism genotype sets and statistical analysis methods as described above, the present invention consistently achieves a high predictive performance in directly predicting a clinical response. For instance, Example 1 describes a study with clinical data from 300 enrolled patients, wherein 150 polymorphism genotypes were used in a method for predicting the clinical treatment response of subjects to a treatment with a CRHR1 antagonist. Therein, a sensitivity of about 78% and a specificity of about 73% was observed, which is considered to reflect a superior reliability in predicting both true positive responses and true negative responses. Further, Example 2 provides examples of minimal subsets of only one, two, four or eight polymorphism genotypes selected from the group of polymorphism genotypes disclosed in Table 2, achieving a performance of predicting a clinical treatment response with values for specificity and sensitivity which are still higher than 60%, or even higher than 70%. Predictive performance in terms of sensitivity and specificity can be further increased to at least 75% each, e.g., by including specific combinations of 32 polymorphism genotypes, as is also shown in Example 2.

Furthermore, in patients with depressive symptoms and/or anxiety symptoms, another embodiment of the method for predicting a treatment response to CRHR1 antagonists, the method of predicting a treatment response as described above may be also accompanied by analyzing the rapid-eye-movement (REM) sleep, e.g. during night sleep of a patient in a sleep EEC. In some embodiments, an alteration in REM sleep may serve as an additional biomarker to identify subjects who would benefit from treatment with a CRHR1 antagonist. REM sleep typically comprises a characteristic coincidence of nearly complete muscle atonia, a waking-like pattern of brain oscillations and rapid eye movements (REMs). The amount of REMs during consecutive REM sleep episodes is usually increasing throughout the night. Single and short REMs with low amplitude can be characteristic for initial parts of REM sleep. The amount of REMs, in particular within the first REM sleep episode, can be of clinical relevance. Recent clinical and animal data supports the correlation of REM density with an increased CRH activity. For example, Kimura et al. (Mol. Psychiatry, 2010) showed that mice overexpressing CRH in the forebrain exhibit constantly increased rapid eye movement (REM) sleep compared to wildtype mice. In addition, it could be shown that treatment with the CRHR1 antagonist DMP696 could reverse the REM enhancement. Further, the polymorphism analysis and REM density analysis as described herein may be combined for predicting the response of patients with depressive symptoms and/or anxiety symptoms to treatment with a CRHR1 antagonist. The REM analysis may be carried out before, concomitant or after the polymorphism analysis. For example, the REM density analysis may be carried out on subjects that where identified by the polymorphism analysis as responding, or having an increased likelihood of responding to the treatment with a CRHR1 antagonist; or as not responding, or having a decreased likelihood of responding to the treatment with a CRHR1 antagonist. The recording of a “sleep-EEG” (also referred to “polysomatic recordings”) may comprise electroencephalography (EEG), vertical and horizontal elecrooculography (EOG), electromyography (EMG) and/or electrocardiography (ECG). In EOG, muscle activities of right and left eye may be recorded by electrooculograms (one or typically two channels) in order to visualize the phasic components of REM sleep. “REM analysis” or “analyzing the rapid-eye-movement (REM)” may refer to a method comprising recoding of muscle activities of right and left eye by EOG and then analyzing the electrooculogram. The recognition of REM in the electrooculogram may be done manually, for example by standard guidelines Rechtschaffen and Kales, 1968, Bethesda, Md.: National Institute of Neurological Diseases and Blindness, incorporated herein by reference in its entirety.

### Methods of Treatment

In a further aspect, the present invention also provides methods of treating a condition treatable by treatment with a CRHR1 antagonist in a subject in need thereof, comprising administering an effective amount of a CRHR1 antagonist to a subject in need thereof, wherein the subject has been predicted to respond, or has an increased likelihood of responding, to treatment with a CRHR1 antagonist, as determined by the method described above, and wherein the CRHR1 antagonist is a compound of Formula I, as defined herein, or any one of “formulae I-VI” as disclosed at pages 2-6 and 12-48 of WO 2013/160317 (A2) (PCT/EP2013/058413). Likewise the invention features a CRHR1 antagonist for use in treating a condition treatable by treatment with a CRHR1 antagonist in a subject in need thereof, wherein the subject has been predicted to respond, or has an increased likelihood of responding, to treatment with a CRHR1 antagonist, as determined by the method described above, and wherein the CRHR1 antagonist is a compound of Formula I, as defined herein, or any one of “formulae I-VI” as disclosed at pages 2-6 and 12-48 of WO 2013/160317 (A2) (PCT/EP2013/058413).

Conditions which are treatable by a treatment with a CRHR1 antagonist are generally defined above. Specific conditions comprise, but are not limited to, behavioural disorders, psychiatric disorders, mood disorders, neurological disorders, neurodegenerative disorders, inflammatory or stress-induced immune disorders, CRH-related cardiovascular diseases or metabolic diseases. Specifically, such conditions comprise anxiety symptoms, generalized anxiety disorder, panic, phobias, obsessive-compulsive disorder, post-traumatic stress disorder, sleep disorders induced by stress, pain perception such as fibromyalgia, mood disorders such as depressive symptoms, including major depression, single episode depression, recurrent depression, child abuse induced depression, mood disorders associated with premenstrual syndrome, and postpartum depression, dysthymia, bipolar disorders, cyclothymia, chronic fatigue syndrome, stress-induced headache, eating disorders such as anorexia and bulimia nervosa, hemorrhagic stress, stress-induced psychotic episodes, euthyroid sick syndrome, syndrome of inappropriate antidiarrhetic hormone (ADH), obesity, infertility, head traumas, spinal cord trauma, ischemic neuronal damage (e.g., cerebral ischemia such as cerebral hippocampal ischemia), excitotoxic neuronal damage, epilepsy, senile dementia of the Alzheimers type, multi-infarct dementia, amyotrophic lateral sclerosis, chemical dependencies and addictions (e.g., dependencies on alcohol, nicotine, cocaine, heroin, benzodiazepines, or other drugs), drug and alcohol withdrawal symptoms, hypertension, tachycardia, congestive heart failure, osteoporosis, premature birth, and hypoglycaemia, inflammatory disorders such as rheumatoid arthritis and osteoarthritis, pain, asthma, psoriasis and allergies, irritable bowel syndrome, Crohn's disease, spastic colon, post-operative ileus, ulcer, diarrhea, stress-induced fever, human immunodeficiency virus (HIV) infections, neurodegenerative diseases such as Alzheimer's disease, Parkinson's disease and Huntington's disease, gastrointestinal diseases, stroke, stress induced immune dysfunctions, muscular spasms, urinary incontinence. In a preferred embodiment, the condition is selected from the groups consisting of depressive symptoms, anxiety symptoms or both depressive symptoms and anxiety symptoms.

Any CRHR1 antagonist as generally defined by Formula I herein, or any one of “formulae I-VI” as disclosed at pages 2-6 and 12-48 of WO 2013/160317 (A2) (PCT/EP2013/058413) can be used in the method of treatment. In a specific embodiment of the method of treatment, the CRHR1 antagonist is selected from the group consisting of a Type I CRHR1 antagonist, a bicyclic Type II CRHR1 antagonist, an atypical CRHR1 antagonist or a cyclohexyl amide CRHR1 antagonist. In another specific embodiment of the method of treatment, the CRHR1 antagonist is selected from the group consisting of GW876008 (Emicerfont), GSK-561679 (NBI-77860, Verucerfont), GSK586529, BMS-562,086 (Pexacerfont), NBI-30775 (R-121919), NBI-34101, CP-316,311, CP-376,395, PF-00572778, NVP-AAG561, Ono-2333MS, E2508, E2009, R317573 (JNJ19567470, CRA5626), R278995 (CRA0450), CRA-1000, CRA-1001, CP154,526, Antalarmin, DMP-695, DMP-696, DMP-904, SC-241, BMS-561388, NB130545, PD-171729, NB134041, NB135965, SN003, NBI-27914, trans-2-chloro-N-(4-((5-fluoro-4-methyl-pyridin-2-ylamino)-methyl)-cyclohexyl)-5-(trifluoromethyl)-benzamide, or a pharmaceutically acceptable salt thereof. In one embodiment of the method of treatment, the CRHR1 antagonist is not SSR-125543.

It will be appreciated that reference to treatment is intended to include prevention as well as the partial alleviation, or full remission of symptoms.

CRHR1 antagonists may be administered as the raw chemical but the active ingredient is preferably formulated in a pharmaceutical composition suitable for administration by any convenient route, preferably in a form suitable for use in human medicine. The treatment can comprise any suitable route of administration, such as oral, buccal, parenteral, topical (including ophthalmic and nasal), depot or rectal administration or in a form suitable for administration by inhalation or insufflation (either through the mouth or nose) administration of the CRHR1 antagonist.

CRHR1 antagonists can be administered at any suitable efficacious dose, which one skilled in the art will readily adapt, e.g., to the specific condition to be treated. For many therapeutic indications as encompassed herein, a dose of about 1 mg to about 2000 mg per day, about 2 mg to about 1000 mg per day, about 5 mg to about 500 mg per day, about 10 mg to about 250 mg, or about 20 to about 100 mg daily will be efficacious. It will be appreciated that it may be necessary to make routine variations to the dosage, depending on the age and condition of the patient and the precise dosage will be ultimately at the discretion of the attendant physician or veterinarian. The dosage will also depend on the route of administration and the particular compound selected. Thus, for parenteral administration a daily dose will typically be in the range of 1 to about 100 mg, preferably 1 to 80 mg per day. For oral administration a daily dose will typically be within the range of 1 to 300 mg e.g. 1 to 100 mg of a CRHR1 antagonist. For instance, in treating depressive symptoms and/or anxiety symptoms, daily oral doses of about 10 mg, about 20 mg, or about 100 mg of a CRHR1 antagonist can be efficacious.

### Compositions, Kits and Arrays and Uses Thereof

The disclosure further provides compositions comprising polynucleotides (e.g., probes), as well as kits and arrays. Polynucleotide compositions, kits, and arrays are useful in, e.g., detecting the presence of (a) one or more polymorphism genotypes as disclosed in Table 2, (b) one or more polymorphism genotypes being in linkage disequilibrium with any one of the polymorphism genotypes of (a), or a combination of (a) and (b). The compositions, kits and arrays are further useful for predicting the treatment response of a subject to treatment with a CRHR1 antagonist.

The compositions, kits or arrays can include at least one polynucleotide capable of specifically hybridizing to a nucleic acid comprising: (a) at least one polymorphism genotype as disclosed in Table 2; (b) at least one polymorphism genotype being in linkage disequilibrium with any one of the polymorphism genotypes of (a), or (c) a combination of (a) and (b). In one embodiment, the at least one polynucleotide comprises less than 100,000, less than 90,000, less than 80,000, less than 70,000, less than 60,000, less than 50,000, less than 40,000, less than 30,000, less than 20,000, less than 15,000, less than 10,000, less than 5,000, less than 4,000, less than 3,000, less than 2,000, less than 1,500, less than 1,000, less than 750, less than 500, less than 200, less than 100, or less than 50 different polynucleotides in total. Specifically, the compositions, kits or arrays can include at least two, at least three, at least four, at least five, at least six, at least seven, at least eight, at least nine, at least 10, at least 11, at least 12, at least 15, at least 20, or at least 30, or at least 50, or at least 100, or at least 200, or 274 polynucleotides capable of specifically hybridizing to each of at least two, at least three, at least four, at least five, at least six, at least seven, at least eight, at least nine, at least 10, at least 11, at least 12, at least 15, at least 20, or at least 30, or at least 50, or at least 100, or at least 200, or 274 of (a) at least one polymorphism genotype as disclosed in Table 2; (b) at least one polymorphism genotype being in linkage disequilibrium with any one of the polymorphism genotypes of (a), or (c) a combination of (a) and (b).

A polynucleotide can include a coding sequence or non-coding sequence (e.g., exons, introns, or 5′ or 3′ regulatory sequences). The polynucleotide can also be single or double-stranded and of variable length. In some embodiments, the length of one strand of a polynucleotide capable of specifically hybridizing to a nucleic acid comprising: (a) at least one a polymorphism genotype as disclosed in Table 2; (b) at least one polymorphism genotype being in linkage disequilibrium with any one of the polymorphism genotypes of (a), or (c) a combination of (a) and (b) can be about six nucleotides (e.g., about seven nucleotides, about eight nucleotides, about nine nucleotides, about 10 nucleotides, about 12 nucleotides, about 13 nucleotides, about 14 nucleotides, about 15 nucleotides, about 20 nucleotides, about 25 nucleotides, about 30 nucleotides, about 35 nucleotides, about 40 nucleotides, about 50 nucleotides, about 75 nucleotides, about 100 nucleotides, or about 150 or more nucleotides) in length. As is commonly known in the art, a longer polynucleotide often allows for higher stringency hybridization and wash conditions. The polynucleotide can be DNA, RNA, modified DNA or RNA, or a hybrid, where the nucleic acid contains any combination of deoxyribo- and ribo-nucleotides, and any combination of uracil, adenine, thymine, cytosine and guanine, as well as other bases such as inosine, xanthine, and hypoxanthine.

The polynucleotides can be attached to a solid support, e.g., a porous or non-porous material that is insoluble. The polynucleotides can be arranged in an array on the solid support, e.g., in a microarray. A solid support can be composed of a natural or synthetic material, an organic or inorganic material. The composition of the solid support on which the polynucleotide sequences are attached by either 5′ or 3′ terminal attachment generally depend on the method of attachment (e.g., covalent attachment). Suitable solid supports include, but are not limited to, plastics, resins, polysaccharides, silica or silica-based materials, functionalized glass, modified silicon, carbon, metals, inorganic glasses, membranes, nylon, natural fibers such as silk, wool and cotton, or polymers. The material comprising the solid support can have reactive groups such as carboxy, amino, or hydroxyl groups, which are used for attachment of the polynucleotides. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, or polymethylpentene (see, e.g., U.S. Pat. No. 5,427,779, the disclosure of which is hereby incorporated by reference in its entirety). Alternatively, polynucleotides can be attached to the solid support without the use of such functional groups.

Arrays of polynucleotides can also be conjugated to solid support particles. Many suitable solid support particles are known in the art and illustratively include, e.g., particles, such as Luminex®-type encoded particles, magnetic particles, and glass particles. Exemplary particles that can be used can have a variety of sizes and physical properties. Particles can be selected to have a variety of properties useful for particular experimental formats. For example, particles can be selected that remain suspended in a solution of desired viscosity or to readily precipitate in a solution of desired viscosity. Particles can be selected for ease of separation from sample constituents, for example, by including purification tags for separation with a suitable tag-binding material, paramagnetic properties for magnetic separation, and the like. In some embodiments, encoded particles are used. Each particle includes a unique code (such as a bar code, luminescence code, fluorescence code, a nucleic acid code, and the like). Encoding can be used to provide particles for evaluating different nucleic acids in a single biological sample. The code is embedded (for example, within the interior of the particle) or otherwise attached to the particle in a manner that is stable through hybridization and analysis. The code can be provided by any detectable means, such as by holographic encoding, by a fluorescence property, color, shape, size, weight, light emission, quantum dot emission and the like to identify particle and thus the capture probes immobilized thereto. Encoding can also be the ratio of two or more dyes in one particle that is different than the ratio present in another particle. For example, the particles may be encoded using optical, chemical, physical, or electronic tags. Examples of such coding technologies are optical bar codes fluorescent dyes, or other means. In some embodiments, the particle code is a nucleic acid, e.g., a single stranded nucleic acid.

Different encoded particles can be used to detect or measure multiple nucleic acids (e.g., polymorphism genotypes or mRNAs) in parallel, so long as the encoding can be used to identify the polynucleotide (corresponding to an analyte nucleic acid) on a particular particle, and hence the presence or amount of the analyte nucleic acid (e.g., a polymorphism genotypes or mRNA from a biological sample) being evaluated. A sample can be contacted with a plurality of such coded particles. When the particles are evaluated, e.g., using a fluorescent scanner, the particle code is read as is the fluorescence associated with the particle from any probe used to evaluate modification of the intact substrate associated with the particles.

One exemplary platform utilizes mixtures of fluorescent dyes impregnated into polymer particles as the means to identify each member of a particle set to which a specific capture probe has been immobilized. Another exemplary platform uses holographic barcodes to identify cylindrical glass particles. For example, Chandler et al. (U.S. Pat. No. 5,981,180) describes a particle-based system in which different particle types are encoded by mixtures of various proportions of two or more fluorescent dyes impregnated into polymer particles. Soini (U.S. Pat. No. 5,028,545) describes a particle-based multiplexed assay system that employs time-resolved fluorescence for particle identification. Fulwyler (U.S. Pat. No. 4,499,052) describes an exemplary method for using particle distinguished by color and/or size. U.S. Publication Nos. 2004-0179267, 2004-0132205, 2004-0130786, 2004-0130761, 2004-0126875, 2004-0125424, and 2004-0075907 describe exemplary particles encoded by holographic barcodes.

U.S. Pat. No. 6,916,661 describes polymeric microparticles that are associated with nanoparticles that have dyes that provide a code for the particles. The polymeric microparticles can have a diameter of less than one millimeter, e.g., a size ranging from about 0.1 to about 1,000 micrometers in diameter, e.g., 3-25 μm or about 6-12 μm. The nanoparticles can have, e.g., a diameter from about 1 nanometer (nm) to about 100,000 nm in diameter, e.g., about 10-1,000 nm or 200-500 nm.

An “array”, as used herein, refers to a plurality of polynucleotides comprised in the composition or kit being immobilized at predetermined positions on a solid support such that each polynucleotide can be identified by its position.

The compositions, kits and arrays can be, but are not necessarily used in genome-wide genotyping analysis, but for efficient, low cost, and application-specific genotyping analysis, tailored to be used in the methods of predicting a treatment response to a treatment with a CRHR1 antagonist, as disclosed herein. Thus, in some embodiments of any of the compositions, kits and arrays described herein, the array of polynucleotides has less than 100,000 (e.g., less than 90,000; less than 80,000; less than 70,000; less than 60,000; less than 50,000; less than 40,000; less than 30,000; less than 20,000; less than 15,000; less than 10,000; less than 5,000; less than 4,000; less than 3,000; less than 2,000; less than 1,500; less than 1,000; less than 750; less than 500, less than 200, less than 100, or less than 50) different polynucleotides.

The kits described above can, optionally, contain instructions for detecting the presence or absence of the at least one polymorphism genotype in a sample obtained from a subject. In some embodiments, the kits can include one or more reagents for processing a biological sample. For example, a kit can include reagents for isolating mRNA or genomic DNA from a biological sample and/or reagents for amplifying isolated mRNA (e.g., reverse transcriptase, primers for reverse transcription or PCR amplification, or dNTPs) and/or genomic DNA. The kits can also, optionally, contain one or more reagents for detectably-labeling an mRNA, mRNA amplicon, genomic DNA or DNA amplicon, which reagents can include, e.g., an enzyme such as a Klenow fragment of DNA polymerase, T4 polynucleotide kinase, one or more detectably-labeled dNTPs, or detectably-labeled gamma phosphate ATP (e.g., 33P-ATP). In some embodiments, the kits can include a software package for analyzing the results of, e.g., a microarray analysis. The kits described herein can also, optionally, include instructions for administering a CRHR1 antagonist where presence or absence of one or more polymorphism genotypes detectable by the plurality of polynucleotides or the array predicts that a subject will response to a CRHR1 antagonist.

The following are examples of the practice of the invention. They are not to be construed as limiting the scope of the invention in any way.

### EXAMPLES

### Example 1

Based on basic science studies, the role of CRH was recognized as causal for signs and symptoms prevalent in depression, rendering blocking of CRH/CRHR1 signalling as a viable treatment option. Further clinical findings have found that CRH is elevated in a subgroup of patients with depression, where CRH causes core symptoms. Compound SSR-125543 has been developed elsewhere as a specific CRHR1 antagonist blocking the effect of CRH. A clinical trial evaluating the efficacy and tolerability of SSR-125543 in comparison to placebo and a standard antidepressant has been carried out previously without having predicted the treatment response according to the invention. However, based on additional studies (not published), it was recognized that among patients diagnosed with major depression, only a fraction of 20-30% has central CRH over-activity. Thus, a substantial fraction of non-stratified patients might not show a treatment response, in view of about 70-80% of patients treated with the CRHR1 antagonist not having a central CRH increase. Given the pharmacological specificity, only patients with central CRH-over-activity are likely to benefit from CRHR1 antagonists, such as SSR-125543.

Here, a method of predicting a clinical treatment response (e.g., as measured by the HAM-D score) has been devised, which detects one or more polymorphism genotypes selected from the polymorphism genotypes disclosed in Table 2, using a chip containing probes specific for these polymorphism genotypes, allowing for identification of depressive patients being likely to respond to a treatment with a CRHR1 antagonist such as SSR-125543. DNA samples obtained from 300 subjects enrolled in the earlier clinical trial, as mentioned above, were extensively analyzed by polymorphism genotyping. Using a machine-learning algorithm as described herein, polymorphism genotypes predictive of a response to SSR-125543 were identified, as disclosed in Table 2. Further, 150 or more polymorphism genotypes of this set were used to further “train” the algorithm, assisted by common machine-learning algorithms as described herein, and to test the prediction. Thus, having the set of useful polymorphism genotypes as disclosed in Table 2, at hand, a prediction algorithm can be readily devised, which provides superior prediction of a clinical response with high sensitivity and specificity. As is shown in Table 3, test predictions of a clinical response with a sensitivity of about 78% and a specificity of about 73% have been achieved.

TABLE 3

| | | Observed phenotype | |
|---|---|---|---|
| | | Good | Poor |
| | | response | response |
| Test prediction | Good | 21 | 13 |
| | response | | |
| | Poor | 6 | 36 |
| | response | | |
| | | Sensitivity 78% | Specificity 73% |

To exclude the possibility that the polymorphism genotypes disclosed herein are merely identifying patients that are good responders to any kind of drug intervention, the performance of the method among patients treated with the standard antidepressant escitalopram used as comparator in the earlier clinical trial has also been tested. The sensitivity was 50%, and specificity was 43%, and thus insensitive and unspecific regarding prediction of response to a standard antidepressant, see Table 4. Therefore, the present method is to be considered highly specific for predicting the response to CRHR1 antagonists.

TABLE 4

| | | Observed phenotype | |
|---|---|---|---|
| | | Good | Poor |
| | | response | response |
| Test prediction | Good | 23 | 17 |
| | response | | |
| | Poor | 23 | 13 |
| | response | | |
| | | Sensitivity 50% | Specificity 43% |

The above results were further challenged by considering a “lucky split” between the training and the testing cohort. Another 10.000 random splits were calculated which corroborated the initial result, achieving an odds ratio of 5, which indicates that chances of non-response are 5 times higher if the CRH genotyping analysis described herein predicts poor response. Transforming these findings into a time course curve where those depressed patients that where tested positive in the CRH genotyping analysis and treated with SSR-125543 were compared with patients treated by placebo resulted in a clear superiority of the investigational drug, see FIG. 1. The time course curves revealed a marked difference between placebo and SSR-125543 beginning after 2 weeks of treatment, as measured using, e.g., the HAM-D scale. The difference in response between patients treated with SSR-125543 and those under placebo is significant (p<0.01). In essence, subjects which are tested positive using the method of prediction described herein, based on a CRH genotyping analysis using 150 of the polymorphism genotypes disclosed in Table 2, constitute 28% of the overall patient sample and 78% patients from this sample were responders when treated with SSR-125543.

### Example 2

To further evaluate the usefulness of the set of polymorphism genotypes provided in Table 2, further predictions have been tested using minimal subsets selected as prediction variables. As few as singular polymorphism genotypes selected from Table 2, as well as subsets of two, four or eight polymorphism genotypes selected from Table 2 proved useful in the method of predicting a clinical response, e.g., as measured by the HAM-D scale.

Treatment response to an anti-depressant therapy comprising SSR-125543 was predicted based on the same patient data of the earlier clinical trial and polymorphism genotyping set as described above, using statistical tools selected from the group consisting of random forests, support vector machines, neural networks, linear discriminant analyses, clustering methods such as k-nearest neighbours and their respective derivatives, linear models and their derivatives, as well as their combinations.

Surprisingly, even this univariate, bivariate, quadrivariate or octovariate analyses using combinations of polymorphism genotypes as disclosed in Tables 2, 5-7 herein, yielded clinical response predictions of a quality significantly better (i.e. both sensitivity and specificity >50%) than randomness, based on assessing the P-value of concordance between observed and predicted outcome in a 10-fold cross-validation procedure.

In particular, a total number of 78 singular polymorphism genotypes was identified with nominally significant P-values. Of those, 46 yielded a specificity and sensitivity of >50% each in predicting a clinical response. One singular polymorphism yielded both a sensitivity and specificity of higher than 60% each in predicting a clinical response.

Of all tested combinations of two of the univariate significantly predicting polymorphisms, 237 exhibited both a sensitivity and specificity of at least 60% each in predicting a clinical response. Finally, a number of 46 tested combinations of two of the univariate significantly predicting polymorphism genotypes yielded a sensitivity and specificity beyond 65% each in predicting a clinical response, see Table 5.

TABLE 5
Bivariate sets of polymorphism genotypes

| P_ID1 | P_ID2 | rs_p1 | p2 | p-value | sensitivity | specificity |
|---|---|---|---|---|---|---|
| 11 | 181 | rs74888440 | rs9813396 | 0.00027897 | 0.62857143 | 0.6626506 |
| 11 | 192 | rs74888440 | rs72693005 | 0.00060709 | 0.67142857 | 0.60240964 |
| 11 | 207 | rs74888440 | rs220806 | 0.00010088 | 0.68571429 | 0.62650602 |
| 11 | 218 | rs74888440 | rs1944887 | 0.00015583 | 0.62857143 | 0.6746988 |
| 11 | 226 | rs74888440 | rs532996 | 0.00082753 | 0.62857143 | 0.63855422 |
| 11 | 227 | rs74888440 | rs9571939 | 0.00082753 | 0.62857143 | 0.63855422 |
| 11 | 228 | rs74888440 | rs2173530 | 0.00082753 | 0.62857143 | 0.63855422 |
| 11 | 244 | rs74888440 | rs2044070 | 0.00352822 | 0.62857143 | 0.60240964 |
| 11 | 245 | rs74888440 | rs920640 | 0.00352822 | 0.62857143 | 0.60240964 |
| 112 | 175 | rs2260882 | rs7648662 | 2.12E−05 | 0.64285714 | 0.69879518 |
| 112 | 237 | rs2260882 | rs12917505 | 0.00090174 | 0.61428571 | 0.65060241 |
| 112 | 238 | rs2260882 | rs16977818 | 2.19E−05 | 0.71428571 | 0.62650602 |
| 112 | 240 | rs2260882 | rs10851628 | 0.00039921 | 0.65714286 | 0.62650602 |
| 112 | 243 | rs2260882 | rs6493965 | 0.00137357 | 0.62857143 | 0.62650602 |
| 112 | 245 | rs2260882 | rs920640 | 0.0006793 | 0.65714286 | 0.61445783 |
| 112 | 246 | rs2260882 | rs920638 | 0.00202984 | 0.64285714 | 0.60240964 |
| 112 | 250 | rs2260882 | rs735164 | 0.00383837 | 0.61428571 | 0.61445783 |
| 112 | 277 | rs2260882 | rs2044230 | 0.00048656 | 0.62857143 | 0.65060241 |
| 116 | 179 | rs2257474 | rs6549407 | 0.00352822 | 0.62857143 | 0.60240964 |
| 116 | 182 | rs2257474 | rs12489026 | 0.00030332 | 0.61428571 | 0.6746988 |
| 116 | 191 | rs2257474 | rs1383699 | 7.55E−05 | 0.71428571 | 0.60240964 |
| 116 | 234 | rs2257474 | rs8042817 | 0.00011443 | 0.67142857 | 0.63855422 |
| 116 | 235 | rs2257474 | rs28811003 | 0.00039921 | 0.65714286 | 0.62650602 |
| 121 | 127 | rs2028629 | rs79320848 | 0.00383837 | 0.61428571 | 0.61445783 |
| 121 | 184 | rs2028629 | rs11715827 | 0.00015583 | 0.62857143 | 0.6746988 |
| 121 | 185 | rs2028629 | rs58882373 | 0.00015583 | 0.62857143 | 0.6746988 |
| 121 | 191 | rs2028629 | rs1383699 | 0.00082753 | 0.62857143 | 0.63855422 |
| 121 | 202 | rs2028629 | rs4836256 | 2.30E−05 | 0.62857143 | 0.71084337 |
| 121 | 233 | rs2028629 | rs929610 | 4.11E−05 | 0.64285714 | 0.68674699 |
| 121 | 237 | rs2028629 | rs12917505 | 7.00E−05 | 0.65714286 | 0.6626506 |
| 121 | 238 | rs2028629 | rs16977818 | 7.75E−05 | 0.64285714 | 0.6746988 |
| 121 | 239 | rs2028629 | rs11071351 | 0.00112948 | 0.65714286 | 0.60240964 |
| 121 | 240 | rs2028629 | rs10851628 | 3.32E−06 | 0.7 | 0.6746988 |
| 121 | 241 | rs2028629 | rs930473 | 7.72E−06 | 0.68571429 | 0.6746988 |
| 121 | 242 | rs2028629 | rs1441824 | 0.00011443 | 0.67142857 | 0.63855422 |
| 121 | 243 | rs2028629 | rs6493965 | 1.53E−05 | 0.68571429 | 0.6626506 |
| 121 | 244 | rs2028629 | rs2044070 | 3.32E−06 | 0.7 | 0.6746988 |
| 121 | 245 | rs2028629 | rs920640 | 3.72E−05 | 0.65714286 | 0.6746988 |
| 121 | 246 | rs2028629 | rs920638 | 3.32E−06 | 0.7 | 0.6746988 |
| 123 | 218 | rs4812040 | rs1944887 | 0.00125068 | 0.64285714 | 0.61445783 |
| 123 | 235 | rs4812040 | rs28811003 | 0.00052981 | 0.61428571 | 0.6626506 |
| 127 | 192 | rs79320848 | rs72693005 | 0.00035634 | 0.67142857 | 0.61445783 |
| 127 | 207 | rs79320848 | rs220806 | 0.00025408 | 0.64285714 | 0.65060241 |
| 127 | 218 | rs79320848 | rs1944887 | 0.00030332 | 0.61428571 | 0.6746988 |
| 132 | 184 | rs6026567 | rs11715827 | 2.69E−06 | 0.61428571 | 0.75903614 |
| 132 | 185 | rs6026567 | rs58882373 | 1.22E−05 | 0.61428571 | 0.73493976 |
| 132 | 213 | rs6026567 | rs2935752 | 0.00030332 | 0.61428571 | 0.6746988 |
| 132 | 214 | rs6026567 | rs2935751 | 0.00030332 | 0.61428571 | 0.6746988 |
| 132 | 237 | rs6026567 | rs12917505 | 4.82E−05 | 0.61428571 | 0.71084337 |
| 132 | 238 | rs6026567 | rs16977818 | 9.16E−05 | 0.61428571 | 0.69879518 |
| 132 | 239 | rs6026567 | rs11071351 | 0.00242522 | 0.61428571 | 0.62650602 |
| 132 | 240 | rs6026567 | rs10851628 | 7.00E−05 | 0.65714286 | 0.6626506 |
| 132 | 241 | rs6026567 | rs930473 | 0.00030332 | 0.61428571 | 0.6746988 |
| 132 | 244 | rs6026567 | rs2044070 | 0.00027897 | 0.62857143 | 0.6626506 |
| 133 | 190 | rs968519 | rs1383707 | 0.00016904 | 0.61428571 | 0.68674699 |
| 133 | 238 | rs968519 | rs16977818 | 0.00052981 | 0.61428571 | 0.6626506 |
| 133 | 240 | rs968519 | rs10851628 | 9.16E−05 | 0.61428571 | 0.69879518 |
| 133 | 241 | rs968519 | rs930473 | 9.16E−05 | 0.61428571 | 0.69879518 |
| 133 | 243 | rs968519 | rs6493965 | 9.16E−05 | 0.61428571 | 0.69879518 |
| 133 | 245 | rs968519 | rs920640 | 0.00052981 | 0.61428571 | 0.6626506 |
| 141 | 157 | rs6092704 | rs2242071 | 0.0006793 | 0.65714286 | 0.61445783 |
| 141 | 181 | rs6092704 | rs9813396 | 4.11E−05 | 0.71428571 | 0.61445783 |
| 141 | 187 | rs6092704 | rs10034039 | 0.00012826 | 0.65714286 | 0.65060241 |
| 141 | 190 | rs6092704 | rs1383707 | 0.00012826 | 0.65714286 | 0.65060241 |
| 141 | 191 | rs6092704 | rs1383699 | 0.00202984 | 0.64285714 | 0.60240964 |
| 141 | 212 | rs6092704 | rs3133622 | 0.00383837 | 0.61428571 | 0.61445783 |
| 141 | 259 | rs6092704 | rs487011 | 0.00149683 | 0.61428571 | 0.63855422 |
| 155 | 207 | rs7523266 | rs220806 | 0.00090174 | 0.61428571 | 0.65060241 |
| 156 | 207 | rs6686061 | rs220806 | 0.00090174 | 0.61428571 | 0.65060241 |
| 157 | 215 | rs2242071 | rs4570614 | 0.00352822 | 0.62857143 | 0.60240964 |
| 168 | 192 | rs809482 | rs72693005 | 0.00352822 | 0.62857143 | 0.60240964 |
| 176 | 234 | rs616870 | rs8042817 | 0.00593832 | 0.61428571 | 0.60240964 |
| 179 | 223 | rs6549407 | rs876270 | 0.00039921 | 0.65714286 | 0.62650602 |
| 179 | 224 | rs6549407 | rs11834041 | 0.00020436 | 0.67142857 | 0.62650602 |
| 179 | 248 | rs6549407 | rs7165629 | 0.00015717 | 0.7 | 0.60240964 |
| 180 | 187 | rs6766242 | rs10034039 | 4.11E−05 | 0.71428571 | 0.61445783 |
| 180 | 220 | rs6766242 | rs7121326 | 0.00082753 | 0.62857143 | 0.63855422 |
| 180 | 223 | rs6766242 | rs876270 | 7.75E−05 | 0.64285714 | 0.6746988 |
| 180 | 224 | rs6766242 | rs11834041 | 3.72E−05 | 0.65714286 | 0.6746988 |
| 180 | 227 | rs6766242 | rs9571939 | 0.00593832 | 0.61428571 | 0.60240964 |
| 180 | 234 | rs6766242 | rs8042817 | 0.00030332 | 0.61428571 | 0.6746988 |
| 180 | 235 | rs6766242 | rs28811003 | 0.00090174 | 0.61428571 | 0.65060241 |
| 182 | 187 | rs12489026 | rs10034039 | 2.94E−05 | 0.68571429 | 0.65060241 |
| 182 | 188 | rs12489026 | rs17616338 | 0.00052981 | 0.61428571 | 0.6626506 |
| 182 | 218 | rs12489026 | rs1944887 | 0.00383837 | 0.61428571 | 0.61445783 |
| 182 | 224 | rs12489026 | rs11834041 | 4.82E−05 | 0.61428571 | 0.71084337 |
| 184 | 218 | rs11715827 | rs1944887 | 0.00082753 | 0.62857143 | 0.63855422 |
| 184 | 219 | rs11715827 | rs10894873 | 0.00383837 | 0.61428571 | 0.61445783 |
| 184 | 236 | rs11715827 | rs894342 | 4.48E−05 | 0.62857143 | 0.69879518 |
| 184 | 237 | rs11715827 | rs12917505 | 2.30E−05 | 0.62857143 | 0.71084337 |
| 184 | 238 | rs11715827 | rs16977818 | 2.30E−05 | 0.62857143 | 0.71084337 |
| 184 | 239 | rs11715827 | rs11071351 | 8.72E−06 | 0.67142857 | 0.68674699 |
| 184 | 240 | rs11715827 | rs10851628 | 1.14E−05 | 0.62857143 | 0.72289157 |
| 184 | 241 | rs11715827 | rs930473 | 4.82E−05 | 0.61428571 | 0.71084337 |
| 184 | 242 | rs11715827 | rs1441824 | 2.30E−05 | 0.62857143 | 0.71084337 |
| 184 | 243 | rs11715827 | rs6493965 | 9.16E−05 | 0.61428571 | 0.69879518 |
| 184 | 244 | rs11715827 | rs2044070 | 4.48E−05 | 0.62857143 | 0.69879518 |
| 184 | 245 | rs11715827 | rs920640 | 1.06E−05 | 0.64285714 | 0.71084337 |
| 184 | 246 | rs11715827 | rs920638 | 2.12E−05 | 0.64285714 | 0.69879518 |
| 185 | 219 | rs58882373 | rs10894873 | 0.00137357 | 0.62857143 | 0.62650602 |
| 185 | 234 | rs58882373 | rs8042817 | 0.00149683 | 0.61428571 | 0.63855422 |
| 185 | 236 | rs58882373 | rs894342 | 0.00015583 | 0.62857143 | 0.6746988 |
| 185 | 237 | rs58882373 | rs12917505 | 1.14E−05 | 0.62857143 | 0.72289157 |
| 185 | 238 | rs58882373 | rs16977818 | 2.30E−05 | 0.62857143 | 0.71084337 |
| 185 | 239 | rs58882373 | rs11071351 | 8.72E−06 | 0.67142857 | 0.68674699 |
| 185 | 240 | rs58882373 | rs10851628 | 1.14E−05 | 0.62857143 | 0.72289157 |
| 185 | 241 | rs58882373 | rs930473 | 4.82E−05 | 0.61428571 | 0.71084337 |
| 185 | 242 | rs58882373 | rs1441824 | 2.30E−05 | 0.62857143 | 0.71084337 |
| 185 | 243 | rs58882373 | rs6493965 | 4.48E−05 | 0.62857143 | 0.69879518 |
| 185 | 244 | rs58882373 | rs2044070 | 4.48E−05 | 0.62857143 | 0.69879518 |
| 185 | 245 | rs58882373 | rs920640 | 4.48E−05 | 0.62857143 | 0.69879518 |
| 185 | 246 | rs58882373 | rs920638 | 4.48E−05 | 0.62857143 | 0.69879518 |
| 186 | 236 | rs12490095 | rs894342 | 2.57E−06 | 0.62857143 | 0.74698795 |
| 187 | 188 | rs10034039 | rs17616338 | 8.78E−05 | 0.7 | 0.61445783 |
| 187 | 193 | rs10034039 | rs1170303 | 0.00090174 | 0.61428571 | 0.65060241 |
| 187 | 198 | rs10034039 | rs66624622 | 0.00015583 | 0.62857143 | 0.6746988 |
| 187 | 215 | rs10034039 | rs4570614 | 0.00052981 | 0.61428571 | 0.6626506 |
| 187 | 216 | rs10034039 | rs4758040 | 0.00014215 | 0.64285714 | 0.6626506 |
| 187 | 239 | rs10034039 | rs11071351 | 0.00030332 | 0.61428571 | 0.6746988 |
| 188 | 191 | rs17616338 | rs1383699 | 0.00018028 | 0.68571429 | 0.61445783 |
| 189 | 218 | rs80049044 | rs1944887 | 0.00018028 | 0.68571429 | 0.61445783 |
| 190 | 193 | rs1383707 | rs1170303 | 0.00039921 | 0.65714286 | 0.62650602 |
| 190 | 212 | rs1383707 | rs3133622 | 1.61E−06 | 0.75714286 | 0.62650602 |
| 190 | 216 | rs1383707 | rs4758040 | 0.00039921 | 0.65714286 | 0.62650602 |
| 190 | 234 | rs1383707 | rs8042817 | 1.53E−05 | 0.74285714 | 0.60240964 |
| 190 | 237 | rs1383707 | rs12917505 | 0.00027897 | 0.62857143 | 0.6626506 |
| 190 | 242 | rs1383707 | rs1441824 | 0.00149683 | 0.61428571 | 0.63855422 |
| 190 | 252 | rs1383707 | rs4610906 | 0.00090174 | 0.61428571 | 0.65060241 |
| 191 | 216 | rs1383699 | rs4758040 | 0.00137357 | 0.62857143 | 0.62650602 |
| 191 | 234 | rs1383699 | rs8042817 | 0.00015717 | 0.7 | 0.60240964 |
| 191 | 235 | rs1383699 | rs28811003 | 0.00031476 | 0.68571429 | 0.60240964 |
| 191 | 237 | rs1383699 | rs12917505 | 2.19E−05 | 0.71428571 | 0.62650602 |
| 191 | 238 | rs1383699 | rs16977818 | 4.03E−06 | 0.74285714 | 0.62650602 |
| 191 | 240 | rs1383699 | rs10851628 | 2.19E−05 | 0.71428571 | 0.62650602 |
| 191 | 241 | rs1383699 | rs930473 | 0.00010088 | 0.68571429 | 0.62650602 |
| 191 | 242 | rs1383699 | rs1441824 | 0.00112948 | 0.65714286 | 0.60240964 |
| 191 | 243 | rs1383699 | rs6493965 | 1.14E−05 | 0.71428571 | 0.63855422 |
| 191 | 244 | rs1383699 | rs2044070 | 0.0006793 | 0.65714286 | 0.61445783 |
| 191 | 245 | rs1383699 | rs920640 | 1.14E−05 | 0.71428571 | 0.63855422 |
| 191 | 246 | rs1383699 | rs920638 | 3.27E−06 | 0.75714286 | 0.61445783 |
| 191 | 259 | rs1383699 | rs487011 | 4.11E−05 | 0.71428571 | 0.61445783 |
| 192 | 252 | rs72693005 | rs4610906 | 0.00202984 | 0.64285714 | 0.60240964 |
| 192 | 259 | rs72693005 | rs487011 | 1.53E−05 | 0.74285714 | 0.60240964 |
| 193 | 218 | rs1170303 | rs1944887 | 0.00112948 | 0.65714286 | 0.60240964 |
| 193 | 259 | rs1170303 | rs487011 | 0.00137357 | 0.62857143 | 0.62650602 |
| 198 | 226 | rs66624622 | rs532996 | 0.00039921 | 0.65714286 | 0.62650602 |
| 198 | 227 | rs66624622 | rs9571939 | 0.00039921 | 0.65714286 | 0.62650602 |
| 198 | 228 | rs66624622 | rs2173530 | 0.00137357 | 0.62857143 | 0.62650602 |
| 199 | 259 | rs72784444 | rs487011 | 0.00149683 | 0.61428571 | 0.63855422 |
| 201 | 237 | rs62377761 | rs12917505 | 0.00060709 | 0.67142857 | 0.60240964 |
| 201 | 238 | rs62377761 | rs16977818 | 0.00137357 | 0.62857143 | 0.62650602 |
| 201 | 244 | rs62377761 | rs2044070 | 0.00052981 | 0.61428571 | 0.6626506 |
| 202 | 206 | rs4836256 | rs730976 | 0.00593832 | 0.61428571 | 0.60240964 |
| 202 | 218 | rs4836256 | rs1944887 | 0.00016904 | 0.61428571 | 0.68674699 |
| 202 | 225 | rs4836256 | rs67959715 | 0.00044281 | 0.64285714 | 0.63855422 |
| 202 | 236 | rs4836256 | rs894342 | 7.00E−05 | 0.65714286 | 0.6626506 |
| 202 | 237 | rs4836256 | rs12917505 | 1.82E−06 | 0.68571429 | 0.69879518 |
| 202 | 238 | rs4836256 | rs16977818 | 2.12E−05 | 0.64285714 | 0.69879518 |
| 202 | 239 | rs4836256 | rs11071351 | 0.00044281 | 0.64285714 | 0.63855422 |
| 202 | 240 | rs4836256 | rs10851628 | 4.11E−05 | 0.64285714 | 0.68674699 |
| 202 | 241 | rs4836256 | rs930473 | 4.27E−06 | 0.67142857 | 0.69879518 |
| 202 | 242 | rs4836256 | rs1441824 | 0.00012826 | 0.65714286 | 0.65060241 |
| 202 | 243 | rs4836256 | rs6493965 | 4.27E−06 | 0.67142857 | 0.69879518 |
| 202 | 244 | rs4836256 | rs2044070 | 1.73E−05 | 0.67142857 | 0.6746988 |
| 202 | 245 | rs4836256 | rs920640 | 8.72E−06 | 0.67142857 | 0.68674699 |
| 202 | 246 | rs4836256 | rs920638 | 8.72E−06 | 0.67142857 | 0.68674699 |
| 206 | 218 | rs730976 | rs1944887 | 0.00242522 | 0.61428571 | 0.62650602 |
| 211 | 235 | rs3735833 | rs28811003 | 8.47E−05 | 0.62857143 | 0.68674699 |
| 213 | 233 | rs2935752 | rs929610 | 0.00149683 | 0.61428571 | 0.63855422 |
| 213 | 236 | rs2935752 | rs894342 | 0.00011443 | 0.67142857 | 0.63855422 |
| 213 | 237 | rs2935752 | rs12917505 | 9.69E−06 | 0.65714286 | 0.69879518 |
| 213 | 238 | rs2935752 | rs16977818 | 4.73E−06 | 0.65714286 | 0.71084337 |
| 213 | 239 | rs2935752 | rs11071351 | 0.00014215 | 0.64285714 | 0.6626506 |
| 213 | 240 | rs2935752 | rs10851628 | 4.73E−06 | 0.65714286 | 0.71084337 |
| 213 | 241 | rs2935752 | rs930473 | 1.06E−05 | 0.64285714 | 0.71084337 |
| 213 | 242 | rs2935752 | rs1441824 | 6.25E−05 | 0.67142857 | 0.65060241 |
| 213 | 243 | rs2935752 | rs6493965 | 1.06E−05 | 0.64285714 | 0.71084337 |
| 213 | 244 | rs2935752 | rs2044070 | 9.69E−06 | 0.65714286 | 0.69879518 |
| 213 | 245 | rs2935752 | rs920640 | 9.69E−06 | 0.65714286 | 0.69879518 |
| 213 | 246 | rs2935752 | rs920638 | 4.48E−05 | 0.62857143 | 0.69879518 |
| 214 | 236 | rs2935751 | rs894342 | 0.00044281 | 0.64285714 | 0.63855422 |
| 214 | 237 | rs2935751 | rs12917505 | 9.69E−06 | 0.65714286 | 0.69879518 |
| 214 | 238 | rs2935751 | rs16977818 | 4.73E−06 | 0.65714286 | 0.71084337 |
| 214 | 239 | rs2935751 | rs11071351 | 0.00014215 | 0.64285714 | 0.6626506 |
| 214 | 240 | rs2935751 | rs10851628 | 2.30E−05 | 0.62857143 | 0.71084337 |
| 214 | 241 | rs2935751 | rs930473 | 1.06E−05 | 0.64285714 | 0.71084337 |
| 214 | 242 | rs2935751 | rs1441824 | 6.25E−05 | 0.67142857 | 0.65060241 |
| 214 | 243 | rs2935751 | rs6493965 | 4.82E−05 | 0.61428571 | 0.71084337 |
| 214 | 244 | rs2935751 | rs2044070 | 4.48E−05 | 0.62857143 | 0.69879518 |
| 214 | 245 | rs2935751 | rs920640 | 9.69E−06 | 0.65714286 | 0.69879518 |
| 214 | 246 | rs2935751 | rs920638 | 9.69E−06 | 0.65714286 | 0.69879518 |
| 215 | 218 | rs4570614 | rs1944887 | 0.00011443 | 0.67142857 | 0.63855422 |
| 215 | 237 | rs4570614 | rs12917505 | 0.00137357 | 0.62857143 | 0.62650602 |
| 215 | 240 | rs4570614 | rs10851628 | 0.00593832 | 0.61428571 | 0.60240964 |
| 215 | 246 | rs4570614 | rs920638 | 0.00149683 | 0.61428571 | 0.63855422 |
| 216 | 237 | rs4758040 | rs12917505 | 0.00202984 | 0.64285714 | 0.60240964 |
| 216 | 240 | rs4758040 | rs10851628 | 0.00112948 | 0.65714286 | 0.60240964 |
| 216 | 244 | rs4758040 | rs2044070 | 0.00352822 | 0.62857143 | 0.60240964 |
| 216 | 245 | rs4758040 | rs920640 | 0.00090174 | 0.61428571 | 0.65060241 |
| 216 | 246 | rs4758040 | rs920638 | 0.00052981 | 0.61428571 | 0.6626506 |
| 218 | 234 | rs1944887 | rs8042817 | 3.33E−05 | 0.67142857 | 0.6626506 |
| 218 | 259 | rs1944887 | rs487011 | 0.00593832 | 0.61428571 | 0.60240964 |
| 223 | 234 | rs876270 | rs8042817 | 0.00022908 | 0.65714286 | 0.63855422 |
| 223 | 235 | rs876270 | rs28811003 | 0.00039921 | 0.65714286 | 0.62650602 |
| 223 | 259 | rs876270 | rs487011 | 0.00075306 | 0.64285714 | 0.62650602 |
| 224 | 234 | rs11834041 | rs8042817 | 0.00011443 | 0.67142857 | 0.63855422 |
| 224 | 235 | rs11834041 | rs28811003 | 0.00020436 | 0.67142857 | 0.62650602 |
| 224 | 248 | rs11834041 | rs7165629 | 0.00039921 | 0.65714286 | 0.62650602 |
| 225 | 246 | rs67959715 | rs920638 | 5.82E−06 | 0.61428571 | 0.74698795 |
| 233 | 236 | rs929610 | rs894342 | 0.0006793 | 0.65714286 | 0.61445783 |
| 233 | 237 | rs929610 | rs12917505 | 7.72E−06 | 0.68571429 | 0.6746988 |
| 233 | 239 | rs929610 | rs11071351 | 0.00039921 | 0.65714286 | 0.62650602 |
| 233 | 240 | rs929610 | rs10851628 | 4.11E−05 | 0.64285714 | 0.68674699 |
| 233 | 243 | rs929610 | rs6493965 | 4.11E−05 | 0.64285714 | 0.68674699 |
| 233 | 244 | rs929610 | rs2044070 | 7.75E−05 | 0.64285714 | 0.6746988 |
| 233 | 245 | rs929610 | rs920640 | 1.73E−05 | 0.67142857 | 0.6746988 |
| 233 | 246 | rs929610 | rs920638 | 1.73E−05 | 0.67142857 | 0.6746988 |
| 234 | 237 | rs8042817 | rs12917505 | 0.00075306 | 0.64285714 | 0.62650602 |
| 234 | 240 | rs8042817 | rs10851628 | 0.00149683 | 0.61428571 | 0.63855422 |
| 237 | 239 | rs12917505 | rs11071351 | 6.46E−06 | 0.75714286 | 0.60240964 |
| 237 | 259 | rs12917505 | rs487011 | 6.73E−06 | 0.7 | 0.6626506 |
| 238 | 239 | rs16977818 | rs11071351 | 3.27E−06 | 0.75714286 | 0.61445783 |
| 238 | 259 | rs16977818 | rs487011 | 5.45E−07 | 0.72857143 | 0.6746988 |
| 239 | 240 | rs11071351 | rs10851628 | 3.27E−06 | 0.75714286 | 0.61445783 |
| 239 | 241 | rs11071351 | rs930473 | 7.95E−06 | 0.74285714 | 0.61445783 |
| 239 | 243 | rs11071351 | rs6493965 | 7.95E−06 | 0.74285714 | 0.61445783 |
| 239 | 244 | rs11071351 | rs2044070 | 3.27E−06 | 0.75714286 | 0.61445783 |
| 239 | 245 | rs11071351 | rs920640 | 3.27E−06 | 0.75714286 | 0.61445783 |
| 239 | 246 | rs11071351 | rs920638 | 3.27E−06 | 0.75714286 | 0.61445783 |
| 240 | 259 | rs10851628 | rs487011 | 5.45E−07 | 0.72857143 | 0.6746988 |
| 241 | 259 | rs930473 | rs487011 | 1.37E−06 | 0.71428571 | 0.6746988 |
| 242 | 259 | rs1441824 | rs487011 | 0.00018028 | 0.68571429 | 0.61445783 |
| 243 | 248 | rs6493965 | rs7165629 | 0.00352822 | 0.62857143 | 0.60240964 |
| 243 | 259 | rs6493965 | rs487011 | 1.73E−05 | 0.67142857 | 0.6746988 |
| 244 | 259 | rs2044070 | rs487011 | 6.73E−06 | 0.7 | 0.6626506 |
| 245 | 259 | rs920640 | rs487011 | 1.16E−06 | 0.72857143 | 0.6626506 |
| 246 | 259 | rs920638 | rs487011 | 1.16E−06 | 0.72857143 | 0.6626506 |

In higher order analyses, using sets of four and eight polymorphism genotypes selected from the group disclosed in Table 2, a complete enumeration becomes unpractical (over a million combinations for the sets of four and over 10¹⁰ for the set of eight polymorphism genotypes). Therefore, randomly sampled sets (1000 combinations each) of such cardinalities k are presented herein.

For k=4, 72.1% of tested polymorphism genotype combinations yield a sensitivity and specificity of higher than 50% each, 20,5% of polymorphism genotype combinations yield a sensitivity and specificity of higher than 60% each, and 5,8% of polymorphism genotype combinations yield a sensitivity and specificity of higher than 65% each in predicting a clinical response. Two quadriavariate combinations even yield at least 70% in both sensitivity and specificity in predicting a clinical response, see Table 6.

TABLE 6
Quadrivariate sets of polymorphism genotypes

| P_ID1 | P_ID2 | P_ID3 | P_ID4 | p-value | sensitivity | specificity |
|---|---|---|---|---|---|---|
| 233 | 123 | 121 | 127 | 8.72E−06 | 0.67142857 | 0.68674699 |
| 236 | 186 | 223 | 215 | 1.82E−06 | 0.68571429 | 0.69879518 |
| 202 | 215 | 184 | 233 | 9.40E−07 | 0.67142857 | 0.72289157 |
| 207 | 171 | 185 | 121 | 1.94E−07 | 0.65714286 | 0.75903614 |
| 240 | 207 | 141 | 157 | 8.01E−08 | 0.65714286 | 0.77108434 |
| 158 | 214 | 133 | 246 | 1.53E−05 | 0.68571429 | 0.6626506 |
| 241 | 219 | 188 | 127 | 8.72E−06 | 0.67142857 | 0.68674699 |
| 233 | 157 | 185 | 158 | 4.58E−08 | 0.78571429 | 0.65060241 |
| 188 | 225 | 223 | 237 | 7.45E−07 | 0.7 | 0.69879518 |
| 225 | 247 | 202 | 179 | 3.72E−05 | 0.65714286 | 0.6746988 |
| 157 | 213 | 219 | 218 | 7.00E−05 | 0.65714286 | 0.6626506 |
| 188 | 242 | 112 | 192 | 6.25E−05 | 0.67142857 | 0.65060241 |
| 237 | 226 | 158 | 216 | 6.25E−05 | 0.67142857 | 0.65060241 |
| 205 | 226 | 156 | 181 | 2.04E−06 | 0.67142857 | 0.71084337 |
| 191 | 239 | 226 | 234 | 0.00012826 | 0.65714286 | 0.65060241 |
| 116 | 243 | 246 | 158 | 2.24E−06 | 0.65714286 | 0.72289157 |
| 193 | 233 | 240 | 198 | 9.69E−06 | 0.65714286 | 0.69879518 |
| 202 | 141 | 204 | 160 | 7.00E−05 | 0.65714286 | 0.6626506 |
| 184 | 233 | 192 | 215 | 3.72E−05 | 0.65714286 | 0.6746988 |
| 191 | 188 | 159 | 243 | 2.94E−05 | 0.68571429 | 0.65060241 |
| 246 | 227 | 238 | 224 | 1.94E−07 | 0.65714286 | 0.75903614 |
| 202 | 241 | 224 | 183 | 3.90E−09 | 0.7 | 0.77108434 |
| 227 | 191 | 112 | 246 | 7.00E−05 | 0.65714286 | 0.6626506 |
| 252 | 161 | 192 | 240 | 4.55E−07 | 0.65714286 | 0.74698795 |
| 161 | 207 | 202 | 160 | 1.68E−07 | 0.75714286 | 0.6626506 |
| 212 | 243 | 190 | 116 | 4.95E−10 | 0.65714286 | 0.8313253 |
| 246 | 184 | 11 | 243 | 3.33E−05 | 0.67142857 | 0.6626506 |
| 184 | 241 | 259 | 187 | 7.00E−05 | 0.65714286 | 0.6626506 |
| 226 | 243 | 190 | 224 | 4.73E−06 | 0.65714286 | 0.71084337 |
| 237 | 157 | 240 | 160 | 1.53E−05 | 0.68571429 | 0.6626506 |
| 223 | 245 | 132 | 184 | 1.03E−06 | 0.65714286 | 0.73493976 |
| 188 | 207 | 182 | 228 | 1.03E−06 | 0.65714286 | 0.73493976 |
| 224 | 205 | 227 | 186 | 7.00E−05 | 0.65714286 | 0.6626506 |
| 223 | 176 | 245 | 206 | 4.73E−06 | 0.65714286 | 0.71084337 |
| 190 | 204 | 234 | 238 | 6.29E−08 | 0.7 | 0.73493976 |
| 201 | 192 | 240 | 187 | 1.73E−05 | 0.67142857 | 0.6746988 |
| 227 | 185 | 190 | 215 | 7.45E−07 | 0.7 | 0.69879518 |
| 185 | 241 | 202 | 186 | 1.93E−05 | 0.65714286 | 0.68674699 |
| 214 | 11 | 157 | 220 | 9.61E−07 | 0.74285714 | 0.65060241 |
| 242 | 190 | 192 | 245 | 2.86E−06 | 0.71428571 | 0.6626506 |
| 121 | 246 | 238 | 190 | 9.69E−06 | 0.65714286 | 0.69879518 |
| 223 | 157 | 241 | 190 | 1.82E−06 | 0.68571429 | 0.69879518 |
| 233 | 116 | 132 | 243 | 3.72E−05 | 0.65714286 | 0.6746988 |
| 218 | 158 | 250 | 244 | 9.40E−07 | 0.67142857 | 0.72289157 |
| 250 | 158 | 141 | 213 | 3.33E−05 | 0.67142857 | 0.6626506 |
| 240 | 215 | 213 | 158 | 9.69E−06 | 0.65714286 | 0.69879518 |
| 235 | 243 | 214 | 208 | 4.73E−06 | 0.65714286 | 0.71084337 |
| 202 | 244 | 234 | 127 | 1.33E−05 | 0.7 | 0.65060241 |
| 175 | 184 | 127 | 219 | 4.27E−06 | 0.67142857 | 0.69879518 |
| 190 | 240 | 212 | 223 | 1.81E−07 | 0.67142857 | 0.74698795 |
| 248 | 219 | 233 | 185 | 6.44E−07 | 0.71428571 | 0.68674699 |
| 184 | 234 | 205 | 244 | 9.69E−06 | 0.65714286 | 0.69879518 |
| 201 | 246 | 192 | 233 | 4.27E−06 | 0.67142857 | 0.69879518 |
| 251 | 245 | 191 | 176 | 1.82E−06 | 0.68571429 | 0.69879518 |
| 233 | 223 | 235 | 225 | 3.72E−05 | 0.65714286 | 0.6746988 |
| 237 | 220 | 236 | 192 | 9.69E−06 | 0.65714286 | 0.69879518 |
| 241 | 236 | 248 | 218 | 4.73E−06 | 0.65714286 | 0.71084337 |
| 252 | 218 | 219 | 239 | 6.98E−08 | 0.68571429 | 0.74698795 |

For k=8, 93.3% of tested polymorphism genotype combinations yield a sensitivity and specificity of higher than 50% each, 32.6% of polymorphism genotype yield a sensitivity and specificity of higher than 60% each, 8.7% of polymorphism genotype combinations yield a sensitivity and specificity of 65% each, and, finally, 0.5% (5 combinations) of octovariate polymorphism genotype combinations yield a sensitivity and specificity at least 70% in sensitivity and specificity in predicting a clinical response, see Table 7.

TABLE 7
Octovariate sets of polymorphism genotypes

| P_ID1 | P_ID2 | P_ID3 | P_ID4 | P_ID5 | P_ID6 | P_ID7 | P_ID8 | p-value | sensitivity | specificity |
|---|---|---|---|---|---|---|---|---|---|---|
| 201 | 198 | 191 | 248 | 176 | 213 | 220 | 239 | 3.85E−06 | 0.65714286 | 0.72289157 |
| 206 | 112 | 186 | 247 | 205 | 171 | 184 | 246 | 2.41E−05 | 0.65714286 | 0.68674699 |
| 188 | 243 | 227 | 191 | 240 | 202 | 242 | 176 | 3.85E−06 | 0.65714286 | 0.72289157 |
| 160 | 193 | 132 | 235 | 121 | 192 | 188 | 236 | 7.10E−07 | 0.67142857 | 0.73493976 |
| 246 | 112 | 237 | 220 | 190 | 185 | 116 | 186 | 6.78E−07 | 0.65714286 | 0.74698795 |
| 116 | 189 | 241 | 246 | 213 | 225 | 191 | 132 | 1.57E−08 | 0.68571429 | 0.77108434 |
| 132 | 202 | 236 | 245 | 184 | 193 | 192 | 198 | 4.11E−08 | 0.67142857 | 0.77108434 |
| 244 | 188 | 225 | 206 | 192 | 214 | 234 | 213 | 2.99E−07 | 0.68571429 | 0.73493976 |
| 235 | 214 | 211 | 156 | 245 | 190 | 188 | 237 | 1.66E−08 | 0.7 | 0.75903614 |
| 185 | 238 | 244 | 206 | 237 | 184 | 183 | 259 | 1.64E−06 | 0.65714286 | 0.73493976 |
| 185 | 168 | 191 | 193 | 184 | 160 | 238 | 141 | 1.93E−05 | 0.65714286 | 0.69879518 |
| 159 | 244 | 202 | 133 | 259 | 243 | 223 | 121 | 4.03E−06 | 0.67142857 | 0.71084337 |
| 211 | 238 | 235 | 158 | 228 | 218 | 214 | 189 | 4.11E−08 | 0.67142857 | 0.77108434 |
| 190 | 238 | 185 | 259 | 213 | 179 | 184 | 188 | 2.71E−07 | 0.65714286 | 0.75903614 |
| 11 | 157 | 223 | 188 | 236 | 185 | 244 | 201 | 2.41E−05 | 0.65714286 | 0.68674699 |
| 188 | 246 | 171 | 242 | 127 | 184 | 234 | 132 | 6.78E−07 | 0.65714286 | 0.74698795 |
| 240 | 158 | 112 | 235 | 259 | 242 | 226 | 205 | 1.06E−05 | 0.68571429 | 0.6746988 |
| 211 | 213 | 205 | 171 | 202 | 185 | 259 | 116 | 1.50E−07 | 0.72857143 | 0.69879518 |
| 187 | 121 | 250 | 116 | 233 | 243 | 198 | 220 | 7.46E−07 | 0.68571429 | 0.72289157 |
| 216 | 168 | 185 | 132 | 183 | 112 | 213 | 238 | 1.49E−08 | 0.67142857 | 0.78313253 |
| 157 | 248 | 236 | 259 | 171 | 238 | 239 | 192 | 4.86E−05 | 0.65714286 | 0.6746988 |
| 234 | 227 | 224 | 251 | 277 | 198 | 187 | 245 | 1.05E−07 | 0.65714286 | 0.77108434 |
| 237 | 223 | 11 | 215 | 116 | 218 | 182 | 233 | 1.49E−08 | 0.67142857 | 0.78313253 |
| 201 | 220 | 127 | 234 | 157 | 219 | 186 | 141 | 6.78E−07 | 0.65714286 | 0.74698795 |
| 218 | 247 | 193 | 241 | 192 | 236 | 224 | 186 | 2.84E−07 | 0.67142857 | 0.74698795 |
| 233 | 201 | 158 | 226 | 235 | 132 | 223 | 190 | 3.85E−06 | 0.65714286 | 0.72289157 |
| 225 | 186 | 156 | 241 | 204 | 214 | 218 | 212 | 2.71E−07 | 0.65714286 | 0.75903614 |
| 116 | 179 | 112 | 184 | 190 | 259 | 239 | 215 | 1.64E−06 | 0.65714286 | 0.73493976 |
| 121 | 252 | 186 | 189 | 241 | 133 | 141 | 223 | 1.41E−08 | 0.65714286 | 0.79518072 |
| 250 | 248 | 241 | 184 | 159 | 206 | 187 | 192 | 7.10E−07 | 0.67142857 | 0.73493976 |
| 168 | 277 | 250 | 238 | 245 | 218 | 227 | 184 | 1.57E−08 | 0.68571429 | 0.77108434 |
| 212 | 181 | 184 | 159 | 237 | 223 | 179 | 213 | 2.84E−07 | 0.67142857 | 0.74698795 |
| 241 | 219 | 175 | 187 | 156 | 233 | 157 | 184 | 2.99E−07 | 0.68571429 | 0.73493976 |
| 224 | 192 | 206 | 121 | 202 | 214 | 241 | 239 | 5.48E−09 | 0.68571429 | 0.78313253 |
| 241 | 192 | 214 | 141 | 179 | 227 | 212 | 121 | 8.76E−06 | 0.65714286 | 0.71084337 |
| 212 | 241 | 239 | 121 | 191 | 187 | 224 | 238 | 5.48E−09 | 0.68571429 | 0.78313253 |
| 245 | 225 | 236 | 132 | 160 | 211 | 244 | 238 | 2.85E−09 | 0.65714286 | 0.81927711 |
| 121 | 237 | 234 | 205 | 132 | 244 | 190 | 238 | 1.22E−07 | 0.7 | 0.73493976 |
| 121 | 220 | 241 | 245 | 219 | 214 | 248 | 132 | 8.76E−06 | 0.65714286 | 0.71084337 |
| 240 | 220 | 252 | 250 | 157 | 214 | 218 | 245 | 5.48E−09 | 0.68571429 | 0.78313253 |
| 193 | 211 | 179 | 132 | 185 | 246 | 238 | 240 | 2.85E−09 | 0.65714286 | 0.81927711 |
| 243 | 241 | 252 | 237 | 192 | 141 | 259 | 190 | 7.10E−07 | 0.67142857 | 0.73493976 |
| 227 | 190 | 213 | 250 | 191 | 218 | 214 | 248 | 4.91E−09 | 0.65714286 | 0.80722892 |
| 242 | 214 | 239 | 179 | 201 | 190 | 181 | 192 | 1.78E−11 | 0.7 | 0.8313253 |
| 224 | 121 | 259 | 246 | 207 | 228 | 204 | 219 | 3.85E−06 | 0.65714286 | 0.72289157 |
| 236 | 186 | 116 | 187 | 184 | 204 | 219 | 121 | 2.84E−07 | 0.67142857 | 0.74698795 |
| 179 | 11 | 239 | 184 | 159 | 202 | 123 | 185 | 4.33E−08 | 0.68571429 | 0.75903614 |
| 248 | 127 | 240 | 141 | 133 | 233 | 156 | 201 | 1.05E−07 | 0.65714286 | 0.77108434 |
| 185 | 237 | 188 | 191 | 247 | 189 | 216 | 158 | 2.84E−07 | 0.67142857 | 0.74698795 |
| 219 | 132 | 176 | 191 | 277 | 214 | 236 | 175 | 1.49E−08 | 0.67142857 | 0.78313253 |
| 133 | 241 | 214 | 220 | 189 | 191 | 233 | 211 | 6.78E−07 | 0.65714286 | 0.74698795 |
| 202 | 182 | 233 | 259 | 218 | 127 | 243 | 159 | 2.71E−07 | 0.65714286 | 0.75903614 |
| 189 | 238 | 216 | 223 | 214 | 158 | 190 | 179 | 2.85E−09 | 0.65714286 | 0.81927711 |
| 123 | 112 | 243 | 141 | 202 | 121 | 190 | 116 | 1.76E−08 | 0.71428571 | 0.74698795 |
| 237 | 193 | 116 | 185 | 228 | 202 | 186 | 132 | 2.71E−07 | 0.65714286 | 0.75903614 |
| 190 | 11 | 237 | 182 | 202 | 132 | 214 | 246 | 1.10E−07 | 0.67142857 | 0.75903614 |
| 214 | 237 | 224 | 218 | 250 | 181 | 155 | 160 | 3.92E−08 | 0.65714286 | 0.78313253 |
| 237 | 252 | 234 | 133 | 185 | 250 | 239 | 188 | 5.48E−09 | 0.68571429 | 0.78313253 |
| 188 | 228 | 245 | 185 | 248 | 234 | 161 | 224 | 4.03E−06 | 0.67142857 | 0.71084337 |
| 204 | 228 | 188 | 202 | 212 | 223 | 168 | 141 | 2.71E−07 | 0.65714286 | 0.75903614 |
| 206 | 238 | 186 | 245 | 191 | 220 | 155 | 192 | 6.78E−07 | 0.65714286 | 0.74698795 |
| 237 | 246 | 168 | 188 | 141 | 198 | 192 | 190 | 3.85E−06 | 0.65714286 | 0.72289157 |
| 223 | 252 | 190 | 160 | 205 | 212 | 184 | 233 | 4.03E−06 | 0.67142857 | 0.71084337 |
| 141 | 187 | 121 | 188 | 246 | 193 | 185 | 133 | 1.16E−07 | 0.68571429 | 0.74698795 |
| 218 | 238 | 228 | 234 | 184 | 213 | 132 | 248 | 1.10E−07 | 0.67142857 | 0.75903614 |
| 11 | 213 | 238 | 219 | 246 | 112 | 187 | 248 | 2.30E−05 | 0.67142857 | 0.6746988 |
| 121 | 190 | 160 | 213 | 184 | 239 | 246 | 189 | 1.10E−07 | 0.67142857 | 0.75903614 |
| 168 | 225 | 176 | 251 | 236 | 189 | 190 | 218 | 4.11E−08 | 0.67142857 | 0.77108434 |
| 235 | 116 | 187 | 250 | 168 | 220 | 238 | 190 | 6.78E−07 | 0.65714286 | 0.74698795 |
| 216 | 214 | 246 | 116 | 244 | 182 | 240 | 186 | 7.10E−07 | 0.67142857 | 0.73493976 |
| 208 | 188 | 187 | 218 | 245 | 238 | 199 | 157 | 1.64E−06 | 0.65714286 | 0.73493976 |
| 239 | 112 | 176 | 185 | 246 | 250 | 219 | 202 | 4.86E−05 | 0.65714286 | 0.6746988 |
| 250 | 220 | 233 | 127 | 224 | 116 | 226 | 237 | 1.72E−06 | 0.67142857 | 0.72289157 |
| 156 | 212 | 204 | 259 | 214 | 237 | 240 | 191 | 1.05E−07 | 0.65714286 | 0.77108434 |
| 259 | 204 | 213 | 228 | 180 | 218 | 242 | 193 | 1.72E−06 | 0.67142857 | 0.72289157 |
| 218 | 250 | 227 | 211 | 171 | 185 | 251 | 133 | 1.05E−07 | 0.65714286 | 0.77108434 |
| 176 | 202 | 185 | 187 | 277 | 248 | 233 | 189 | 1.72E−06 | 0.67142857 | 0.72289157 |
| 112 | 277 | 218 | 155 | 156 | 237 | 235 | 244 | 5.67E−10 | 0.67142857 | 0.81927711 |
| 187 | 252 | 240 | 116 | 175 | 184 | 239 | 242 | 2.84E−07 | 0.67142857 | 0.74698795 |
| 182 | 227 | 206 | 181 | 132 | 224 | 244 | 188 | 1.10E−07 | 0.67142857 | 0.75903614 |
| 239 | 238 | 214 | 223 | 242 | 218 | 186 | 192 | 1.66E−08 | 0.7 | 0.75903614 |
| 185 | 188 | 277 | 241 | 219 | 193 | 201 | 176 | 1.64E−06 | 0.65714286 | 0.73493976 |
| 116 | 233 | 199 | 247 | 183 | 238 | 214 | 180 | 4.11E−08 | 0.67142857 | 0.77108434 |
| 180 | 242 | 116 | 239 | 158 | 238 | 243 | 240 | 7.46E−07 | 0.68571429 | 0.72289157 |
| 234 | 237 | 193 | 235 | 224 | 179 | 190 | 233 | 3.92E−08 | 0.65714286 | 0.78313253 |

For k=32, 99.9% of tested polymorphism genotype combinations yield a sensitivity and specificity of higher than 50% each in specificity and sensitivity, 98.9% of tested polymorphism genotype combinations yield a sensitivity and specificity of higher than 60% each, 72.8% of tested polymorphism genotype combinations yield a sensitivity and specificity of higher than 65% each, 15.6% of tested polymorphism genotype combinations yield a sensitivity and specificity of higher than 70% each in predicting a clinical response. Finally, some of the tested polymorphism genotype combinations (0.3%) even yield a sensitivity and specificity of higher than 75% each (data not shown).

As will be understood from the above explanations and data in Table 5, Table 6, and Table 7, even minimal subsets of polymorphism genotypes selected from the particularly useful set of polymorphism genotypes disclosed in Table 2 already allow for predictions of a clinical response significantly better than 50% (“coin-flip”). Therefore, while the present invention ideally aims at predicting the treatment response to a CRHR1 antagonist with sensitivity and specificity of at least 75% each, at least 80% each, at least 85% each, or even at least 90% each, methods of prediction using smaller subsets, e.g., of only one, two, four, or eight polymorphism genotypes selected from the group consisting of the polymorphism genotypes disclosed in Table 2 already provide a significant performance in predicting clinical responses. A subset of k=32 polymorphism genotypes already comprises combinations yielding a sensitivity and specificity of at least 75% each in predicting a clinical response. The predictive performance can be further increased by including, e.g., 150 polymorphism genotypes, as has been done in Example 1, 200 polymorphism genotypes, 250 polymorphism genotypes or all polymorphism genotypes as disclosed in Table 2.

### EQUIVALENTS

The foregoing exemplary embodiments are to be considered illustrative of, and not limiting to, the invention disclosed herein. It will be apparent to those skilled in the art that various modifications may be made without departing from the scope or spirit of the invention. Therefore, it will be appreciated that the scope of the present invention is primarily defined by the appended claims, and is not limited by the specific embodiments which have been presented as examples. All changes which come within the meaning and range of equivalency of the claims are intended to be encompassed.

1. A method for predicting a treatment response of a subject to a treatment with a non-peptidic CRHR1 antagonist, the method comprising:
detecting the presence or absence of one or more polymorphism genotypes in a biological sample from the subject, wherein the one or more polymorphism genotypes comprise:
at least one polymorphism genotype selected from the group consisting of rs34169260 (A/G), rs796287 (A/C), rs56149945 (A/G), rs6190 (T/C), rs7179092 (T/C), rs7614867 (A/G), rs920640 (T/C), rs7167722 (T/C), rs920638 (T/C), rs7165629 (T/C), rs80049044 (T/A), rs16941058 (A/G), rs112015971 (A/G), rs10894873 (T/C), rs117455294 (T/G), rs1170303 (T/C), rs16940681 (C/G), rs968519 (T/C), rs28381866 (T/C), rs79320848 (T/G), rs114653646 (T/G), rs2589496 (T/C), rs10482650 (A/G), rs17614642 (A/G), rs73200317 (T/C), rs1380146 (T/A), rs735164 (T/C), rs730976 (T/G), rs55934524 (T/G), rs4570614 (A/G), rs4458044 (C/G), rs77850169 (A/G), rs35339359 (A/G), rs34800935 (T/C), rs72945439 (T/C), rs113959523 (A/G), rs116798177 (A/G), rs11247577 (T/G), rs75869266 (T/C), rs74372553 (T/C), rs11691508 (A/G), rs6493965 (A/G), rs4869476 (T/C), rs3730170 (T/C), rs2145288 (A/C), rs2935752 (A/C), rs146512400 (A/G), rs62057097 (T/C), rs115061314 (T/C), rs34113594 (T/G), rs61751173 (A/G), rs74338736 (A/C), rs10851726 (T/C), rs4610906 (T/C), rs59485211 (T/C), rs7060015 (T/G), rs75710780 (T/G), rs6520908 (T/C), rs487011 (T/G), rs1383699 (A/C), rs67516871 (A/G), rs114106519 (T/C), rs7220091 (A/G), rs12489026 (A/G), rs876270 (T/C), rs4968161 (T/C), rs62056907 (A/G), rs2235013 (T/C), rs16878812 (A/G), rs6549407 (A/G), rs28381848 (A/G), rs79723704 (A/C), rs72814052 (A/G), rs10152908 (T/C), rs172769 (A/C), rs78596668 (T/C), rs73307922 (T/C), rs3842 (A/G), rs7210584 (A/C), rs62402121 (T/C), rs55709291 (A/G), rs72747088 (A/G), rs929610 (G/C), rs6766242 (T/C), rs1468552 (G/C), rs78838114 (T/C), rs62489862 (T/C), rs894342 (A/G), rs58882373 (T/C), rs3811939 (A/G), rs6984688 (T/G), rs1018160 (T/C), rs76602912 (A/G), rs80067508 (A/G), rs74888440 (T/C), rs12481583 (T/C), rs66794218 (A/G), rs16946701 (A/G), rs75726724 (A/G), rs67959715 (T/A), rs11871392 (T/G), rs2044070 (A/G), rs77612799 (T/C), rs6743702 (T/C), rs616870 (T/C), rs79590198 (A/G), rs75715199 (A/G), rs13087555 (T/C), rs4869618 (T/C), rs117397046 (A/G), rs8042817 (A/G), rs2258097 (T/C), rs2260882 (C/G), rs532996 (A/G), rs11747040 (T/C), rs10034039 (T/G), rs116909369 (A/G), rs79134986 (A/G), rs117615688 (T/C), rs8032253 (T/C), rs12818653 (T/A), rs4587884 (A/C), rs77122853 (T/C), rs117615061 (T/C), rs74682905 (A/G), rs2257468 (T/C), rs2032582 (T/G), rs2235015 (T/G), rs2729794 (T/C), rs77549514 (A/G), rs74790420 (A/C), rs73129579 (T/C), rs12913346 (A/C), rs117560908 (T/C), rs72747091 (A/G), rs2935751 (A/G), rs4331446 (A/G), rs7523266 (T/C), rs7648662 (T/C), rs117034065 (A/G), rs4836256 (T/C), rs80238698 (T/C), rs3730173 (T/C), rs11687884 (T/C), rs72693005 (T/C), rs2589476 (T/C), rs9813396 (T/C), rs10482667 (A/G), rs72784444 (A/G), rs75074511 (T/C), rs7951003 (A/G), rs79584784 (A/G), rs2214102 (T/C), rs28811003 (A/G), rs6100261 (A/T), rs77152456 (A/G), rs66624622 (T/G), rs140302965 (A/G), rs11653269 (T/C), rs74405057 (A/G), rs7121 (A/G), rs16977818 (A/C), rs12490095 (T/C), rs118003903 (A/G), rs62377761 (T/C), P1_M_061510_6_34 M (−/CACTTAC CTTCTTTGTGCCACAGTTTCCCTATCTAAAACACAAGGTTATCAGTTATCAAC ATCTCTTGGGATTGTGAGGACTAAAGTAATGCACATAAAG), rs375115639 (−/AAATTACCCTGTTAGGTTTCAATGAAACACCTTTTCTCTTGTAACAAACATCT CC TCCAAGCTAGAATTTCAAAACAG), rs1002204 (A/C), rs10062367 (A/G), rs10482642 (A/G), rs10482658 (A/G), rs1053989 (A/C), rs10851628 (T/C), rs10947562 (T/C), rs11069612 (A/G), rs11071351 (T/C), rs11091175 (A/G), rs11638450 (T/C), rs11715827 (T/G), rs11745958 (T/C), rs11834041 (A/G), rs1202180 (T/C), rs12054781 (A/G), rs12539395 (A/G), rs12720066 (T/G), rs1279754 (A/C), rs12872047 (T/C), rs12876742 (A/C), rs12917505 (A/G), rs13066950 (T/G), rs13229143 (C/G), rs1383707 (T/C), rs1441824 (T/C), rs1652311 (A/G), rs17064 (T/A), rs17100236 (A/G), rs17149699 (A/G), rs1724386 (A/G), rs17250255 (A/G), rs17327624 (T/G), rs17616338 (A/G), rs17687796 (A/G), rs17740874 (T/C), rs17763104 (T/C), rs1880748 (T/C), rs1882478 (A/G), rs1944887 (T/C), rs2028629 (A/G), rs2143404 (A/G), rs2173530 (T/C), rs220806 (T/C), rs2235047 (A/C), rs2242071 (A/G), rs2257474 (T/C), rs2295583 (A/T), rs234629 (T/C), rs234630 (A/G), rs2436401 (A/G), rs258750 (T/C), rs2589487 (T/C), rs28364018 (T/G), rs28381774 (T/C), rs28381784 (A/G), rs2963155 (A/G), rs3133622 (T/G), rs32897 (T/C), rs33388 (A/T), rs3730168 (T/C), rs3735833 (T/G), rs3777747 (A/G), rs3786066 (T/C), rs3798346 (T/C), rs3822736 (A/G), rs389035 (T/C), rs3924144 (A/G), rs4148737 (T/C), rs4148749 (G/C), rs417968 (T/C), rs4458144 (T/C), rs4515335 (T/C), rs4728699 (A/G), rs4758040 (A/G), rs4812040 (A/G), rs4912650 (T/G), rs4957891 (T/C), rs5906392 (A/G), rs6026561 (T/C), rs6026565 (T/A), rs6026567 (A/G), rs6026593 (A/G), rs6092704 (T/G), rs6100260 (A/G), rs6128461 (T/C), rs6415328 (T/C), rs6610868 (T/C), rs6686061 (A/C), rs6730350 (T/G), rs6746197 (T/C), rs6963426 (T/C), rs7121326 (T/C), rs7721799 (A/G), rs7787082 (T/C), rs7799592 (A/C), rs796245 (T/C), rs809482 (A/C), rs8125112 (T/C), rs919196 (A/G), rs920750 (T/C), rs9332385 (A/G), rs930473 (T/G), rs9324921 (A/C), rs9348979 (A/G), rs9571939 (A/C), and rs9892359 (T/C).

2. The method of claim 1, wherein the predicting step comprises:
(a) determining whether the subject will respond, or has an increased likelihood of responding to the treatment with a CRHR1 antagonist; and/or
(b) determining whether the subject will not respond, or has a decreased likelihood of responding to the treatment with a CRHR1 antagonist.

3. The method of claim 2, wherein the determining step comprises one or more statistical analysis method selected from the group consisting of artificial neural network learning, decision tree learning, decision tree forest learning, linear discriminant analysis, non-linear discriminant analysis, genetic expression programming, relevance vector machines, linear models, generalized linear models, generalized estimating equations, generalized linear mixed models, the elastic net, the lasso support vector machine learning, Bayesian network learning, probabilistic neural network learning, clustering, and regression analysis, optionally wherein the statistical analysis method is computer-implemented.

4. The method of claim 1, wherein the one or more polymorphism genotypes comprise:
(a) at least two;
(b) at least four;
(c) at least eight;
(d) at least sixteen;
(e) at least thirty-two; or
(f) all
of the polymorphism genotypes as defined in claim 1.

5. The method of claim 1, wherein the one or more polymorphism genotypes comprise:
(a) at least two polymorphism genotypes selected from the combinations of polymorphism genotypes disclosed in Table 5;
(b) at least four polymorphism genotypes selected from the combinations of polymorphism genotypes disclosed in Table 6;
(c) at least eight polymorphism genotypes selected from the combinations of polymorphism genotypes disclosed in Table 7;
(d) all of the polymorphism genotypes disclosed in Table 2.

6. The method of claim 1, wherein the treatment response to treatment with the CRHR1 antagonist is a clinical response.

7. The method of claim 1, wherein the subject has depressive symptoms, anxiety symptoms or both depressive symptoms and anxiety symptoms or a sleep disorder; and/or
wherein the treatment is a treatment of depressive symptoms, anxiety symptoms or both depressive symptoms and anxiety symptoms or a sleep disorder.

8. The method of claim 7, wherein the treatment response to treatment with the CRHR1 antagonist is a clinical response, and wherein the clinical response is a prevention, alteration, alleviation or complete remission of depressive symptoms and/or anxiety symptoms or a sleep disorder.

9. The method of claim 8, wherein the clinical response is a prevention, alteration, alleviation or complete remission of depressive symptoms and/or anxiety symptoms as determined using a scale selected from the group consisting of HAM-D, BDI, MADRS, GDS, ZSRDS, HAM-A and STAI.

10. The method of claim 1, wherein the biological sample is a buccal or a blood sample.

11. The method of claim 1, wherein detecting comprises the use of one or more polynucleotides capable of specifically hybridizing to at least one nucleic acid comprising the one or more polymorphism genotypes.

12. The method of claim 1, wherein after the subject has been administered a CRHR1 antagonist, further comprising comparing the prediction that the subject will respond to a treatment with a CRHR1 antagonist with the treatment response of the subject to administration of the CRHR1 antagonist.

13. The method of claim 1, wherein the prediction that the subject will respond to a treatment with a CRHR1 antagonist has a sensitivity of higher than 50% and a specificity of higher than 50%.

14. The method of claim 1, wherein the CRHR1 antagonist is selected from the group consisting of GW876008 (Emicerfont), GSK-561679 (NBI-77860, Verucerfont), GSK586529, BMS-562,086 (Pexacerfont), NBI-30775 (R-121919), NBI-34101, CP-316,311, CP-376,395, PF-00572778, NVP-AAG561, Ono-2333MS, E2508, E2009, R317573 (JNJ19567470, CRA5626, TAI-041), R278995 (CRA0450), CRA-1000, CRA-1001, CP154,526, Antalarmin, DMP-695, DMP-696, DMP-904, SC-241, BMS-561388, NBI30545, PD-171729, NBI34041, NBI35965, SN003, NBI-27914, trans-2-chloro-N-(4-((5-fluoro-4-methyl-pyridin-2-ylamino)-methyl)-cyclohexyl)-5-(trifluoromethyl)-benzamide, SSR-125543, or a pharmaceutically acceptable salt thereof.

15. A method of treating a condition characterized, caused or accompanied by CRH overproduction or over-activity in a subject in need thereof, wherein the method comprises administering an effective amount of a CRHR1 antagonist to the subject, wherein the subject has been predicted to respond, or has an increased likelihood of responding, to a treatment with a CRHR1 antagonist, as determined by the method according to claim 1, and wherein the CRHR1 antagonist is:
(a) a compound of Formula (I), as defined herein; or
(b) selected from the group consisting of a Type I CRHR1 antagonist, a bicyclic Type II CRHR1 antagonist, an atypical CRHR1 antagonist, and a cyclohexyl amide CRHR1 antagonist.

16. The method of claim 15, wherein the condition is selected from the group consisting of depressive symptoms, anxiety symptoms, both depressive symptoms and anxiety symptoms, and a sleep disorder.

17. The of claim 15, wherein the CRHR1 antagonist is selected from the group consisting of GW876008 (Emicerfont), GSK-561679 (NBI-77860, Verucerfont), GSK586529, BMS-562,086 (Pexacerfont), NBI-30775 (R-121919), NBI-34101, CP-316,311, CP-376,395, PF-00572778, NVP-AAG561, Ono-2333MS, E2508, E2009, R317573 (JNJ19567470, CRA5626, TAI-041), R278995 (CRA0450), CRA-1000, CRA-1001, CP154,526, Antalarmin, DMP-695, DMP-696, DMP-904, SC-241, BMS-561388, NBI30545, PD-171729, NBI34041, NBI35965, SN003, NBI-27914, trans-2-chloro-N-(4-((5-fluoro-4-methyl-pyridin-2-ylamino)-methyl)-cyclohexyl)-5-(trifluoromethyl)-benzamide, or a pharmaceutically acceptable salt thereof.

18. Use of a composition comprising at least one polynucleotide capable of specifically hybridizing to a nucleic acid comprising:
at least one polymorphism genotype selected from the group consisting of rs34169260 (A/G), rs796287 (A/C), rs56149945 (A/G), rs6190 (T/C), rs7179092 (T/C), rs7614867 (A/G), rs920640 (T/C), rs7167722 (T/C), rs920638 (T/C), rs7165629 (T/C), rs80049044 (T/A), rs16941058 (A/G), rs112015971 (A/G), rs10894873 (T/C), rs117455294 (T/G), rs1170303 (T/C), rs16940681 (C/G), rs968519 (T/C), rs28381866 (T/C), rs79320848 (T/G), rs114653646 (T/G), rs2589496 (T/C), rs10482650 (A/G), rs17614642 (A/G), rs73200317 (T/C), rs1380146 (T/A), rs735164 (T/C), rs730976 (T/G), rs55934524 (T/G), rs4570614 (A/G), rs4458044 (C/G), rs77850169 (A/G), rs35339359 (A/G), rs34800935 (T/C), rs72945439 (T/C), rs113959523 (A/G), rs116798177 (A/G), rs11247577 (T/G), rs75869266 (T/C), rs74372553 (T/C), rs11691508 (A/G), rs6493965 (A/G), rs4869476 (T/C), rs3730170 (T/C), rs2145288 (A/C), rs2935752 (A/C), rs146512400 (A/G), rs62057097 (T/C), rs115061314 (T/C), rs34113594 (T/G), rs61751173 (A/G), rs74338736 (A/C), rs10851726 (T/C), rs4610906 (T/C), rs59485211 (T/C), rs7060015 (T/G), rs75710780 (T/G), rs6520908 (T/C), rs487011 (T/G), rs1383699 (A/C), rs67516871 (A/G), rs114106519 (T/C), rs7220091 (A/G), rs12489026 (A/G), rs876270 (T/C), rs4968161 (T/C), rs62056907 (A/G), rs2235013 (T/C), rs16878812 (A/G), rs6549407 (A/G), rs28381848 (A/G), rs79723704 (A/C), rs72814052 (A/G), rs10152908 (T/C), rs172769 (A/C), rs78596668 (T/C), rs73307922 (T/C), rs3842 (A/G), rs7210584 (A/C), rs62402121 (T/C), rs55709291 (A/G), rs72747088 (A/G), rs929610 (G/C), rs6766242 (T/C), rs1468552 (G/C), rs78838114 (T/C), rs62489862 (T/C), rs894342 (A/G), rs58882373 (T/C), rs3811939 (A/G), rs6984688 (T/G), rs1018160 (T/C), rs76602912 (A/G), rs80067508 (A/G), rs74888440 (T/C), rs12481583 (T/C), rs66794218 (A/G), rs16946701 (A/G), rs75726724 (A/G), rs67959715 (T/A), rs11871392 (T/G), rs2044070 (A/G), rs77612799 (T/C), rs6743702 (T/C), rs616870 (T/C), rs79590198 (A/G), rs75715199 (A/G), rs13087555 (T/C), rs4869618 (T/C), rs117397046 (A/G), rs8042817 (A/G), rs2258097 (T/C), rs2260882 (C/G), rs532996 (A/G), rs11747040 (T/C), rs10034039 (T/G), rs116909369 (A/G), rs79134986 (A/G), rs117615688 (T/C), rs8032253 (T/C), rs12818653 (T/A), rs4587884 (A/C), rs77122853 (T/C), rs117615061 (T/C), rs74682905 (A/G), rs2257468 (T/C), rs2032582 (T/G), rs2235015 (T/G), rs2729794 (T/C), rs77549514 (A/G), rs74790420 (A/C), rs73129579 (T/C), rs12913346 (A/C), rs117560908 (T/C), rs72747091 (A/G), rs2935751 (A/G), rs4331446 (A/G), rs7523266 (T/C), rs7648662 (T/C), rs117034065 (A/G), rs4836256 (T/C), rs80238698 (T/C), rs3730173 (T/C), rs11687884 (T/C), rs72693005 (T/C), rs2589476 (T/C), rs9813396 (T/C), rs10482667 (A/G), rs72784444 (A/G), rs75074511 (T/C), rs7951003 (A/G), rs79584784 (A/G), rs2214102 (T/C), rs28811003 (A/G), rs6100261 (A/T), rs77152456 (A/G), rs66624622 (T/G), rs140302965 (A/G), rs11653269 (T/C), rs74405057 (A/G), rs7121 (A/G), rs16977818 (A/C), rs12490095 (T/C), rs118003903 (A/G), rs62377761 (T/C), P1_M_061510_6_34_M (−/CACTTAC CTTCTTTGTGCCACAGTTTCCCTATCTAAAACACAAGGTTATCAGTTATCAAC ATCTCTTGGGATTGTGAGGACTAAAGTAATGCACATAAAG), rs375115639 (−/AAATTACCCTGTTAGGTTTCAATGAAACACCTTTTCTCTTGTAACAAACATCT CC TCCAAGCTAGAATTTCAAAACAG), rs1002204 (A/C), rs10062367 (A/G), rs10482642 (A/G), rs10482658 (A/G), rs1053989 (A/C), rs10851628 (T/C), rs10947562 (T/C), rs11069612 (A/G), rs11071351 (T/C), rs11091175 (A/G), rs11638450 (T/C), rs11715827 (T/G), rs11745958 (T/C), rs11834041 (A/G), rs1202180 (T/C), rs12054781 (A/G), rs12539395 (A/G), rs12720066 (T/G), rs1279754 (A/C), rs12872047 (T/C), rs12876742 (A/C), rs12917505 (A/G), rs13066950 (T/G), rs13229143 (C/G), rs1383707 (T/C), rs1441824 (T/C), rs1652311 (A/G), rs17064 (T/A), rs17100236 (A/G), rs17149699 (A/G), rs1724386 (A/G), rs17250255 (A/G), rs17327624 (T/G), rs17616338 (A/G), rs17687796 (A/G), rs17740874 (T/C), rs17763104 (T/C), rs1880748 (T/C), rs1882478 (A/G), rs1944887 (T/C), rs2028629 (A/G), rs2143404 (A/G), rs2173530 (T/C), rs220806 (T/C), rs2235047 (A/C), rs2242071 (A/G), rs2257474 (T/C), rs2295583 (A/T), rs234629 (T/C), rs234630 (A/G), rs2436401 (A/G), rs258750 (T/C), rs2589487 (T/C), rs28364018 (T/G), rs28381774 (T/C), rs28381784 (A/G), rs2963155 (A/G), rs3133622 (T/G), rs32897 (T/C), rs33388 (A/T), rs3730168 (T/C), rs3735833 (T/G), rs3777747 (A/G), rs3786066 (T/C), rs3798346 (T/C), rs3822736 (A/G), rs389035 (T/C), rs3924144 (A/G), rs4148737 (T/C), rs4148749 (G/C), rs417968 (T/C), rs4458144 (T/C), rs4515335 (T/C), rs4728699 (A/G), rs4758040 (A/G), rs4812040 (A/G), rs4912650 (T/G), rs4957891 (T/C), rs5906392 (A/G), rs6026561 (T/C), rs6026565 (T/A), rs6026567 (A/G), rs6026593 (A/G), rs6092704 (T/G), rs6100260 (A/G), rs6128461 (T/C), rs6415328 (T/C), rs6610868 (T/C), rs6686061 (A/C), rs6730350 (T/G), rs6746197 (T/C), rs6963426 (T/C), rs7121326 (T/C), rs7721799 (A/G), rs7787082 (T/C), rs7799592 (A/C), rs796245 (T/C), rs809482 (A/C), rs8125112 (T/C), rs919196 (A/G), rs920750 (T/C), rs9332385 (A/G), rs930473 (T/G), rs9324921 (A/C), rs9348979 (A/G), rs9571939 (A/C), and rs9892359 (T/C),
in a method for predicting the treatment response of a subject to a treatment with a non-peptidic CRHR1 antagonist.

19. A method of using a kit comprising at least one polynucleotide capable of specifically hybridizing to a nucleic acid comprising:
at least one polymorphism genotype selected from the group consisting of rs34169260 (A/G), rs796287 (A/C), rs56149945 (A/G), rs6190 (T/C), rs7179092 (T/C), rs7614867 (A/G), rs920640 (T/C), rs7167722 (T/C), rs920638 (T/C), rs7165629 (T/C), rs80049044 (T/A), rs16941058 (A/G), rs112015971 (A/G), rs10894873 (T/C), rs117455294 (T/G), rs1170303 (T/C), rs16940681 (C/G), rs968519 (T/C), rs28381866 (T/C), rs79320848 (T/G), rs114653646 (T/G), rs2589496 (T/C), rs10482650 (A/G), rs17614642 (A/G), rs73200317 (T/C), rs1380146 (T/A), rs735164 (T/C), rs730976 (T/G), rs55934524 (T/G), rs4570614 (A/G), rs4458044 (C/G), rs77850169 (A/G), rs35339359 (A/G), rs34800935 (T/C), rs72945439 (T/C), rs113959523 (A/G), rs116798177 (A/G), rs11247577 (T/G), rs75869266 (T/C), rs74372553 (T/C), rs11691508 (A/G), rs6493965 (A/G), rs4869476 (T/C), rs3730170 (T/C), rs2145288 (A/C), rs2935752 (A/C), rs146512400 (A/G), rs62057097 (T/C), rs115061314 (T/C), rs34113594 (T/G), rs61751173 (A/G), rs74338736 (A/C), rs10851726 (T/C), rs4610906 (T/C), rs59485211 (T/C), rs7060015 (T/G), rs75710780 (T/G), rs6520908 (T/C), rs487011 (T/G), rs1383699 (A/C), rs67516871 (A/G), rs114106519 (T/C), rs7220091 (A/G), rs12489026 (A/G), rs876270 (T/C), rs4968161 (T/C), rs62056907 (A/G), rs2235013 (T/C), rs16878812 (A/G), rs6549407 (A/G), rs28381848 (A/G), rs79723704 (A/C), rs72814052 (A/G), rs10152908 (T/C), rs172769 (A/C), rs78596668 (T/C), rs73307922 (T/C), rs3842 (A/G), rs7210584 (A/C), rs62402121 (T/C), rs55709291 (A/G), rs72747088 (A/G), rs929610 (G/C), rs6766242 (T/C), rs1468552 (G/C), rs78838114 (T/C), rs62489862 (T/C), rs894342 (A/G), rs58882373 (T/C), rs3811939 (A/G), rs6984688 (T/G), rs1018160 (T/C), rs76602912 (A/G), rs80067508 (A/G), rs74888440 (T/C), rs12481583 (T/C), rs66794218 (A/G), rs16946701 (A/G), rs75726724 (A/G), rs67959715 (T/A), rs11871392 (T/G), rs2044070 (A/G), rs77612799 (T/C), rs6743702 (T/C), rs616870 (T/C), rs79590198 (A/G), rs75715199 (A/G), rs13087555 (T/C), rs4869618 (T/C), rs117397046 (A/G), rs8042817 (A/G), rs2258097 (T/C), rs2260882 (C/G), rs532996 (A/G), rs11747040 (T/C), rs10034039 (T/G), rs116909369 (A/G), rs79134986 (A/G), rs117615688 (T/C), rs8032253 (T/C), rs12818653 (T/A), rs4587884 (A/C), rs77122853 (T/C), rs117615061 (T/C), rs74682905 (A/G), rs2257468 (T/C), rs2032582 (T/G), rs2235015 (T/G), rs2729794 (T/C), rs77549514 (A/G), rs74790420 (A/C), rs73129579 (T/C), rs12913346 (A/C), rs117560908 (T/C), rs72747091 (A/G), rs2935751 (A/G), rs4331446 (A/G), rs7523266 (T/C), rs7648662 (T/C), rs117034065 (A/G), rs4836256 (T/C), rs80238698 (T/C), rs3730173 (T/C), rs11687884 (T/C), rs72693005 (T/C), rs2589476 (T/C), rs9813396 (T/C), rs10482667 (A/G), rs72784444 (A/G), rs75074511 (T/C), rs7951003 (A/G), rs79584784 (A/G), rs2214102 (T/C), rs28811003 (A/G), rs6100261 (A/T), rs77152456 (A/G), rs66624622 (T/G), rs140302965 (A/G), rs11653269 (T/C), rs74405057 (A/G), rs7121 (A/G), rs16977818 (A/C), rs12490095 (T/C), rs118003903 (A/G), rs62377761 (T/C), P1_M_061510_6_34_M (−/CACTTAC CTTCTTTGTGCCACAGTTTCCCTATCTAAAACACAAGGTTATCAGTTATCAAC ATCTCTTGGGATTGTGAGGACTAAAGTAATGCACATAAAG), rs375115639 (−/AAATTACCCTGTTAGGTTTCAATGAAACACCTTTTCTCTTGTAACAAACATCT CC TCCAAGCTAGAATTTCAAAACAG), rs1002204 (A/C), rs10062367 (A/G), rs10482642 (A/G), rs10482658 (A/G), rs1053989 (A/C), rs10851628 (T/C), rs10947562 (T/C), rs11069612 (A/G), rs11071351 (T/C), rs11091175 (A/G), rs11638450 (T/C), rs11715827 (T/G), rs11745958 (T/C), rs11834041 (A/G), rs1202180 (T/C), rs12054781 (A/G), rs12539395 (A/G), rs12720066 (T/G), rs1279754 (A/C), rs12872047 (T/C), rs12876742 (A/C), rs12917505 (A/G), rs13066950 (T/G), rs13229143 (C/G), rs1383707 (T/C), rs1441824 (T/C), rs1652311 (A/G), rs17064 (T/A), rs17100236 (A/G), rs17149699 (A/G), rs1724386 (A/G), rs17250255 (A/G), rs17327624 (T/G), rs17616338 (A/G), rs17687796 (A/G), rs17740874 (T/C), rs17763104 (T/C), rs1880748 (T/C), rs1882478 (A/G), rs1944887 (T/C), rs2028629 (A/G), rs2143404 (A/G), rs2173530 (T/C), rs220806 (T/C), rs2235047 (A/C), rs2242071 (A/G), rs2257474 (T/C), rs2295583 (A/T), rs234629 (T/C), rs234630 (A/G), rs2436401 (A/G), rs258750 (T/C), rs2589487 (T/C), rs28364018 (T/G), rs28381774 (T/C), rs28381784 (A/G), rs2963155 (A/G), rs3133622 (T/G), rs32897 (T/C), rs33388 (A/T), rs3730168 (T/C), rs3735833 (T/G), rs3777747 (A/G), rs3786066 (T/C), rs3798346 (T/C), rs3822736 (A/G), rs389035 (T/C), rs3924144 (A/G), rs4148737 (T/C), rs4148749 (G/C), rs417968 (T/C), rs4458144 (T/C), rs4515335 (T/C), rs4728699 (A/G), rs4758040 (A/G), rs4812040 (A/G), rs4912650 (T/G), rs4957891 (T/C), rs5906392 (A/G), rs6026561 (T/C), rs6026565 (T/A), rs6026567 (A/G), rs6026593 (A/G), rs6092704 (T/G), rs6100260 (A/G), rs6128461 (T/C), rs6415328 (T/C), rs6610868 (T/C), rs6686061 (A/C), rs6730350 (T/G), rs6746197 (T/C), rs6963426 (T/C), rs7121326 (T/C), rs7721799 (A/G), rs7787082 (T/C), rs7799592 (A/C), rs796245 (T/C), rs809482 (A/C), rs8125112 (T/C), rs919196 (A/G), rs920750 (T/C), rs9332385 (A/G), rs930473 (T/G), rs9324921 (A/C), rs9348979 (A/G), rs9571939 (A/C), and rs9892359 (T/C),
and
one or more additional reagents for detecting the presence or absence of the one or more polymorphism genotypes;
optionally comprising instructions for detecting the presence or absence of the at least one polymorphism genotype in a sample obtained from a subject;
the method comprising predicting the treatment response of a subject to a treatment with a non-peptidic CRHR1 antagonist.

20. The method of claim 18, wherein the at least one polynucleotide comprises:
(a) at least two polynucleotides capable of specifically hybridizing to nucleic acids comprising each of at least two of said polymorphism genotypes;
(b) at least four polynucleotides capable of specifically hybridizing to nucleic acids comprising each of at least four of said polymorphism genotypes;
(c) at least eight polynucleotides capable of specifically hybridizing to nucleic acids comprising each of at least eight of said polymorphism genotypes;
(d) at least 16 polynucleotides capable of specifically hybridizing to nucleic acids comprising each of at least 16 of said polymorphism genotypes;
(e) at least 32 polynucleotides capable of specifically hybridizing to nucleic acids comprising each of at least 32 of said polymorphism genotypes; or
(f) 274 polynucleotides capable of specifically hybridizing to nucleic acids comprising each of said polymorphism genotypes.

21. The method of claim 18, wherein
the at least one polymorphism genotype comprises:
(a) at least two polynucleotides capable of specifically hybridizing to nucleic acids comprising each of at least two polymorphism genotypes selected from the combinations of polymorphism genotypes disclosed in Table 5;
(b) at least four polynucleotides capable of specifically hybridizing to nucleic acids comprising each of at least four polymorphism genotypes selected from the combinations of polymorphism genotypes disclosed in Table 6;
(c) at least eight polynucleotides capable of specifically hybridizing to nucleic acids comprising each of at least eight polymorphism genotypes selected from the combinations of polymorphism genotypes disclosed in Table 7; or
(d) 274 polynucleotides capable of specifically hybridizing to nucleic acids comprising each of the polymorphism genotypes disclosed in Table 2.

22. The method of claim 18, wherein the at least one polynucleotide is bound to a solid support.

23. The method of claim 18, wherein the at least one polynucleotide is bound to the solid support in the form of an array.

24. The method of claim 19, further comprising one or more reagents for isolating a nucleic acid from a sample.

25. The method of claim 19, further comprising a means for amplifying a nucleic acid.

26. The method of claim 19, wherein the composition or kit is used to detect the presence or absence of one or more polymorphism genotypes within a sample obtained from a subject.

27. A method for detecting a polymorphism genotype associated with a treatment response of a subject to treatment with a non-peptidic CRHR1 antagonist, the method comprising:
detecting the presence or absence of one or more polymorphism genotypes in a biological sample obtained from the subject, wherein the one or more polymorphism genotypes comprise: (a) at least one polymorphism genotype selected from the group consisting of rs34169260 (A/G), rs796287 (A/C), rs56149945 (A/G), rs6190 (T/C), rs7179092 (T/C), rs7614867 (A/G), rs920640 (T/C), rs7167722 (T/C), rs920638 (T/C), rs7165629 (T/C), rs80049044 (T/A), rs16941058 (A/G), rs112015971 (A/G), rs10894873 (T/C), rs117455294 (T/G), rs1170303 (T/C), rs16940681 (C/G), rs968519 (T/C), rs28381866 (T/C), rs79320848 (T/G), rs114653646 (T/G), rs2589496 (T/C), rs10482650 (A/G), rs17614642 (A/G), rs73200317 (T/C), rs1380146 (T/A), rs735164 (T/C), rs730976 (T/G), rs55934524 (T/G), rs4570614 (A/G), rs4458044 (C/G), rs77850169 (A/G), rs35339359 (A/G), rs34800935 (T/C), rs72945439 (T/C), rs113959523 (A/G), rs116798177 (A/G), rs11247577 (T/G), rs75869266 (T/C), rs74372553 (T/C), rs11691508 (A/G), rs6493965 (A/G), rs4869476 (T/C), rs3730170 (T/C), rs2145288 (A/C), rs2935752 (A/C), rs146512400 (A/G), rs62057097 (T/C), rs115061314 (T/C), rs34113594 (T/G), rs61751173 (A/G), rs74338736 (A/C), rs10851726 (T/C), rs4610906 (T/C), rs59485211 (T/C), rs7060015 (T/G), rs75710780 (T/G), rs6520908 (T/C), rs487011 (T/G), rs1383699 (A/C), rs67516871 (A/G), rs114106519 (T/C), rs7220091 (A/G), rs12489026 (A/G), rs876270 (T/C), rs4968161 (T/C), rs62056907 (A/G), rs2235013 (T/C), rs16878812 (A/G), rs6549407 (A/G), rs28381848 (A/G), rs79723704 (A/C), rs72814052 (A/G), rs10152908 (T/C), rs172769 (A/C), rs78596668 (T/C), rs73307922 (T/C), rs3842 (A/G), rs7210584 (A/C), rs62402121 (T/C), rs55709291 (A/G), rs72747088 (A/G), rs929610 (G/C), rs6766242 (T/C), rs1468552 (G/C), rs78838114 (T/C), rs62489862 (T/C), rs894342 (A/G), rs58882373 (T/C), rs3811939 (A/G), rs6984688 (T/G), rs1018160 (T/C), rs76602912 (A/G), rs80067508 (A/G), rs74888440 (T/C), rs12481583 (T/C), rs66794218 (A/G), rs16946701 (A/G), rs75726724 (A/G), rs67959715 (T/A), rs11871392 (T/G), rs2044070 (A/G), rs77612799 (T/C), rs6743702 (T/C), rs616870 (T/C), rs79590198 (A/G), rs75715199 (A/G), rs13087555 (T/C), rs4869618 (T/C), rs117397046 (A/G), rs8042817 (A/G), rs2258097 (T/C), rs2260882 (C/G), rs532996 (A/G), rs11747040 (T/C), rs10034039 (T/G), rs116909369 (A/G), rs79134986 (A/G), rs117615688 (T/C), rs8032253 (T/C), rs12818653 (T/A), rs4587884 (A/C), rs77122853 (T/C), rs117615061 (T/C), rs74682905 (A/G), rs2257468 (T/C), rs2032582 (T/G), rs2235015 (T/G), rs2729794 (T/C), rs77549514 (A/G), rs74790420 (A/C), rs73129579 (T/C), rs12913346 (A/C), rs117560908 (T/C), rs72747091 (A/G), rs2935751 (A/G), rs4331446 (A/G), rs7523266 (T/C), rs7648662 (T/C), rs117034065 (A/G), rs4836256 (T/C), rs80238698 (T/C), rs3730173 (T/C), rs11687884 (T/C), rs72693005 (T/C), rs2589476 (T/C), rs9813396 (T/C), rs10482667 (A/G), rs72784444 (A/G), rs75074511 (T/C), rs7951003 (A/G), rs79584784 (A/G), rs2214102 (T/C), rs28811003 (A/G), rs6100261 (A/T), rs77152456 (A/G), rs66624622 (T/G), rs140302965 (A/G), rs11653269 (T/C), rs74405057 (A/G), rs7121 (A/G), rs16977818 (A/C), rs12490095 (T/C), rs118003903 (A/G), rs62377761 (T/C), P1_M_061510_6_34_M (−/CACTTACCTTCTTTGTGCCACAGTTTCCCTATCTAAAACACAAGGTTATCAGT TATC AACATCTCTTGGGATTGTGAGGACTAAAGTAATGCACATAAAG), rs375115639 (−/AAATTACCCTGTTAGGTTTCAATGAAACACCTTTTCTCTTGTAACAAACATCT C CTCCAAGCTAGAATTTCAAAACAG), rs1002204 (A/C), rs10062367 (A/G), rs10482642 (A/G), rs10482658 (A/G), rs1053989 (A/C), rs10851628 (T/C), rs10947562 (T/C), rs11069612 (A/G), rs11071351 (T/C), rs11091175 (A/G), rs11638450 (T/C), rs11715827 (T/G), rs11745958 (T/C), rs11834041 (A/G), rs1202180 (T/C), rs12054781 (A/G), rs12539395 (A/G), rs12720066 (T/G), rs1279754 (A/C), rs12872047 (T/C), rs12876742 (A/C), rs12917505 (A/G), rs13066950 (T/G), rs13229143 (C/G), rs1383707 (T/C), rs1441824 (T/C), rs1652311 (A/G), rs17064 (T/A), rs17100236 (A/G), rs17149699 (A/G), rs1724386 (A/G), rs17250255 (A/G), rs17327624 (T/G), rs17616338 (A/G), rs17687796 (A/G), rs17740874 (T/C), rs17763104 (T/C), rs1880748 (T/C), rs1882478 (A/G), rs1944887 (T/C), rs2028629 (A/G), rs2143404 (A/G), rs2173530 (T/C), rs220806 (T/C), rs2235047 (A/C), rs2242071 (A/G), rs2257474 (T/C), rs2295583 (A/T), rs234629 (T/C), rs234630 (A/G), rs2436401 (A/G), rs258750 (T/C), rs2589487 (T/C), rs28364018 (T/G), rs28381774 (T/C), rs28381784 (A/G), rs2963155 (A/G), rs3133622 (T/G), rs32897 (T/C), rs33388 (A/T), rs3730168 (T/C), rs3735833 (T/G), rs3777747 (A/G), rs3786066 (T/C), rs3798346 (T/C), rs3822736 (A/G), rs389035 (T/C), rs3924144 (A/G), rs4148737 (T/C), rs4148749 (G/C), rs417968 (T/C), rs4458144 (T/C), rs4515335 (T/C), rs4728699 (A/G), rs4758040 (A/G), rs4812040 (A/G), rs4912650 (T/G), rs4957891 (T/C), rs5906392 (A/G), rs6026561 (T/C), rs6026565 (T/A), rs6026567 (A/G), rs6026593 (A/G), rs6092704 (T/G), rs6100260 (A/G), rs6128461 (T/C), rs6415328 (T/C), rs6610868 (T/C), rs6686061 (A/C), rs6730350 (T/G), rs6746197 (T/C), rs6963426 (T/C), rs7121326 (T/C), rs7721799 (A/G), rs7787082 (T/C), rs7799592 (A/C), rs796245 (T/C), rs809482 (A/C), rs8125112 (T/C), rs919196 (A/G), rs920750 (T/C), rs9332385 (A/G), rs930473 (T/G), rs9324921 (A/C), rs9348979 (A/G), rs9571939 (A/C), and rs9892359 (T/C).

28. The method of claim 27, wherein the method further comprises predicting the treatment response from the presence or absence of the polymorphism genotypes of (a), (b), or (c).

29. The method of claim 27, wherein detecting a polymorphism genotype associated with a treatment response comprises:
(a) determining whether the subject will respond, or has an increased likelihood of responding to the treatment with a CRHR1 antagonist; and/or
(b) determining whether the subject will not respond, or has a decreased likelihood of responding to the treatment with a CRHR1 antagonist.

30. The method of claim 29, wherein the determining step comprises one or more statistical analysis method selected from the group consisting of artificial neural network learning, decision tree learning, decision tree forest learning, linear discriminant analysis, non-linear discriminant analysis, genetic expression programming, relevance vector machines, linear models, generalized linear models, generalized estimating equations, generalized linear mixed models, the elastic net, the lasso support vector machine learning, Bayesian network learning, probabilistic neural network learning, clustering, and regression analysis, optionally wherein the statistical analysis method is computer-implemented.

31. The method of claim 27, wherein the one or more polymorphism genotypes comprise:
(a) at least two;
(b) at least four;
(c) at least eight;
(d) at least sixteen;
(e) at least thirty-two; or
(f) all
of the polymorphism genotypes as defined in claim 27.

32. The method of claim 27, wherein the one or more polymorphism genotypes comprise:
(a) at least two polymorphism genotypes selected from the combinations of polymorphism genotypes disclosed in Table 5;
(b) at least four polymorphism genotypes selected from the combinations of polymorphism genotypes disclosed in Table 6;
(c) at least eight polymorphism genotypes selected from the combinations of polymorphism genotypes disclosed in Table 7;
(d) all of the polymorphism genotypes disclosed in Table 2.

33. The method of claim 27, wherein the treatment response to treatment with the CRHR1 antagonist is a clinical response.

34. The method of claim 27, wherein the subject has depressive symptoms, anxiety symptoms or both depressive symptoms and anxiety symptoms or a sleep disorder; and/or
wherein the treatment is a treatment of depressive symptoms, anxiety symptoms or both depressive symptoms and anxiety symptoms or a sleep disorder.

35. The method of claim 34, wherein the treatment response to treatment with the CRHR1 antagonist is a clinical response, and wherein the clinical response is a prevention, alteration, alleviation or complete remission of depressive symptoms and/or anxiety symptoms or a sleep disorder.

36. The method of claim 35, wherein the clinical response is a prevention, alteration, alleviation or complete remission of depressive symptoms and/or anxiety symptoms as determined using a scale selected from the group consisting of HAM-D, BDI, MADRS, GDS, ZSRDS, HAM-A and STAI.

37. The method of claim 27, wherein the biological sample is a buccal or a blood sample.

38. The method of claim 27, wherein detecting comprises the use of one or more polynucleotides capable of specifically hybridizing to at least one nucleic acid comprising the one or more polymorphism genotypes.

39. The method of claim 27, wherein after the subject has been administered a CRHR1 antagonist, further comprising comparing the prediction that the subject will respond to a treatment with a CRHR1 antagonist with the treatment response of the subject to administration of the CRHR1 antagonist.

40. The method of claim 27, wherein the prediction that the subject will respond to a treatment with a CRHR1 antagonist has a sensitivity of higher than 50% and a specificity of higher than 50%.

41. The method of claim 27, wherein the CRHR1 antagonist is selected from the group consisting of GW876008 (Emicerfont), GSK-561679 (NBI-77860, Verucerfont), GSK586529, BMS-562,086 (Pexacerfont), NBI-30775 (R-121919), NBI-34101, CP-316,311, CP-376,395, PF-00572778, NVP-AAG561, Ono-2333MS, E2508, E2009, R317573 (JNJ19567470, CRA5626, TAI-041), R278995 (CRA0450), CRA-1000, CRA-1001, CP154,526, Antalarmin, DMP-695, DMP-696, DMP-904, SC-241, BMS-561388, NBI30545, PD-171729, NBI34041, NBI35965, SN003, NBI-27914, trans-2-chloro-N-(4-((5-fluoro-4-methyl-pyridin-2-ylamino)-methyl)-cyclohexyl)-5-(trifluoromethyl)-benzamide, SSR-125543, or a pharmaceutically acceptable salt thereof.
